(19) United States
(12) Patent Application Publication
(10) Pub. No.: US 2022/0144961 A1
(43) Pub. Date: May 12, 2022

(54) CD27-BINDING ANTIBODIES AND USES THEREOF

(71) Applicant: XYENCE THERAPEUTICS, INC., Menlo Park, CA (US)

(72) Inventors: Ryan Lewis KELLY, Menlo Park, CA (US); Frances LIU, Menlo Park, CA (US); Vivian HSIEH, Menlo Park, CA (US); Cheryl CHANG, Menlo Park, CA (US); Claudine TAHMIN, Menlo Park, CA (US)

(21) Appl. No.: 17/438,409

(22) PCT Filed: Mar. 11, 2020
(86) PCT No.: PCT/US2020/022173

§ 371 Date: Sep. 10, 2021

Related U.S. Application Data
(60) Provisional application No. 62/816,800, filed on Mar. 11, 2019.
(60) Provisional application No. 62/873,689, filed on Jul. 12, 2019.

(51) Int. Cl. C07K 16/28; A61P 35/00

(52) U.S. Cl. CPC C07K 16/2878; A61K 2039/507; A61P 35/00

(57) ABSTRACT

The invention provides novel anti-CD27 antibodies, pharmaceutical compositions comprising such antibodies, and therapeutic methods of using such antibodies and pharmaceutical compositions for the treatment of diseases such as cancer.

### CROSS-REFERENCE TO RELATED APPLICATIONS

The present application claims the benefit of U.S. Provisional Application No. 62/816,800, filed Mar. 11, 2019, and claims the benefit of U.S. Provisional Application No. 62/873,689, filed Jul. 12, 2019, both of which are expressly incorporated by reference in their entireties for all purposes.

### TECHNICAL FIELD

The present disclosure relates to novel anti-CD27 antibodies and pharmaceutical compositions comprising such antibodies for use in modulating immune response, as well as treatment of diseases such as cancer.

### BACKGROUND OF THE INVENTION

The treatment of diseases by modulating an immune response is referred to as immunotherapy. Immunotherapy has demonstrated increasing effectiveness in treating cancer. Much immunotherapeutic success in cancer treatment is based on the use of immune-modulating antibodies that target immune checkpoints.

CD27 (TNFRSF7) is a co-stimulatory immune checkpoint molecule and a member of the tumor necrosis factor receptor superfamily. CD27 interacts with ligand CD70 to regulate immune cell activation through TRAF2 and TRAF5 mediated activation of the NFκB pathway. CD27 is expressed on many different lymphoid cell types, including T cells, B cells, and NK cells. CD27 is also overexpressed in cancer, including ovarian cancer, colorectal cancer renal cell carcinoma, glioblastoma, and B and T cell lymphomas.

The present invention provides novel monotherapies and combination therapies for use in treatment of diseases.

### SUMMARY OF THE INVENTION

In one aspect, the present invention relates to novel anti-CD27 antibodies. In some embodiments, the anti-CD27 antibodies include a heavy chain variable region comprising an amino acid sequence of SEQ ID NO:1 and a light chain variable region comprising an amino acid sequence of SEQ ID NO:5. In some embodiments, the anti-CD27 antibodies include a heavy chain variable region comprising an amino acid sequence of SEQ ID NO:9 and a light chain variable region comprising an amino acid sequence of SEQ ID NO:13. In some embodiments, the anti-CD27 antibodies include a heavy chain variable region comprising an amino acid sequence of SEQ ID NO:17 and a light chain variable region comprising an amino acid sequence of SEQ ID NO:21. In some embodiments, the anti-CD27 antibodies include a heavy chain variable region comprising an amino acid sequence of SEQ ID NO:25 and a light chain variable region comprising an amino acid sequence of SEQ ID NO:29. In some embodiments, the anti-CD27 antibodies include a heavy chain variable region comprising an amino acid sequence of SEQ ID NO:105 and a light chain variable region comprising an amino acid sequence of SEQ ID NO:109. In some embodiments, the anti-CD27 antibodies include a heavy chain variable region comprising an amino acid sequence of SEQ ID NO:113 and a light chain variable region comprising an amino acid sequence of SEQ ID NO:117. In some embodiments, the anti-CD27 antibodies include a heavy chain variable region comprising an amino acid sequence of SEQ ID NO:225 and a light chain variable region comprising an amino acid sequence of SEQ ID NO:229. In some embodiments, the anti-CD27 antibodies include a heavy chain variable region comprising an amino acid sequence of SEQ ID NO:273 and a light chain variable region comprising an amino acid sequence of SEQ ID NO:277.

In some embodiments, the anti-CD27 antibodies include a vhCDR1 comprising SEQ ID NO:2, a vhCDR2 comprising SEQ ID NO:3, a vhCDR3 comprising SEQ ID NO:4, a vlCDR1 comprising SEQ ID NO:6, a vlCDR2 comprising SEQ ID NO:7, and a vlCDR3 comprising SEQ ID NO:8. In some embodiments, the anti-CD27 antibodies include a vhCDR1 comprising SEQ ID NO:10, a vhCDR2 comprising SEQ ID NO:11, a vhCDR3 comprising SEQ ID NO:12, a vlCDR1 comprising SEQ ID NO:14, a vlCDR2 comprising SEQ ID NO:15, and a vlCDR3 comprising SEQ ID NO:16. In some embodiments, the anti-CD27 antibodies include a vhCDR1 comprising SEQ ID NO:18, a vhCDR2 comprising SEQ ID NO:19, a vhCDR3 comprising SEQ ID NO:20, a vlCDR1 comprising SEQ ID NO:22, a vlCDR2 comprising SEQ ID NO:23, and a vlCDR3 comprising SEQ ID NO:24. In some embodiments, the anti-CD27 antibodies include a vhCDR1 comprising SEQ ID NO:26, a vhCDR2 comprising SEQ ID NO:27, a vhCDR3 comprising SEQ ID NO:28, a vlCDR1 comprising SEQ ID NO:30, a vlCDR2 comprising SEQ ID NO:31, and a vlCDR3 comprising SEQ ID NO:32. In some embodiments, the anti-CD27 antibodies include a vhCDR1 comprising SEQ ID NO:106, a vhCDR2 comprising SEQ ID NO:107, a vhCDR3 comprising SEQ ID NO:108, a vlCDR1 comprising SEQ ID NO:110, a vlCDR2 comprising SEQ ID NO:111, and a vlCDR3 comprising SEQ ID NO:112. In some embodiments, the anti-CD27 antibodies include a vhCDR1 comprising SEQ ID NO:114, a vhCDR2 comprising SEQ ID NO:115, a vhCDR3 comprising SEQ ID NO:116, a vlCDR1 comprising SEQ ID NO:118, a vlCDR2 comprising SEQ ID NO:119, and a vlCDR3 comprising SEQ ID NO:120. In some embodiments, the anti-CD27 antibodies include a vhCDR1 comprising SEQ ID NO:226, a vhCDR2 comprising SEQ ID NO:227, a vhCDR3 comprising SEQ ID NO:228, a vlCDR1 comprising SEQ ID NO:230, a vlCDR2 comprising SEQ ID NO:231, and a vlCDR3 comprising SEQ ID NO:232.

In some embodiments, the anti-CD27 antibodies comprise a vhCDR1, a vhCDR2, a vhCDR3, a vlCDR1, a vlCDR2, and a vlCDR3 as described in FIG. 1 and/or FIG. 19.

In some embodiments, the anti-CD27 antibodies comprise a vhCDR1, a vhCDR2, a vhCDR3, a vlCDR1, a vlCDR2, and a vlCDR3 from and antibody selected from the group consisting of TP01, TP02, TP03, TP04, TP05, TP06, TP07, TP08, TP09, TP10, TP11, TP12, TP13, TP14, TP15, TP16, TP17, TP18, TP19, TP20, TP21, TP22, TP23, TP24, TP25, TP26, TP27, TP28, TP29, TP30, TP31, and TP32.

In some embodiments, the anti-CD27 antibodies described herein include a constant region with an amino acid sequence at least 90% identical to a human IgG. In some embodiments, the IgG is selected from an IgG1, IgG2, IgG3 or IgG4. In some embodiments, the IgG is an IgG4. In other embodiments, the IgG is an IgG1.

In another aspect, the present invention relates to a nucleic acid composition comprising a first nucleic acid encoding any one of the heavy chain variable regions described herein and a second nucleic acid encoding any one of the light chain variable regions described herein.

Another aspect of the present invention relates to an expression vector composition that includes any one of the nucleic acid compositions described herein. In some embodiments, the first nucleic acid is contained in a first expression vector and the second nucleic acid is contained in a second expression vector. In some other embodiments, the first nucleic acid and the second nucleic acid are contained in a single expression vector.

Another aspect of the present invention relates to a host cell that includes any one of the expression vectors described herein. Also presented is a method of making anti-CD27 antibodies, and the method includes culturing the host cell under conditions wherein the antibodies expressed, and recovering the antibodies.

In another aspect, the present invention relates to a composition that includes any one of the anti-CD27 antibodies described herein, and a pharmaceutical acceptable carrier or diluent.

Also described is a method of modulating an immune response in a subject, and the method includes administering to the subject an effective amount of any one of the anti-CD27 antibodies described herein, or any one of the compositions described herein. In some embodiments, the method induces or enhances an immune response in the subject and the method includes administering to the subject an effective amount of an anti-CD27 antibody that serves as a CD27 agonist, or a pharmaceutical composition thereof. In some embodiments, the method inhibits an immune response in the subject and the method includes administering to the subject an effective amount of an anti-CD27 antibody that serves as a CD27 antagonist, or a pharmaceutical composition thereof.

In some embodiments, the method modulates an immune response in the subject, and the method includes administering to the subject an effective amount of an anti-CD27 antibody, wherein the anti-CD27 antibody includes a heavy chain variable region comprising an amino acid sequence of SEQ ID NO:1 or SEQ ID NO: 105 and a light chain variable region comprising an amino acid sequence of SEQ ID NO:5 or SEQ ID NO: 109; and/or a vhCDR1 comprising SEQ ID NO:2, a vhCDR2 comprising SEQ ID NO:3, a vhCDR3 comprising SEQ ID NO:4, a vlCDR1 comprising SEQ ID NO:6, a vlCDR2 comprising SEQ ID NO:7, and a vlCDR3 comprising SEQ ID NO:8; and/or a vhCDR1 comprising SEQ ID NO: 106, a vhCDR2 comprising SEQ ID NO:107, a vhCDR3 comprising SEQ ID NO:108, a vlCDR1 comprising SEQ ID NO:110, a vlCDR2 comprising SEQ ID NO:111, and a vlCDR3 comprising SEQ ID NO:112.

In some embodiments, the method modulates an immune response in the subject, and the method includes administering to the subject an effective amount of an anti-CD27 antibody, wherein the anti-CD27 antibody includes a heavy chain variable region comprising an amino acid sequence of SEQ ID NO:9 or SEQ ID NO: 113 and a light chain variable region comprising an amino acid sequence of SEQ ID NO:13 or SEQ ID NO: 117; and/or a vhCDR1 comprising SEQ ID NO:10, a vhCDR2 comprising SEQ ID NO:11, a vhCDR3 comprising SEQ ID NO:12, a vlCDR1 comprising SEQ ID NO:14, a vlCDR2 comprising SEQ ID NO:15, and a vlCDR3 comprising SEQ ID NO:16; and/or a vhCDR1 comprising SEQ ID NO:114, a vhCDR2 comprising SEQ ID NO:115, a vhCDR3 comprising SEQ ID NO:116, a vlCDR1 comprising SEQ ID NO:118, a vlCDR2 comprising SEQ ID NO:119, and a vlCDR3 comprising SEQ ID NO:120.

In some embodiments, the method modulates an immune response in the subject, and the method includes administering to the subject an effective amount of an anti-CD27 antibody, wherein the anti-CD27 antibody includes a heavy chain variable region comprising an amino acid sequence of SEQ ID NO:17 or SEQ ID NO:225 and a light chain variable region comprising an amino acid sequence of SEQ ID NO:21 or SEQ ID NO:229; and/or a vhCDR1 comprising SEQ ID NO:18, a vhCDR2 comprising SEQ ID NO:19, a vhCDR3 comprising SEQ ID NO:20, a vlCDR1 comprising SEQ ID NO:22, a vlCDR2 comprising SEQ ID NO:23, and a vlCDR3 comprising SEQ ID NO:24 and/or a vhCDR1 comprising SEQ ID NO:226, a vhCDR2 comprising SEQ ID NO:227, a vhCDR3 comprising SEQ ID NO:228, a vlCDR1 comprising SEQ ID NO:230, a vlCDR2 comprising SEQ ID NO:231, and a vlCDR3 comprising SEQ ID NO:232.

In some embodiments, the method modulates an immune response in the subject, and the method includes administering to the subject an effective amount of an anti-CD27 antibody, wherein the anti-CD27 antibody includes a heavy chain variable region comprising an amino acid sequence of SEQ ID NO:25 and a light chain variable region comprising an amino acid sequence of SEQ ID NO:29; and/or a vhCDR1 comprising SEQ ID NO:26, a vhCDR2 comprising SEQ ID NO:27, a vhCDR3 comprising SEQ ID NO:28, a vlCDR1 comprising SEQ ID NO:30, a vlCDR2 comprising SEQ ID NO:31, and a vlCDR3 comprising SEQ ID NO:32.

In another aspect, the present invention relates to a method of treating cancer in a subject, and the method includes administering to the subject an effective amount of an anti-CD27 antibody described herein, or a composition thereof. In some embodiments, the cancer to be treated upregulates CD27 compared to the corresponding non-cancerous tissue. In some embodiments, the cancer to be treated expresses CD27. The cancer to be treated includes but is not limited to B cell lymphoma, T cell lymphoma, B and T cell lymphoma, colorectal cancer, breast cancer, rectal cancer, lung (including non-small cell lung cancer), non-Hodgkin's lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, multiple myeloma, CD27 Expressing B-cell Malignancies, Hodgkin's Lymphoma, Chronic Lymphocytic Leukemia, Mantle Cell Lymphoma, Marginal Zone B Cell Lymphoma, T-cell Malignancy, Solid Tumors, Metastatic Melanoma, Renal (Clear) Cell Carcinoma, Hormone-refractory Prostate Adenocarcinoma, Ovarian Cancer, Colorectal Adenocarcinoma, Non-small Cell Lung Cancer, Burkett's Lymphoma, Primary Lymphoma of the Central Nervous System, Advanced Breast Carcinoma, Advanced Ovarian Carcinoma, Prostate Cancer, Squamous Cell Carcinoma of the Head and Neck (SCCHN), Ovarian Carcinoma, Colorectal Cancer (CRC), Renal Cell Carcinoma (RCC), Glioblastoma (GBM), Carcinoma, Renal Cell, Kidney Diseases, Kidney Neoplasms, Urogenital Neoplasms, Urologic Diseases, Urologic Neoplasms, Neoplasms, Neoplasms by Histologic Type, Clear-cell Metastatic Renal Cell Carcinoma, Unresectable Stage III or Stage IV Melanoma, Carcinoma, Renal Cell, Kidney Diseases, Kidney Neoplasms, Urogenital Neoplasms, Urologic Diseases, Urologic Neoplasms, Neoplasms by Histologic Type, Neoplasms Clear-cell Metastatic Renal Cell Carcinoma, Melanoma, Triple Negative Breast Cancer, Bladder Cancer, Head and Neck Cancer, Non-small Cell Lung Cancer, Aggressive B-Cell Lymphomas, and B-Cell Lymphomas.

In some embodiments, an anti-CD27 antibody is used in combination with one or more additional therapeutic agents to treat cancer. In some embodiments, such anti-cancer therapeutic agents are other immune checkpoint inhibitors, such as a PD-1 inhibitor, PD-L1 inhibitor, CTLA-inhibitor, TIM-3 inhibitor, and a LAG-3 inhibitor. In some embodiments, such anti-cancer therapeutic agents are tumor targeting antibodies, such as rituximab, trastuzumab, and sunitinib. In some embodiments, the anti-cancer therapeutic agent is an integrin-binding polypeptide-Fc fusion. In some embodiments, the integrin-binding polypeptide-Fc fusion is NOD-201.

In another aspect, the present invention relates to a method of inducing or enhancing an immune response against and antigen in a subject comprising administering to the subject a monoclonal antibody which binds to human CD27, in an amount effective to induce or enhance an immune response against an antigen, wherein the antibody comprises a heavy chain variable region CDR1 comprising SEQ ID NO: 2; a heavy chain variable region CDR2 comprising SEQ ID NO: 3; a heavy chain variable region CDR3 comprising SEQ ID NO: 4; a light chain variable region CDR1 comprising SEQ ID NO: 6; a light chain variable region CDR2 comprising SEQ ID NO: 7; and a light chain variable region CDR3 comprising SEQ ID NO: 8; or wherein the antibody comprises a heavy chain variable region CDR1 comprising SEQ ID NO: 106; a heavy chain variable region CDR2 comprising SEQ ID NO: 107; a heavy chain variable region CDR3 comprising SEQ ID NO: 108; a light chain variable region CDR1 comprising SEQ ID NO: 110; a light chain variable region CDR2 comprising SEQ ID NO: 111; and a light chain variable region CDR3 comprising SEQ ID NO: 112. In some embodiments, the antibody further comprises a heavy chain variable region comprising SEQ ID NO: 1 or SEQ ID NO:105. In some embodiments, the antibody further comprises a light chain variable region comprising SEQ ID NO: 5 or SEQ ID NO:109. In some embodiments, the antibody comprises a heavy chain variable region comprising SEQ ID NO:1 and a light chain variable region comprising SEQ ID NO:5 or a heavy chain variable region comprising SEQ ID NO:105 and a light chain variable region comprising SEQ ID NO:109. In some embodiments, the immune response is an antigen-specific T cell response.

In another aspect, the present invention relates to a method for inducing or enhancing an immune response against an antigen in a subject comprising administering to the subject a monoclonal antibody which binds to human CD27, in an amount effective to induce or enhance an immune response against an antigen, wherein the antibody comprises a heavy chain variable region CDR1 comprising SEQ ID NO: 10; a heavy chain variable region CDR2 comprising SEQ ID NO: 11; a heavy chain variable region CDR3 comprising SEQ ID NO: 12; a light chain variable region CDR1 comprising SEQ ID NO: 14; a light chain variable region CDR2 comprising SEQ ID NO: 15; and a light chain variable region CDR3 comprising SEQ ID NO: 16; or wherein the antibody comprises a heavy chain variable region CDR1 comprising SEQ ID NO: 114; a heavy chain variable region CDR2 comprising SEQ ID NO: 115; a heavy chain variable region CDR3 comprising SEQ ID NO: 116; a light chain variable region CDR1 comprising SEQ ID NO: 118; a light chain variable region CDR2 comprising SEQ ID NO: 119; and a light chain variable region CDR3 comprising SEQ ID NO: 120. In some embodiments, the antibody further comprises a heavy chain variable region comprising SEQ ID NO: 9 or SEQ ID NO:113. In some embodiments, the antibody further comprises a light chain variable region comprising SEQ ID NO: 13 or SEQ ID NO:117. In some embodiments, the antibody comprises a heavy chain variable region comprising SEQ ID NO:9 and a light chain variable region comprising SEQ ID NO:13 or a heavy chain variable region comprising SEQ ID NO:113 and a light chain variable region comprising SEQ ID NO:117. In some embodiments, the immune response is an antigen-specific T cell response.

In another aspect, the present invention relates to a method for inducing or enhancing an immune response against an antigen in a subject comprising administering to the subject a monoclonal antibody which binds to human CD27, in an amount effective to induce or enhance an immune response against an antigen, wherein the antibody comprises a heavy chain variable region CDR1 comprising SEQ ID NO: 18; a heavy chain variable region CDR2 comprising SEQ ID NO: 19; a heavy chain variable region CDR3 comprising SEQ ID NO: 20; a light chain variable region CDR1 comprising SEQ ID NO: 22; a light chain variable region CDR2 comprising SEQ ID NO: 23; and a light chain variable region CDR3 comprising SEQ ID NO: 24; or wherein the antibody comprises a heavy chain variable region CDR1 comprising SEQ ID NO: 226; a heavy chain variable region CDR2 comprising SEQ ID NO: 227; a heavy chain variable region CDR3 comprising SEQ ID NO: 228; a light chain variable region CDR1 comprising SEQ ID NO: 230; a light chain variable region CDR2 comprising SEQ ID NO: 231; and a light chain variable region CDR3 comprising SEQ ID NO: 232. In some embodiments, the antibody further comprises a heavy chain variable region comprising SEQ ID NO:17 or SEQ ID NO:225. In some embodiments, the antibody further comprises a light chain variable region comprising SEQ ID NO: 21 or SEQ ID NO:229. In some embodiments, the antibody comprises a heavy chain variable region comprising SEQ ID NO:17 and a light chain variable region comprising SEQ ID NO:21 or a heavy chain variable region comprising SEQ ID NO:225 and a light chain variable region comprising SEQ ID NO:229. In some embodiments, the immune response is an antigen-specific T cell response.

In another aspect, the present invention relates to a method for inducing or enhancing an immune response against an antigen in a subject comprising administering to the subject a monoclonal antibody which binds to human CD27, in an amount effective to induce or enhance an immune response against an antigen, wherein the antibody comprises a heavy chain variable region CDR1 comprising SEQ ID NO: 26; a heavy chain variable region CDR2 comprising SEQ ID NO: 27; a heavy chain variable region CDR3 comprising SEQ ID NO: 28; a light chain variable region CDR1 comprising SEQ ID NO: 30; a light chain variable region CDR2 comprising SEQ ID NO: 31; and a light chain variable region CDR3 comprising SEQ ID NO: 32; or wherein the antibody comprises a heavy chain variable region CDR1 comprising SEQ ID NO: 274; a heavy chain variable region CDR2 comprising SEQ ID NO: 275; a heavy chain variable region CDR3 comprising SEQ ID NO: 276; a light chain variable region CDR1 comprising SEQ ID NO: 278; a light chain variable region CDR2 comprising SEQ ID NO: 279; and a light chain variable region CDR3 comprising SEQ ID NO: 280. In some embodiments, the antibody further comprises a heavy chain variable region comprising SEQ ID NO: 25 or SEQ ID NO:273. In some embodiments, the antibody further comprises a light chain variable region comprising SEQ ID NO: 29 or SEQ ID NO:277. In some embodiments, the antibody comprises a heavy chain variable region comprising SEQ ID NO:25 and a light chain variable region comprising SEQ ID NO:29 or a heavy chain variable region comprising SEQ ID NO:273 and a light chain variable region comprising SEQ ID NO:277. In some embodiments, the immune response is an antigen-specific T cell response.

In another aspect, the present invention relates to a method of inhibiting growth of CD27 expressing cells comprising contacting the cells with a monoclonal antibody which binds to human CD27, in an amount effective to inhibit growth of CD27 expressing cells, wherein the antibody comprises a heavy chain variable region CDR1 comprising SEQ ID NO: 2; a heavy chain variable region CDR2 comprising SEQ ID NO: 3; a heavy chain variable region CDR3 comprising SEQ ID NO: 4; a light chain variable region CDR1 comprising SEQ ID NO: 6; a light chain variable region CDR2 comprising SEQ ID NO: 7; and a light chain variable region CDR3 comprising SEQ ID NO: 8; or wherein the antibody comprises a heavy chain variable region CDR1 comprising SEQ ID NO: 106; a heavy chain variable region CDR2 comprising SEQ ID NO: 107; a heavy chain variable region CDR3 comprising SEQ ID NO: 108; a light chain variable region CDR1 comprising SEQ ID NO: 110; a light chain variable region CDR2 comprising SEQ ID NO: 111; and a light chain variable region CDR3 comprising SEQ ID NO: 112. In some embodiments, the antibody further comprises a heavy chain variable region comprising SEQ ID NO: 1 or SEQ ID NO:105. In some embodiments, the antibody further comprises a light chain variable region comprising SEQ ID NO: 5 or SEQ ID NO:109. In some embodiments, the antibody comprises a heavy chain variable region comprising SEQ ID NO:1 and a light chain variable region comprising SEQ ID NO:5 or a heavy chain variable region comprising SEQ ID NO:105 and a light chain variable region comprising SEQ ID NO:109. In some embodiments, the immune response is an antigen-specific T cell response.

In another aspect, the present invention relates to a method of inhibiting growth of CD27 expressing cells comprising contacting the cells with a monoclonal antibody which binds to human CD27, in an amount effective to inhibit growth of CD27 expressing cells, wherein the antibody comprises a heavy chain variable region CDR1 comprising SEQ ID NO: 10; a heavy chain variable region CDR2 comprising SEQ ID NO: 11; a heavy chain variable region CDR3 comprising SEQ ID NO: 12; a light chain variable region CDR1 comprising SEQ ID NO: 14; a light chain variable region CDR2 comprising SEQ ID NO: 15; and a light chain variable region CDR3 comprising SEQ ID NO: 16; or wherein the antibody comprises a heavy chain variable region CDR1 comprising SEQ ID NO: 114; a heavy chain variable region CDR2 comprising SEQ ID NO: 115; a heavy chain variable region CDR3 comprising SEQ ID NO: 116; a light chain variable region CDR1 comprising SEQ ID NO: 118; a light chain variable region CDR2 comprising SEQ ID NO: 119; and a light chain variable region CDR3 comprising SEQ ID NO: 120. In some embodiments, the antibody further comprises a heavy chain variable region comprising SEQ ID NO: 9 or SEQ ID NO:113. In some embodiments, the antibody further comprises a light chain variable region comprising SEQ ID NO: 13 or SEQ ID NO:117. In some embodiments, the antibody comprises a heavy chain variable region comprising SEQ ID NO:9 and a light chain variable region comprising SEQ ID NO:13 or a heavy chain variable region comprising SEQ ID NO:113 and a light chain variable region comprising SEQ ID NO:117. In some embodiments, the immune response is an antigen-specific T cell response.

In another aspect, the present invention relates to a method of inhibiting growth of CD27 expressing cells comprising contacting the cells with a monoclonal antibody which binds to human CD27, in an amount effective to inhibit growth of CD27 expressing cells, wherein the antibody comprises a heavy chain variable region CDR1 comprising SEQ ID NO: 18; a heavy chain variable region CDR2 comprising SEQ ID NO: 19; a heavy chain variable region CDR3 comprising SEQ ID NO: 20; a light chain variable region CDR1 comprising SEQ ID NO: 22; a light chain variable region CDR2 comprising SEQ ID NO: 23; and a light chain variable region CDR3 comprising SEQ ID NO: 24; or wherein the antibody comprises a heavy chain variable region CDR1 comprising SEQ ID NO: 226; a heavy chain variable region CDR2 comprising SEQ ID NO: 227; a heavy chain variable region CDR3 comprising SEQ ID NO: 228; a light chain variable region CDR1 comprising SEQ ID NO: 230; a light chain variable region CDR2 comprising SEQ ID NO: 231; and a light chain variable region CDR3 comprising SEQ ID NO: 232. In some embodiments, the antibody further comprises a heavy chain variable region comprising SEQ ID NO:17 or SEQ ID NO:225. In some embodiments, the antibody further comprises a light chain variable region comprising SEQ ID NO: 21 or SEQ ID NO:229. In some embodiments, the antibody comprises a heavy chain variable region comprising SEQ ID NO:17 and a light chain variable region comprising SEQ ID NO:21 or a heavy chain variable region comprising SEQ ID NO:225 and a light chain variable region comprising SEQ ID NO:229. In some embodiments, the immune response is an antigen-specific T cell response.

In another aspect, the present invention relates to a method of inhibiting growth of CD27 expressing cells comprising contacting the cells with a monoclonal antibody which binds to human CD27, in an amount effective to inhibit growth of CD27 expressing cells, wherein the antibody comprises a heavy chain variable region CDR1 comprising SEQ ID NO: 26; a heavy chain variable region CDR2 comprising SEQ ID NO: 27; a heavy chain variable region CDR3 comprising SEQ ID NO: 28; a light chain variable region CDR1 comprising SEQ ID NO: 30; a light chain variable region CDR2 comprising SEQ ID NO: 31; and a light chain variable region CDR3 comprising SEQ ID NO: 32; or wherein the antibody comprises a heavy chain variable region CDR1 comprising SEQ ID NO: 274; a heavy chain variable region CDR2 comprising SEQ ID NO: 275; a heavy chain variable region CDR3 comprising SEQ ID NO: 276; a light chain variable region CDR1 comprising SEQ ID NO: 278; a light chain variable region CDR2 comprising SEQ ID NO: 279; and a light chain variable region CDR3 comprising SEQ ID NO: 280. In some embodiments, the antibody further comprises a heavy chain variable region comprising SEQ ID NO: 25 or SEQ ID NO:273. In some embodiments, the antibody further comprises a light chain variable region comprising SEQ ID NO: 29 or SEQ ID NO:277. In some embodiments, the antibody comprises a heavy chain variable region comprising SEQ ID NO:25 and a light chain variable region comprising SEQ ID NO:29 or a heavy chain variable region comprising SEQ ID NO:273 and a light chain variable region comprising SEQ ID NO:277. In some embodiments, the immune response is an antigen-specific T cell response.

In another aspect, the present invention relates to a method of inhibiting the binding of CD70 to CD27 on cells in a subject having a disorder by administering to the subject a monoclonal antibody which binds to human CD27, wherein the antibody comprises a heavy chain variable region CDR1 comprising SEQ ID NO: 2; a heavy chain variable region CDR2 comprising SEQ ID NO: 3; a heavy chain variable region CDR3 comprising SEQ ID NO: 4; a light chain variable region CDR1 comprising SEQ ID NO: 6; a light chain variable region CDR2 comprising SEQ ID NO: 7; and a light chain variable region CDR3 comprising SEQ ID NO: 8; or wherein the antibody comprises a heavy chain variable region CDR1 comprising SEQ ID NO: 106; a heavy chain variable region CDR2 comprising SEQ ID NO: 107; a heavy chain variable region CDR3 comprising SEQ ID NO: 108; a light chain variable region CDR1 comprising SEQ ID NO: 110; a light chain variable region CDR2 comprising SEQ ID NO: 111; and a light chain variable region CDR3 comprising SEQ ID NO: 112. In some embodiments, the antibody further comprises a heavy chain variable region comprising SEQ ID NO: 1 or SEQ ID NO:105. In some embodiments, the antibody further comprises a light chain variable region comprising SEQ ID NO: 5 or SEQ ID NO:109. In some embodiments, the antibody comprises a heavy chain variable region comprising SEQ ID NO:1 and a light chain variable region comprising SEQ ID NO:5 or a heavy chain variable region comprising SEQ ID NO:105 and a light chain variable region comprising SEQ ID NO:109. In some embodiments, the immune response is an antigen-specific T cell response.

In another aspect, the present invention relates to a method of inhibiting the binding of CD70 to CD27 on cells in a subject having a disorder by administering to the subject a monoclonal antibody which binds to human CD27, wherein the antibody comprises a heavy chain variable region CDR1 comprising SEQ ID NO: 10; a heavy chain variable region CDR2 comprising SEQ ID NO: 11; a heavy chain variable region CDR3 comprising SEQ ID NO: 12; a light chain variable region CDR1 comprising SEQ ID NO: 14; a light chain variable region CDR2 comprising SEQ ID NO: 15; and a light chain variable region CDR3 comprising SEQ ID NO: 16; or wherein the antibody comprises a heavy chain variable region CDR1 comprising SEQ ID NO: 114; a heavy chain variable region CDR2 comprising SEQ ID NO: 115; a heavy chain variable region CDR3 comprising SEQ ID NO: 116; a light chain variable region CDR1 comprising SEQ ID NO: 118; a light chain variable region CDR2 comprising SEQ ID NO: 119; and a light chain variable region CDR3 comprising SEQ ID NO: 120. In some embodiments, the antibody further comprises a heavy chain variable region comprising SEQ ID NO: 9 or SEQ ID NO:113. In some embodiments, the antibody further comprises a light chain variable region comprising SEQ ID NO: 13 or SEQ ID NO:117. In some embodiments, the antibody comprises a heavy chain variable region comprising SEQ ID NO:9 and a light chain variable region comprising SEQ ID NO:13 or a heavy chain variable region comprising SEQ ID NO:113 and a light chain variable region comprising SEQ ID NO:117. In some embodiments, the immune response is an antigen-specific T cell response.

In another aspect, the present invention relates to a method of inhibiting the binding of CD70 to CD27 on cells in a subject having a disorder by administering to the subject a monoclonal antibody which binds to human CD27, wherein the antibody comprises a heavy chain variable region CDR1 comprising SEQ ID NO: 18; a heavy chain variable region CDR2 comprising SEQ ID NO: 19; a heavy chain variable region CDR3 comprising SEQ ID NO: 20; a light chain variable region CDR1 comprising SEQ ID NO: 22; a light chain variable region CDR2 comprising SEQ ID NO: 23; and a light chain variable region CDR3 comprising SEQ ID NO: 24; or wherein the antibody comprises a heavy chain variable region CDR1 comprising SEQ ID NO: 226; a heavy chain variable region CDR2 comprising SEQ ID NO: 227; a heavy chain variable region CDR3 comprising SEQ ID NO: 228; a light chain variable region CDR1 comprising SEQ ID NO: 230; a light chain variable region CDR2 comprising SEQ ID NO: 231; and a light chain variable region CDR3 comprising SEQ ID NO: 232. In some embodiments, the antibody further comprises a heavy chain variable region comprising SEQ ID NO:17 or SEQ ID NO:225. In some embodiments, the antibody further comprises a light chain variable region comprising SEQ ID NO: 21 or SEQ ID NO:229. In some embodiments, the antibody comprises a heavy chain variable region comprising SEQ ID NO:17 and a light chain variable region comprising SEQ ID NO:21 or a heavy chain variable region comprising SEQ ID NO:225 and a light chain variable region comprising SEQ ID NO:229. In some embodiments, the immune response is an antigen-specific T cell response.

In another aspect, the present invention relates to a method of inhibiting the binding of CD70 to CD27 on cells in a subject having a disorder by administering to the subject a monoclonal antibody which binds to human CD27, wherein the antibody comprises a heavy chain variable region CDR1 comprising SEQ ID NO: 26; a heavy chain variable region CDR2 comprising SEQ ID NO: 27; a heavy chain variable region CDR3 comprising SEQ ID NO: 28; a light chain variable region CDR1 comprising SEQ ID NO: 30; a light chain variable region CDR2 comprising SEQ ID NO: 31; and a light chain variable region CDR3 comprising SEQ ID NO: 32; or wherein the antibody comprises a heavy chain variable region CDR1 comprising SEQ ID NO: 274; a heavy chain variable region CDR2 comprising SEQ ID NO: 275; a heavy chain variable region CDR3 comprising SEQ ID NO: 276; a light chain variable region CDR1 comprising SEQ ID NO: 278; a light chain variable region CDR2 comprising SEQ ID NO: 279; and a light chain variable region CDR3 comprising SEQ ID NO: 280. In some embodiments, the antibody further comprises a heavy chain variable region comprising SEQ ID NO: 25 or SEQ ID NO:273. In some embodiments, the antibody further comprises a light chain variable region comprising SEQ ID NO: 29 or SEQ ID NO:277. In some embodiments, the antibody comprises a heavy chain variable region comprising SEQ ID NO:25 and a light chain variable region comprising SEQ ID NO:29 or a heavy chain variable region comprising SEQ ID NO:273 and a light chain variable region comprising SEQ ID NO:277. In some embodiments, the immune response is an antigen-specific T cell response.

In another aspect, the present invention relates to a method for inducing or enhancing an immune response against an antigen in a subject comprising administering to the subject a monoclonal antibody which binds to human CD27, in an amount effective to induce or enhance an immune response against an antigen, wherein the antibody binds to the same epitope as an antibody comprising heavy and light chain variable region sequences as set forth in SEQ ID NO: 1 and SEQ ID NO: 5, or sequences as set forth in SEQ ID NO: 105 and SEQ ID NO: 109, respectively.

In another aspect, the present invention relates to a method for inducing or enhancing an immune response against an antigen in a subject comprising administering to the subject a monoclonal antibody which binds to human CD27, in an amount effective to induce or enhance an immune response against an antigen, wherein the antibody binds to the same epitope as an antibody comprising heavy and light chain variable region sequences as set forth in SEQ ID NO: 9 and SEQ ID NO: 13, or sequences as set forth in SEQ ID NO: 113 and SEQ ID NO: 117, respectively.

In another aspect, the present invention relates to a method for inducing or enhancing an immune response against an antigen in a subject comprising administering to the subject a monoclonal antibody which binds to human CD27, in an amount effective to induce or enhance an immune response against an antigen, wherein the antibody binds to the same epitope as an antibody comprising heavy and light chain variable region sequences as set forth SEQ ID NO: 17 and SEQ ID NO: 21, or sequences as set forth in SEQ ID NO: 225 and SEQ ID NO: 229, respectively.

In another aspect, the present invention relates to a method for inducing or enhancing an immune response against an antigen in a subject comprising administering to the subject a monoclonal antibody which binds to human CD27, in an amount effective to induce or enhance an immune response against an antigen, wherein the antibody binds to the same epitope as an antibody comprising heavy and light chain variable region sequences as set forth in SEQ ID NO: 25 and SEQ ID NO: 29, or sequences as set forth in SEQ ID NO: 273 and SEQ ID NO: 277, respectively.

In another aspect, the present invention relates to a method for inducing or enhancing an immune response against an antigen in a subject comprising administering to the subject a monoclonal antibody which binds to human CD27, in an amount effective to induce or enhance an immune response against an antigen, wherein the antibody competes for binding to human CD27 with an antibody comprising heavy and light chain variable region sequences as set forth in SEQ ID NO: 1 and 5, or sequences as set forth in SEQ ID NO: 105 and SEQ ID NO: 109, respectively.

In another aspect, the present invention relates to a method for inducing or enhancing an immune response against an antigen in a subject comprising administering to the subject a monoclonal antibody which binds to human CD27, in an amount effective to induce or enhance an immune response against an antigen, wherein the antibody competes for binding to human CD27 with an antibody comprising heavy and light chain variable region sequences as set forth in SEQ ID NO: 9 and SEQ ID NO: 13, or sequences as set forth in SEQ ID NO: 113 and SEQ ID NO: 117, respectively.

In another aspect, the present invention relates to a method for inducing or enhancing an immune response against an antigen in a subject comprising administering to the subject a monoclonal antibody which binds to human CD27, in an amount effective to induce or enhance an immune response against an antigen, wherein the antibody competes for binding to human CD27 with an antibody comprising heavy and light chain variable region sequences as set forth in SEQ ID NO: 17 and 21, or sequences as set forth in SEQ ID NO: 225 and SEQ ID NO: 229, respectively.

In another aspect, the present invention relates to a method for inducing or enhancing an immune response against an antigen in a subject comprising administering to the subject a monoclonal antibody which binds to human CD27, in an amount effective to induce or enhance an immune response against an antigen, wherein the antibody competes for binding to human CD27 with an antibody comprising heavy and light chain variable region sequences as set forth in SEQ ID NO: 25 and 29, or sequences as set forth in SEQ ID NO: 273 and SEQ ID NO: 277, respectively.

In another aspect, the present invention relates to a method for inducing or enhancing an immune response against an antigen in a subject comprising administering to the subject a monoclonal antibody which binds to human CD27, in an amount effective to induce or enhance an immune response against an antigen, wherein the antibody comprises heavy and light chain variable region sequences having at least 95% identity to SEQ ID NO: 1 and SEQ ID NO: 5, or sequences as set forth in SEQ ID NO: 105 and SEQ ID NO: 109, respectively.

In another aspect, the present invention relates to a method for inducing or enhancing an immune response against an antigen in a subject comprising administering to the subject a monoclonal antibody which binds to human CD27, in an amount effective to induce or enhance an immune response against an antigen, wherein the antibody comprises heavy and light chain variable region sequences having at least 95% identity to SEQ ID NO: 9 and SEQ ID NO: 13, or sequences as set forth in SEQ ID NO: 113 and SEQ ID NO: 117, respectively.

In another aspect, the present invention relates to a method for inducing or enhancing an immune response against an antigen in a subject comprising administering to the subject a monoclonal antibody which binds to human CD27, in an amount effective to induce or enhance an immune response against an antigen, wherein the antibody comprises heavy and light chain variable region sequences having at least 95% identity to SEQ ID NO: 17 and SEQ ID NO: 21, or sequences as set forth in SEQ ID NO: 225 and SEQ ID NO: 229, respectively.

In another aspect, the present invention relates to a method for inducing or enhancing an immune response against an antigen in a subject comprising administering to the subject a monoclonal antibody which binds to human CD27, in an amount effective to induce or enhance an immune response against an antigen, wherein the antibody comprises heavy and light chain variable region sequences having at least 95% identity to SEQ ID NO: 25 and SEQ ID NO: 29, or sequences as set forth in SEQ ID NO: 273 and SEQ ID NO: 277, respectively.

In another aspect, the present invention relates to a method for inducing or enhancing an immune response in a subject comprising administering to the subject a monoclonal antibody which binds to human CD27, in an amount effective to induce or enhance an immune response, wherein the antibody comprises a heavy chain variable region CDR1 comprising SEQ ID NO: 2; a heavy chain variable region CDR2 comprising SEQ ID NO: 3; a heavy chain variable region CDR3 comprising SEQ ID NO: 4; a light chain variable region CDR1 comprising SEQ ID NO: 6; a light chain variable region CDR2 comprising SEQ ID NO: 7; and a light chain variable region CDR3 comprising SEQ ID NO: 8; or wherein the antibody comprises a heavy chain variable region CDR1 comprising SEQ ID NO: 106; a heavy chain variable region CDR2 comprising SEQ ID NO: 107; a heavy chain variable region CDR3 comprising SEQ ID NO: 108; a light chain variable region CDR1 comprising SEQ ID NO: 110; a light chain variable region CDR2 comprising SEQ ID NO: 111; and a light chain variable region CDR3 comprising SEQ ID NO: 112.

In another aspect, the present invention relates to a method for inducing or enhancing an immune response in a subject comprising administering to the subject a monoclonal antibody which binds to human CD27, in an amount effective to induce or enhance an immune response, wherein the antibody comprises a heavy chain variable region CDR1 comprising SEQ ID NO: 10; a heavy chain variable region CDR2 comprising SEQ ID NO: 11; a heavy chain variable region CDR3 comprising SEQ ID NO: 12; a light chain variable region CDR1 comprising SEQ ID NO: 14; a light chain variable region CDR2 comprising SEQ ID NO: 15; and a light chain variable region CDR3 comprising SEQ ID NO: 16; or wherein the antibody comprises a heavy chain variable region CDR1 comprising SEQ ID NO: 114; a heavy chain variable region CDR2 comprising SEQ ID NO: 115; a heavy chain variable region CDR3 comprising SEQ ID NO: 116; a light chain variable region CDR1 comprising SEQ ID NO: 118; a light chain variable region CDR2 comprising SEQ ID NO: 119; and a light chain variable region CDR3 comprising SEQ ID NO: 120.

In another aspect, the present invention relates to a method for inducing or enhancing an immune response in a subject comprising administering to the subject a monoclonal antibody which binds to human CD27, in an amount effective to induce or enhance an immune response, wherein the antibody comprises a heavy chain variable region CDR1 comprising SEQ ID NO: 18; a heavy chain variable region CDR2 comprising SEQ ID NO: 19; a heavy chain variable region CDR3 comprising SEQ ID NO: 20; a light chain variable region CDR1 comprising SEQ ID NO: 22; a light chain variable region CDR2 comprising SEQ ID NO: 23; and a light chain variable region CDR3 comprising SEQ ID NO: 24; or wherein the antibody comprises a heavy chain variable region CDR1 comprising SEQ ID NO: 226; a heavy chain variable region CDR2 comprising SEQ ID NO: 227; a heavy chain variable region CDR3 comprising SEQ ID NO: 228; a light chain variable region CDR1 comprising SEQ ID NO: 230; a light chain variable region CDR2 comprising SEQ ID NO: 231; and a light chain variable region CDR3 comprising SEQ ID NO: 232.

In another aspect, the present invention relates to a method for inducing or enhancing an immune response in a subject comprising administering to the subject a monoclonal antibody which binds to human CD27, in an amount effective to induce or enhance an immune response, wherein the antibody comprises a heavy chain variable region CDR1 comprising SEQ ID NO: 26; a heavy chain variable region CDR2 comprising SEQ ID NO: 27; a heavy chain variable region CDR3 comprising SEQ ID NO: 28; a light chain variable region CDR1 comprising SEQ ID NO: 30; a light chain variable region CDR2 comprising SEQ ID NO: 31; and a light chain variable region CDR3 comprising SEQ ID NO: 32; or wherein the antibody comprises a heavy chain variable region CDR1 comprising SEQ ID NO: 274; a heavy chain variable region CDR2 comprising SEQ ID NO: 275; a heavy chain variable region CDR3 comprising SEQ ID NO: 276; a light chain variable region CDR1 comprising SEQ ID NO: 278; a light chain variable region CDR2 comprising SEQ ID NO: 279; and a light chain variable region CDR3 comprising SEQ ID NO: 280.

In another aspect, the present invention relates to a method of treating a non-cancerous disease in a subject comprising administering to the subject an effective amount of any of the antibodies described herein.

In some embodiments, the non-cancer disease is selected from the group consisting of kidney disease or urologic disease. In some embodiments, the cells causing the non-cancerous disease are expressing CD-27.

In another aspect, the present invention relates to a method for inducing or enhancing an immune response against an antigen in a subject comprising administering to the subject a monoclonal antibody which binds to human CD27, in an amount effective to induce or enhance an immune response against an antigen, wherein the antibody comprises a vhCDR1, a vhCDR2, a vhCDR3, a vlCDR1, a vlCDR2, and a vlCDR3 as described in FIG. 1 and/or FIG. 19.

In another aspect, the present invention relates to a method of inhibiting growth of CD27 expressing cells comprising contacting the cells with a monoclonal antibody which binds to human CD27, in an amount effective to inhibit growth of CD27 expressing cells, wherein the antibody comprises a vhCDR1, a vhCDR2, a vhCDR3, a vlCDR1, a vlCDR2, and a vlCDR3 as described in FIG. 1 and/or FIG. 19.

In another aspect, the present invention relates to a method of inhibiting the binding of CD70 to CD27 on cells in a subject having a disorder by administering to the subject a monoclonal antibody which binds to human CD27, wherein the antibody comprises a vhCDR1, a vhCDR2, a vhCDR3, a vlCDR1, a vlCDR2, and a vlCDR3 as described in FIG. 1 and/or FIG. 19.

In another aspect, the present invention relates to a method for inducing or enhancing an immune response against an antigen in a subject comprising administering to the subject a monoclonal antibody which binds to human CD27, in an amount effective to induce or enhance an immune response against an antigen, wherein the antibody binds to the same epitope as an antibody comprising heavy and light chain variable region sequences as set forth in FIG. 1 and/or FIG. 19.

In another aspect, the present invention relates to a method for inducing or enhancing an immune response against an antigen in a subject comprising administering to the subject a monoclonal antibody which binds to human CD27, in an amount effective to induce or enhance an immune response against an antigen, wherein the antibody competes for binding to human CD27 with an antibody comprising heavy and light chain variable region sequences as set forth in FIG. 1 and/or FIG. 19.

In another aspect, the present invention relates to a method for inducing or enhancing an immune response against an antigen in a subject comprising administering to the subject a monoclonal antibody which binds to human CD27, in an amount effective to induce or enhance an immune response against an antigen, wherein the antibody comprises a vhCDR1, a vhCDR2, a vhCDR3, a vlCDR1, a vlCDR2, and a vlCDR3 as described in FIG. 1 and/or FIG. 19.

In another aspect, the present invention relates to a method for inducing or enhancing an immune response in a subject comprising administering to the subject a monoclonal antibody which binds to human CD27, in an amount effective to induce or enhance an immune response, wherein the antibody comprises a vhCDR1, a vhCDR2, a vhCDR3, a vlCDR1, a vlCDR2, and a vlCDR3 as described in FIG. 1 and/or FIG. 19.

In some embodiments, the anti-CD27 antibody comprises an anti-CD27 antibody selected from the group consisting of TP01, TP02, TP03, TP04, TP05, TP06, TP07, TP08, TP09, TP10, TP11, TP12, TP13, TP14, TP15, TP16, TP17, TP18, TP19, TP20, TP21, TP22, TP23, TP24, TP25, TP26, TP27, TP28, TP29, TP30, TP31, and TP32.

In some embodiments, the anti-CD27 antibody comprises a constant region with an amino acid sequence at least 90% identical to a human IgG. In some embodiments, the human IgG is selected from the group consisting of IgG1, IgG2, IgG3, and IgG4. In some embodiments, the IgG is an IgG1 or IgG4.

### BRIEF DESCRIPTION OF THE DRAWINGS

These and other features, aspects, and advantages of the present invention will become better understood with regard to the following description, and accompanying drawings.

FIG. 1A-FIG. 1AJ provides the variable heavy and light chains and corresponding vhCDR1, vhCDR2, vhCDR3, vlCDR1, vlCDR2, and vlCDR3 sequences for the TP01, TP02, TP03, TP04, TP05, TP06, TP07, TP08, TP09, TP10, TP11, TP12, TP13, TP14, TP15, TP16, TP17, TP18, TP19, TP20, TP21, TP22, TP23, TP24, TP25, TP26, TP27, TP28, TP29, TP30, TP31, and TP32 anti-CD27 antibodies.

FIG. 2A-FIG. 2B provide examples of IgG1, IgG2, IgG3, and IgG4 sequences.

FIG. 3 provides flow cytometry data demonstrating rapid isolation of high affinity anti-CD27 populations.

FIG. 4A-FIG. 4C provide human and muring CD27 binding data and Kd of anti-CD27 antibody clones.

FIG. 5A provides data showing early activation of cytotoxic T cells for each of the anti-CD27 antibody clones.

FIG. 5B provides data showing proliferation of cytotoxic T cells for each of the anti-CD27 antibody clones.

FIG. 6A-FIG. 6B provides data showing 4 of the anti-CD27 cross-reactive antibody clones demonstrate robust and dose-dependent agonist activity in activating T cells in vitro.

FIG. 7 provides EC50 data for anti-CD27 cross-reactive antibody clones.

FIG. 8 provides the antibody format for the anti-CD27 antibody clones.

FIG. 9 provides data showing anti-CD27 antibody clone binding to human mouse and cyno CD27 as well as polyspecificity reagent.

FIG. 10 provides Kaplan Meier Survival data for BALB/c mice treated with Vehicle (PBS), TP10 (anti-CD27 antibody), XC201 (knottin Fc fusion), TP10+anti-PD-1 (combination with RMP1-14 clone), or TP10+NOD201. Ten animals were used for each treatment arm. Tumors were implanted 12 days prior to the start of treatment with 3×10⁵ CT26 tumor cells. p-values were adjusted for multiple comparison testing: i.e. Bonferroni correction was applied by multiplying original p-values by the number of tests performed (4). For Kaplan Meier—“endpoint” for each mouse was linearly interpolated to determine when tumors reached sizes of 2000 mm³. At study start mean tumor size was ˜100 mm³ and range was 75-144 mm³. Treatment dosing was performed on days 1, 7, 13, 19. Anti-PD-1 dosing was performed on days 1, 4, 8, 11.

FIG. 11A-FIG. 11B provide tumor growth and tumor mass for mice treated with Vehicle (PBS), TP10 (anti-CD27 antibody), XC201 (knottin Fc fusion), TP10+anti-PD-1 (combination with RMP1-14 clone), or TP10+NOD201. Tumors were implanted 12 days prior to the start of treatment with 3×10⁵ CT26 tumor cells. FIG. 11A shows tumor volume (mm³) and FIG. 11B provides tumor mass (mg). Mice were sacked when tumor growth exceeded 2000 mm³ (dotted line). Tumors were excised from 3 randomly selected mice from each group on day 16. Tumors were subsequently analyzed for mass (right) and on flow cytometry. At study start mean tumor size was ˜100 mm³ and range was 75-144 mm³. Treatment dosing was performed on days 1, 7, 13, 19. Anti-PD-1 dosing was performed on days 1, 4, 8, 11.

FIG. 12 provides individual tumor growth plots for the mice treated with one of four treatment arms compared to the mice treated with vehicle (PBS). The treatment arms include TP10 (anti-CD27 antibody), XC201 (knottin Fc fusion), TP10+anti-PD-1 (combination with RMP1-14 clone), or TP10+NOD201. Tumors were implanted 12 days prior to the start of treatment with 3×10⁵ CT26 tumor cells. At study start mean tumor size was ˜100 mm³ and range was 75-144 mm³. Treatment dosing was performed on days 1, 7, 13, 19. Anti-PD-1 dosing was performed on days 1, 4, 8, 11.

FIG. 13A-FIG. 13B provide T cell subsets in the tumor for mice treated with Vehicle (PBS), TP10 (anti-CD27 antibody), XC201 (knottin Fc fusion), TP10+anti-PD-1 (combination with RMP1-14 clone), or TP10+NOD201. Tumors were implanted 12 days prior to the start of treatment with 3×10⁵ CT26 tumor cells. FIG. 13A provides percent cytotoxic T lymphocytes (CD3+CD8+) of live cells. FIG. 13B provides percent helper T lymphocytes (CD3+CD4+) of live cells. Mice were sacked when tumor growth exceeded 2000 mm³ (dotted line). Tumors were excised from 3 randomly selected mice from each group on day 16. Tumors were subsequently analyzed for mass (right) and on flow cytometry. See, also, FIG. 10.

FIG. 14A-FIG. 14B provide data on T-cell activation in the tumor of mice treated with Vehicle (PBS), TP10 (anti-CD27 antibody), XC201 (knottin Fc fusion), TP10+anti-PD-1 (combination with RMP1-14 clone), or TP10+NOD201. Tumors were implanted 12 days prior to the start of treatment with 3×10⁵ CT26 tumor cells. Mice were sacked when tumor growth exceeded 2000 mm³ (dotted line). Tumors were excised from 3 randomly selected mice from each group on day 16. Tumors were subsequently analyzed for mass (right) and on flow cytometry. FIG. 14A provides percent CD44hi of cytotoxic T lymphocytes (CD3+CD8+) and FIG. 14B provides percent CD44hi of helper T lymphocytes (CD3+CD4+).

FIG. 15A-FIG. 15B provide spleen mass and CD45 content in mice treated with Vehicle (PBS), TP10 (anti-CD27 antibody), XC201 (knottin Fc fusion), TP10+anti-PD-1 (combination with RMP1-14 clone), or TP10+NOD201. Tumors were implanted 12 days prior to the start of treatment with 3×10⁵ CT26 tumor cells. FIG. 15A provides spleen mass and FIG. 15B provides percent CD45+ of live cells. Spleens were excised from 3 randomly selected mice from each group on day 16. Spleens were subsequently analyzed for mass (left) and on flow cytometry (right). Spleen mass did not correlate with tumor growth. No significant differences in spleen mass or CD45+ proportion.

FIG. 16 provides cell type distribution from spleen cells in mice treated with Vehicle (PBS), TP10 (anti-CD27 antibody), XC201 (knottin Fc fusion), TP10+anti-PD-1 (combination with RMP1-14 clone), or TP10+NOD201. Tumors were implanted 12 days prior to the start of treatment with 3×10⁵ CT26 tumor cells. Spleens were excised from 3 randomly selected mice from each group on day 16.

FIG. 17 provides checkpoint marker expression on different subsets of T cells from spleens of mice treated with Vehicle (PBS), TP10 (anti-CD27 antibody), XC201 (knottin Fc fusion), TP10+anti-PD-1 (combination with RMP1-14 clone), or TP10+NOD201. Tumors were implanted 12 days prior to the start of treatment with 3×10⁵ CT26 tumor cells. Spleens were excised from 3 randomly selected mice from each group on day 16.

FIG. 18 provides T-cell activation marker expression on different subsets of T cells from spleens of mice treated with Vehicle (PBS), TP10 (anti-CD27 antibody), XC201 (knottin Fc fusion), TP10+anti-PD-1 (combination with RMP1-14 clone), or TP10+NOD201. Tumors were implanted 12 days prior to the start of treatment with 3×10⁵ CT26 tumor cells. Spleens were excised from 3 randomly selected mice from each group on day 16.

FIG. 19A-FIG. 19H provides the variable heavy and light chains and corresponding vhCDR1, vhCDR2, vhCDR3, vlCDR1, vlCDR2, and vlCDR3 sequences for the TP01, TP02, TP03, TP04, TP05, TP06, TP07, TP08, TP09, TP10, TP11, TP12, TP13, TP14, TP15, TP16, TP17, TP18, TP19, TP20, TP21, TP22, TP23, TP24, TP25, TP26, TP27, TP28, TP29, TP30, TP31, and TP32 anti-CD27 antibodies.

### DETAILED DESCRIPTION

### I. Introduction

The present disclosure provides novel anti-CD27 antibodies. The anti-CD27 antibodies described herein bind human CD27. In some embodiments, the anti-CD27 antibodies bind human CD27 with high affinities. In some embodiments, the anti-CD27 antibodies act as functional CD27 agonists, and upon binding to CD27 they induce or enhance an immune response. In some embodiments, the anti-CD27 antibodies act as functional CD27 antagonists, and upon binding to CD27 they block interaction of CD27 with CD70, and inhibit an immune response. Also provided in the present disclosure are methods of using such antibodies to modulate an immune response in a subject, and, for example, to treat cancer. In addition, nucleic acids encoding these antibodies, as well as host cells that include such nucleic acids are described in the present disclosure.

### II. Definitions

To facilitate an understanding of the present invention, a number of terms and phrases are defined below.

As used herein, each of the following terms has the meaning associated with it in this section.

Terms used in the claims and specification are defined as set forth below unless otherwise specified. In the case of direct conflict with a term used in a parent provisional patent application, the term used in the instant specification shall control.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, meaning one of the 20 naturally occurring amino acids that are coded for by DNA and RNA, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid. Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, the present larger “peptide insertions,” can be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution M252Y refers to a variant polypeptide, in this case an Fc variant, in which the methionine at position 252 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

“Polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, “protein” herein is meant at least two covalently attached amino acids, which includes proteins, polypeptides, oligopeptides and peptides. The peptidyl group may comprise naturally occurring amino acids and peptide bonds.

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., Biol. Chem. 260:2605-2608, 1985; and Cassol et al, 1992; Rossolini et al, Mol. Cell. Probes 8:91-98, 1994). For arginine and leucine, modifications at the second base can also be conservative. The term nucleic acid is used interchangeably with gene, cDNA, and mRNA encoded by a gene. Polynucleotides used herein can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The term “nucleotide sequence” includes the ordering of nucleotides in an oligonucleotide or polynucleotide in a single-stranded form of nucleic acid.

By “nucleic acid construct” it is meant a nucleic acid sequence that has been constructed to comprise one or more functional units not found together in nature. Examples include circular, linear, double-stranded, extrachromosomal DNA molecules (plasmids), cosmids (plasmids containing COS sequences from lambda phage), viral genomes including non-native nucleic acid sequences, and the like.

The terms “oligonucleotide,” “polynucleotide,” and “nucleic acid molecule”, used interchangeably herein, refer to a polymeric forms of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

The term “antibody” is used in the broadest sense and includes, for example, an intact immunoglobulin or an antigen binding portion. Antigen binding portions may be produced by recombinant DNA techniques or by enzymatic or chemical cleavage of intact antibodies. Thus the term antibody includes traditional tetrameric antibodies of two heavy chains and two light chains, as well as antigen binding fragments such as Fv, Fab and scFvs. In some cases, the invention provides bispecific antibodies that include at least one antigen binding domain as outlined herein.

As used herein, the term “PK” is an acronym for “pharmacokinetic” and encompasses properties of a compound including, by way of example, absorption, distribution, metabolism, and elimination by a subject. As used herein, an “extended-PK group” refers to a protein, peptide, or moiety that increases the circulation half-life of a biologically active molecule when fused to or administered together with the biologically active molecule. Examples of an extended-PK group include PEG, human serum albumin (HSA) binders (as disclosed in U.S. Publication Nos. 2005/0287153 and 2007/0003549, PCT Publication Nos. WO 2009/083804 and WO 2009/133208, and SABA molecules as described in US Publication No. 2012/094909), human serum albumin, Fc or Fc fragments and variants thereof, and sugars (e.g., sialic acid). Other exemplary extended-PK groups are disclosed in Kontermann et al., Current Opinion in Biotechnology 2011; 22:868-876, which is herein incorporated by reference in its entirety.

The term “Kassoc” or “Ka”, as used herein, is intended to refer to the association rate of a particular antibody-antigen interaction, whereas the term “Kdis” or “Kd,” as used herein, is intended to refer to the dissociation rate of a particular antibody-antigen interaction. The term “K_{D}”, as used herein, is intended to refer to the dissociation constant, which is obtained from the ratio of Kd to Ka (i.e., Kd/Ka) and is expressed as a molar concentration (M). K_{D} values for antibodies can be determined using methods well established in the art. In some embodiments, the method for determining the K_{D} of an antibody is by using surface plasmon resonance, for example, by using a biosensor system such as a BIACORE® system. In some embodiments, the K_{D} of an antibody is determined by Bio-Layer Interferometry. In some embodiments, the K_{D} value is measured with the immobilized. In other embodiments, the K_{D} value is measured with the antibody (e.g., parent mouse antibody, chimeric antibody, or humanized antibody variants) immobilized. In certain embodiments, the K_{D} value is measured in a bivalent binding mode. In other embodiments, the K_{D} value is measured in a monovalent binding mode.

In certain aspects, the polypeptide described can employ one or more “linker domains,” such as polypeptide linkers. As used herein, the term “linker” or “linker domain” refers to a sequence which connects two or more domains in a linear sequence. As used herein, the term “polypeptide linker” refers to a peptide or polypeptide sequence (e.g., a synthetic peptide or polypeptide sequence) which connects two or more domains in a linear amino acid sequence of a polypeptide chain. For example, polypeptide linkers may be used to connect a polypeptide to an Fc domain or other PK-extender such as HSA. In some embodiments, such polypeptide linkers can provide flexibility to the polypeptide molecule. Exemplary linkers include Gly-Ser linkers, such as but not limited to [Gly4Ser], comprising 4 glycines followed by 1 serine and [Gly4Ser3], comprising 4 glycines followed by 3 serines. The term “linker” herein can also refer to a linker used in scFv and/or other antibody structures. Generally, there are a number of suitable scFv linkers that can be used, including traditional peptide bonds, generated by recombinant techniques. The linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n, (GSGGS)n, (GGGGS)n, and (GGGS)n, where n is an integer of at least one (and generally from 3 to 4), glycine-alanine polymers, alanine-serine polymers, and other flexible linkers. Alternatively, a variety of non-proteinaceous polymers, including but not limited to polyethylene glycol (PEG), polypropylene glycol, polyoxyalkylenes, or copolymers of polyethylene glycol and polypropylene glycol, may find use as linkers, that is may find use as linkers. Other linker sequences may include any sequence of any length of CL/CH1 domain but not all residues of CL/CH1 domain; for example, the first 5-12 amino acid residues of the CL/CH1 domains. Linkers can be derived from immunoglobulin light chain, for example Cκ or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences may also be derived from other proteins such as Ig-like proteins (e.g., TCR, FcR, KIR), hinge region-derived sequences, and other natural sequences from other proteins. In some embodiments, the linker is a “domain linker”, used to link any two domains as outlined herein together. While any suitable linker can be used, many embodiments utilize a glycine-serine polymer, including for example (GS)n, (GSGGS)n, (GGGGS)n, and (GGGS)n, where n is an integer of at least one (and generally from 3 to 4 to 5) as well as any peptide sequence that allows for recombinant attachment of the two domains with sufficient length and flexibility to allow each domain to retain its biological function.

As used herein, the terms “linked,” “fused”, or “fusion” are used interchangeably. These terms refer to the joining together of two or more elements or components or domains, by whatever means including chemical conjugation or recombinant means. Methods of chemical conjugation (e.g., using heterobifunctional crosslinking agents) are known in the art.

The term “integrin” means a transmembrane heterodimeric protein important for cell adhesion. Integrins comprise an α and β subunit. These proteins bind to extracellular matrix components (e.g., fibronectin, collagen, laminin, etc.) and respond by inducing signaling cascades. Integrins bind to extracellular matrix components by recognition of an Arg-Gly-Asp (RGD) motif. Certain integrins are found on the surface of tumor cells and therefore make promising therapeutic targets. In certain embodiments, the integrins being targeted are αᵥβ3, αᵥβ5, and α5β1, individually or in combination.

The term “integrin-binding polypeptide” refers to a polypeptide which includes an integrin-binding domain or loop within a knottin polypeptide scaffold. The integrin binding domain or loop includes at least one RGD peptide. In certain embodiments, the RGD peptide is recognized by αᵥβ₁, αᵥβ₃, αᵥβ₅, αᵥβ₆, and αᵥβ₁ integrins. In certain embodiments the RGD peptide binds to a combination of αᵥβ₁, αᵥβ₃, αᵥβ₅, αᵥβ₆, and αᵥβ₁ integrins. These specific integrins are found on tumor cells and their vasculature and are therefore the targets of interest.

Integrins are a family of extracellular matrix adhesion proteins that noncovalently associate into α and β heterodimers with distinct cellular and adhesive specificities (Hynes, 1992; Luscinskas and Lawler, 1994). Cell adhesion, mediated though integrin-protein interactions, is responsible for cell motility, survival, and differentiation. Each α and β subunit of the integrin receptor contributes to ligand binding and specificity.

Protein binding to many different cell surface integrins can be mediated through the short peptide motif Arg-Gly-Asp (RGD) (Pierschbacher and Ruoslahti, 1984). These peptides have dual functions: They promote cell adhesion when immobilized onto a surface, and they inhibit cell adhesion when presented to cells in solution. Adhesion proteins that contain the RGD sequence include: fibronectin, vitronectin, osteopontin, fibrinogen, von Willebrand factor, thrombospondin, laminin, entactin, tenascin, and bone sialoprotein (Ruoslahti, 1996). The RGD sequence displays specificity to about half of the 20 known integrins including the α₅β₁, α₈β₁, αᵥβ₁, αᵥβ₃, αᵥβ₅, αᵥβ₆, αᵥβ₈, and αᵥβ₃ integrins, and, to a lesser extent, the α₂β₁, α₃β₁, α₄β₁, and α₇β₁ integrins (Ruoslahti, 1996). In particular, the αᵥβ₃ integrin is capable of binding to a large variety of RGD containing proteins including fibronectin, fibrinogen, vitronectin, osteopontin, von Willebrand factor, and thrombospondin (Ruoslahti, 1996; Haubner et al., 1997), while the α₅β₁ integrin is more specific and has only been shown to bind to fibronectin (D'Souza et al., 1991).

The linear peptide sequence RGD has a much lower affinity for integrins than the proteins from which it is derived (Hautanen et al., 1989). This due to conformational specificity afforded by folded protein domains not present in linear peptides. Increased functional integrin activity has resulted from preparation of cyclic RGD motifs, alteration of the residues flanking the RGD sequence, and synthesis of small molecule mimetics (reviewed in (Ruoslahti, 1996; Haubner et al., 1997)).

The term “loop domain” refers to an amino acid subsequence within a peptide chain that has no ordered secondary structure, and resides generally on the surface of the peptide. The term “loop” is understood in the art as referring to secondary structures that are not ordered as in the form of an alpha helix, beta sheet, etc.

The term “integrin-binding loop” refers to a primary sequence of about 9-13 amino acids which is typically created ab initio through experimental methods such as directed molecular evolution to bind to integrins. In certain embodiments, the integrin-binding loop includes an RGD peptide sequence, or the like, placed between amino acids which are particular to the scaffold and the binding specificity desired. The RGD-containing peptide or similar peptide (such as RYD, etc.) is generally not simply taken from a natural binding sequence of a known protein. The integrin-binding loop is preferably inserted within a knottin polypeptide scaffold between cysteine residues, and the length of the loop adjusted for optimal integrin-binding depending on the three-dimensional spacing between cysteine residues. For example, if the flanking cysteine residues in the knottin scaffold are linked to each other, the optimal loop may be shorter than if the flanking cysteine residues are linked to cysteine residues separated in primary sequence. Otherwise, particular amino acid substitutions can be introduced to constrain a longer RGD-containing loop into an optimal conformation for high affinity integrin binding. The knottin polypeptide scaffolds used herein may contain certain modifications made to truncate the native knottin, or to remove a loop or unnecessary cysteine residue or disulfide bond.

Incorporation of integrin-binding sequences into a molecular (e.g., knottin polypeptide) scaffold provides a framework for ligand presentation that is more rigid and stable than linear or cyclic peptide loops. In addition, the conformational flexibility of small peptides in solution is high, and results in large entropic penalties upon binding. Such constructs have also been described in detail in International Patent Publication WO 2016/025642, incorporated herein by reference in its entirety.

Incorporation of an integrin-binding sequence into a knottin polypeptide scaffold provides conformational constraints that are required for high affinity integrin binding. Furthermore, the scaffold provides a platform to carry out protein engineering studies such as affinity or stability maturation.

As used herein, the term “knottin protein” refers to a structural family of small proteins, typically 25-40 amino acids, which bind to a range of molecular targets like proteins, sugars and lipids. Their three-dimensional structure is essentially defined by a peculiar arrangement of three to five disulfide bonds. A characteristic knotted topology with one disulfide bridge crossing the macro-cycle limited by the two other intra-chain disulfide bonds, which was found in several different microproteins with the same cystine network, lent its name to this class of biomolecules. Although their secondary structure content is generally low, the knottins share a small triple-stranded antiparallel β-sheet, which is stabilized by the disulfide bond framework. Biochemically well-defined members of the knottin family, also called cystine knot proteins, include the trypsin inhibitor EETI-II from Ecballium elaterium seeds, the neuronal N-type Ca²⁺ channel blocker co-conotoxin from the venom of the predatory cone snail Conus geographus, agouti-related protein (AgRP, See Millhauser et al., “Loops and Links: Structural Insights into the Remarkable Function of the Agouti-Related Protein,” Ann. N.Y. Acad. ScL, Jun. 1, 2003; 994(1): 27-35), the omega agatoxin family, etc. A suitable agatoxin sequence [SEQ ID NO: 41] is given in U.S. Pat. No. 8,536,301, having a common inventor with the present application. Other agatoxin sequences suitable for use in the methods disclosed herein include, but are not limited to Omega-agatoxin-Aa4b (GenBank Accession number P37045) and Omega-agatoxin-Aa3b (GenBank Accession number P81744). Other knottin sequences suitable for use in the methods disclosed herein include, knottin [Bemisia tabaci] (GenBank Accession number FJ601218.1), Omega-lycotoxin (Genbank Accession number P85079), mu-O conotoxin MrVIA=voltage-gated sodium channel blocker (Genbank Accession number AAB34917) and Momordica cochinchinensis Trypsin Inhibitor I (MCoTI-I) or II (MCoTI-II) (Uniprot Accession numbers P82408 and P82409, respectively).

Knottin proteins have a characteristic disulfide linked structure. This structure is also illustrated in Gelly et al., “The KNOTTIN website and database: a new information system dedicated to the knottin scaffold,” Nucleic Acids Research, 2004, Vol. 32, Database issue D156-D159. A triple-stranded β-sheet is present in many knottins. The spacing between cysteine residues is important, as is the molecular topology and conformation of the integrin-binding loop.

The term “molecular scaffold” means a polymer having a predefined three-dimensional structure, into which an integrin-binding loop is incorporated, such as an RGD peptide sequence as described herein. The term “molecular scaffold” has an art-recognized meaning (in other contexts), which is also intended here. For example, a review by Skerra, “Engineered protein scaffolds for molecular recognition,” J. Mol. Recognit. 2000; 13: 167-187 describes the following scaffolds: single domains of antibodies of the immunoglobulin superfamily, protease inhibitors, helix-bundle proteins, disulfide-knotted peptides and lipocalins. Guidance is given for the selection of an appropriate molecular scaffold.

The term “knottin polypeptide scaffold” refers to a knottin protein suitable for use as a molecular scaffold, as described herein. Characteristics of a desirable knottin polypeptide scaffold for engineering include 1) high stability in vitro and in vivo, 2) the ability to replace amino acid regions of the scaffold with other sequences without disrupting the overall fold, 3) the ability to create multifunctional or bispecific targeting by engineering separate regions of the molecule, and 4) a small size to allow for chemical synthesis and incorporation of non-natural amino acids if desired. Scaffolds derived from human proteins are favored for therapeutic applications to reduce toxicity or immunogenicity concerns, but are not always a strict requirement. Other scaffolds that have been used for protein design include fibronectin (Koide et al., 1998), lipocalin (Beste et al., 1999), cytotoxic T lymphocyte-associated antigen 4 (CTLA-4) (Hufton et al, 2000), and tendamistat (McConnell and Hoess, 1995; Li et al, 2003). While these scaffolds have proved to be useful frameworks for protein engineering, molecular scaffolds such as knottins have distinct advantages: their small size and high stability.

As used herein, the term “NOD201” refers to an integrin-binding polypeptide-Fc fusion comprising the following sequence: GCPRPRGDNPPLTCSQDSDCLAGCVCGPNGFCG (SEQ ID NO:382; 2.5F peptide) and having no linker between the 2.5F peptide and the Fc domain. In some embodiments, the Fc domain is from IgG1, IgG2, IgG3, or IgG4 and can be mouse or human derived.

As used herein, the term “NOD201modK” refers to an integrin-binding polypeptide-Fc fusion comprising the following sequence: GCPRPRGDNPPLTCKQDSDCLAGCVCGPNGFCG (SEQ ID NO:383; 2.5FmodK peptide) and having no linker between the 2.5FmodK peptide and the Fc domain. In some embodiments, the Fc domain is from IgG1, IgG2, IgG3, or IgG4 and can be mouse or human derived.

As used herein, the term “NOD203” refers to an integrin-binding polypeptide-Fc fusion comprising the following sequence: GCPRPRGDNPPLTCSQDSDCLAGCVCGPNGFCGGGGGS (SEQ ID NO:384; 2.5F peptide) and having a Gly₄Ser linker between the 2.5F peptide and the Fc domain. In some embodiments, the Fc domain is from IgG1, IgG2, IgG3, or IgG4 and can be mouse or human derived.

As used herein, the term “NOD203modK” refers to an integrin-binding polypeptide-Fc fusion comprising the following sequence: GCPRPRGDNPPLTCKQDSDCLAGCVCGPNGFCGGGGGS (SEQ ID NO:385; 2.5FmodK peptide) and having a Gly₄Ser linker between the 2.5FmodK peptide and the Fc domain. In some embodiments, the Fc domain is from IgG1, IgG2, IgG3, or IgG4 and can be mouse or human derived.

As used herein, the term “NOD204” refers to an integrin-binding polypeptide-FC fusion comprising the following sequence: GCPRPRGDNPPLTCSQDSDCLAGCVCGPNGFCGGGGGSGGGGSGGGGS (SEQ ID NO:386; 2.5F peptide) and having a Gly₄Ser₃ linker between the 2.5F peptide and the Fc domain. In some embodiments, the Fc domain is from IgG1, IgG2, IgG3, or IgG4 and can be mouse or human derived.

As used herein, the term “NOD204modK” refers to an integrin-binding polypeptide-FC fusion comprising the following sequence: CPRPRGDNPPLTCKQDSDCLAGCVCGPNGFCGGGGGSGGGGSGGGGS (SEQ ID NO:387; 2.5FmodK peptide) and having a Gly₄Ser₃ linker between the 2.5FmodK peptide and the Fc domain. In some embodiments, the Fc domain is from IgG1, IgG2, IgG3, or IgG4 and can be mouse or human derived.

As used herein, the term “AgRP” means PDB entry 1HYK. Its entry in the Knottin database is SwissProt AGRP HUMAN, where the full-length sequence of 129 amino acids may be found. It comprises the sequence beginning at amino acid 87. An additional G is added to this construct. It also includes a CI 05 A mutation described in Jackson, et al. 2002 Biochemistry, 41, 7565, as well as International Patent Publication WO 2016/025642, incorporated by reference in its entirety; bold and underlined portion, from loop 4, is replaced by the RGD sequences described herein. Loops 1 and 3 are shown between brackets.

As used herein, “integrin-binding polypeptide-Fc fusion” is used interchangeably with “knottin-Fc” and refers to an integrin-binding polypeptide that includes an integrin-binding amino acid sequence within a knottin polypeptide scaffold and is operably linked to an Fc domain. In some embodiments, the Fc domain is fused to the N-terminus of the integrin-binding polypeptide. In some embodiments, the Fc domain is fused to the C-terminus of the integrin-binding polypeptide. In some embodiments, the Fc domain is operably linked to the integrin-binding polypeptide via a linker.

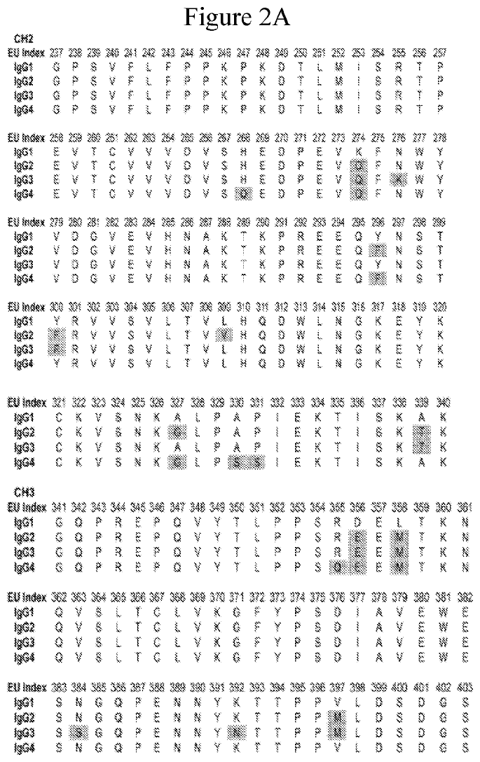

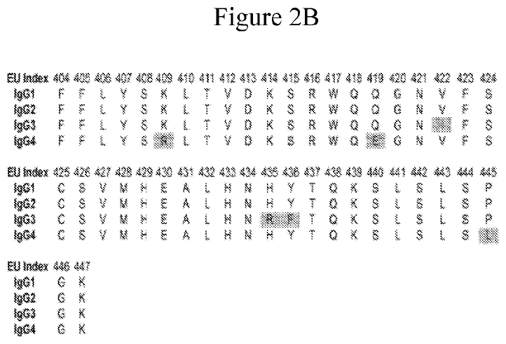

As used herein, the term “Fc region” refers to the portion of a native immunoglobulin formed by the respective Fc domains (or Fc moieties) of its two heavy chains. As used herein, the term “Fc domain” refers to a portion of a single immunoglobulin (Ig) heavy chain wherein the Fc domain does not comprise an Fv domain. As such, an Fc domain can also be referred to as “Ig” or “IgG.” In certain embodiments, an Fc domain begins in the hinge region just upstream of the papain cleavage site and ends at the C-terminus of the antibody. Accordingly, a complete Fc domain comprises at least a hinge domain, a CH₂ domain, and a CH₃ domain. In certain embodiments, an Fc domain comprises at least one of: a hinge (e.g., upper, middle, and/or lower hinge region) domain, a CH₂ domain, a CH₃ domain, a CH₄ domain, or a variant, portion, or fragment thereof. In other embodiments, an Fc domain comprises a complete Fc domain (i.e., a hinge domain, a CH₂ domain, and a CH₃ domain). In one embodiment, an Fc domain comprises a hinge domain (or portion thereof) fused to a CH₃ domain (or portion thereof). In another embodiment, an Fc domain comprises a CH₂ domain (or portion thereof) fused to a CH₃ domain (or portion thereof). In another embodiment, an Fc domain consists of a CH₃ domain or portion thereof. In another embodiment, an Fc domain consists of a hinge domain (or portion thereof) and a CH₃ domain (or portion thereof). In another embodiment, an Fc domain consists of a CH₂ domain (or portion thereof) and a CH₃ domain. In another embodiment, an Fc domain consists of a hinge domain (or portion thereof) and a CH₂ domain (or portion thereof). In one embodiment, an Fc domain lacks at least a portion of a CH₂ domain (e.g., all or part of a CH₂ domain). An Fc domain herein generally refers to a polypeptide comprising all or part of the Fc domain of an immunoglobulin heavy-chain. This includes, but is not limited to, polypeptides comprising the entire CH₁, hinge, CH₂, and/or CH₃ domains as well as fragments of such peptides comprising only, e.g., the hinge, CH₂, and CH₃ domain. The Fc domain may be derived from an immunoglobulin of any species and/or any subtype, including, but not limited to, a human IgG1, IgG2, IgG3, IgG4, IgD, IgA, IgE, or IgM antibody. A human IgG1 constant region can be found at Uniprot P01857 and in FIG. 2. The Fc domain of human IgG1 with a deletion of the upper hinge region can be found in Table 2, SEQ ID NO: 3 from International Patent Publication No. WO 2016/025642. The Fc domain encompasses native Fc and Fc variant molecules. As with Fc variants and native Fc's, the term Fc domain includes molecules in monomeric or multimeric (e.g., dimeric) form, whether digested from whole antibody or produced by other means. The assignment of amino acid residue numbers to an Fc domain is in accordance with the definitions of Kabat. See, e.g., Sequences of Proteins of Immunological Interest (Table of Contents, Introduction and Constant Region Sequences sections), 5th edition, Bethesda, Md.: NIH vol. 1:647-723 (1991); Kabat et al., “Introduction” Sequences of Proteins of Immunological Interest, US Dept of Health and Human Services, NIH, 5^{th} edition, Bethesda, Md. vol. 1:xiii-xcvi (1991); Chothia & Lesk, J. Mol. Biol. 196:901-917 (1987); Chothia et al, Nature 342:878-883 (1989), each of which is herein incorporated by reference for all purposes. With regard to the integrin-binding polypeptide-Fc fusions described herein, any Fc domain from any IgG as described herein or known can be employed as part of the Fc fusion, including mouse, human and variants thereof, such as hinge deleted (EPKSC deleted; see, SEQ ID NO: 3 from International Patent Publication No. WO 2016/025642).

As set forth herein, it will be understood by one of ordinary skill in the art that any Fc domain may be modified such that it varies in amino acid sequence from the native Fc domain of a naturally occurring immunoglobulin molecule. In certain exemplary embodiments, the Fc domain has increased effector function (e.g., FcγR binding).

The Fc domains of a polypeptide of the invention may be derived from different immunoglobulin molecules. For example, an Fc domain of a polypeptide may comprise a CH₂ and/or CH₃ domain derived from an IgG1 molecule and a hinge region derived from an IgG3 molecule. In another example, an Fc domain can comprise a chimeric hinge region derived, in part, from an IgG1 molecule and, in part, from an IgG3 molecule. In another example, an Fc domain can comprise a chimeric hinge derived, in part, from an IgG1 molecule and, in part, from an IgG4 molecule.

A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence. Polypeptides derived from another peptide may have one or more mutations relative to the starting polypeptide, e.g., one or more amino acid residues which have been substituted with another amino acid residue or which has one or more amino acid residue insertions or deletions.

A polypeptide can comprise an amino acid sequence which is not naturally occurring. Such variants necessarily have less than 100% sequence identity or similarity with the starting IL-2 or knottin protein. In some embodiments, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and in some embodiments from about 95% to less than 100%, e.g., over the length of the variant molecule.

In one embodiment, there is one amino acid difference between a starting polypeptide sequence and the sequence derived therefrom. Identity or similarity with respect to this sequence is defined herein as the percentage of amino acid residues in the candidate sequence that are identical (i.e., same residue) with the starting amino acid residues, after aligning the sequences and introducing gaps, if necessary, to achieve the maximum percent sequence identity.

TABLE 1
Sequence Summary

| SEQ | | |
|---|---|---|
| ID | | |
| NO | Description | Sequence |
| 289 | Human | ASTKGPSVFPLAPSSKSTSGGTAALGCLVKDYFPEPVTVSWNSGALTSGVHTFPAVLQS |
| | IgG1 | SGLYSLSSVVTVPSSSLGTQTYICNVNHKPSNTKVDKKVEPKSCDKTHTCPPCPAPELL |
| | constant | GGPSVFLFPPKPKDTLMISRTPEVTCVVVDVSHEDPEVKFNWYVDGVEVHNAKTKPREE |
| | region | QYNSTYRVVSVLTVLHQDWLNGKEYKCKVSNKALPAPIEKTISKAKGQPREPQVYTLPP |
| | (amino acid | SRDELTKNQVSLTCLVKGFYPSDIAVEWESNGQPENNYKTTPPVLDSDGSFFLYSKLTV |
| | sequence) | DKSRWQQGNVFSCSVMHEALHNHYTQKSLSLSPGK |
| 290 | Human | EPKSCDKTHTCPPCPAPELLGGPSVFLFPPKPKDTLMISRTPEVTCVVVDVSHEDPEVK |
| | IgG1 Fc | FNWYVDGVEVHNAKTKPREEQYNSTYRVVSVLTVLHQDWLNGKEYKCKVSNKALPAPIE |
| | domain | KTISKAKGQPREPQVYTLPPSRDELTKNQVSLTCLVKGFYPSDIAVEWESNGQPENNYK |
| | (amino acid | TTPPVLDSDGSFFLYSKLTVDKSRWQQGNVFSCSVMHEALHNHYTQKSLSLSPGK |
| | sequence) | |
| 291 | Human | DKTHTCPPCPAPELLGGPSVFLFPPKPKDTLMISRTPEVTCVVVDVSHEDPEVKFNWYV |
| | IgG1 Fc | DGVEVHNAKTKPREEQYNSTYRVVSVLTVLHQDWLNGKEYKCKVSNKALPAPIEKTISK |
| | domain | AKGQPREPQVYTLPPSRDELTKNQVSLTCLVKGFYPSDIAVEWESNGQPENNYKTTPPV |
| | (amino acid | LDSDGSFFLYSKLTVDKSRWQQGNVFSCSVMHEALHNHYTQKSLSLSPGK |
| | sequence) | |
| | Deletion | |
| | (ΔEPKSC) | |
| | Upper | |
| | Hinge | |
| 292 | Human | MDMRVPAQLLGLLLLWLPGARCADAHKSEVAHRFKDLGEENFKALVLIAFAQYLQQCPF |
| | serum | EDHVKLVNEVTEFAKTCVADESAENCDKSLHTLFGDKLCTVATLRETYGEMADCCAKQE |
| | albumin | PERNECFLQHKDDNPNLPRLVRPEVDVMCTAFHDNEETFLKKYLYEIARRHPYFYAPEL |
| | (amino acid | LFFAKRYKAAFTECCQAADKAACLLPKLDELRDEGKASSAKQRLKCASLQKFGERAFKA |
| | sequence) | WAVARLSQRFPKAEFAEVSKLVTDLTKVHTECCHGDLLECADDRADLAKYICENQDSIS |
| | | SKLKECCEKPLLEKSHCIAEVENDEMPADLPSLAADFVESKDVCKNYAEAKDVFLGMFL |
| | | YEYARRHPDYSVVLLLRLAKTYETTLEKCCAAADPHECYAKVFDEFKPLVEEPQNLIKQ |
| | | NCELFEQLGEYKFQNALLVRYTKKVPQVSTPTLVEVSRNLGKVGSKCCKHPEAKRMPCA |
| | | EDYLSVVLNQLCVLHEKTPVSDRVTKCCTESLVNRRPCFSALEVDETYVPKEFNAETFT |
| | | FHADICTLSEKERQIKKQTALVELVKHKPKATKEQLKAVMDDFAAFVEKCCKADDKETC |
| | | FAEEGKKLVAASQAALGLGGGSAPTSSSTKKTQLQLEHLLLDLQMILNGINNYKNPKLT |
| | | RMLTFKFYMPKKATELKHLQCLEEELKPLEEVLNLAQSKNFHLRPRDLISNINVIVLEL |
| | | KGSETTFMCEYADETATIVEFLNRWITFCQSIISTLTGGGS |
| 293 | Mature | DAHKSEVAHRFKDLGEENFKALVLIAFAQYLQQCPFEDHVKLVNEVTEFAKTCVADESA |
| | HSA (amino | ENCDKSLHTLFGDKLCTVATLRETYGEMADCCAKQEPERNECFLQHKDDNPNLPRLVRP |
| | acid | EVDVMCTAFHDNEETFLKKYLYEIARRHPYFYAPELLFFAKRYKAAFTECCQAADKAAC |
| | sequence) | LLPKLDELRDEGKASSAKQRLKCASLQKFGERAFKAWAVARLSQRFPKAEFAEVSKLVT |
| | | DLTKVHTECCHGDLLECADDRADLAKYICENQDSISSKLKECCEKPLLEKSHCIAEVEN |
| | | DEMPADLPSLAADFVESKDVCKNYAEAKDVFLGMFLYEYARRHPDYSVVLLLRLAKTYE |
| | | TTLEKCCAAADPHECYAKVFDEFKPLVEEPQNLIKQNCELFEQLGEYKFQNALLVRYTK |
| | | KVPQVSTPTLVEVSRNLGKVGSKCCKHPEAKRMPCAEDYLSVVLNQLCVLHEKTPVSDR |
| | | VTKCCTESLVNRRPCFSALEVDETYVPKEFNAETFTFHADICTLSEKERQIKKQTALVE |
| | | LVKHKPKATKEQLKAVMDDFAAFVEKCCKADDKETCFAEEGKKLVAASQAALGLGGGSA |
| | | PTSSSTKKTQLQLEHLLLDLQMILNGINNYKNPKLTRMLTFKFYMPKKATELKHLQCLE |
| | | EELKPLEEVLNLAQSKNFHLRPRDLISNINVIVLELKGSETTFMCEYADETATIVEFLN |
| | | RWITFCQSIISTLTGGGS |
| 294 | Human | ATGGATATGCGGGTGCCTGCTCAGCTGCTGGGACTGCTGCTGCTGTGGCTGCCTGGGGC |
| | serum | TAGATGCGCCGATGCTCACAAAAGCGAAGTCGCACACAGGTTCAAAGATCTGGGGGAGG |
| | albumin | AAAACTTTAAGGCTCTGGTGCTGATTGCATTCGCCCAGTACCTGCAGCAGTGCCCCTTT |
| | (nucleic | GAGGACCACGTGAAACTGGTCAACGAAGTGACTGAGTTCGCCAAGACCTGCGTGGCCGA |
| | acid | CGAATCTGCTGAGAATTGTGATAAAAGTCTGCATACTCTGTTTGGGGATAAGCTGTGTA |
| | sequence) | CAGTGGCCACTCTGCGAGAAACCTATGGAGAGATGGCAGACTGCTGTGCCAAACAGGAA |
| | | CCCGAGCGGAACGAATGCTTCCTGCAGCATAAGGACGATAACCCCAATCTGCCTCGCCT |
| | | GGTGCGACCTGAGGTGGACGTCATGTGTACAGCCTTCCACGATAATGAGGAAACTTTTC |
| | | TGAAGAAATACCTGTACGAAATCGCTCGGAGACATCCTTACTTTTATGCACCAGAGCTG |
| | | CTGTTCTTTGCCAAACGCTACAAGGCCGCTTTCACCGAGTGCTGTCAGGCAGCCGATAA |
| | | AGCTGCATGCCTGCTGCCTAAGCTGGACGAACTGAGGGATGAGGGCAAGGCCAGCTCCG |
| | | CTAAACAGCGCCTGAAGTGTGCTAGCCTGCAGAAATTCGGGGAGCGAGCCTTCAAGGCT |
| | | TGGGCAGTGGCACGGCTGAGTCAGAGATTCCCAAAGGCAGAATTTGCCGAGGTCTCAAA |
| | | ACTGGTGACCGACCTGACAAAGGTGCACACCGAATGCTGTCATGGCGACCTGCTGGAGT |
| | | GCGCCGACGATCGAGCTGATCTGGCAAAGTATATTTGTGAGAACCAGGACTCCATCTCT |
| | | AGTAAGCTGAAAGAATGCTGTGAGAAACCACTGCTGGAAAAGTCTCACTGCATTGCCGA |
| | | AGTGGAGAACGACGAGATGCCAGCTGATCTGCCCTCACTGGCCGCTGACTTCGTCGAAA |
| | | GCAAAGATGTGTGTAAGAATTACGCTGAGGCAAAGGATGTGTTCCTGGGAATGTTTCTG |
| | | TACGAGTATGCCAGGCGCCACCCAGACTACTCCGTGGTCCTGCTGCTGAGGCTGGCTAA |
| | | AACATATGAAACCACACTGGAGAAGTGCTGTGCAGCCGCTGATCCCCATGAATGCTATG |
| | | CCAAAGTCTTCGACGAGTTTAAGCCCCTGGTGGAGGAACCTCAGAACCTGATCAAACAG |
| | | AATTGTGAACTGTTTGAGCAGCTGGGCGAGTACAAGTTCCAGAACGCCCTGCTGGTGCG |
| | | CTATACCAAGAAAGTCCCACAGGTGTCCACACCCACTCTGGTGGAGGTGAGCCGGAATC |
| | | TGGGCAAAGTGGGGAGTAAATGCTGTAAGCACCCTGAAGCCAAGAGGATGCCATGCGCT |
| | | GAGGATTACCTGAGTGTGGTCCTGAATCAGCTGTGTGTCCTGCATGAAAAAACACCTGT |
| | | CAGCGACCGGGTGACAAAGTGCTGTACTGAGTCACTGGTGAACCGACGGCCCTGCTTTA |
| | | GCGCCCTGGAAGTCGATGAGACTTATGTGCCTAAAGAGTTCAACGCTGAGACCTTCACA |
| | | TTTCACGCAGACATTTGTACCCTGAGCGAAAAGGAGAGACAGATCAAGAAACAGACAGC |
| | | CCTGGTCGAACTGGTGAAGCATAAACCCAAGGCCACAAAAGAGCAGCTGAAGGCTGTCA |
| | | TGGACGATTTCGCAGCCTTTGTGGAAAAATGCTGTAAGGCAGACGATAAGGAGACTTGC |
| | | TTTGCCGAGGAAGGAAAGAAACTGGTGGCTGCATCCCAGGCAGCTCTGGGACTGGGAGG |
| | | AGGATCTGCCCCTACCTCAAGCTCCACTAAGAAAACCCAGCTGCAGCTGGAGCACCTGC |
| | | TGCTGGACCTGCAGATGATTCTGAACGGGATCAACAATTACAAAAATCCAAAGCTGACC |
| | | CGGATGCTGACATTCAAGTTTTATATGCCCAAGAAAGCCACAGAGCTGAAACACCTGCA |
| | | GTGCCTGGAGGAAGAGCTGAAGCCTCTGGAAGAGGTGCTGAACCTGGCCCAGAGCAAGA |
| | | ATTTCCATCTGAGACCAAGGGATCTGATCTCCAACATTAATGTGATCGTCCTGGAACTG |
| | | AAGGGATCTGAGACTACCTTTATGTGCGAATACGCTGACGAGACTGCAACCATTGTGGA |
| | | GTTCCTGAACAGATGGATCACCTTCTGCCAGTCCATCATTTCTACTCTGACAGGCGGGG |
| | | GGAGC |
| 295 | EETI-II | GC PRILMR CKQDSDCLAGCVCGPNGFCG |
| | from | |
| | Knottin | |
| | Database | |
| 296 | AgRP from | GCVRLHESCLGQQVPCCDPCATCYCRFFNAFCYCR-KLGTAMNPCSRT |
| | Knottin | |
| | Database | |
| | “-” indicates | |
| | where mini | |
| | protein can | |
| | be formed | |
| 297 | Omega | EDN--CIAEDYGKCTWGGTKCCRGRPCRC SMIGTN CECTPRLIMEGLSFA |
| | agatoxin | |
| | from | |
| | Knottin | |
| | Database | |
| | “-” indicates | |
| | where mini | |
| | protein can | |
| | be formed | |
| 298 | EETI-II | GCXXXRGDXXXXXCKQDSDCLAGCVCGPNGFCG |
| | Library | |
| 299 | EETI-II | GCXXXRGDXXXXXCSQDSDCLAGCVCGPNGFCG |
| | KI5S | |
| | Mutation | |
| | Library | |
| 300 | 2.5F- | GGTTGTCCAAGACCAAGAGGTGATAATCCACCATTGACTTGTTCTCAAGATTCTGATTG |
| | (K15S) | TTTGGCTGGTTGTGTTTGTGGTCCAAATGGTTTTTGTGGTGGTCGACTAGAGCCCAGAG |
| | mIgG2aFc | TGCCCATAACACAGAACCCCTGTCCTCCACTCAAAGAGTGTCCCCCATGCGCAGCTCCA |
| | Nucleic | GACCTCTTGGGTGGACCATCCGTCTTCATCTTCCCTCCAAAGATCAAGGATGTACTCAT |
| | Acid | GATCTCCCTGAGCCCCATGGTCACATGTGTGGTGGTGGATGTGAGCGAGGATGACCCAG |
| | Sequence | ACGTCCAGATCAGCTGGTTTGTGAACAACGTGGAAGTACACACAGCTCAGACACAAACC |
| | | CATAGAGAGGATTACAACAGTACTCTCCGGGTGGTCAGTGCCCTCCCCATCCAGCACCA |
| | | GGACTGGATGAGTGGCAAGGAGTTCAAATGCAAGGTCAACAACAGAGCCCTCCCATCCC |
| | | CCATCGAGAAAACCATCTCAAAACCCAGAGGGCCAGTAAGAGCTCCACAGGTATATGTC |
| | | TTGCCTCCACCAGCAGAAGAGATGACTAAGAAAGAGTTCAGTCTGACCTGCATGATCAC |
| | | AGGCTTCTTACCTGCCGAAATTGCTGTGGACTGGACCAGCAATGGGCGTACAGAGCAAA |
| | | ACTACAAGAACACCGCAACAGTCCTGGACTCTGATGGTTCTTACTTCATGTACAGCAAG |
| | | CTCAGAGTACAAAAGAGCACTTGGGAAAGAGGAAGTCTTTTCGCCTGCTCAGTGGTCCA |
| | | CGAGGGTCTGCACAATCACCTTACGACTAAGACCATCTCCCGGTCTCTGGGTAAA |
| 301 | 2.5F- | GCPRPRGDNPPLTCSQDSDCLAGCVCGPNGFCGEPRVPITQNPCPPLKECPPCAAPDLL |
| | (K15S) | GGPSVFIFPPKIKDVLMISLSPMVTCVVVDVSEDDPDVQISWFVNNVEVHTAQTQTHRE |
| | mIgG2aFc | DYNSTLRVVSALPIQHQDWMSGKEFKCKVNNRALPSPIEKTISKPRGPVRAPQVYVLPP |
| | Amino | PAEEMTKKEFSLTCMITGFLPAEIAVDWTSNGRTEQNYKNTATVLDSDGSYFMYSKLRV |
| | Acid | QKSTWERGSLFACSVVHEGLHNHLTTKTISRSLGK |
| | Sequence | |
| 302 | 2.5D- | GGTTGTCCACAAGGCAGAGGTGATTGGGCTCCAACTTCTTGTTCTCAAGATTCTGATTG |
| | (K15S) | TTTGGCTGGTTGTGTTTGTGGTCCAAATGGTTTTTGTGGTGGTCGACTAGAGCCCAGAG |
| | mIgG2aFc | TGCCCATAACACAGAACCCCTGTCCTCCACTCAAAGAGTGTCCCCCATGCGCAGCTCCA |
| | Nucleic | GACCTCTTGGGTGGACCATCCGTCTTCATCTTCCCTCCAAAGATCAAGGATGTACTCAT |
| | Acid | GATCTCCCTGAGCCCCATGGTCACATGTGTGGTGGTGGATGTGAGCGAGGATGACCCAG |
| | Sequence | ACGTCCAGATCAGCTGGTTTGTGAACAACGTGGAAGTACACACAGCTCAGACACAAACC |
| | | CATAGAGAGGATTACAACAGTACTCTCCGGGTGGTCAGTGCCCTCCCCATCCAGCACCA |
| | | GGACTGGATGAGTGGCAAGGAGTTCAAATGCAAGGTCAACAACAGAGCCCTCCCATCCC |
| | | CCATCGAGAAAACCATCTCAAAACCCAGAGGGCCAGTAAGAGCTCCACAGGTATATGTC |
| | | TTGCCTCCACCAGCAGAAGAGATGACTAAGAAAGAGTTCAGTCTGACCTGCATGATCAC |
| | | AGGCTTCTTACCTGCCGAAATTGCTGTGGACTGGACCAGCAATGGGCGTACAGAGCAAA |
| | | ACTACAAGAACACCGCAACAGTCCTGGACTCTGATGGTTCTTACTTCATGTACAGCAAG |
| | | CTCAGAGTACAAAAGAGCACTTGGGAAAGAGGAAGTCTTTTCGCCTGCTCAGTGGTCCA |
| | | CGAGGGTCTGCACAATCACCTTACGACTAAGACCATCTCCCGGTCTCTGGGTAAA |
| 303 | 2.5D- | GCPQGRGDWAPTSCSQDSDCLAGCVCGPNGFCGEPRVPITQNPCPPLKECPPCAAPDLL |
| | (K15S) | GGPSVFIFPPKIKDVLMISLSPMVTCVVVDVSEDDPDVQISWFVNNVEVHTAQTQTHRE |
| | mIgG2aFc | DYNSTLRVVSALPIQHQDWMSGKEFKCKVNNRALPSPIEKTISKPRGPVRAPQVYVLPP |
| | Amino | PAEEMTKKEFSLTCMITGFLPAEIAVDWTSNGRTEQNYKNTATVLDSDGSYFMYSKLRV |
| | Acid | QKSTWERGSLFACSVVHEGLHNHLTTKTISRSLGK |
| | Sequence | |
| 304 | 2.5F- | GCPRPRGDNPPLTCSQDSDCLAGCVCGPNGFCGEPKSCDKTHTCPPCPAPELLGGPSVF |
| | (K15S) | LFPPKPKDTLMISRTPEVTCVVVDVSHEDPEVKFNWYVDGVEVHNAKTKPREEQYNSTY |
| | hIgG1Fc | RVVSVLTVLHQDWLNGKEYKCKVSNKALPAPIEKTISKAKGQPREPQVYTLPPSREEMT |
| | Amino | KNQVSLTCLVKGFYPSDIAVEWESNGQPENNYKTTPPVLDSDGSFFLYSKLTVDKSRWQ |
| | Acid | QGNVFSCSVMHEALHNHYTQKSLSLSPGK |
| | Sequence | |
| 305 | 2.5F- | GCPRPRGDNPPLTCSQDSDCLAGCVCGPNGFCGDKTHTCPPCPAPELLGGPSVFLFPPK |
| | (K15S) | PKDTLMISRTPEVTCVVVDVSHEDPEVKFNWYVDGVEVHNAKTKPREEQYNSTYRVVSV |
| | hIgG1Fc | LTVLHQDWLNGKEYKCKVSNKALPAPIEKTISKAKGQPREPQVYTLPPSREEMTKNQVS |
| | Fc Upper | LTCLVKGFYPSDIAVEWESNGQPENNYKTTPPVLDSDGSFFLYSKLTVDKSRWQQGNVF |
| | Hinge | SCSVMHEALHNHYTQKSLSLSPGK |
| | Deletion | |
| | (ΔEPKSC) | |
| | Amino Acid | |
| | Sequence | |
| 306 | 2.5D- | GCPQGRGDWAPTSCSQDSDCLAGCVCGPNGFCGEPKSCDKTHTCPPCPAPELLGGPSVF |
| | (K15S) | LFPPKPKDTLMISRTPEVTCVVVDVSHEDPEVKFNWYVDGVEVHNAKTKPREEQYNSTY |
| | hIgG1Fc | RVVSVLTVLHQDWLNGKEYKCKVSNKALPAPIEKTISKAKGQPREPQVYTLPPSREEMT |
| | Amino | KNQVSLTCLVKGFYPSDIAVEWESNGQPENNYKTTPPVLDSDGSFFLYSKLTVDKSRWQ |
| | Acid | QGNVFSCSVMHEALHNHYTQKSLSLSPGK |
| | Sequence | |
| 307 | 2.5D- | GCPQGRGDWAPTSCSQDSDCLAGCVCGPNGFCGDKTHTCPPCPAPELLGGPSVFLFPPK |
| | (K15S) | PKDTLMISRTPEVTCVVVDVSHEDPEVKFNWYVDGVEVHNAKTKPREEQYNSTYRVVSV |
| | hIgG1Fc | LTVLHQDWLNGKEYKCKVSNKALPAPIEKTISKAKGQPREPQVYTLPPSREEMTKNQVS |
| | Fc Upper | LTCLVKGFYPSDIAVEWESNGQPENNYKTTPPVLDSDGSFFLYSKLTVDKSRWQQGNVF |
| | Hinge | SCSVMHEALHNHYTQKSLSLSPGK |
| | Deletion | |
| | (ΔEPKSC) | |
| | Amino | |
| | Acid | |
| | Sequence | |
| 308 | hPD-1 | MQIPQAPWPVVWAVLQLGWRPGWFLDSPDPWNPPTFFPALLVVTEGDNATFTCSFSNTS |
| | amino acid | ESFVLNWYRMSPSNQTDKLAAFPEDRSQPGQDCRFRVTQLPNGRDFHMSVVRARRNDSG |
| | sequence | TYLCGAISLAPKAQIKESLRAELRVTERRAEVPTAHPSPSPRPAGQFQTLVVGVVGGLL |
| | | GSLVLLVWVLAVICSRAARGTIGARRTGQPLKEDPSAVPVFSVDYGELDFQWREKTPEP |
| | | PVPCVPEQTEYATIVFPSGMGTSSPARRGSADGPRSAQPLRPEDGHCSWPL |
| 309 | hPD-L1 | MRIFAVFIFMTYWHLLNAFTVTVPKDLYVVEYGSNMTIECKFPVEKQLDLAALIVYWEM |
| | amino acid | EDKNIIQFVHGEEDLKVQHSSYRQRARLLKDQLSLGNAALQITDVKLQDAGVYRCMISY |
| | sequence | GGADYKRITVKVNAPYNKINQRILVVDPVTSEHELTCQAEGYPKAEVIWTSSDHQVLSG |
| | | KTTTTNSKREEKLFNVTSTLRINTTTNEIFYCTFRRLDPEENHTAELVIPELPLAHPPN |
| | | ERTHLVILGAILLCLGVALTFIFRLRKGRMMDVKKCGIQDTNSKKQSDTHLEET |
| 310 | hCTLA-4 | MACLGFQRHKAQLNLATRTWPCTLLFFLLFIPVFCKAMHVAQPAVVLASS |
| | amino acid | RGIASFVCEYASPGKATEVRVTVLRQADSQVTEVCAATYM |
| | sequence | MGNELTFLDDSICTGTSSGNQVNLTIQGLRAMDTGLYICKVELMYPPPYY |
| | | LGIGNGTQIY VIDPEPCPDS DFLLWILAAVSSGLFFYSFL |
| | | LTAVSLSKML KKRSPLTTGVYVKMPPTEPE CEKQFQPYFI PIN |
| 311 | hLAG-3 | MWEAQFLGLLFLQPLWVAPVKPLQPGAEVPVVWAQEGAPAQLPCSPTIPLQDLSLLRRA |
| | amino acid | GVTWQHQPDSGPPAAAPGHPLAPGPHPAAPSSWGPRPRRYTVLSVGPGGLRSGRLPLQP |
| | sequence | RVQLDERGRQRGDFSLWLRPAR |
| | | RADAGEYRAAVHLRDRALSCRLRLRLGQASMTASPPGSLR |
| | | ASDWVILNCSFSRPDRPASVHWFRNRGQGRVPVRESPHHHLAESFLFLPQVSPMDSGPW |
| | | GCILTYRDGFNVSIMYNLTVLGLEPPTPLTVYAGAGSRVGLPCRLPAGVGTRSFLTAKW |
| | | TPPGGGPDLLVTGDNGDFTLRL EDVSQAQAGT YTCHIHLQEQ QLNATVTLAI |
| | | ITVTPKSFGS |
| | | PGSLGKLLCEVTPVSGQERFVWSSLDTPSQRSFSGPWLEAQEAQLLSQPWQCQLYQGER |
| | | LLGAAVYFTEL SSPGAQRSGR APGALPAGHL LLFLILGVLS LLLLVTGAFG |
| | | FHLWRRQWRPRRFSALEQGIHPPQAQSKIEELEQEPEPEPEPEPEPEPEPEPEQL |
| 312 | hTIM-3 | MFSHLPFDCVLLLLLLLLTRSSEVEYRAEVGQNAYLPCFYTPAAPGNLVPVCWGKGACP |
| | amino acid | VFECGNVVLRTDERDVNYWTSRYWLNGDFRKGDVSLTIENVTLADSGIYCCRIQIPGIM |
| | sequence | NDEKFNLKLVIKPAKVTPAPTR QRDFTAAFPR MLTTRGHGPA ETQTLGSLPD |
| | | INLTQISTLA |
| | | NELRDSRLANDLRDSGATIRGIYIGAGICAGLALALIFGALIFKWYSHSKEKIQNLSLI |
| | | SLANLPPSGLANAVAEGIRSEENIYTIEENVYEVEEPNEYYCYVSSRQQPSQPLGCRFA |
| | | MP |
| 313 | hB7-H3 | MLRRRGSPGMGVHVGAALGALWFCLTGALEVQVPEDPVVALVGTDATLCC |
| | amino acid | SFSPEPGFSLQLNLIWQLT DTKQLVHSFA EGQDQGSAYA |
| | sequence | NRTALFPDLLAQGNASLRLQRVRVADEGSFCFVSIRDFGSAAVSLQVAA |
| | | PYSKPSMTLE PNKDLRPGDT VTITCSSYQG YPEAEVFWQD |
| | | GQGVPLTGNVTTSQMANEQGLFDVHSILRVVLGANGTYSCLVRNPVLQQD |
| | | AHSSVTITPQRSPTGAVEVQVPEDPVVALVGTDATLRCSF |
| | | SPEPGFSLAQLNLIWQLTDTKQLVHSFTEGRDQGSAYANRTALFPDLLAQ |
| | | GNASLRLQRV RVADEGSFTC FVSIRDFGSA AVSLQVAAPY |
| | | SKPSMTLEPNKDLRPGDTVTITCSSYRGYPEAEVFWQDGQGVPLTGNVTT |
| | | SQMANEQGLFDVHSVLRVVLGANGTYSCLVRNPVLQQDAH |
| | | GSVTITGQPMTFPPEALWVTVGLSVCLIALLVALAFVCWRKIKQSCEEEN |
| | | AGAEDQDGEGEGSKTALQPLKHSDSKEDDGQEIA |
| 314 | hB7-H4 | MASLGQILFWSIISIIIILAGAIALIIGFGISAFSMPEVNVDYNASSETLRCEAPRWFP |
| | amino acid | QPTVVWASQVDQGANFSEVSNTSFELNSENVTMKVVSVLYN VTINNTYSCM |
| | sequence | IENDIAKATGDIKVTESEIKRRSHLQLLNS KASLCVSSFFAISWALLPLSPYLMLK |

In one embodiment, an integrin-binding polypeptide or a variant thereof, consists of, consists essentially of, or comprises an amino acid sequence selected from SEQ ID NOs: 59-127. In an embodiment, a polypeptide includes an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence selected from SEQ ID Nos: 59-127. In an embodiment, a polypeptide includes a contiguous amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to a contiguous amino acid sequence selected from SEQ ID Nos: 59-127. In an embodiment, a polypeptide includes an amino acid sequence having at least 10, 15, 20, 25, 30, 35, 40, 45, 50, 55, 60, 65, 70, 75, 80, 85, 90, 95, 100, 200, 300, 400, or 500 (or any integer within these numbers) contiguous amino acids of an amino acid sequence selected from SEQ ID NOs: 59-127.

TABLE 2
Integrin Binding Knottin Sequences

| SEQ ID | Peptide | | Sequence (RGD motif is underlined |
|---|---|---|---|
| NO: | Identifier | Scaffold | with flanking residues) |
| 315 | 1.4A | EETI-II | GCAEPRGDMPWTWCKQDSDCLAGCVCGPNGFCG |
| 316 | 1.4B | EETI-II | GCVGGRGDWSPKWCKQDSDCPAGCVCGPNGFCG |
| 317 | 1.4C | EETI-II | GC AELRGDRSYPE CKQDSDCLAGCVCGPNGFCG |
| 318 | 1.4E | EETI-II | GC RLPRGDVPRPH CKQDSDCQAGCVCGPNGFCG |
| 319 | 1.4H | EETI-II | GC YPLRGDNPYAA CKQDSDCRAGCVCGPNGFCG |
| 320 | 1.5B | EETI-II | GC TIGRGDWAPSE CKQDSDCLAGCVCGPNGFCG |
| 321 | 1.5F | EETI-II | GC HPPRGDNPPVT CKQDSDCLAGCVCGPNGFCG |
| 322 | 2.3A | EETI-II | GC PEPRGDNPPPS CKQDSDCRAGCVCGPNGFCG |
| 323 | 2.3B | EETI-II | GC LPPRGDNPPPS CKQDSDCQAGCVCGPNGFCG |
| 324 | 2.3C | EETI-II | GCHLGRGDWAPVGCKQDSDCPAGCVCGPNGFCG |
| 325 | 2.3D | EETI-II | GC NVGRGDWAPSECKQDSDCPAGCVCGPNGFCG |
| 326 | 2.3E | EETI-II | GC FPGRGDWAPSSCKQDSDCRAGCVCGPNGFCG |
| 327 | 2.3F | EETI-II | GC PLPRGDNPPTE CKQDSDCQAGCVCGPNGFCG |
| 328 | 2.3G | EETI-II | GC SEARGDNPRLS CKQDSDCRAGCVCGPNGFCG |
| 329 | 2.3H | EETI-II | GCLLGRGDWAPEACKQDSDCRAGCVCPNGFCG |
| 330 | 2.3I | EETI-II | GCHVGRGDWAPLKCKQDSDCQAGCVCGPNGFCG |
| 331 | 2.3J | EETI-II | GC VRGRGDWAPPSCKQDSDCPAGCVCGPNGFCG |
| 332 | 2.4A | EETI-II | GCLGGRGDWAPPACKQDSDCRAGCVCGPNGFCG |
| 333 | 2.4C | EETI-II | GC FVGRGDWAPLTCKQDSDCQAGCVCGPNGFCG |
| 334 | 2.4D | EETI-II | GC PVGRGDWSPASCKQDSDCRAGCVCGPNGFCG |
| 335 | 2.4E | EETI-II | GC PRPRGDNPPLT CKQDSDCLAGCVCGPNGFCG |
| 336 | 2.4F | EETI-II | GC YQGRGDWSPSSCKQDSDCPAGCVCGPNGFCG |
| 337 | 2.4G | EETI-II | GC APGRGDWAPSECKQDSDCQAGCVCGPNGFCG |
| 338 | 2.4J | EETI-II | GC VQGRGDWSPPSCKQDSDCPAGCVCGPNGFCG |
| 339 | 2.5A | EETI-II | GC HVGRGDWAPEECKQDSDCQAGCVCGPNGFCG |
| 340 | 2.5C | EETI-II | GC DGGRGDWAPPACKQDSDCRAGCVCGPNGFCG |
| 341 | 2.5D | EETI-II | GC PQGRGDWAPTSCKQDSDCRAGCVCGPNGFCG |
| 342 | 2.5F | EETI-II | GC PRPRGDNPPLT CKQDSDCLAGCVCGPNGFCG |
| 343 | 2.5D K15S | EETI-II | GCPQGRGDWAPTSCSQDSDCLAGCVCGPNGFCG |
| | Mutant | | |
| 344 | 2.5F K15S | EETI-II | GCPRPRGDNPPLTCSQDSDCLAGCVCGPNGFCG |
| | Mutant | | |
| 345 | 2.5H | EETI-II | GCPQGRGDWAPEWCKQDSDCPAGCVCGPNGFCG |
| 346 | 2.5J | EETI-II | GCPRGRGDWSPPACKQDSDCQAGCVCGPNGFCG |
| 347 | 3A | AgRp | GCVRLHESCLGQQVPCCDPAATCYCVVRGDWRKRC |
| | | | YCR |
| 348 | 3B | AgRp | GCVRLHESCLGQQVPCCDPAATCYC |
| | | | EERGDMLEKCYCR |
| 349 | 3C | AgRp | GCVRLHESCLGQQVPCCDPAATCYC |
| | | | ETRGDGKEKCYCR |
| 350 | 3D | AgRp | GCVRLHESCLGQQVPCCDPAATCYCQWRGDGDVKC |
| | | | YCR |
| 351 | 3E | AgRp | GCVRLHESCLGQQVPCCDPAATCYCSRRGDMRERC |
| | | | YCR |
| 352 | 3F | AgRp | GCVRLHESCLGQQVPCCDPAATCYCQYRGDGMKHC |
| | | | YCR |
| 353 | 3G | AgRp | GCVRLHESCLGQQVPCCDPAATCYC |
| | | | TGRGDTKVLCYCR |
| 354 | 3H | AgRp | GCVRLHESCLGQQVPCCDPAATCYCVERGDMKRRC |
| | | | YCR |
| 355 | 3I | AgRp | GCVRLHESCLGQQVPCCDPAATCYC |
| | | | TGRGDVRMNCYCR |
| 356 | 3J | AgRp | GCVRLHESCLGQQVPCCDPAATCYCVERGDGMSKC |
| | | | YCR |
| 357 | 4A | AgRp | GCVRLHESCLGQQVPCCDPAATCYCRGRGDMRREC |
| | | | YCR |
| 358 | 4B | AgRp | GCVRLHESCLGQQVPCCDPAATCYC |
| | | | EGRGDVKVNCYCR |
| 359 | 4C | AgRp | GCVRLHESCLGQQVPCCDPAATCYCVGRGDEKMSC |
| | | | YCR |
| 360 | 4D | AgRp | GCVRLHESCLGQQVPCCDPAATCYCVSRGDMRKRC |
| | | | YCR |
| 361 | 4E | AgRp | GCVRLHESCLGQQVPCCDPAATCYC |
| | | | ERRGDSVKKCYCR |
| 362 | 4F | AgRp | GCVRLHESCLGQQVPCCDPAATCYC |
| | | | EGRGDTRRRCYCR |
| 363 | 4G | AgRp | GCVRLHESCLGQQVPCCDPAATCYC |
| | | | EGRGDVVRRCYCR |
| 364 | 4H | AgRp | GCVRLHESCLGQQVPCCDPAATCYCKGRGDNKRKC |
| | | | YCR |
| 365 | 4I | AgRp | GCVRLHESCLGQQVPCCDPAXTCYC |
| | | | KGRGDVRRVCYCR |
| 366 | 4J | AgRp | GCVRLHESCLGQQVPCCDPAATCYC |
| | | | VGRGDNKVKCYCR |
| 367 | 5A | AgRp | GCVRLHESCLGQQVPCCDPAATCYC |
| | | | VGRGDNRLKCYCR |
| 368 | 5B | AgRp | GCVRLHESCLGQQVPCCDPAATCYCVERGDGMKKC |
| | | | YCR |
| 369 | 5C | AgRp | GCVRLHESCLGQQVPCCDPAATCYCEGRGDMRRRC |
| | | | YCR |
| 370 | 5D | AgRp | GCVRLHESCLGQQVPCCDPAATCYCQGRGDGDVKC |
| | | | YCR |
| 371 | 5E | AgRp | GCVRLHESCLGQQVPCCDPAATCYC |
| | | | SGRGDNDLVCYCR |
| 372 | 5F | AgRp | GCVRLHESCLGQQVPCCDPAATCYC |
| | | | VERGDGMIRCYCR |
| 373 | 5G | AgRp | GCVRLHESCLGQQVPCCDPAATCYC |
| | | | SGRGDNDLVCYCR |
| 374 | 5H | AgRp | GCVRLHESCLGQQVPCCDPAATCYCEGRGDMKMKC |
| | | | YCR |
| 375 | 5I | AgRp | GCVRLHESCLGQQVPCCDPAATCYC |
| | | | IGRGDVRRRCYCR |
| 376 | 5J | AgRp | GCVRLHESCLGQQVPCCDPAATCYC |
| | | | EERGDGRKKCYCR |
| 377 | 6B | AgRp | GCVRLHESCLGQQVPCCDPAATCYCEGRGDRDMKC |
| | | | YCR |
| 378 | 6C | AgRp | GCVRLHESCLGQQVPCCDPAATCYC |
| | | | TGRGDEKLRCYCR |
| 379 | 6E | AgRp | GCVRLHESCLGQQVPCCDPAATCYC |
| | | | VERGDGNRRCYCR |
| 380 | 6F | AgRp | GCVRLHESCLGQQVPCCDPAATCYC |
| | | | ESRGDVVRKCYCR |
| 381 | 7C | AgRp | GCVRLHESCLGQQVPCCDPAATCYCYGRGDNDLRC |
| | | | YCR |

TABLE 3
Integrin Binding Polypeptide Sequences, Signal Sequences, Linkers,
Fc fusions

| | Peptide | |
|---|---|---|
| SEQ ID | Identifier | |
| NO: | Scaffold | Sequence |
| 382 | NOD201 - 2.5F | GCPRPRGDNPPLTCSQDSDCLAGCVCGPNGFCG |
| 383 | NOD201modK - | GCPRPRGDNPPLTCKQDSDCLAGCVCGPNGFCG |
| | 2.5FmodK | |
| 384 | N0D203 - 2.5F | GCPRPRGDNPPLTCSQDSDCLAGCVCGPNGFCGGGGGS |
| | w/GGGGS | |
| 385 | NOD203modK - | GCPRPRGDNPPLTCKQDSDCLAGCVCGPNGFCGGGGGS |
| | 2.5FmodK | |
| | w/GGGGS | |
| 386 | N0D204 - 2.5F | GCPRPRGDNPPLTCSQDSDCLAGCVCGPNGFCGGGGGSGGGGSGGGGS |
| | w/GGGGSGGGGSGG | |
| | GGS | |
| 387 | NOD204modK - | GCPRPRGDNPPLTCKQDSDCLAGCVCGPNGFCGGGGGSGGGGSGGGGS |
| | 2.5FmodK w/ | |
| | GGGGSGGGGSGGGG | |
| | S | |
| 388 | Linker (short) | GGGGS |
| | (linker for | |
| | use with any | |
| | sequnces | |
| | disclosed | |
| | herein) | |
| 389 | Linker (long) | GGGGSGGGGSGGGGS |
| | (linker for | |
| | use with any | |
| | sequnces | |
| | disclosed | |
| | herein) | |
| 390 | Signal | MTRLTVLALLAGLLASSR |
| | sequence | |
| | (signal | |
| | peptide A) | |
| | (signal | |
| | peptide for | |
| | use with any | |
| | sequnces | |
| | disclosed | |
| | herein, | |
| | including SEQ | |
| | ID Nos: 139, | |
| | 140, 141, 142, | |
| | and 143) | |
| 391 | NOD201 (human | GCPRPRGDNPPLTCSQDSDCLAGCVCGPNGFCGEPKSSDKTHTCPPCPA |
| | Fc; no linker) | PELLGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNKALP |
| | | APIEKTISKAKGQPREPQVYTLPPSRDELTKNQVSLTCLVKGFYPSDIA |
| | | VEWESNGQPENNYKTTPPVLDSDGSFFLYSKLTVDKSRWQQGNVFSCSV |
| | | MHEALHNHYTQKSLSLSPG |
| 392 | NOD201X | GCVTGRDGSPASSCSQDSDCLAGCVCGPNGFCGEPKSSDKTHTCPPCPA |
| | (control | PELLGGPSVFLFPPKPKDTLMISRTPEVTCVVVDVSHEDPEVKFNWYVD |
| | sequence - | GVEVHNAKTKPREEQYNSTYRVVSVLTVLHQDWLNGKEYKCKVSNKALP |
| | NOD201 with | APIEKTISKAKGQPREPQVYTLPPSRDELTKNQVSLTCLVKGFYPSDIA |
| | scrambled seq, | VEWESNGQPENNYKTTPPVLDSDGSFFLYSKLTVDKSRWQQGNVFSCSV |
| | human Fc; no | MHEALHNHYTQKSLSLSPG |
| | linker) | |
| | Theoretical | |
| | pI/Mw: 6.19/ | |
| | 58065.44 | |
| 393 | NOD201M | GCPRPRGDNPPLTCSQDSDCLAGCVCGPNGFCGEPRVPITQNPCPPLKE |
| | (NOD201 with | CPPCAAPDLLGGPSVFIFPPKIKDVLMISLSPMVTCVVVDVSEDDPDVQ |
| | murine Fc | ISWFVNNVEVHTAQTQTHREDYNSTLRVVSALPIQHQDWMSGKEFKCKV |
| | domain; no | NNRALPSPIEKTISKPRGPVRAPQVYVLPPPAEEMTKKEFSLTCMITGF |
| | linker) | LPAEIAVDWTSNGRTEQNYKNTATVLDSDGSYFMYSKLRVQKSTWERGS |
| | Theoretical | LFACSVVHEGLHNHLTTKTISRSLG |
| | pI/Mw: 6.34/ | |
| | 59357.92 | |
| | Ext. | |
| | coefficient | |
| | 60525 | |
| | Abs 0.1% (=1 | |
| | g/l) 1.020, | |
| | assuming all | |
| | pairs of Cys | |
| | residues form | |
| | cystines | |
| 394 | NOD203 | GCPRPRGDNPPLTCSQDSDCLAGCVCGPNGFCGGGGGSEPKSSDKTHTC |
| | complete | PPCPAPELLGGPSVFLFPPKPKDTLMISRTPEVTCVVVDVSHEDPEVKF |
| | (Gly₄Ser | NWYVDGVEVHNAKTKPREEQYNSTYRVVSVLTVLHQDWLNGKEYKCKVS |
| | linker) | NKALPAPIEKTISKAKGQPREPQVYTLPPSRDELTKNQVSLTCLVKGFY |
| | | PSDIAVEWESNGQPENNYKTTPPVLDSDGSFFLYSKLTVDKSRWQQGNV |
| | | FSCSVMHEALHNHYTQKSLSLSPG |
| 395 | NOD204 | GCPRPRGDNPPLTCSQDSDCLAGCVCGPNGFCGGGGGSGGGGSGGGGSE |
| | complete | PKSSDKTHTCPPCPAPELLGGPSVFLFPPKPKDTLMISRTPEVTCVVVD |
| | ([Gly₄Ser]₃ | VSHEDPEVKFNWYVDGVEVHNAKTKPREEQYNSTYRVVSVLTVLHQDWL |
| | linker) | NGKEYKCKVSNKALPAPIEKTISKAKGQPREPQVYTLPPSRDELTKNQV |
| | | SLTCLVKGFYPSDIAVEWESNGQPENNYKTTPPVLDSDGSFFLYSKLTV |
| | | DKSRWQQGNVFSCSVMHEALHNHYTQKSLSLSPG |

TABLE 4
Exemplary IgG sequences:

| SEQ | | | |
|---|---|---|---|
| ID | | | |
| NO: | Name | Sequence | |
| 396 | IgG1 | ASTKGPSVFP LAPSSKSTSG GTAALGCLVK DYFPEPVTVS WNSGALTSGV HTFPAVLQSS | 60 |
| | | GLYSLSSVVT VPSSSLGTQT YICNVNHKPS NTKVDKKVEP KSCDKTHTCP PCPAPELLGG | 120 |
| | | PSVFLFPPKP KDTLMISRTP EVTCVVVDVS HEDPEVKFNW YVDGVEVHNA KTKPREEQYN | 180 |
| | | STYRVVSVLT VLHQDWLNGK EYKCKVSNKA LPAPIEKTIS KAKGQPREPQ VYTLPPSRDE | 240 |
| | | LTKNQVSLTC LVKGFYPSDI AVEWESNGQP ENNYKTTPPV LDSDGSFFLY SKLTVDKSRW | 300 |
| | | QQGNVFSCSV MHEALHNHYT QKSLSLSPGK | 330 |
| 397 | IgG2 | ASTKGPSVFP LAPCSRSTSE STAALGCLVK DYFPEPVTVS WNSGALTSGV HTFPAVLQSS | 60 |
| | | GLYSLSSVVT VPSSNFGTQT YTCNVDHKPS NTKVDKTVER KCCVECPPCP APPVAGPSVF | 120 |
| | | LFPPKPKDTL MISRTPEVTC VVVDVSHEDP EVQFNWYVDG VEVHNAKTKP REEQFNSTFR | 180 |
| | | VVSVLTVVHQ DWLNGKEYKC KVSNKGLPAP IEKTISKTKG QPREPQVYTL PPSREEMTKN | 240 |
| | | QVSLTCLVKG FYPSDIAVEW ESNGQPENNY KTTPPMLDSD GSFFLYSKLT VDKSRWQQGN | 300 |
| | | VFSCSVMHEA LHNHYTQKSL SLSPGK | 326 |
| 398 | IgG3 | ASTKGPSVFP LAPCSRSTSG GTAALGCLVK DYFPEPVTVS WNSGALTSGV HTFPAVLQSS | 60 |
| | | GLYSLSSVVT VPSSSLGTQT YTCNVNHKPS NTKVDKRVEL KTPLGDTTHT CPRCPEPKSC | 120 |
| | | DTPPPCPRCP EPKSCDTPPP CPRCPEPKSC DTPPPCPRCP APELLGGPSV FLFPPKPKDT | 180 |
| | | LMISRTPEVT CVVVDVSHED PEVQFKWYVD GVEVHNAKTK PREEQYNSTF RVVSVLTVLH | 240 |
| | | QDWLNGKEYK CKVSNKALPA PIEKTISKTK GQPREPQVYT LPPSREEMTK NQVSLTCLVK | 300 |
| | | GFYPSDIAVE WESSGQPENN YNTTPPMLDS DGSFFLYSKL TVDKSRWQQG NIFSCSVMHE | 360 |
| | | ALHNRFTQKS LSLSPGK | 377 |
| 399 | IgG4 | ASTKGPSVFP LAPCSRSTSE STAALGCLVK DYFPEPVTVS WNSGALTSGV HTFPAVLQSS | 60 |
| | | GLYSLSSVVT VPSSSLGTKT YTCNVDHKPS NTKVDKRVES KYGPPCPSCP APEFLGGPSV | 120 |
| | | FLFPPKPKDT LMISRTPEVT CVVVDVSQED PEVQFNWYVD GVEVHNAKTK PREEQFNSTY | 180 |
| | | RVVSVLTVLH QDWLNGKEYK CKVSNKGLPS SIEKTISKAK GQPREPQVYT LPPSQEEMTK | 240 |
| | | NQVSLTCLVK GFYPSDIAVE WESNGQPENN YKTTPPVLDS DGSFFLYSRL TVDKSRWQEG | 300 |
| | | NVFSCSVMHE ALHNHYTQKS LSLSLGK | 327 |

It will also be understood by one of ordinary skill in the art that the polypeptides, including the integrin-binding polypeptide-Fc fusions, used herein may be altered such that they vary in sequence from the naturally occurring or native sequences from which they were derived, while retaining the desirable activity of the native sequences. For example, nucleotide or amino acid substitutions leading to conservative substitutions or changes at “non-essential” amino acid residues may be made. Mutations may be introduced by standard techniques, such as site-directed mutagenesis and PCR-mediated mutagenesis.

The polypeptides described herein (e.g., knottin, Fc, knottin-Fc, integrin-binding polypeptide-Fc fusion, and the like) may comprise conservative amino acid substitutions at one or more amino acid residues, e.g., at essential or non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagines, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a binding polypeptide is preferably replaced with another amino acid residue from the same side chain family. In another embodiment, a string of amino acids can be replaced with a structurally similar string that differs in order and/or composition of side chain family members. Alternatively, in another embodiment, mutations may be introduced randomly along all or part of a coding sequence, such as by saturation mutagenesis, and the resultant mutants can be incorporated into binding polypeptides of the invention and screened for their ability to bind to the desired target.

The “Programmed Death-1 (PD-1)” receptor refers to an immuno-inhibitory receptor belonging to the CD28 family. PD-1 is expressed predominantly on previously activated T-cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. AAC51773 (SEQ ID NO: 52 from International Publication No. WO 2016/025642).

“Programmed Death Ligand-1 (PD-L1)” is one of two cell surface glycoprotein ligands for PD-1 (the other being PD-L2) that downregulates T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7 (SEQ ID NO: 53 from International Publication No. WO 2016/025642).

“Cytotoxic T Lymphocyte Associated Antigen-4 (CTLA-4)” is a T cell surface molecule and is a member of the immunoglobulin superfamily. This protein downregulates the immune system by binding to CD80 and CD86. The term “CTLA-4” as used herein includes human CTLA-4 (hCTLA-4), variants, isoforms, and species homologs of hCTLA-4, and analogs having at least one common epitope with hCTLA-4. The complete hCTLA-4 sequence can be found under GenBank Accession No. P16410 (SEQ ID NO: 54 from International Publication No. WO 2016/025642):

“Lymphocyte Activation Gene-3 (LAG-3)” is an inhibitory receptor associated with inhibition of lymphocyte activity by binding to MHC class II molecules. This receptor enhances the function of Treg cells and inhibits CD8+ effector T cell function. The term “LAG-3” as used herein includes human LAG-3 (hLAG-3), variants, isoforms, and species homologs of hLAG-3, and analogs having at least one common epitope. The complete hLAG-3 sequence can be found under GenBank Accession No. P18627 (SEQ ID NO: 55 from International Publication No. WO 2016/025642).

“T-Cell Membrane Protein-3 (TIM-3)” is an inhibitory receptor involved in the inhibition of lymphocyte activity by inhibition of T-cell and B-cell responses. Its ligand is galectin 9, which is upregulated in various types of cancers. The term “TIM-3” as used herein includes human TIM-3 (hTIM-3), variants, isoforms, and species homologs of hTIM-3, and analogs having at least one common epitope. The complete hTIM-3 sequence can be found under GenBank Accession No. Q8TDQ0 (SEQ ID NO: 56 from International Publication No. WO 2016/025642).

The “B7 family” refers to inhibitory ligands with undefined receptors. The B7 family encompasses B7-H3 and B7-H4, both upregulated on tumor cells and tumor infiltrating cells. The complete hB7-H3 and hB7-H4 sequence can be found under GenBank Accession Nos. Q5ZPR3 and AAZ17406 (SEQ ID NOs: 57 and 58 from International Publication No. WO 2016/025642) respectively.

“Vascular Endothelial Growth Factor (VEGF)” is a secreted disulfide-linked homodimer that selectively stimulates endothelial cells to proliferate, migrate, and produce matrix-degrading enzymes, all of which are processes required for the formation of new vessels. In addition to being the only known endothelial cell specific mitogen, VEGF is unique among angiogenic growth factors in its ability to induce a transient increase in blood vessel permeability to macromolecules. The term “VEGF” or “VEGF-A” is used to refer to the 165-amino acid human vascular endothelial cell growth factor and related 121-, 145-, 189-, and 206-amino acid human vascular endothelial cell growth factors, as described by, e.g., Leung et al. Science, 246: 1306 (1989), and Houck et al. Mol. Endocrin., 5: 1806 (1991), together with the naturally occurring allelic and processed forms thereof. VEGF-A is part of a gene family including VEGF-B, VEGF-C, VEGF-D, VEGF-E, VEGF-F, and P1GF. VEGF-A primarily binds to two high affinity receptor tyrosine kinases, VEGFR-1 (Fit-1) and VEGFR-2 (Flk-1 KDR), the latter being the major transmitter of vascular endothelial cell mitogenic signals of VEGF-A.

“T-cell immunoreceptor with Ig and ITIM domains (TIGIT)”, is an immune receptor found on T-cells and natural killer cells (NK cells), as described by Yu X, et al., Nat Immunol. 10 (1): 48-57 (2009). It is also referred to as WUCAM and Vstm3. TIGIT binds to CD155(PVR) with high affinity on, for example, dendritic cells (DCs) and macrophages. TIGIT also binds to CD112(PVRL2), but with lower affinity. See, also, Anderson, A., et al., Immunity, 44(5):989-1004 (2016). The human TIGIT sequence can be found on UniProtKB under accession number Q495A1.

As used herein, “immune checkpoint” refers to stimulatory and inhibitory signals that regulate the amplitude and quality of T cell receptor recognition of an antigen. In certain embodiments, the immune checkpoint is an inhibitory signal. In certain embodiments, the inhibitory signal is the interaction between PD-1 and PD-L1. In certain embodiments, the inhibitory signal is the interaction between CTLA-4 and CD80 or CD86 to displace CD28 binding. In certain embodiments the inhibitory signal is the interaction between LAG-3 and MHC class II molecules. In certain embodiments, the inhibitory signal is the interaction between TIM-3 and galectin 9. In certain embodiments, the inhibitory signal is the interaction between TIGIT and CD155.

As used herein, “immune checkpoint blocker” or “immune checkpoint inhibitor” or “immune checkpoint modulator” refers to a molecule that reduces, inhibits, interferes with or modulates one or more checkpoint proteins or other proteins in the immune system pathways. In certain embodiments, the immune checkpoint inhibitor prevents inhibitory signals associated with the immune checkpoint. In certain embodiments, the immune checkpoint inhibitor is an antibody, or fragment thereof, that disrupts inhibitory signaling associated with the immune checkpoint. In certain embodiments, the immune checkpoint inhibitor is a small molecule that disrupts inhibitory signaling. In certain embodiments, the immune checkpoint inhibitor is an antibody, fragment thereof, or antibody mimic, that prevents the interaction between checkpoint blocker proteins, e.g., an antibody, or fragment thereof, that prevents the interaction between PD-1 and PD-L1. In certain embodiments, the immune checkpoint inhibitor is an antibody, or fragment thereof, that prevents the interaction between CTLA-4 and CD80 or CD86. In certain embodiments, the immune checkpoint inhibitor is an antibody, or fragment thereof, that prevents the interaction between LAG-3 and MHC class II molecules. In certain embodiments the, the immune checkpoint inhibitor is an antibody, or fragment thereof, that prevents the interaction between TIM-3 and galectin9. The checkpoint blocker may also be in the form of the soluble form of the molecules (or mutation thereof) themselves, e.g., a soluble PD-L1 or PD-L1 fusion, as well as a soluble TIGIT or TIGIT fusion.

The term “ameliorating” refers to any therapeutically beneficial result in the treatment of a disease state, e.g., cancer, including prophylaxis, lessening in the severity or progression, remission, or cure thereof.

The term “in vivo” refers to processes that occur in a living organism.

The term “mammal” or “subject” or “patient” as used herein includes both humans and non-humans and include but is not limited to humans, non-human primates, canines, felines, murines, bovines, equines, and porcines.

By “individual” or “host” or “subject” or “patient” is meant any mammalian subject for whom diagnosis, treatment, or therapy is desired, particularly humans. Other subjects may include cynomolgus monkey, cattle, dogs, cats, guinea pigs, rabbits, rats, mice, horses, and so on.

As used herein, the term “mammal” refers to any mammal, including, but not limited to, mammals of the order Rodentia, such as mice and hamsters, and mammals of the order Logomorpha, such as rabbits. In some embodiments, the mammals are from the order Carnivora, including felines (cats) and canines (dogs). In some embodiments, the mammals are from the order Artiodactyla, including bovines (cows) and swines (pigs) or of the order Perssodactyla, including Equines (horses). It is most preferred that the mammals are of the order Primates, Ceboids, or Simoids (monkeys) or of the order Anthropoids (humans and apes). In some embodiments, the mammal is a human. In some embodiments, the mammal is cynomolgus monkey.

The term “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FAST A, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al, J. Mol. Biol. 215:403-410 (1990). Software for performing BLAST analyses is publicly available through the National Center for Biotechnology Information website.

As used herein, the term “gly-ser polypeptide linker” refers to a peptide that consists of glycine and serine residues. An exemplary gly-ser polypeptide linker comprises the amino acid sequence Ser(Gly₄Ser)n. In one embodiment, n=1. In one embodiment, n=2. In another embodiment, n=3, i.e., Ser(Gly₄Ser)3. In another embodiment, n=4, i.e., Ser(Gly₄Ser)4. In another embodiment, n=5. In yet another embodiment, n=6. In another embodiment, n=7. In yet another embodiment, n=8. In another embodiment, n=9. In yet another embodiment, n=10. Another exemplary gly-ser polypeptide linker comprises the amino acid sequence (Gly₄Ser)n. In one embodiment, n=1. In one embodiment, n=2. In a preferred embodiment, n=3. In another embodiment, n=4. In another embodiment, n=5. In yet another embodiment, n=6. Another exemplary gly-ser polypeptide linker comprises the amino acid sequence (Gly₃Ser)n. In one embodiment, n=1. In one embodiment, n=2. In a preferred embodiment, n=3. In another embodiment, n=4. In another embodiment, n=5. In yet another embodiment, n=6.

As used herein, “half-life” refers to the time taken for the serum or plasma concentration of a polypeptide to reduce by 50%, in vivo, for example due to degradation and/or clearance or sequestration by natural mechanisms. The extended-PK IL-2 used herein is stabilized in vivo and its half-life increased by, e.g., fusion to HSA, MSA or Fc, through PEGylation, or by binding to serum albumin molecules (e.g., human serum albumin) which resist degradation and/or clearance or sequestration. The half-life can be determined in any manner known per se, such as by pharmacokinetic analysis. Suitable techniques will be clear to the person skilled in the art, and may for example generally involve the steps of suitably administering a suitable dose of the amino acid sequence or compound of the invention to a subject; collecting blood samples or other samples from said subject at regular intervals; determining the level or concentration of the amino acid sequence or compound of the invention in said blood sample; and calculating, from (a plot of) the data thus obtained, the time until the level or concentration of the amino acid sequence or compound of the invention has been reduced by 50% compared to the initial level upon dosing. Further details are provided in, e.g., standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, 2^{nd} Rev. Edition, Marcel Dekker (1982).

As used herein, a “small molecule” is a molecule with a molecular weight below about 500 Daltons.

As used herein, “therapeutic protein” refers to any polypeptide, protein, protein variant, fusion protein and/or fragment thereof which may be administered to a subject as a medicament. An exemplary therapeutic protein is an interleukin, e.g., IL-7.

As used herein, “synergy” or “synergistic effect” with regard to an effect produced by two or more individual components refers to a phenomenon in which the total effect produced by these components, when utilized in combination, is greater than the sum of the individual effects of each component acting alone.

The term “sufficient amount” or “amount sufficient to” means an amount sufficient to produce a desired effect, e.g., an amount sufficient to reduce the size of a tumor.

The term “therapeutically effective amount” is an amount that is effective to ameliorate a symptom of a disease. A therapeutically effective amount can be a “prophylactically effective amount” as prophylaxis can be considered therapy.

An “effective amount” or “therapeutically effective amount” of a composition includes that amount of the composition which is sufficient to provide a beneficial effect to the subject to which the composition is administered. An “effective amount” of a delivery vehicle includes that amount sufficient to effectively bind or deliver a composition.

As used herein, “combination therapy” embraces administration of each agent or therapy in a sequential manner in a regiment that will provide beneficial effects of the combination and co-administration of these agents or therapies in a substantially simultaneous manner. Combination therapy also includes combinations where individual elements may be administered at different times and/or by different routes but which act in combination to provide a beneficial effect by co-action or pharmacokinetic and pharmacodynamics effect of each agent or tumor treatment approaches of the combination therapy.

The term “in combination with” as used herein refers to uses where, for example, a first therapy is administered during the entire course of administration of a second therapy; where the first therapy is administered for a period of time that is overlapping with the administration of the second therapy, e.g., where administration of the first therapy begins before the administration of the second therapy and the administration of the first therapy ends before the administration of the second therapy ends; where the administration of the second therapy begins before the administration of the first therapy and the administration of the second therapy ends before the administration of the first therapy ends; where the administration of the first therapy begins before administration of the second therapy begins and the administration of the second therapy ends before the administration of the first therapy ends; where the administration of the second therapy begins before administration of the first therapy begins and the administration of the first therapy ends before the administration of the second therapy ends. As such, “in combination” can also refer to regimen involving administration of two or more therapies. “In combination with” as used herein also refers to administration of two or more therapies which may be administered in the same or different formulations, by the same or different routes, and in the same or different dosage form type.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

The articles “a”, “an”, and “the” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

By “ADCC” or “antibody dependent cell-mediated cytotoxicity” as used herein is meant the cell-mediated reaction wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity. As is discussed herein, many embodiments of the invention ablate ADCC activity entirely.

By “ADCP” or antibody dependent cell-mediated phagocytosis as used herein is meant the cell-mediated reaction wherein nonspecific cytotoxic cells that express FcγRs recognize bound antibody on a target cell and subsequently cause phagocytosis of the target cell.

By “antigen binding domain” or “ABD” herein is meant a set of six Complementary Determining Regions (CDRs) that, when present as part of a polypeptide sequence, specifically binds a target antigen as discussed herein. Thus, an “antigen binding domain” binds a target antigen as outlined herein. As is known in the art, these CDRs are generally present as a first set of variable heavy CDRs (vhCDRs or VHCDRs or CDR-HC) and a second set of variable light CDRs (vlCDRs or VLCDRs or CDR-LC), each comprising three CDRs: vhCDR1, vhCDR2, vhCDR3 for the heavy chain and vlCDR1, vlCDR2 and vlCDR3 for the light chain. The CDRs are present in the variable heavy and variable light domains, respectively, and together form an Fv region. Thus, in some cases, the six CDRs of the antigen binding domain are contributed by a variable heavy and variable light chain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the VH and VL domains are covalently attached, generally through the use of a linker as outlined herein, into a single polypeptide sequence, which can be either (starting from the N-terminus) vh-linker-vl or vl-linker-vh, with the former being generally preferred (including optional domain linkers on each side, depending on the format used. As is understood in the art, the CDRs are separated by framework regions in each of the variable heavy and variable light domains: for the light variable region, these are FR1-vlCDR1-FR2-vlCDR2-FR3-vlCDR3-FR4, and for the heavy variable region, these are FR1-vhCDR1-FR2-vhCDR2-FR3-vhCDR3-FR4, with the framework regions showing high identity to human germline sequences. Antigen binding domains of the invention include, Fab, Fv and scFv.

By “modification” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “variant protein” or “protein variant”, or “variant” as used herein is meant a protein that differs from that of a parent protein by virtue of at least one amino acid modification. Protein variant may refer to the protein itself, a composition comprising the protein, or the amino sequence that encodes it. Preferably, the protein variant has at least one amino acid modification compared to the parent protein, e.g., from about one to about seventy amino acid modifications, and preferably from about one to about five amino acid modifications compared to the parent. As described below, in some embodiments the parent polypeptide, for example an Fc parent polypeptide, is a human wild type sequence, such as the Fc region from IgG1, IgG2, IgG3 or IgG4. The protein variant sequence herein will preferably possess at least about 80% identity with a parent protein sequence, and most preferably at least about 90% identity, more preferably at least about 95%-98%-99% identity. Variant protein can refer to the variant protein itself, compositions comprising the protein variant, or the DNA sequence that encodes it.

Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain. The Fc variants of the present invention are defined according to the amino acid modifications that compose them. Thus, for example M252Y or 252Y is an Fc variant with the substitution tyrosine at position 252 relative to the parent Fc polypeptide, wherein the numbering is according to the EU index. Likewise, M252Y/S254T/T256E defines an Fc variant with the substitutions M252Y, S254T and T256E relative to the parent Fc polypeptide. The identity of the wild type amino acid may be unspecified, in which case the aforementioned variant is referred to as 252Y/254T/256E. It is noted that the order in which substitutions are provided is arbitrary, that is to say that, for example, 252Y/254T/256E is the same Fc variant as 254T/252Y/256E, and so on. For all positions discussed in the present invention that relate to antibodies, unless otherwise noted, amino acid position numbering is according to Kabat for the variable region numbering and is according to the EU index for the constant regions, including the Fc region. The EU index or EU index as in Kabat or EU numbering scheme refers to the numbering of the EU antibody (Edelman et al., 1969, Proc Natl Acad Sci USA 63:78-85, hereby entirely incorporated by reference.) The modification can be an addition, deletion, or substitution. Substitutions can include naturally occurring amino acids and, in some cases, synthetic amino acids.

By “Fv” or “Fv fragment” or “Fv region” as used herein is meant a polypeptide that comprises the VL and VH domains of a single antigen binding domain (ABD). As will be appreciated by those in the art, these generally are made up of two chains, or can be combined (generally with a linker as discussed herein) to form a scFv.

By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein.

By “effector function” as used herein is meant a biochemical event that results from the interaction of an antibody Fc region with an Fc receptor or ligand. Effector functions include but are not limited to ADCC, ADCP, and CDC.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. In some cases, as outlined herein, binding to one or more of the FcγR receptors is reduced or ablated. For example, reducing binding to FcγRIIIa reduces ADCC, and in some cases, reducing binding to FcγRIIIa and FcγRIIb is desired.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin.

By “parent polypeptide” as used herein is meant a starting polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below.

By “heavy constant region” herein is meant the CH1-hinge-CH2-CH3 portion of an antibody, generally from human IgG1, IgG2 or IgG4.

By “target antigen” as used herein is meant the molecule that is bound specifically by the variable region of a given antibody. In the present case, the target antigen is a CD27 protein.

By “target cell” as used herein is meant a cell that expresses a target antigen.

By “variable region” as used herein is meant the region of an immunoglobulin that comprises one or more Ig domains substantially encoded by any of the V.kappa., V.lamda., and/or VH genes that make up the kappa, lambda, and heavy chain immunoglobulin genetic loci respectively.

By “wild type or WT” herein is meant an amino acid sequence or a nucleotide sequence that is found in nature, including allelic variations. A WT protein has an amino acid sequence or a nucleotide sequence that has not been intentionally modified.

By “position” as used herein is meant a location in the sequence of a protein. Positions may be numbered sequentially, or according to an established format, for example the EU index for antibody numbering.

By “residue” as used herein is meant a position in a protein and its associated amino acid identity. For example, Asparagine 297 (also referred to as Asn297 or N297) is a residue at position 297 in the human antibody IgG1.

The antibodies of the present invention are generally recombinant. “Recombinant” means the antibodies are generated using recombinant nucleic acid techniques in exogenous host cells.

The term “recombinant,” as applied to a polynucleotide means the polynucleotide is the product of various combinations of cloning, restriction or ligation steps, and other procedures resulting in a construct distinct and/or different from a polynucleotide found in nature. The terms respectively include replicates of the original polynucleotide construct and progeny of the original virus construct.

“Percent (%) amino acid sequence identity” with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. One particular program is the ALIGN-2 program outlined at paragraphs [0279] to [0280] of US Pub. No. 20160244525, hereby incorporated by reference. Another approximate alignment for nucleic acid sequences is provided by the local homology algorithm of Smith and Waterman, Advances in Applied Mathematics, 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986).

An example of an implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages, the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the internet address located by placing http:// in front of blast.ncbi.nlm.nih.gov/Blast.cgi.

The degree of identity between an amino acid sequence of the present invention (“invention sequence”) and the parental amino acid sequence is calculated as the number of exact matches in an alignment of the two sequences, divided by the length of the “invention sequence,” or the length of the parental sequence, whichever is the shortest. The result is expressed in percent identity.

In some embodiments, two or more amino acid sequences are at least 50%, 60%, 70%, 80%, or 90% identical. In some embodiments, two or more amino acid sequences are at least 95%, 97%, 98%, 99%, or even 100% identical.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

A “disease” includes a state of health of an animal, including a human, wherein the animal cannot maintain homeostasis, and wherein if the disease is not ameliorated then the animal's health continues to deteriorate.

In contrast, a “disorder” in an animal, including a human, includes a state of health in which the animal is able to maintain homeostasis, but in which the animal's state of health is less favorable than it would be in the absence of the disorder. Left untreated, a disorder does not necessarily cause a further decrease in the animal's state of health.

The terms “treatment”, “treating”, “treat”, and the like, refer to obtaining a desired pharmacologic and/or physiologic effect. The effect may be prophylactic in terms of completely or partially preventing a disease or symptom thereof or reducing the likelihood of a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine.

The term “regression,” as well as words stemming therefrom, as used herein, does not necessarily imply 100% or complete regression. Rather, there are varying degrees of regression of which one of ordinary skill in the art recognizes as having a potential benefit or therapeutic effect. In this respect, the disclosed methods can provide any amount of any level of regression of a cancer in a mammal. Furthermore, the regression provided by the inventive method can include regression of one or more conditions or symptoms of the disease, e.g., a cancer. Also, for purposes herein, “regression” can encompass delaying the onset of the disease, delaying the onset of a symptom, and/or delaying the onset of a condition thereof. With respect to progressive diseases and disorders, “regression” can encompass slowing the progression of the disease or disorder, slowing the progression of a symptom of the disease or disorder, and/or slowing the progression of a condition thereof.

“Encoding” includes the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if, for example, transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “operably linked” as used herein includes a polynucleotide in functional relationship with a second polynucleotide, e.g., a single-stranded or double-stranded nucleic acid moiety comprising the two polynucleotides arranged within the nucleic acid moiety in such a manner that at least one of the two polynucleotides is able to exert a physiological effect by which it is characterized, upon the other. By way of example, a promoter operably linked to the coding region of a gene is able to promote transcription of the coding region. The order specified when indicating operably linkage is not important. For example, the phrases: “the promoter is operably linked to the nucleotide sequence” and “the nucleotide sequence is operably linked to the promoter” are used interchangeably herein and are considered equivalent. In some cases, when the nucleic acid encoding the desired protein further comprises a promoter/regulatory sequence, the promoter/regulatory sequence is positioned at the 5′ end of the desired protein coding sequence such that it drives expression of the desired protein in a cell.

The term “promoter” as used herein includes a DNA sequence operably linked to a nucleic acid sequence to be transcribed such as a nucleic acid sequence encoding a desired molecule. A promoter is generally positioned upstream of a nucleic acid sequence to be transcribed and provides a site for specific binding by RNA polymerase and other transcription factors.

A “vector” is capable of transferring gene sequences to target-cells. Typically, “vector construct,” “expression vector,” and “gene transfer vector,” mean any nucleic acid construct capable of directing the expression of a gene of interest and which can transfer gene sequences to target-cells, which can be accomplished by genomic integration of all or a portion of the vector, or transient or inheritable maintenance of the vector as an extrachromosomal element. Thus, the term includes cloning, and expression vehicles, as well as integrating vectors.

The term “regulatory element” as used herein includes a nucleotide sequence which controls some aspect of the expression of nucleic acid sequences. Examples of regulatory elements illustratively include an enhancer, an internal ribosome entry site (IRES), an intron, an origin of replication, a polyadenylation signal (pA), a promoter, an enhancer, a transcription termination sequence, and an upstream regulatory domain, which contribute to the replication, transcription, and/or post-transcriptional processing of a nucleic acid sequence. In cases, regulatory elements can also include cis-regulatory DNA elements as well as transposable elements (TEs). Those of ordinary skill in the art are capable of selecting and using these and other regulatory elements in an expression construct with no more than routine experimentation. Expression constructs can be generated using a genetic recombinant approach or synthetically using well-known methodology.

A “control element” or “control sequence” is a nucleotide sequence involved in an interaction of molecules contributing to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

The statement that an amino acid residue is “phosphorylated” used herein means that a phosphate group is ester-linked to the side chain of the amino acid residue. Typical amino acid residues that may be phosphorylated include serine (Ser), threonine (Thr), and tyrosine (Tyr).

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. [1975].

As used herein, the term “enhancing an immune response” and “inducing an immune response” are used interchangeably and refer to the stimulation of an immune response.

As used herein, the term “inhibiting an immune response” means blocking the stimulation of an immune response. The blockade can be partial or complete.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

Various aspects of the invention are set forth below in sections; however, aspects of the invention described in one particular section are not to be limited to any particular section.

### III. Anti-CD27 Antibodies

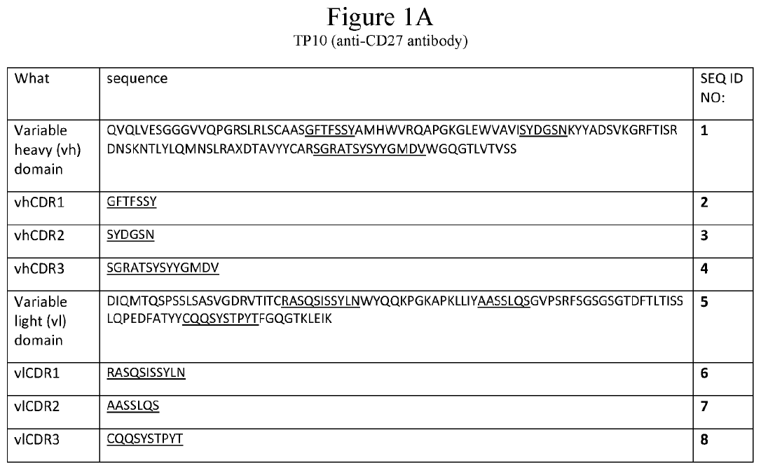

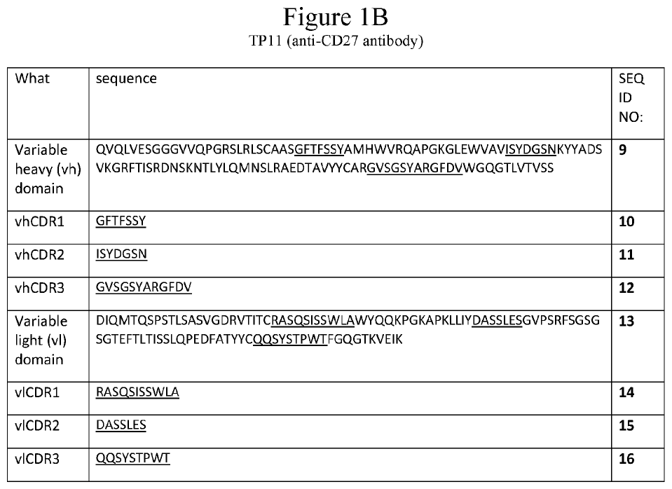

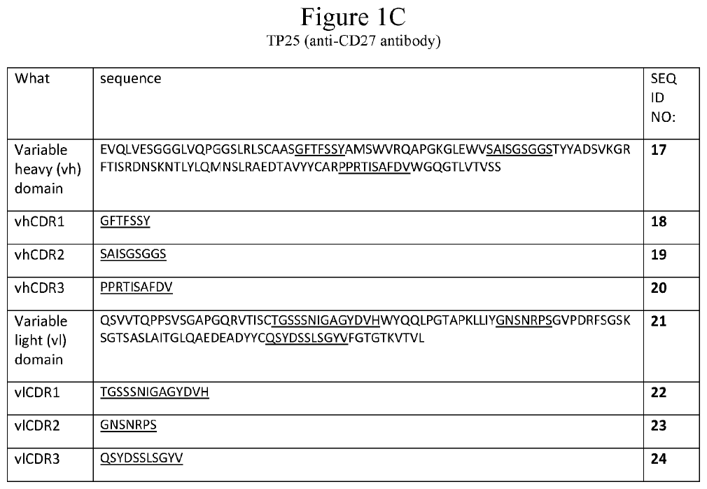

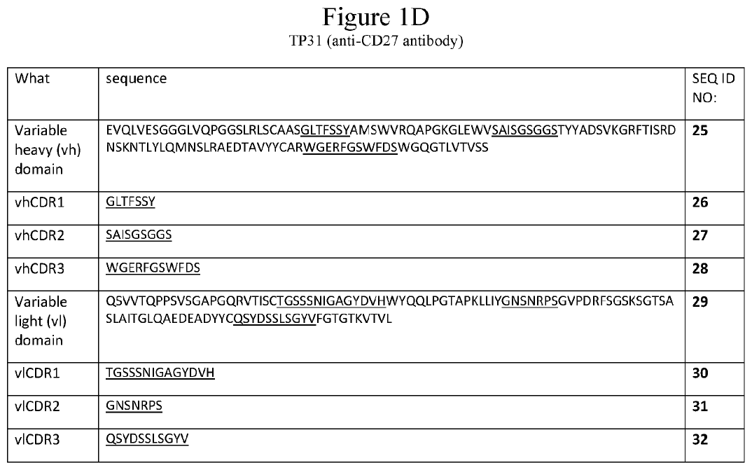

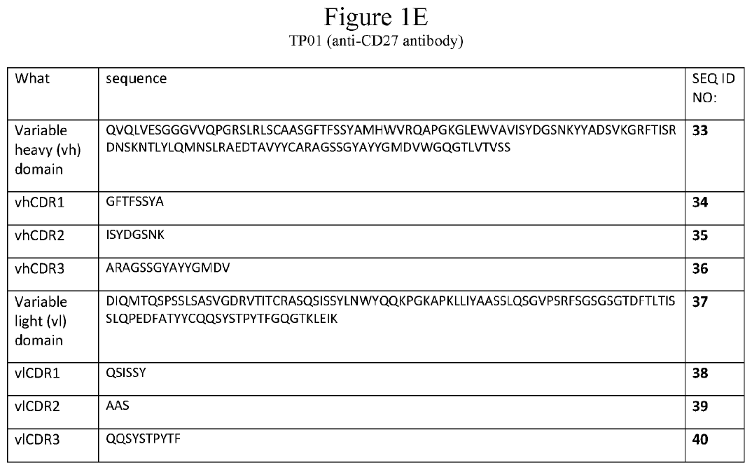

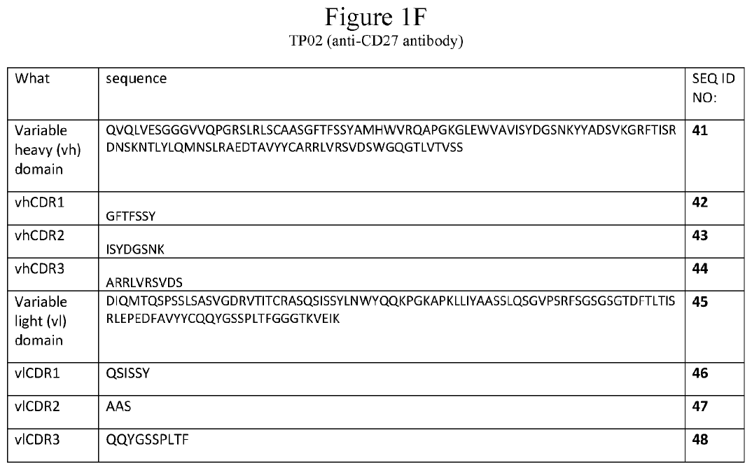

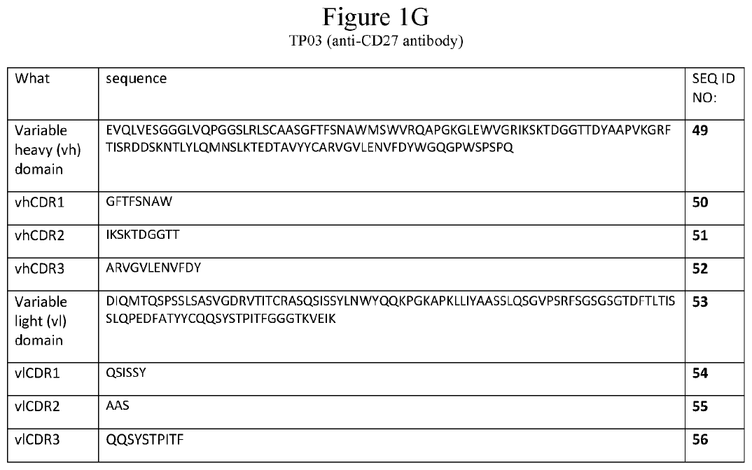

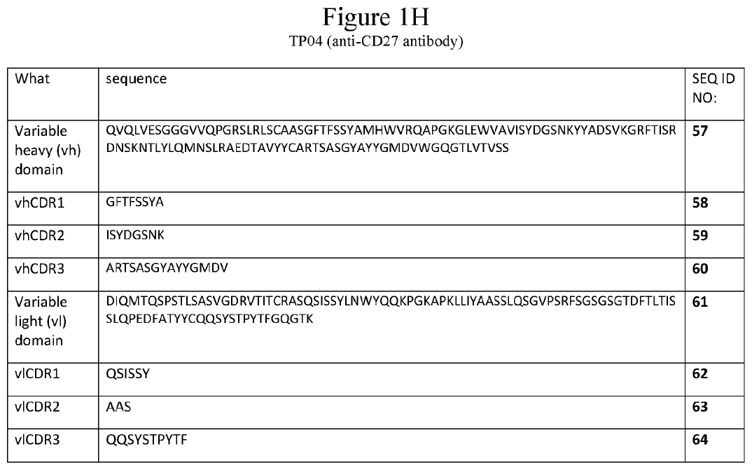

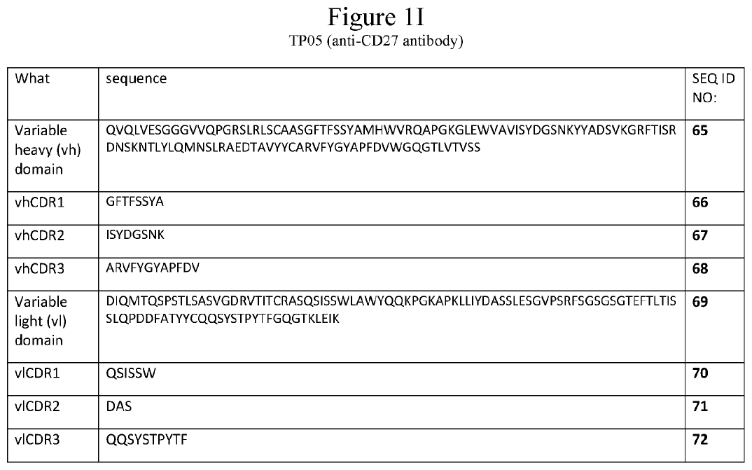

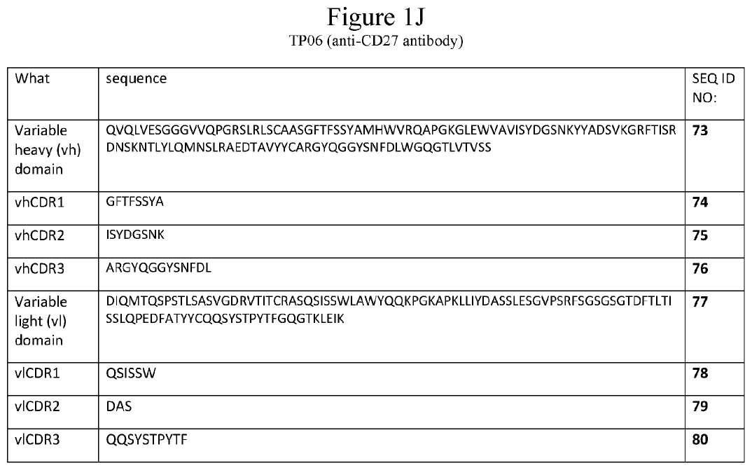

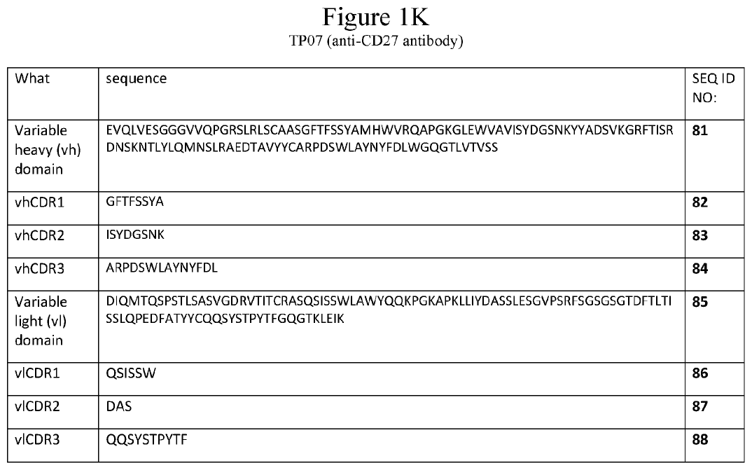

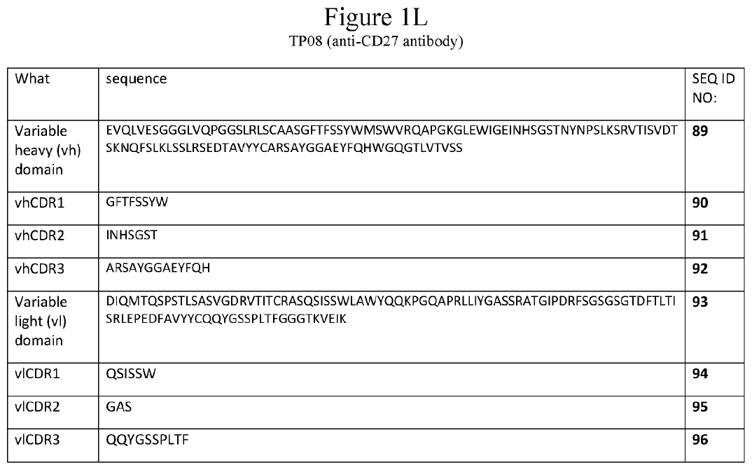

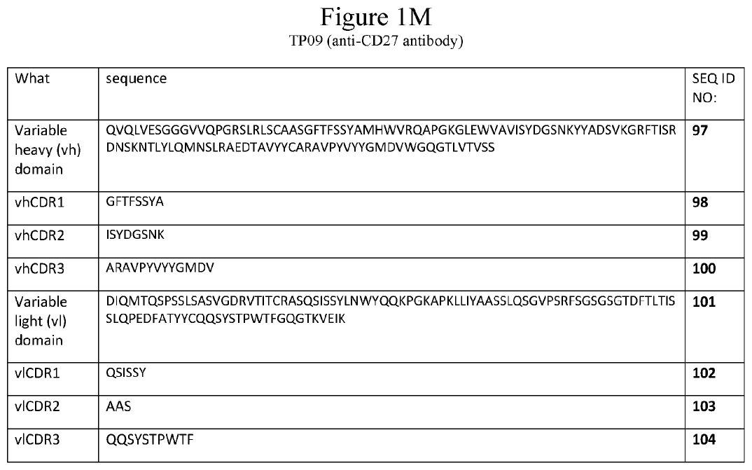

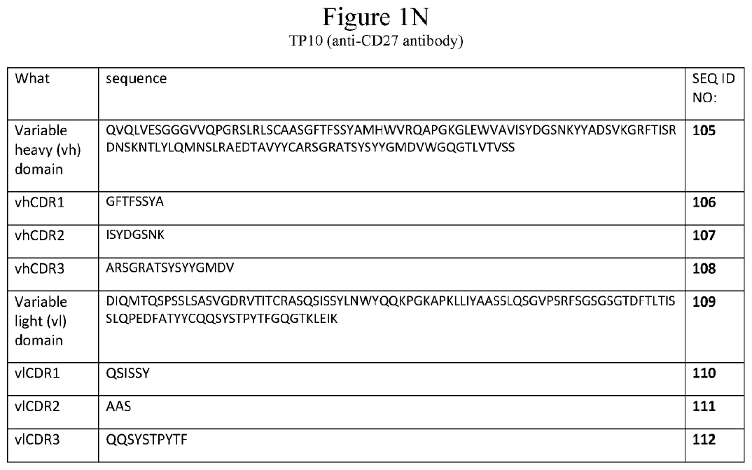

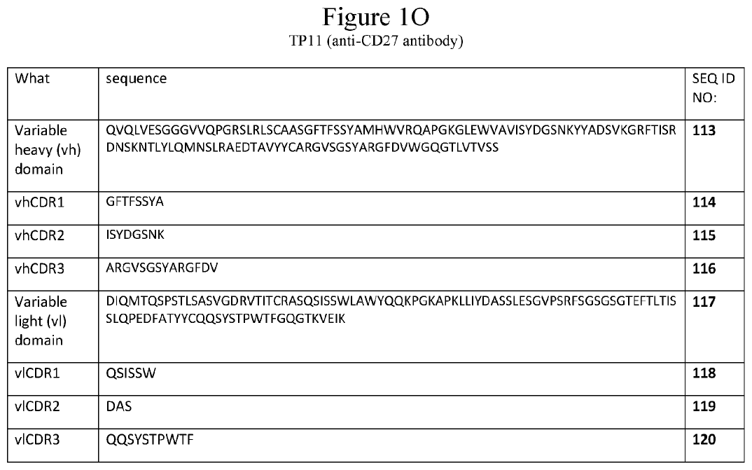

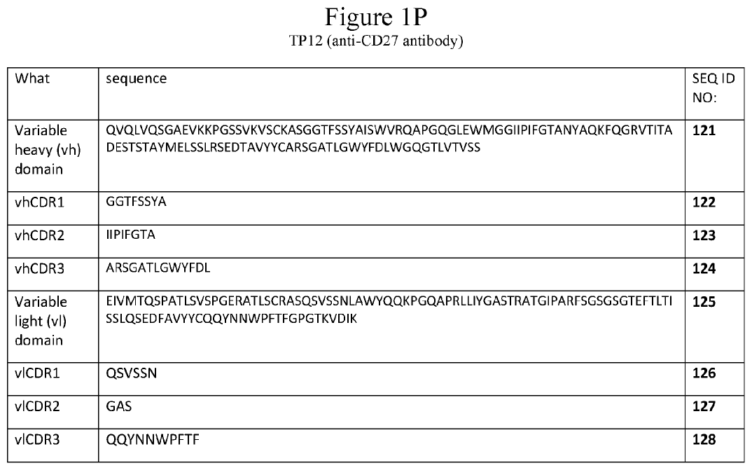

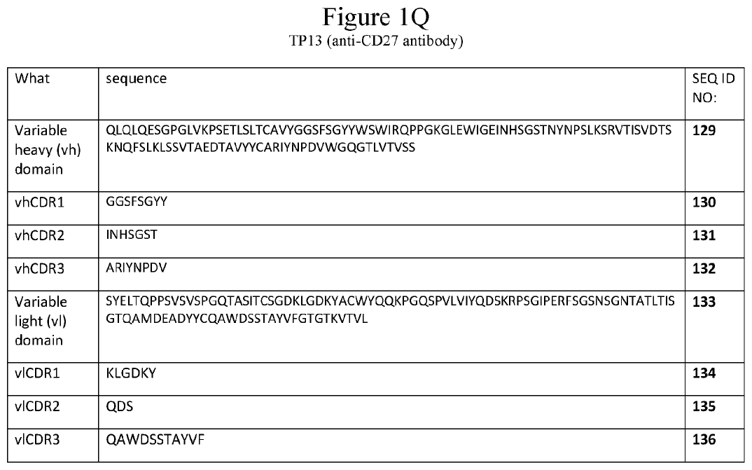

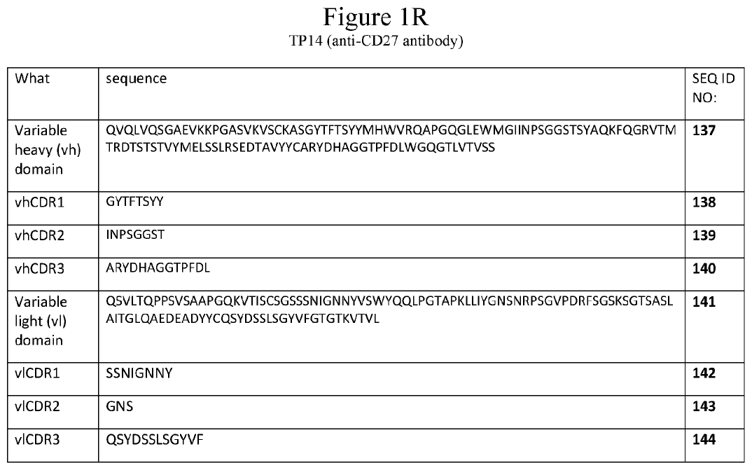

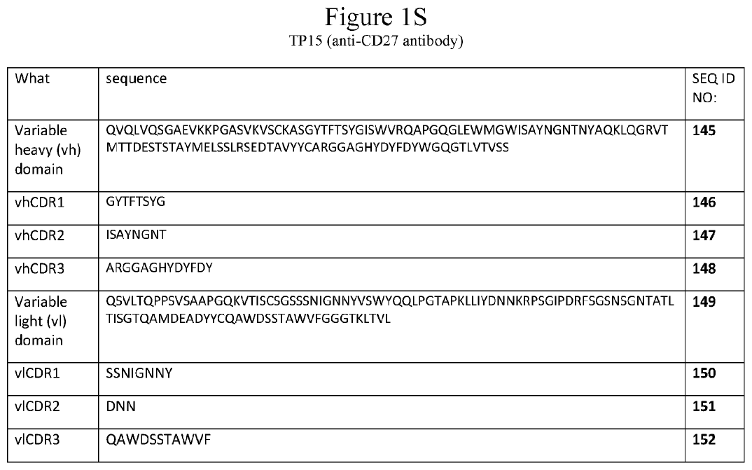

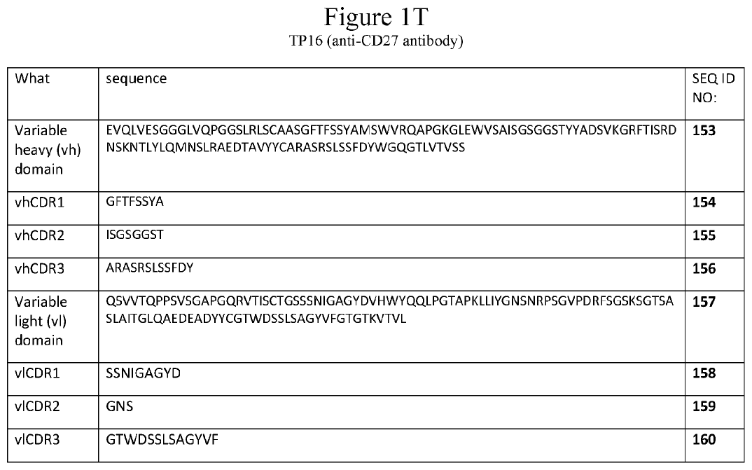

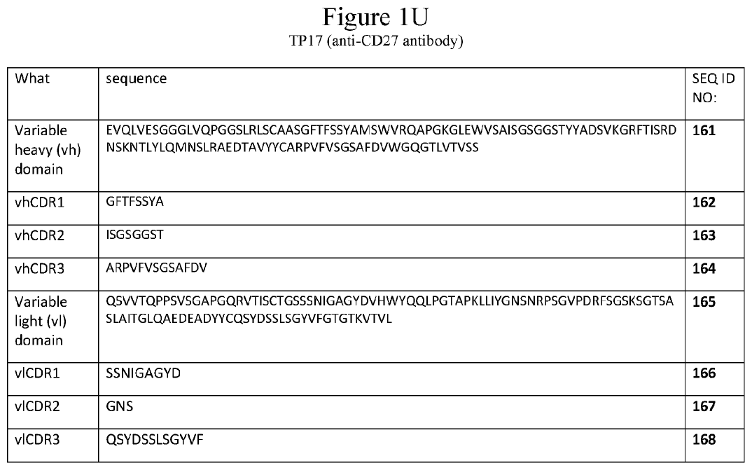

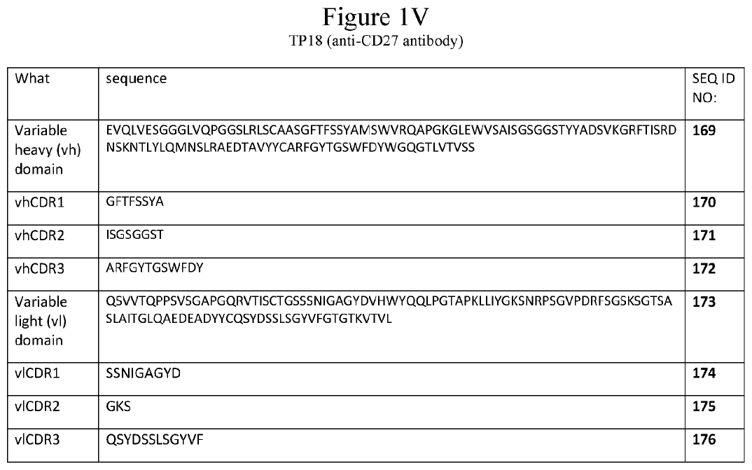

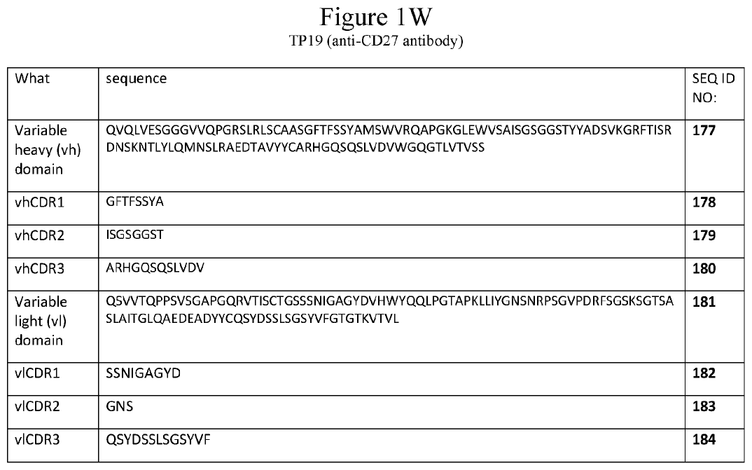

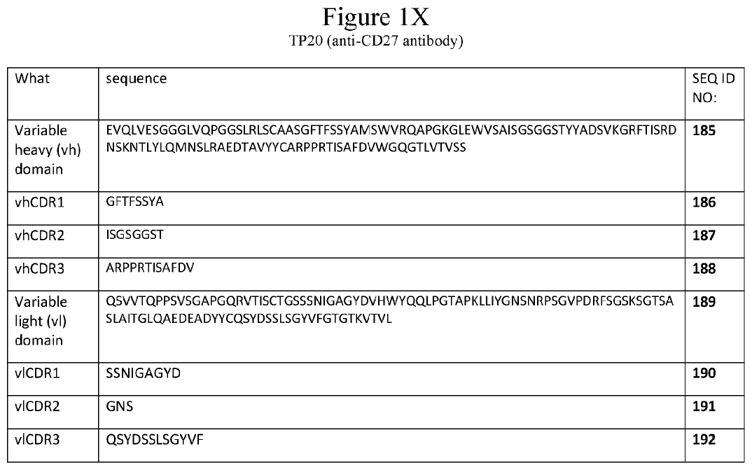

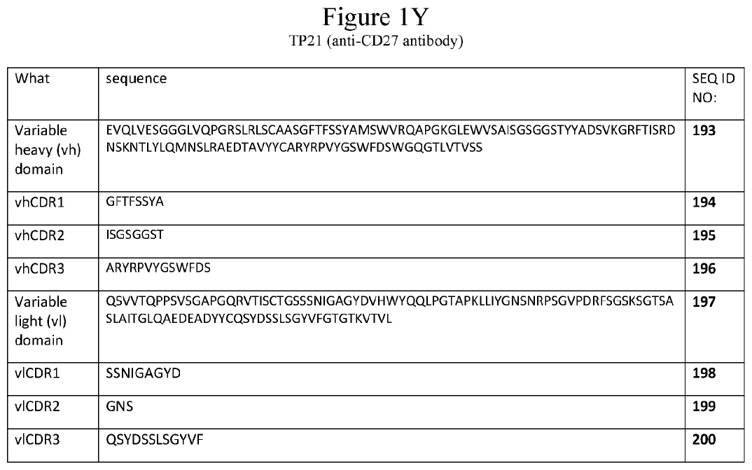

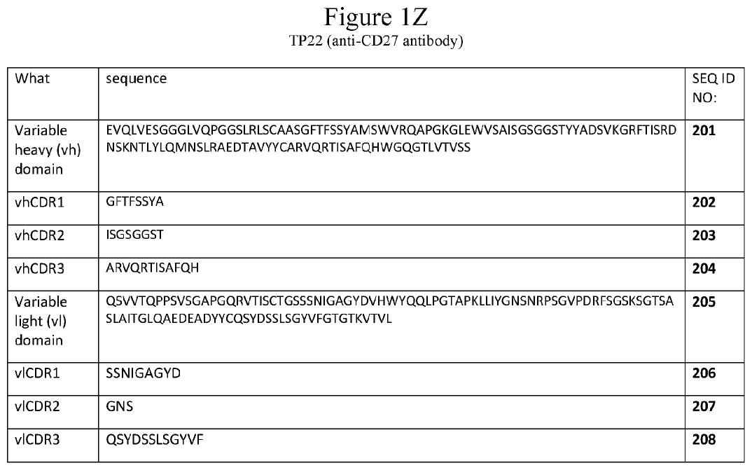

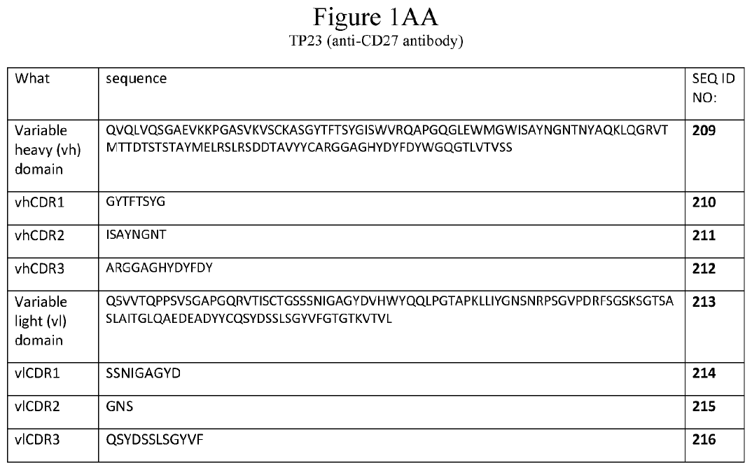

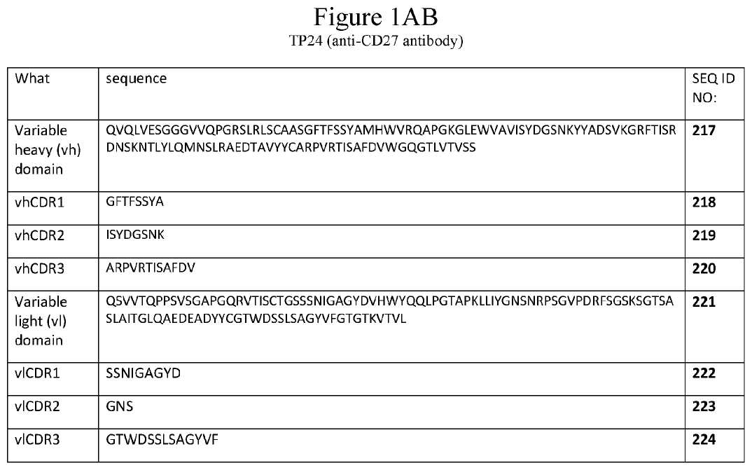

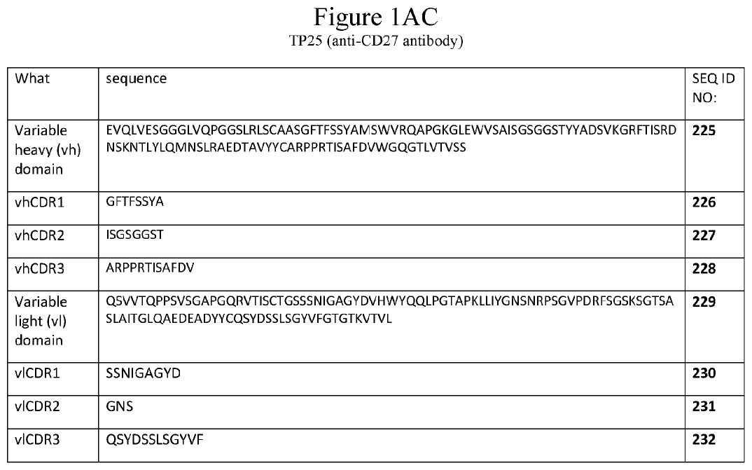

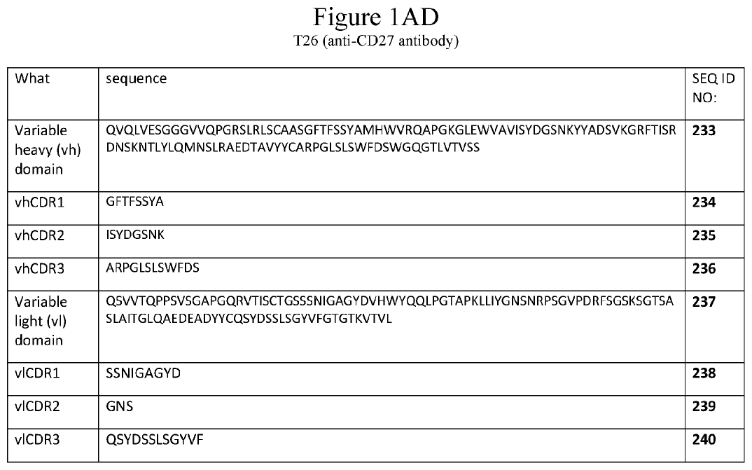

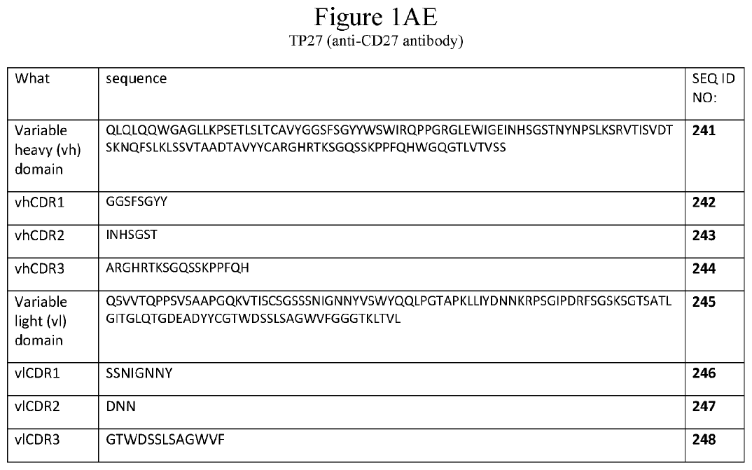

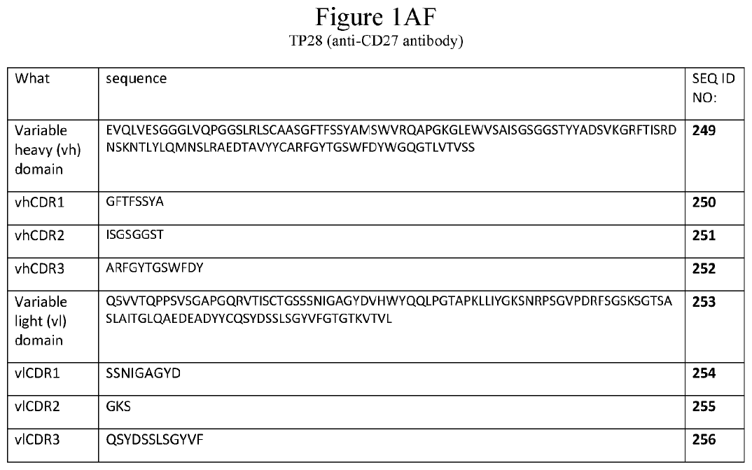

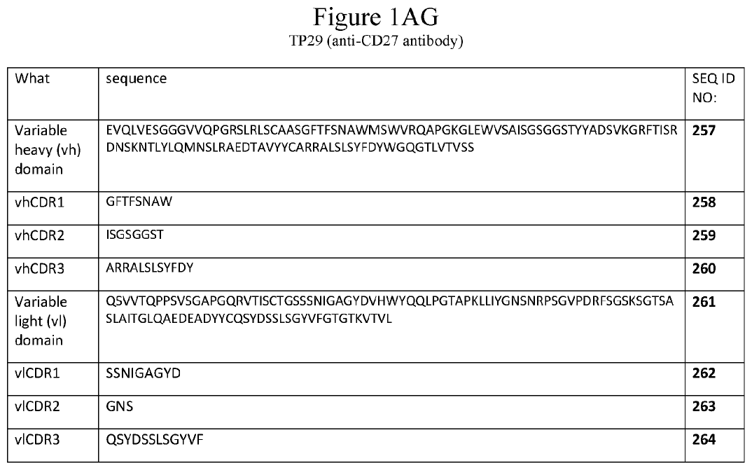

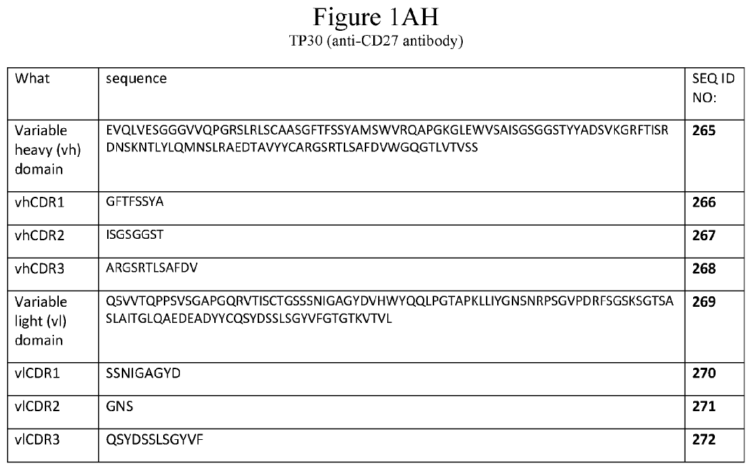

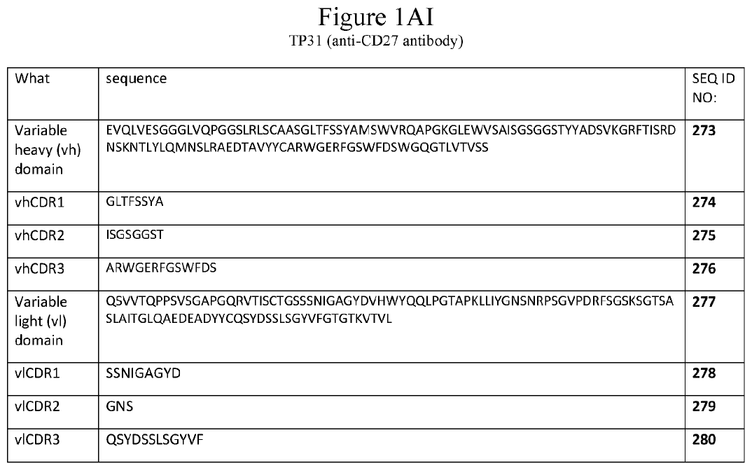

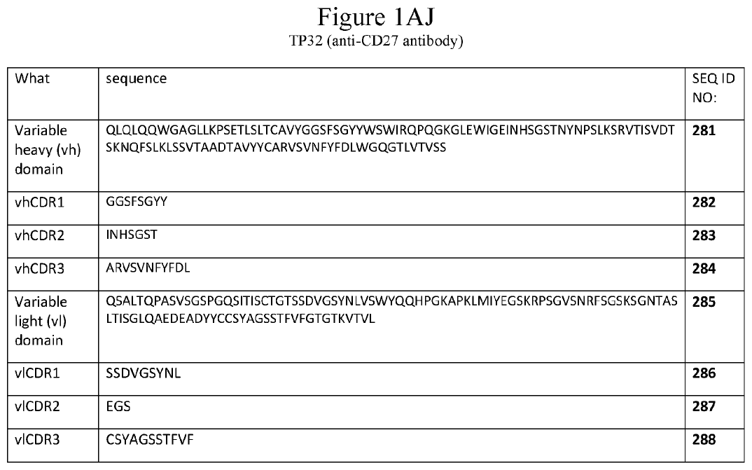

The present disclosure provides novel anti-CD27 antibodies. Such antibodies bind human CD27. FIG. 1 lists peptide sequences of heavy chain variable regions and light chain variable regions that, in combination as designated in FIG. 1, can bind to human CD27. In some embodiments, the heavy chain variable region and the light chain variable region are arranged in a Fab format. In some embodiments, the heavy chain variable region and the light chain variable region are fused together to from an scFv.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to any heavy chain variable region described in FIG. 1 and any light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to any light chain variable region described in FIG. 1.

In some embodiments, the anti-CD27 antibodies include a vhCDR1, a vhCDR2, a vhCDR3, a vlCDR1, a vlCDR2, and a vlCDR3 a described in FIG. 1. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human CD27.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:1 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:5.

In some embodiments, the anti-CD27 antibodies include a vhCDR1 comprising SEQ ID NO:1, a vhCDR2 comprising SEQ ID NO:3, a vhCDR3 comprising SEQ ID NO:4, a vlCDR1 comprising SEQ ID NO:6, a vlCDR2 comprising SEQ ID NO:7, and a vlCDR3 comprising SEQ ID NO:8. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human CD27.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:9 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:13.

In some embodiments, the anti-CD27 antibodies include a vhCDR1 comprising SEQ ID NO:10, a vhCDR2 comprising SEQ ID NO:11, a vhCDR3 comprising SEQ ID NO:12, a vlCDR1 comprising SEQ ID NO:14, a vlCDR2 comprising SEQ ID NO:15, and a vlCDR3 comprising SEQ ID NO:16. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human CD27.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:17 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:21.

In some embodiments, the anti-CD27 antibodies include a vhCDR1 comprising SEQ ID NO:18, a vhCDR2 comprising SEQ ID NO:19, a vhCDR3 comprising SEQ ID NO:20, a vlCDR1 comprising SEQ ID NO:22, a vlCDR2 comprising SEQ ID NO:23, and a vlCDR3 comprising SEQ ID NO:24. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human CD27.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:25 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:29.

In some embodiments, the anti-CD27 antibodies include a vhCDR1 comprising SEQ ID NO:26, a vhCDR2 comprising SEQ ID NO:27, a vhCDR3 comprising SEQ ID NO:28, a vlCDR1 comprising SEQ ID NO:30, a vlCDR2 comprising SEQ ID NO:31, and a vlCDR3 comprising SEQ ID NO:32. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:33 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:37.

In some embodiments, the anti-CD27 antibodies include a vhCDR1 comprising SEQ ID NO:34, a vhCDR2 comprising SEQ ID NO:35, a vhCDR3 comprising SEQ ID NO:36, a vlCDR1 comprising SEQ ID NO:38, a vlCDR2 comprising SEQ ID NO:39, and a vlCDR3 comprising SEQ ID NO:40. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:41 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:45.

In some embodiments, the anti-CD27 antibodies include a vhCDR1 comprising SEQ ID NO:42, a vhCDR2 comprising SEQ ID NO:43, a vhCDR3 comprising SEQ ID NO:44, a vlCDR1 comprising SEQ ID NO:45, a vlCDR2 comprising SEQ ID NO:46, and a vlCDR3 comprising SEQ ID NO:47. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO49 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:53.

In some embodiments, the anti-CD27 antibodies include a vhCDR1 comprising SEQ ID NO:50, a vhCDR2 comprising SEQ ID NO:51, a vhCDR3 comprising SEQ ID NO:52, a vlCDR1 comprising SEQ ID NO:53, a vlCDR2 comprising SEQ ID NO:54, and a vlCDR3 comprising SEQ ID NO:55. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:57 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:61.

In some embodiments, the anti-CD27 antibodies include a vhCDR1 comprising SEQ ID NO:58, a vhCDR2 comprising SEQ ID NO:59, a vhCDR3 comprising SEQ ID NO:60, a vlCDR1 comprising SEQ ID NO:62, a vlCDR2 comprising SEQ ID NO:63, and a vlCDR3 comprising SEQ ID NO:64. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:365 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:69.

In some embodiments, the anti-CD27 antibodies include a vhCDR1 comprising SEQ ID NO:66, a vhCDR2 comprising SEQ ID NO:67, a vhCDR3 comprising SEQ ID NO:68, a vlCDR1 comprising SEQ ID NO:70, a vlCDR2 comprising SEQ ID NO:71, and a vlCDR3 comprising SEQ ID NO:72. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:73 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:77.

In some embodiments, the anti-CD27 antibodies include a vhCDR1 comprising SEQ ID NO:74, a vhCDR2 comprising SEQ ID NO:75, a vhCDR3 comprising SEQ ID NO:76, a vlCDR1 comprising SEQ ID NO:78, a vlCDR2 comprising SEQ ID NO:79, and a vlCDR3 comprising SEQ ID NO:80. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:81 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:85.

In some embodiments, the anti-CD27 antibodies include a vhCDR1 comprising SEQ ID NO:82, a vhCDR2 comprising SEQ ID NO:83, a vhCDR3 comprising SEQ ID NO:84, a vlCDR1 comprising SEQ ID NO:86, a vlCDR2 comprising SEQ ID NO:87, and a vlCDR3 comprising SEQ ID NO:88. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:89 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:93.

In some embodiments, the anti-CD27 antibodies include a vhCDR1 comprising SEQ ID NO:90, a vhCDR2 comprising SEQ ID NO:91, a vhCDR3 comprising SEQ ID NO:92, a vlCDR1 comprising SEQ ID NO:94, a vlCDR2 comprising SEQ ID NO:95, and a vlCDR3 comprising SEQ ID NO:96. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:97 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:101.

In some embodiments, the anti-CD27 antibodies include a vhCDR1 comprising SEQ ID NO:98, a vhCDR2 comprising SEQ ID NO:99, a vhCDR3 comprising SEQ ID NO:100, a vlCDR1 comprising SEQ ID NO:102, a vlCDR2 comprising SEQ ID NO:103, and a vlCDR3 comprising SEQ ID NO:104. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:105 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:109.

In some embodiments, the anti-CD27 antibodies include a vhCDR1 comprising SEQ ID NO:106, a vhCDR2 comprising SEQ ID NO:107, a vhCDR3 comprising SEQ ID NO:108, a vlCDR1 comprising SEQ ID NO:110, a vlCDR2 comprising SEQ ID NO:111, and a vlCDR3 comprising SEQ ID NO:112. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:113 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:117.

In some embodiments, the anti-CD27 antibodies include a vhCDR1 comprising SEQ ID NO:114, a vhCDR2 comprising SEQ ID NO:115, a vhCDR3 comprising SEQ ID NO:116, a vlCDR1 comprising SEQ ID NO:118, a vlCDR2 comprising SEQ ID NO:119, and a vlCDR3 comprising SEQ ID NO:120. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:121 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:125.

In some embodiments, the anti-CD27 antibodies include a vhCDR1 comprising SEQ ID NO:122, a vhCDR2 comprising SEQ ID NO:123, a vhCDR3 comprising SEQ ID NO:124, a vlCDR1 comprising SEQ ID NO:126, a vlCDR2 comprising SEQ ID NO:127, and a vlCDR3 comprising SEQ ID NO:128. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:121 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:125.

In some embodiments, the anti-CD27 antibodies include a vhCDR1 comprising SEQ ID NO:122, a vhCDR2 comprising SEQ ID NO:123, a vhCDR3 comprising SEQ ID NO:124, a vlCDR1 comprising SEQ ID NO:126, a vlCDR2 comprising SEQ ID NO:127, and a vlCDR3 comprising SEQ ID NO:128. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:129 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:133.

In some embodiments, the anti-CD27 antibodies include a vhCDR1 comprising SEQ ID NO:130, a vhCDR2 comprising SEQ ID NO:131, a vhCDR3 comprising SEQ ID NO:133, a vlCDR1 comprising SEQ ID NO:134, a vlCDR2 comprising SEQ ID NO:135, and a vlCDR3 comprising SEQ ID NO:136. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:137 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:141.

In some embodiments, the anti-CD27 antibodies include a vhCDR1 comprising SEQ ID NO:138, a vhCDR2 comprising SEQ ID NO:139, a vhCDR3 comprising SEQ ID NO:140, a vlCDR1 comprising SEQ ID NO:142, a vlCDR2 comprising SEQ ID NO:143, and a vlCDR3 comprising SEQ ID NO:144. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:145 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:149.

In some embodiments, the anti-CD27 antibodies include a vhCDR1 comprising SEQ ID NO:146, a vhCDR2 comprising SEQ ID NO:147, a vhCDR3 comprising SEQ ID NO:148, a vlCDR1 comprising SEQ ID NO:150, a vlCDR2 comprising SEQ ID NO:151, and a vlCDR3 comprising SEQ ID NO:152. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:153 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:157.

In some embodiments, the anti-CD27 antibodies include a vhCDR1 comprising SEQ ID NO:154, a vhCDR2 comprising SEQ ID NO:155, a vhCDR3 comprising SEQ ID NO:156, a vlCDR1 comprising SEQ ID NO:158, a vlCDR2 comprising SEQ ID NO:159, and a vlCDR3 comprising SEQ ID NO:160. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:161 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:165.

In some embodiments, the anti-CD27 antibodies include a vhCDR1 comprising SEQ ID NO:162, a vhCDR2 comprising SEQ ID NO:163, a vhCDR3 comprising SEQ ID NO:164, a vlCDR1 comprising SEQ ID NO:166, a vlCDR2 comprising SEQ ID NO:167, and a vlCDR3 comprising SEQ ID NO:168. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:169 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:173.

In some embodiments, the anti-CD27 antibodies include a vhCDR1 comprising SEQ ID NO:170, a vhCDR2 comprising SEQ ID NO:171, a vhCDR3 comprising SEQ ID NO:172, a vlCDR1 comprising SEQ ID NO:174, a vlCDR2 comprising SEQ ID NO:175, and a vlCDR3 comprising SEQ ID NO:176. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:177 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:181.

In some embodiments, the anti-CD27 antibodies include a vhCDR1 comprising SEQ ID NO:178, a vhCDR2 comprising SEQ ID NO:179, a vhCDR3 comprising SEQ ID NO:180, a vlCDR1 comprising SEQ ID NO:182, a vlCDR2 comprising SEQ ID NO:183, and a vlCDR3 comprising SEQ ID NO:184. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:185 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:189.

In some embodiments, the anti-CD27 antibodies include a vhCDR1 comprising SEQ ID NO:186, a vhCDR2 comprising SEQ ID NO:187, a vhCDR3 comprising SEQ ID NO:188, a vlCDR1 comprising SEQ ID NO:190, a vlCDR2 comprising SEQ ID NO:191, and a vlCDR3 comprising SEQ ID NO:192. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:193 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:197.

In some embodiments, the anti-CD27 antibodies include a vhCDR1 comprising SEQ ID NO:194, a vhCDR2 comprising SEQ ID NO:195, a vhCDR3 comprising SEQ ID NO:196, a vlCDR1 comprising SEQ ID NO:198, a vlCDR2 comprising SEQ ID NO:199, and a vlCDR3 comprising SEQ ID NO:200. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:201 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:205.

In some embodiments, the anti-CD27 antibodies include a vhCDR1 comprising SEQ ID NO:202, a vhCDR2 comprising SEQ ID NO:203, a vhCDR3 comprising SEQ ID NO:204, a vlCDR1 comprising SEQ ID NO:206, a vlCDR2 comprising SEQ ID NO:207, and a vlCDR3 comprising SEQ ID NO:208. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:209 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:213.

In some embodiments, the anti-CD27 antibodies include a vhCDR1 comprising SEQ ID NO:210, a vhCDR2 comprising SEQ ID NO:211, a vhCDR3 comprising SEQ ID NO:212, a vlCDR1 comprising SEQ ID NO:214, a vlCDR2 comprising SEQ ID NO:215, and a vlCDR3 comprising SEQ ID NO:216. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:217 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:221.

In some embodiments, the anti-CD27 antibodies include a vhCDR1 comprising SEQ ID NO:218, a vhCDR2 comprising SEQ ID NO:219, a vhCDR3 comprising SEQ ID NO:220, a vlCDR1 comprising SEQ ID NO:222, a vlCDR2 comprising SEQ ID NO:223, and a vlCDR3 comprising SEQ ID NO:224. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:225 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:229.

In some embodiments, the anti-CD27 antibodies include a vhCDR1 comprising SEQ ID NO:226, a vhCDR2 comprising SEQ ID NO:227, a vhCDR3 comprising SEQ ID NO:228, a vlCDR1 comprising SEQ ID NO:230, a vlCDR2 comprising SEQ ID NO:231, and a vlCDR3 comprising SEQ ID NO:232. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:233 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:237.

In some embodiments, the anti-CD27 antibodies include a vhCDR1 comprising SEQ ID NO:234, a vhCDR2 comprising SEQ ID NO:235, a vhCDR3 comprising SEQ ID NO:236, a vlCDR1 comprising SEQ ID NO:238, a vlCDR2 comprising SEQ ID NO:239, and a vlCDR3 comprising SEQ ID NO:240. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:241 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:245.

In some embodiments, the anti-CD27 antibodies include a vhCDR1 comprising SEQ ID NO:242, a vhCDR2 comprising SEQ ID NO:243, a vhCDR3 comprising SEQ ID NO:244, a vlCDR1 comprising SEQ ID NO:246, a vlCDR2 comprising SEQ ID NO:247, and a vlCDR3 comprising SEQ ID NO:248. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:249 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:253.

In some embodiments, the anti-CD27 antibodies include a vhCDR1 comprising SEQ ID NO:250, a vhCDR2 comprising SEQ ID NO:251, a vhCDR3 comprising SEQ ID NO:252, a vlCDR1 comprising SEQ ID NO:254, a vlCDR2 comprising SEQ ID NO:255, and a vlCDR3 comprising SEQ ID NO:256. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:257 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:264.

In some embodiments, the anti-CD27 antibodies include a vhCDR1 comprising SEQ ID NO:258, a vhCDR2 comprising SEQ ID NO:259, a vhCDR3 comprising SEQ ID NO:260, a vlCDR1 comprising SEQ ID NO:262, a vlCDR2 comprising SEQ ID NO:263, and a vlCDR3 comprising SEQ ID NO:264. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:265 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:269.

In some embodiments, the anti-CD27 antibodies include a vhCDR1 comprising SEQ ID NO:266, a vhCDR2 comprising SEQ ID NO:267, a vhCDR3 comprising SEQ ID NO:268, a vlCDR1 comprising SEQ ID NO:270, a vlCDR2 comprising SEQ ID NO:271, and a vlCDR3 comprising SEQ ID NO:272. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:273 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:277.

In some embodiments, the anti-CD27 antibodies include a vhCDR1 comprising SEQ ID NO:274, a vhCDR2 comprising SEQ ID NO:275, a vhCDR3 comprising SEQ ID NO:276, a vlCDR1 comprising SEQ ID NO:278, a vlCDR2 comprising SEQ ID NO:279, and a vlCDR3 comprising SEQ ID NO:280. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In some embodiments, the anti-CD27 antibodies in the present disclosure include a heavy chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:281 and a light chain variable region having an amino acid sequence at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or 100%) identical to SEQ ID NO:285.

In some embodiments, the anti-CD27 antibodies include a vhCDR1 comprising SEQ ID NO:282, a vhCDR2 comprising SEQ ID NO:283, a vhCDR3 comprising SEQ ID NO:284, a vlCDR1 comprising SEQ ID NO:286, a vlCDR2 comprising SEQ ID NO:287, and a vlCDR3 comprising SEQ ID NO:288. In some embodiments, one or more of such 6 CDRs have from 1, 2, 3, 4 or 5 amino acid modifications. In further embodiments, a single CDR contains 1 or 2 amino acid substitutions, and the modified anti-CD27 antibodies retain binding to human C.

In addition to the sequence variants described herein in the heavy chain and light chain variable regions and/or CDRs, changes in the framework region(s) of the heavy and/or light variable region(s) can be made. In some embodiment, variants in the framework regions (e.g., excluding the CDRs) retain at least about 80, 85, 90 or 95% identity to a germline sequence. Variants can be made to retain at least about 80, 85, 90 or 95% identity to any one of the light chain V-GENE, light chain J-GENE, heavy chain V-GENE, heavy chain J-GENE, and heavy chain D-GENE alleles.

In some embodiments, variations are made in the framework regions that retain at least 80, 85, 90 or 95% identity to the germline gene sequences, while keeping 6 CDRs unchanged.

In some embodiments, variations are made in both the framework regions that retain at least 80, 85, 90 or 95% identity to the germline gene sequences, and the 6 CDRs. The CDRs can have amino acid modifications (e.g., from 1, 2, 3, 4 or 5 amino acid modifications in the set of CDRs (that is, the CDRs can be modified as long as the total number of changes in the set of 6 CDRs is less than 6 amino acid modifications, with any combination of CDRs being changed; e.g., there may be one change in vlCDR1, two in vhCDR2, none in vhCDR3, etc.).

By selecting amino acid sequences of CDRs and/or variable regions of a heavy chain and a light chain from those described herein and combining them with amino acid sequences of framework regions and/or constant regions of a heavy chain and a light chain of an antibody as appropriate, a person skilled in the art will be able to design an anti-CD27 antibody according to the present invention. The antibody framework regions and/or constant region (Fc domain) described in the current invention can derive from an antibody of any species, such as from human, rabbit, dog, cat, mouse, horse or monkey.

In some embodiments, the constant region is derived from human, and includes a heavy chain constant region derived from those of IgG, IgA, IgM, IgE, and IgD subtypes or variants thereof, and a light chain constant region derived from kappa or lambda subtypes or variants thereof. In some embodiments, the heavy chain constant region is derived from a human IgG, including IgG1, IgG2, IgG3, and IgG4. In some embodiments, the amino acid sequence of the heavy chain constant region is at least 80%, 85%, 90%, or 95% identical to a human IgG1, IgG2, IgG3, or IgG4 constant region. In some other embodiments, the amino acid sequence of the constant region is at least 80%, 85%, 90%, or 95% identical to an antibody constant region from another mammal, such as rabbit, dog, cat, mouse, horse or monkey. In some embodiments, the antibody constant region includes a hinge, a CH2 domain, a CH3 domain and optionally a CH1 domain.

In some embodiments, the antibodies described herein can be derived from a mixture from different species, e.g., forming a chimeric antibody and/or a humanized antibody. In general, both “chimeric antibodies” and “humanized antibodies” refer to antibodies that combine regions from more than one species. For example, “chimeric antibodies” traditionally comprise variable region(s) from a mouse (or rat, in some cases) and the constant region(s) from a human. “Humanized antibodies” generally refer to non-human antibodies that have had the variable-domain framework regions swapped for sequences found in human antibodies. Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536, all entirely incorporated by reference. “Backmutation” of selected acceptor framework residues to the corresponding donor residues is often required to regain affinity that is lost in the initial grafted construct (U.S. Pat. Nos. 5,530,101; 5,585,089; 5,693,761; 5,693,762; 6,180,370; 5,859,205; 5,821,337; 6,054,297; 6,407,213, all entirely incorporated by reference). The humanized antibody optimally also will comprise at least a portion of an immunoglobulin constant region, typically that of a human immunoglobulin, and thus will typically comprise a human Fc region. Humanized antibodies can also be generated using mice with a genetically engineered immune system, as described for example in Roque et al., 2004, Biotechnol. Prog. 20:639-654, entirely incorporated by reference. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein, all entirely incorporated by reference). Humanization methods include but are not limited to methods described in Jones et al., 1986, Nature 321:522-525; Riechmann et al., 1988; Nature 332:323-329; Verhoeyen et al., 1988, Science, 239:1534-1536; Queen et al., 1989, Proc Natl Acad Sci, USA 86:10029-33; He et al., 1998, J. Immunol. 160: 1029-1035; Carter et al., 1992, Proc Natl Acad Sci, USA 89:4285-9, Presta et al., 1997, Cancer Res. 57(20):4593-9; Gorman et al., 1991, Proc. Natl. Acad. Sci. USA 88:4181-4185; O'Connor et al., 1998, Protein Eng 11:321-8, all entirely incorporated by reference. Humanization or other methods of reducing the immunogenicity of nonhuman antibody variable regions may include resurfacing methods, as described for example in Roguska et al., 1994, Proc. Natl. Acad. Sci. USA 91:969-973, entirely incorporated by reference. Other humanization methods may involve the grafting of only parts of the CDRs, including but not limited to methods described in Tan et al., 2002, J. Immunol. 169:1119-1125; De Pascalis et al., 2002, J. Immunol. 169:3076-3084, all entirely incorporated by reference.

In some embodiments, the antibodies of the current invention comprise a heavy chain variable region derived from a particular human germline heavy chain immunoglobulin gene and/or a light chain variable region derived from a particular human germline light chain immunoglobulin gene. Such antibodies may contain amino acid differences as compared to the human germline sequences, due to, for example, naturally-occurring somatic mutations or intentional introduction of site-directed mutation. However, a humanized antibody typically is at least 80% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the human germline immunoglobulin gene. Typically, a humanized antibody derived from a particular human germline sequence will display no more than 10-20 amino acid differences from the amino acid sequence encoded by the human germline immunoglobulin gene. In certain cases, the humanized antibody may display no more than 5, or even no more than 4, 3, 2, or 1 amino acid difference from the amino acid sequence encoded by the germline immunoglobulin gene.

In some embodiments, the antibodies of the current disclosure are humanized and affinity matured, as is known in the art. Structure-based methods may be employed for humanization and affinity maturation, for example as described in U.S. Pat. No. 7,657,380. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference.

### IV. Characteristics of the Anti-CD27 Antibodies

In some embodiments, the anti-CD27 antibodies described herein bind to human CD27. In some embodiments, binding of the anti-CD27 antibodies to human CD27 is measured by ELISA or any other method known to a person skilled in the art.

In some embodiments, the anti-CD27 antibodies described herein bind human CD27 with high affinities. The K_{D} value can be measured with the antigen immobilized or with the antibody immobilized. The K_{D} value can also be measured in a monovalent or a bivalent binding mode.

In some embodiments, the anti-CD27 antibodies display low immunogenicity when administered into human subjects. These antibodies can contain an Fc domain derived from human IgG1, human IgG2, human IgG3, or human IgG4. In some embodiments, these antibodies are humanized using the framework regions derived from human immunoglobulins.

Effects of the anti-CD27 antibodies on T cell function can be assayed using a variety of methods known in the art and described herein. Accordingly, the anti-CD27 antibodies can serve as CD27 agonists or CD27 antagonists.

In some embodiments, anti-CD27 antibodies described act as CD27 agonists, and as a result, such anti-CD27 antibodies induce or enhance an immune response. In some embodiments, anti-CD27 antibodies described act by inducing or enhancing an immune response against an antigen. In some embodiments, inducing or enhancing an immune response means activating immune cells. In some embodiments, inducing or enhancing an immune response means activating immune cells to a particular antigen. In some embodiments, inducing or enhancing an immune response means providing a co-stimulatory signal. In some embodiments, inducing or enhancing an immune response means activating T cells. In some embodiments, inducing or enhancing an immune response means activating B cells. In some embodiments, inducing or enhancing an immune response means increasing the cytotoxic T lymphocyte response. In some embodiments, inducing or enhancing an immune response means increasing CD4+T effector cell function. In some embodiments, inducing or enhancing an immune response means increasing CD8+T effector cell function. In some embodiments, inducing or enhancing an immune response means increasing antigen-specific T cell function, proliferation, and/or activation. In some embodiments, inducing or enhancing an immune response means increasing an antigen-specific Th1 response. In some embodiments, inducing or enhancing an immune response means increasing or supporting memory cell formation. In some embodiments, inducing or enhancing an immune response means initiating ADCC. In some embodiments, inducing or enhancing an immune response means initiating ADCP. In some embodiments, ADCC can be induced to cause at least about 10%, 20%, 30%, 40%, 50%, 60%, 70%, 80%, 90%, or 100% lysis of CD27 expressing cells.

In some embodiments, anti-CD27 antibodies described act as CD27 antagonists, and as a result, such anti-CD27 antibodies inhibit the binding of CD70 to CD27. In some embodiments, the anti-CD27 antibodies compete with CD70 for binding to CD27. In some embodiments, inhibition of CD70 by anti-CD27 antibodies may be partial inhibition. In some embodiment, inhibition of CD70 by anti-CD27 antibodies may be full inhibition. In some embodiments, anti-CD27 antibodies inhibit binding by at least 10%, 20%, 30%, 40%, 50%, 60%, 70%, 80%, 90%, or 100%. In some embodiments, inhibiting an immune response means stopping CD27+ cancer cell growth. In some embodiments, inhibiting an immune response means stopping cell growth in order to treat the cancer. In some embodiments, stopping cell growth means stopping growth of CD27+ Tregs in order to treat cancer. In some embodiments, inhibiting cell growth can include inhibiting the growth of a CD27 expressing cancer cell.

Efficacy readouts can include monitoring for changes in αβ and/or γδ T cells, cytotoxic T cell activity, changes in markers such as CD137, CD107a, changes in NK and/or NK/T activity, interferon-γ production, changes in regulatory T-cell (including changes in Treg number), changes in macrophage number, changes in neutrophil pro-tumorigenic activity, T-cell activation, CTL activation, changes in activation markers such as CD45RA or CCR7, as well as cancer cell cytotoxicity assays. Efficacy readouts can also include tumor size reduction, tumor number reduction, reduction in the number of metastases, and decreased disease state (or increased life expectancy). In some embodiments, inhibiting cell growth means tumor inhibition or a reduction in tumor size. In some embodiments, a reduction in tumor size by 10%, 20%, 30%, 40%, 50%, 60%, 70%, 80%, 90%, 95%, or 100% is indicative of therapeutic efficacy. In some embodiments, a reduction in tumor number by 10%, 20%, 30%, 40%, 50%, 60%, 70%, 80%, 90%, 95%, or 100% is indicative of therapeutic efficacy. In some embodiments, a reduction in tumor burden by 10%, 20%, 30%, 40%, 50%, 60%, 70%, 80%, 90%, 95%, or 100% is indicative of therapeutic efficacy. In some embodiments, a reduction in the number of metastases by 10%, 20%, 30%, 40%, 50%, 60%, 70%, 80%, 90%, 95%, or 100% is indicative of therapeutic efficacy.

### V. Nucleic Acids of the Invention

Nucleic acids encoding the anti-CD27 antibodies described herein also encompass the invention, as well as expression vectors containing such nucleic acids and host cells transformed with such nucleic acids and/or expression vectors. As will be appreciated by those in the art, the protein sequences depicted herein can be encoded by any number of possible nucleic acid sequences due to the degeneracy of the genetic code.

Nucleic acid compositions encoding the anti-CD27 antibodies and/or CD27-binding domains also encompass the invention. As will be appreciated by those in the art, in the case of antigen binding domains, the nucleic acid compositions generally include a first nucleic acid encoding the heavy chain variable region and a second nucleic acid encoding the light chain variable region. In the case of scFvs, a single nucleic acid encoding the heavy chain variable region and light chain variable region, separated by a linker described herein, can be made. In the case of traditional antibodies, the nucleic acid compositions generally include a first nucleic acid encoding the heavy chain and a second nucleic acid encoding the light chain, which will, upon expression in a cell, spontaneously assemble into the “traditional” tetrameric format of two heavy chains and two light chains.

As is known in the art, the nucleic acids encoding the components of the invention can be incorporated into expression vectors, and depending on the host cells, used to produce the antibodies of the invention. These two nucleic acids can be incorporated into a single expression vector or into two different expression vectors. Generally, the nucleic acids can be operably linked to any number of regulatory elements (promoters, origin of replication, selectable markers, ribosomal binding sites, inducers, etc.) in an expression vector. The expression vectors can be extra-chromosomal or integrating vectors.

The nucleic acids and/or expression vectors of the current invention can be introduced into any type of host cells, which are well known in the art, including mammalian, bacterial, yeast, insect and fungal cells. After transfection, single cell clones can be isolated for cell bank generation using methods known in the art, such as limited dilution, ELISA, FACS, microscopy, or Clonepix. Clones can be cultured under conditions suitable for bio-reactor scale-up and maintained expression of the antibodies. The antibodies can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

### VI. Therapeutic Applications

The current disclosure provides a method of modulating an immune response in a subject, and the method includes administering to the subject an effective amount of an anti-CD27 antibody described herein, or a pharmaceutical composition containing an anti-CD27 antibody.

In some embodiments, the methods of modulating an immune response encompassed by the present disclosure comprises inhibiting an immune response in a subject, and in further embodiments, such methods comprise administering to the subject an effective amount of an anti-CD27 antibody that acts as a CD27 antagonist, or by administering a pharmaceutical composition containing an antagonistic anti-CD27 antibody.

In some embodiments, the present disclosure provides methods for inducing or enhancing an immune response in a subject, for example, by administering to the subject an effective amount of an anti-CD27 antibody that acts as a CD27 agonist, or by administering to the subject a pharmaceutical composition containing such an agonistic anti-CD27 antibody.

The present disclosure also provides methods of treating cancer in a subject, and such methods include administering to the subject an effective amount of an anti-CD27 antibody that acts as a CD27 antagonist, or a pharmaceutical composition containing such anti-CD27 antibody. In some embodiments, the cancer to be treated expresses CD27 on the cancer cell surface. In some embodiments, the cancer to be treated upregulates CD27 compared to the corresponding non-cancerous tissue. In some embodiments, the subject to be treated expresses CD27 on T cells, such as on CD8+ and/or CD4+ T cells. In some embodiments, the subject to be treated expresses a high level of CD27 on one or more types of immune cells including CD4+ T cells, CD8+ T cells, B cells, natural killer T cells, natural killer cells, macrophages, and dendritic cells. In some embodiments, the cancer to be treated uses the CD27/CD70 pathway to promote tumor growth. In some embodiments, the cancer to treated is non-responsive to existing immune-modulating antibodies targeting other immune checkpoints, such as CTLA-4, PD-1 or PD-L1.

“Cancer therapy” herein refers to any method which prevents or treats cancer or ameliorates one or more of the symptoms of cancer. Typically, such therapies will comprise administration of anti-CD27 alone or in combination (including for example, in combination with integrin-binding polypeptide-Fc fusions), as well as potentially in combination with chemotherapy or radiotherapy or other biologics and for enhancing the activity thereof. In some embodiments, cancer therapy can include or be measured by increased survival. In some embodiments, cancer therapy results in a reduction in tumor volume.

“Cancer,” as used herein, refers broadly to any neoplastic disease (whether invasive non-invasive or metastatic) characterized by abnormal and uncontrolled cell division causing malignant growth or tumor (e.g., unregulated cell growth). As used herein, we may use the terms “cancer” (or “cancerous”), “hyperproliferative,” and “neoplastic” to refer to cells having the capacity for autonomous growth (i.e., an abnormal state or condition characterized by rapidly proliferating cell growth). Non-limiting examples of which are described herein. This includes any physiological condition in mammals that is typically characterized by unregulated cell growth. Examples of cancer are exemplified in the working examples and also are described within the specification. The terms “cancer” or “neoplasm” are used to refer to malignancies of the various organ systems, including those affecting the lung, breast, thyroid, lymph glands and lymphoid tissue, gastrointestinal organs, and the genitourinary tract, as well as to adenocarcinomas which are generally considered to include malignancies such as most colon cancers, renal-cell carcinoma, prostate cancer and/or testicular tumors, non-small cell carcinoma of the lung, cancer of the small intestine, cancer of the esophagus, CD27 Expressing B-cell Malignancies for Example Hodgkin's Lymphoma, Chronic Lymphocytic Leukemia, Mantle Cell Lymphoma, Marginal Zone B Cell Lymphoma), Any T-cell Malignancy, Solid Tumors (Metastatic Melanoma, Renal (Clear) Cell Carcinoma, Hormone-refractory Prostate Adenocarcinoma, Ovarian Cancer, Colorectal Adenocarcinoma, Non-small Cell Lung Cancer), Burkett's Lymphoma, Primary Lymphoma of the Central Nervous System, Advanced Breast Carcinoma, Advanced Ovarian Carcinoma, Prostate Cancer, Squamous Cell Carcinoma of the Head and Neck (SCCHN), Ovarian Carcinoma, Colorectal Cancer (CRC), Renal Cell Carcinoma (RCC), Glioblastoma (GBM), Carcinoma, Renal Cell, Kidney Diseases, Kidney Neoplasms, Urogenital Neoplasms, Urologic Disease, Urologic Neoplasms, Neoplasms, Neoplasms by Histologic Type, Clear-cell Metastatic Renal Cell Carcinoma, Unresectable Stage III or Stage IV Melanoma, Carcinoma, Renal Cell, Kidney Diseases, Kidney Neoplasms, Urogenital Neoplasms, Urologic Diseases, Urologic Neoplasms, Neoplasms by Histologic Type, Neoplasms, Clear-cell Metastatic Renal Cell Carcinoma, Melanoma, Triple Negative Breast Cancer, Bladder Cancer, Head and Neck Cancer, Non-small cell Lung Cancer, Aggressive B-Cell Lymphomas, and B-Cell Lymphomas.

Non-limiting examples of cancers that can be treated using the present disclosure include, but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; multiple myeloma and post-transplant lymphoproliferative disorder (PTLD). In some embodiments, other cancers amenable for treatment by the present invention include, but are not limited to, carcinoma, lymphoma, blastoma, sarcoma, and leukemia or lymphoid malignancies. More particular examples of such cancers include colorectal, bladder, ovarian, melanoma, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Preferably, the cancer is selected from the group consisting of colorectal cancer, breast cancer, rectal cancer, non-small cell lung cancer, non-Hodgkin's lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. In an exemplary embodiment the cancer is an early or advanced (including metastatic) bladder, ovarian or melanoma. In another embodiment the cancer is colorectal cancer. In some embodiments, the methods of the present invention are useful for the treatment of vascularized tumors.

Hyperproliferative and neoplastic disease states may be categorized as pathologic (i.e., characterizing or constituting a disease state), or they may be categorized as non-pathologic (i.e., as a deviation from normal but not associated with a disease state). The terms are meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

Examples of cellular proliferative and/or differentiative disorders include cancer (e.g., carcinoma, sarcoma, metastatic disorders or hematopoietic neoplastic disorders, e.g., leukemias). A metastatic tumor can arise from a multitude of primary tumor types, including but not limited to those of prostate, colon, lung, breast and liver. Accordingly, the compositions used herein and optionally at least one additional therapeutic agent to treat cancer, can be administered to a patient who has cancer.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. In some embodiments, the diseases arise from poorly differentiated acute leukemias (e.g., erythroblastic leukemia and acute megakaryoblastic leukemia). Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit. Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macro globulinemia (WM). Additional forms of malignant lymphomas include, but are not limited to non-Hodgkin lymphoma and variants thereof, peripheral T cell lymphomas, adult T cell leukemia/lymphoma (ATL), cutaneous T cell lymphoma (CTCL), large granular lymphocytic leukemia (LGF), Hodgkin's disease and Reed-Sternberg disease.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. The mutant IL-2 polypeptides can be used to treat patients who have, who are suspected of having, or who may be at high risk for developing any type of cancer, including renal carcinoma or melanoma, or any viral disease. Exemplary carcinomas include those forming from tissue of the cervix, lung, prostate, breast, head and neck, colon and ovary. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

In some embodiments, the cancer to be treated is CD27 Expressing B-cell Malignancies, Hodgkin's Lymphoma, Chronic Lymphocytic Leukemia, Mantle Cell Lymphoma, Marginal Zone B Cell Lymphoma, T-cell Malignancy, Solid Tumors, Metastatic Melanoma, Renal (Clear) Cell Carcinoma, Hormone-refractory Prostate Adenocarcinoma, Ovarian Cancer, Colorectal Adenocarcinoma, Non-small Cell Lung Cancer, Burkett's Lymphoma, Primary Lymphoma of the Central Nervous System, Advanced Breast Carcinoma, Advanced Ovarian Carcinoma, Prostate Cancer, Squamous Cell Carcinoma of the Head and Neck (SCCHN), Ovarian Carcinoma, Colorectal Cancer (CRC), Renal Cell Carcinoma (RCC), Glioblastoma (GBM), Carcinoma, Renal Cell, Kidney Diseases, Kidney Neoplasms, Urogenital Neoplasms, Urologic Diseases, Urologic Neoplasms, Neoplasms, Neoplasms by Histologic Type, Clear-cell Metastatic Renal Cell Carcinoma, Unresectable Stage III or Stage IV Melanoma, Carcinoma, Renal Cell, Kidney Diseases, Kidney Neoplasms, Urogenital Neoplasms, Urologic Diseases, Urologic Neoplasms, Neoplasms by Histologic Type, Neoplasms Clear-cell Metastatic Renal Cell Carcinoma, Melanoma, Triple Negative Breast Cancer, Bladder Cancer, Head and Neck Cancer, Non-small Cell Lung Cancer, Aggressive B-Cell Lymphomas, and/or B-Cell Lymphomas.

In some embodiments, the cancer to be treated using an anti-CD27 monothereapy as described herein is CD27 Expressing B-cell Malignancies, Hodgkin's Lymphoma, Chronic Lymphocytic Leukemia, Mantle Cell Lymphoma, Marginal Zone B Cell Lymphoma, T-cell Malignancy, Solid Tumors, Metastatic Melanoma, Renal (Clear) Cell Carcinoma, Hormone-refractory Prostate Adenocarcinoma, Ovarian Cancer, Colorectal Adenocarcinoma, Non-small Cell Lung Cancer, Burkett's Lymphoma, and/or Primary Lymphoma of the Central Nervous System.

In some embodiments, the cancer to be treated using an anti-CD27 combination therapy, for example as a combination therapy with an immune checkpoint inhibitor as described herein, is Advanced Breast Carcinoma, Advanced Ovarian Carcinoma, Prostate Cancer, Squamous Cell Carcinoma of the Head and Neck (SCCHN), Ovarian Carcinoma, Colorectal Cancer (CRC), Renal Cell Carcinoma (RCC), Glioblastoma (GBM), Carcinoma, Renal Cell, Kidney Diseases, Kidney Neoplasms, Urogenital Neoplasms, Urologic Diseases, Urologic Neoplasms, Neoplasms, Neoplasms by Histologic Type, Clear-cell Metastatic Renal Cell Carcinoma, Unresectable Stage III or Stage IV Melanoma, Carcinoma, Renal Cell, Kidney Diseases, Kidney Neoplasms, Urogenital Neoplasms, Urologic Diseases, Urologic Neoplasms, Neoplasms by Histologic Type, Neoplasms Clear-cell Metastatic Renal Cell Carcinoma, Melanoma, Triple Negative Breast Cancer, Bladder Cancer, Head and Neck Cancer, Non-small Cell Lung Cancer, Aggressive B-Cell Lymphomas, and B-Cell Lymphomas.

### VII. Combination Therapy

Anti-CD27 antibodies described herein can be used in combination with additional therapeutic agents to treat cancer.

It will be appreciated by those skilled in the art that amounts for each of the anti-CD27 antibodies, and optionally at least one or more additional therapeutic agents used to treat cancer, that are sufficient to reduce tumor growth and size, or a therapeutically effective amount, will vary not only on the particular compounds or compositions selected, but also with the route of administration, the nature of the condition being treated, and the age and condition of the patient, and will ultimately be at the discretion of the patient's physician or pharmacist. The length of time during which the compounds used in the instant method will be given varies on an individual basis.

In some embodiments, the one or more additional therapeutic agents used to treat cancer are immune checkpoint inhibitors. As described herein, immune checkpoint inhibitors include anti-PD-1 inhibitors, anti-PD-L1 inhibitors, anti-CTLA-4 inhibitors, anti-TIM-3 inhibitors, anti-LAG-3 inhibitors. Examples of types of immune checkpoint inhibitors include antibodies. In some embodiments, the anti-PD-1 antibody is selected from the group consisting of nivolumab, pembrolizumab, AMP-224, AMP-514 (also known as MEDI0680), spartalizumab (also known as PDR001), and cemiplimab. In some embodiments, the anti-PD-1 antibody is nivolumab. In some embodiments, the anti-PD-1 antibody is pembrolizumab. In some embodiments, the anti-PD-1 antibody is AMP-224. In some embodiments, the anti-PD-1 antibody is AMP-514 (also known as MEDI0680). In some embodiments, the anti-PD-1 antibody is spartalizumab (also known as PDR001). In some embodiments, the anti-PD-1 antibody is cemiplimab. In some embodiments, the anti-CTLA-4 antibody is selected form the group consisting of ipilimumab and tremelimumab. In some embodiments, the anti-PD-L1 antibody is atezolizumab. In some embodiments, the anti-PD-L1 antibody is avelumab. In some embodiments, the anti-PD-L1 antibody is durvalumab. In some embodiments, the anti-PD-L1 antibody is BMS-936559. In some embodiments, the anti-PD-L1 antibody is CK-301. In some embodiments, the anti-LAG-3 is IMP-321.

In some embodiments, the additional therapeutic agent is an anti-CD20 antibody such as rituximab. In some embodiments, the additional therapeutic agent is an anti-Her2/neu antibody, such as trastuzumab. In some embodiments, the additional therapeutic agent is a multi-targeted receptor tyrosine kinase inhibitor, such as sunitinib.

In some embodiments, the one or more additional therapeutic agents used to treat cancer are tumor targeting agents. As described herein, tumor targeting agents include, but are not limited to rituximab, trastuzumab, sunitinib, atezolizumab, olaratumab, daratumumab, elotuzumab, necitumumab, dinutuximab, pertuzumab, panitumumab, cetuximab, and integrin-binding Fc-fusion polypeptides (including, for example, NOD-201. In some embodiments, the one or more additional therapeutic agents used to treat cancer are adjuvants. As described herein, adjuvants include but are not limited to chemotherapy, atezolizumab, ipilimumab, pembrolizumab, nivolumab, and durvalumab. In some embodiments, the one or more additional therapeutic agents used to treat cancer is radiation.

In some embodiments, the combination is specific the particular disease. In some embodiments, an anti-CD27 antibody as described herein is used in combination with an adjuvant for the treatment of Advanced Breast Carcinoma. In some embodiments, an anti-CD27 antibody as described herein is used in combination with an adjuvant for the treatment of Advanced Ovarian Carcinoma. In some embodiments, an anti-CD27 antibody as described herein is used in combination with radiation for the treatment of Prostate Cancer. In some embodiments, an anti-CD27 antibody as described herein is used in combination with nivolumab for the treatment of Squamous Cell Carcinoma of the Head and Neck (SCCHN). In some embodiments, an anti-CD27 antibody as described herein is used in combination with nivolumab for the treatment of Ovarian Carcinoma. In some embodiments, an anti-CD27 antibody as described herein is used in combination with nivolumab for the treatment of Colorectal Cancer (CRC). In some embodiments, an anti-CD27 antibody as described herein is used in combination with nivolumab for the treatment of Renal Cell Carcinoma (RCC). In some embodiments, an anti-CD27 antibody as described herein is used in combination with nivolumab for the treatment of Glioblastoma (GBM). In some embodiments, an anti-CD27 antibody as described herein is used in combination with sunitinib for the treatment of Renal Cell Carcinoma (RCC). In some embodiments, an anti-CD27 antibody as described herein is used in combination with sunitinib for the treatment of Kidney Diseases. In some embodiments, an anti-CD27 antibody as described herein is used in combination with sunitinib for the treatment of Kidney Neoplasms. In some embodiments, an anti-CD27 antibody as described herein is used in combination with sunitinib for the treatment of Urogenital Neoplasms. In some embodiments, an anti-CD27 antibody as described herein is used in combination with sunitinib for the treatment of Urologic Diseases. In some embodiments, an anti-CD27 antibody as described herein is used in combination with sunitinib for the treatment of Urologic Neoplasms. In some embodiments, an anti-CD27 antibody as described herein is used in combination with sunitinib for the treatment of Neoplasms. In some embodiments, an anti-CD27 antibody as described herein is used in combination with sunitinib for the treatment of Neoplasms by Histologic Type. In some embodiments, an anti-CD27 antibody as described herein is used in combination with sunitinib for the treatment of Clear-cell Metastatic Renal Cell Carcinoma. In some embodiments, an anti-CD27 antibody as described herein is used in combination with ipilimumab for the treatment of Unresectable Stage III or Stage IV Melanoma. In some embodiments, an anti-CD27 antibody as described herein is used in combination with atezolizumab for the treatment of Renal Cell Carcinoma (RCC). In some embodiments, an anti-CD27 antibody as described herein is used in combination with atezolizumab for the treatment of Kidney Diseases. In some embodiments, an anti-CD27 antibody as described herein is used in combination with atezolizumab for the treatment of Kidney Neoplasms. In some embodiments, an anti-CD27 antibody as described herein is used in combination with atezolizumab for the treatment of Urogenital Neoplasms. In some embodiments, an anti-CD27 antibody as described herein is used in combination with atezolizumab for the treatment of Urologic Diseases. In some embodiments, an anti-CD27 antibody as described herein is used in combination with atezolizumab for the treatment of Urologic Neoplasms. In some embodiments, an anti-CD27 antibody as described herein is used in combination with atezolizumab for the treatment of Neoplasms by Histologic Type. In some embodiments, an anti-CD27 antibody as described herein is used in combination with atezolizumab for the treatment of Neoplasms. In some embodiments, an anti-CD27 antibody as described herein is used in combination with atezolizumab for the treatment of Clear-cell Metastatic Renal Cell Carcinoma. In some embodiments, an anti-CD27 antibody as described herein is used in combination with atezolizumab for the treatment of Melanoma. In some embodiments, an anti-CD27 antibody as described herein is used in combination with atezolizumab for the treatment of Triple Negative Breast Cancer. In some embodiments, an anti-CD27 antibody as described herein is used in combination with atezolizumab for the treatment of Bladder Cancer. In some embodiments, an anti-CD27 antibody as described herein is used in combination with atezolizumab for the treatment of Head and Neck Cancer. In some embodiments, an anti-CD27 antibody as described herein is used in combination with atezolizumab for the treatment of Non-small Cell Lung Cancer. In some embodiments, an anti-CD27 antibody as described herein is used in combination with nivolumab for the treatment of Aggressive B-Cell Lymphomas. In some embodiments, an anti-CD27 antibody as described herein is used in combination with rituximab for the treatment of B-Cell Lymphomas.

In some embodiments, the anti-CD27 antibodies and at least one or more additional therapeutic agents used to treat cancer inhibit growth and/or proliferation of tumor cells. In some embodiments, the anti-CD27 antibodies and at least one or more additional therapeutic agents used to treat cancer reduce tumor size. In certain embodiments, the anti-CD27 antibodies and at least one or more additional therapeutic agents used to treat cancer inhibit metastases of a primary tumor.

In some embodiments, the anti-CD27 antibodies and at least one or more checkpoint inhibitors inhibit growth and/or proliferation of tumor cells. In some embodiments, the anti-CD27 antibodies and at least one or more checkpoint inhibitors reduce tumor size. In certain embodiments, the anti-CD27 antibodies and at least one or more checkpoint inhibitors inhibit metastases of a primary tumor.

In some embodiments, the anti-CD27 antibodies can be combined with an adjuvant to treat advanced breast carcinoma. In some embodiments, the anti-CD27 antibodies can be combined with an adjuvant to treat advanced ovarian carcinoma.

In some embodiments, the anti-CD27 antibodies are used in conjunction with a surgical method to treat cancer.

In some embodiments, the anti-CD27 antibodies can be combined with an anti-PD-1 inhibitor to treat squamous cell carcinoma of the head and neck (SCCHN). In some embodiments, the anti-CD27 antibodies can be combined with an anti-PD-1 antibody to treat squamous cell carcinoma of the head and neck (SCCHN). In some embodiments, the anti-CD27 antibodies can be combined with nivolumab to treat squamous cell carcinoma of the head and neck (SCCHN).

In some embodiments, the anti-CD27 antibodies can be combined with an anti-PD-1inhibitor to treat ovarian carcinoma. In some embodiments, the anti-CD27 antibodies can be combined with an anti-PD-1 antibody to treat ovarian carcinoma. In some embodiments, the anti-CD27 antibodies can be combined with nivolumab to treat ovarian carcinoma.

In some embodiments, the anti-CD27 antibodies can be combined with an anti-PD-1 inhibitor to treat colorectal cancer (CRC). In some embodiments, the anti-CD27 antibodies can be combined with an anti-PD-1 antibody to treat colorectal cancer (CRC). In some embodiments, the anti-CD27 antibodies can be combined with nivolumab to treat colorectal cancer (CRC).

In some embodiments, the anti-CD27 antibodies can be combined with an anti-PD-1 inhibitor to treat renal cell carcinoma (RCC). In some embodiments, the anti-CD27 antibodies can be combined with an anti-PD-1 antibody to treat renal cell carcinoma (RCC). In some embodiments, the anti-CD27 antibodies can be combined with nivolumab to treat renal cell carcinoma (RCC).

In some embodiments, the anti-CD27 antibodies can be combined with an anti-PD-1 inhibitor to treat glioblastoma (GBM). In some embodiments, the anti-CD27 antibodies can be combined with an anti-PD-1 antibody to treat glioblastoma (GBM). In some embodiments, the anti-CD27 antibodies can be combined with nivolumab to treat glioblastoma (GBM).

In some embodiments, the anti-CD27 antibodies can be combined with a CTLA-4 inhibitor to treat unresectable stage III or stage IV melanoma. In some embodiments, the anti-CD27 antibodies can be combined with a CTLA-4 antibody to treat unresectable stage III or stage IV melanoma. In some embodiments, the anti-CD27 antibodies can be combined with ipilimumab to treat unresectable stage III or stage IV melanoma.

In some embodiments, the anti-CD27 antibodies can be combined with a PD-L1 inhibitor to treat renal cell carcinoma (RCC). In some embodiments, the anti-CD27 antibodies can be combined with a PD-L1 antibody to treat renal cell carcinoma (RCC). In some embodiments, the anti-CD27 antibodies can be combined with atezolizumab to treat renal cell carcinoma (RCC).

In some embodiments, the anti-CD27 antibodies can be combined with a PD-L1 inhibitor to treat kidney disease. In some embodiments, the anti-CD27 antibodies can be combined with a PD-L1 antibody to treat kidney disease. In some embodiments, the anti-CD27 antibodies can be combined with a atezolizumab to treat kidney disease.

In some embodiments, the anti-CD27 antibodies can be combined with a PD-L1 inhibitor to treat kidney neoplasms. In some embodiments, the anti-CD27 antibodies can be combined with a PD-L1 antibody to treat kidney neoplasms. In some embodiments, the anti-CD27 antibodies can be combined with a atezolizumab to treat kidney neoplasms.

In some embodiments, the anti-CD27 antibodies can be combined with a PD-L1 inhibitor to treat urogenital neoplasms. In some embodiments, the anti-CD27 antibodies can be combined with a PD-L1 antibody to treat urogenital neoplasms. In some embodiments, the anti-CD27 antibodies can be combined with a atezolizumab to treat urogenital neoplasms.

In some embodiments, the anti-CD27 antibodies can be combined with a PD-L1 inhibitor to treat urologic diseases. In some embodiments, the anti-CD27 antibodies can be combined with a PD-L1 antibody to treat urologic diseases. In some embodiments, the anti-CD27 antibodies can be combined with a atezolizumab to treat urologic diseases.

In some embodiments, the anti-CD27 antibodies can be combined with a PD-L1 inhibitor to treat urologic neoplasms. In some embodiments, the anti-CD27 antibodies can be combined with a PD-L1 antibody to treat urologic neoplasms. In some embodiments, the anti-CD27 antibodies can be combined with a atezolizumab to treat urologic neoplasms.

In some embodiments, the anti-CD27 antibodies can be combined with a PD-L1 inhibitor to treat neoplasms. In some embodiments, the anti-CD27 antibodies can be combined with a PD-L1 antibody to treat neoplasms. In some embodiments, the anti-CD27 antibodies can be combined with a atezolizumab to treat neoplasms.

In some embodiments, the anti-CD27 antibodies can be combined with a PD-L1 inhibitor to treat neoplasms by histologic type. In some embodiments, the anti-CD27 antibodies can be combined with a PD-L1 antibody to treat neoplasms by histologic type. In some embodiments, the anti-CD27 antibodies can be combined with a atezolizumab to treat neoplasms by histologic type.

In some embodiments, the anti-CD27 antibodies can be combined with a PD-L1 inhibitor to treat clear-cell metastatic renal cell carcinoma. In some embodiments, the anti-CD27 antibodies can be combined with a PD-L1 antibody to treat clear-cell metastatic renal cell carcinoma. In some embodiments, the anti-CD27 antibodies can be combined with a atezolizumab to treat clear-cell metastatic renal cell carcinoma.

In some embodiments, the anti-CD27 antibodies can be combined with a PD-L1 inhibitor to treat melanoma. In some embodiments, the anti-CD27 antibodies can be combined with a PD-L1 antibody to treat melanoma. In some embodiments, the anti-CD27 antibodies can be combined with a atezolizumab to treat melanoma.

In some embodiments, the anti-CD27 antibodies can be combined with a PD-L1 inhibitor to treat triple negative breast cancer. In some embodiments, the anti-CD27 antibodies can be combined with a PD-L1 antibody to treat triple negative breast cancer. In some embodiments, the anti-CD27 antibodies can be combined with a atezolizumab to treat triple negative breast cancer.

In some embodiments, the anti-CD27 antibodies can be combined with a PD-L1 inhibitor to treat bladder cancer. In some embodiments, the anti-CD27 antibodies can be combined with a PD-L1 antibody to treat bladder cancer. In some embodiments, the anti-CD27 antibodies can be combined with a atezolizumab to treat bladder cancer.

In some embodiments, the anti-CD27 antibodies can be combined with a PD-L1 inhibitor to treat head and neck cancer. In some embodiments, the anti-CD27 antibodies can be combined with a PD-L1 antibody to treat head and neck cancer. In some embodiments, the anti-CD27 antibodies can be combined with a atezolizumab to treat head and neck cancer.

In some embodiments, the anti-CD27 antibodies can be combined with a PD-L1 inhibitor to treat non-small cell lung cancer. In some embodiments, the anti-CD27 antibodies can be combined with a PD-L1 antibody to treat non-small cell lung cancer. In some embodiments, the anti-CD27 antibodies can be combined with a atezolizumab to treat non-small cell lung cancer.

In some embodiments, the anti-CD27 antibodies can be combined with a PD-1 inhibitor to treat aggressive B-cell lymphomas. In some embodiments, the anti-CD27 antibodies can be combined with a PD-1 antibody to treat aggressive B-cell lymphomas. In some embodiments, the anti-CD27 antibodies can be combined with a nivolumab to treat aggressive B-cell lymphomas.

In some embodiments, the anti-CD27 antibodies and at least one or more tumor targeting agents inhibit growth and/or proliferation of tumor cells. In some embodiments, the anti-CD27 antibodies and at least one or more tumor targeting agents reduce tumor size. In certain embodiments, the anti-CD27 antibodies and at least one or more tumor targeting agents inhibit metastases of a primary tumor.

In some embodiments, the anti-CD27 antibodies can be combined with rituximab to treat B-cell lymphomas.

In some embodiments, the anti-CD27 antibodies can be combined with sunitinib to treat renal cell carcinoma. In some embodiments, the anti-CD27 antibodies can be combined with sunitinib to treat kidney diseases. In some embodiments, the anti-CD27 antibodies can be combined with sunitinib to treat kidney neoplasms. In some embodiments, the anti-CD27 antibodies can be combined with sunitinib to treat urogenital neoplasms. In some embodiments, the anti-CD27 antibodies can be combined with sunitinib to treat urologic diseases. In some embodiments, the anti-CD27 antibodies can be combined with sunitinib to treat urologic neoplasms. In some embodiments, the anti-CD27 antibodies can be combined with sunitinib to treat neoplasms. In some embodiments, the anti-CD27 antibodies can be combined with sunitinib to treat neoplasms by histologic type. In some embodiments, the anti-CD27 antibodies can be combined with sunitinib to treat clear-cell metastatic renal cell carcinoma.

In some embodiments, the anti-CD27 antibodies can be combined with the integrin-binding polypeptide-Fc fusions described herein to treat cancer. In some embodiments, the anti-CD27 antibodies can be combined with the integrin-binding polypeptide-Fc fusions described herein along with at least one additional therapeutic agent used to treat cancer discussed herein to treat cancer. In some embodiments, the integrin-binding polypeptide-Fc fusions is one as described herein. In some embodiments, the integrin-binding polypeptide-Fc fusions is one as described herein in Table 2. In some embodiments, the integrin-binding polypeptide-Fc fusion comprises a sequence selected from the group consisting of SEQ ID NOs: 59-127. In some embodiments, the integrin-binding polypeptide-Fc fusions is NOD-201. In some embodiments, the integrin-binding polypeptide-Fc fusions is SEQ ID NO: 126. In some embodiments, the integrin-binding polypeptide-Fc fusions is SEQ ID NO: 127.

Efficacy readouts can include monitoring for changes in αβ and/or γδ T cells, cytotoxic T cell activity, changes in markers such as CD137, CD107a, changes in NK and/or NK/T activity, interferon-γ production, changes in regulatory T-cell (including changes in Treg number), changes in macrophage number, changes in neutrophil pro-tumorigenic activity, T-cell activation, CTL activation, changes in activation markers such as CD45RA or CCR7, as well as cancer cell cytotoxicity assays. Efficacy readouts can also include tumor size reduction, tumor number reduction, reduction in the number of metastases, and decreased disease state (or increased life expectancy). In some embodiments, inhibiting cell growth means tumor inhibition or a reduction in tumor size. In some embodiments, a reduction in tumor size by 10%, 20%, 30%, 40%, 50%, 60%, 70%, 80%, 90%, 95%, or 100% is indicative of therapeutic efficacy. In some embodiments, a reduction in tumor number by 10%, 20%, 30%, 40%, 50%, 60%, 70%, 80%, 90%, 95%, or 100% is indicative of therapeutic efficacy. In some embodiments, a reduction in tumor burden by 10%, 20%, 30%, 40%, 50%, 60%, 70%, 80%, 90%, 95%, or 100% is indicative of therapeutic efficacy. In some embodiments, a reduction in the number of metastases by 10%, 20%, 30%, 40%, 50%, 60%, 70%, 80%, 90%, 95%, or 100% is indicative of therapeutic efficacy.

The amount of the antibodies and additional therapeutic agents and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a multi-specific binding protein may be administered during a time when the additional therapeutic agent(s) exerts its prophylactic or therapeutic effect, or vice versa.

### VIII. Pharmaceutical Composition and Administration

The present disclosure also features pharmaceutical compositions/formulations that contain a therapeutically effective amount of an anti-CD27 antibody described herein. The composition can be formulated for use in a variety of drug delivery systems. One or more physiologically acceptable excipients or carriers can also be included in the composition for proper formulation. Suitable formulations for use in the present disclosure are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

The antibodies of the present disclosure can exist in a lyophilized formulation or liquid aqueous pharmaceutical formulation. The aqueous carrier of interest herein is one which is pharmaceutically acceptable (safe and non-toxic for administration to a human) and is useful for the preparation of a liquid formulation. Illustrative carriers include sterile water for injection (SWFI), bacteriostatic water for injection (BWFI), a pH buffered solution (e.g., phosphate-buffered saline), sterile saline solution, Ringer's solution or dextrose solution.

The antibodies of the present disclosure could exist in a lyophilized formulation including the proteins and a lyoprotectant. The lyoprotectant may be sugar, e.g., disaccharides. In certain embodiments, the lyoprotectant is sucrose or maltose. The lyophilized formulation may also include one or more of a buffering agent, a surfactant, a bulking agent, and/or a preservative.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient. It may be administered in the range of 0.1 mg to 1 g and preferably in the range of 0.5 mg to 500 mg of active antibody per administration for adults. Alternatively, a patient's dose can be tailored to the approximate body weight or surface area of the patient. Other factors in determining the appropriate dosage can include the disease or condition to be treated or prevented, the severity of the disease, the route of administration, and the age, sex and medical condition of the patient. Further refinement of the calculations necessary to determine the appropriate dosage for treatment is routinely made by those skilled in the art, especially in light of the dosage information and assays disclosed herein. The dosage can also be determined through the use of known assays for determining dosages used in conjunction with appropriate dose-response data. An individual patient's dosage can be adjusted as the progress of the disease is monitored. Blood levels of the targetable construct or complex in a patient can be measured to see if the dosage needs to be adjusted to reach or maintain an effective concentration. Pharmacogenomics may be used to determine which targetable constructs and/or complexes, and dosages thereof, are most likely to be effective for a given individual (Schmitz et al., Clinica Chimica Acta 308: 43-53, 2001; Steimer et al., Clinica Chimica Acta 308: 33-41, 2001).

Doses may be given once or more times daily, weekly, monthly or yearly, or even once every 2 to 20 years. Persons of ordinary skill in the art can easily estimate repetition rates for dosing based on measured residence times and concentrations of the targetable construct or complex in bodily fluids or tissues. Administration of the present invention could be intravenous, intraarterial, intraperitoneal, intramuscular, subcutaneous, intrapleural, intrathecal, intracavitary, by perfusion through a catheter or by direct intralesional injection. This may be administered once or more times daily, once or more times weekly, once or more times monthly, and once or more times annually.

### EXAMPLES

### Example 1: Anti-CD27 Antibodies

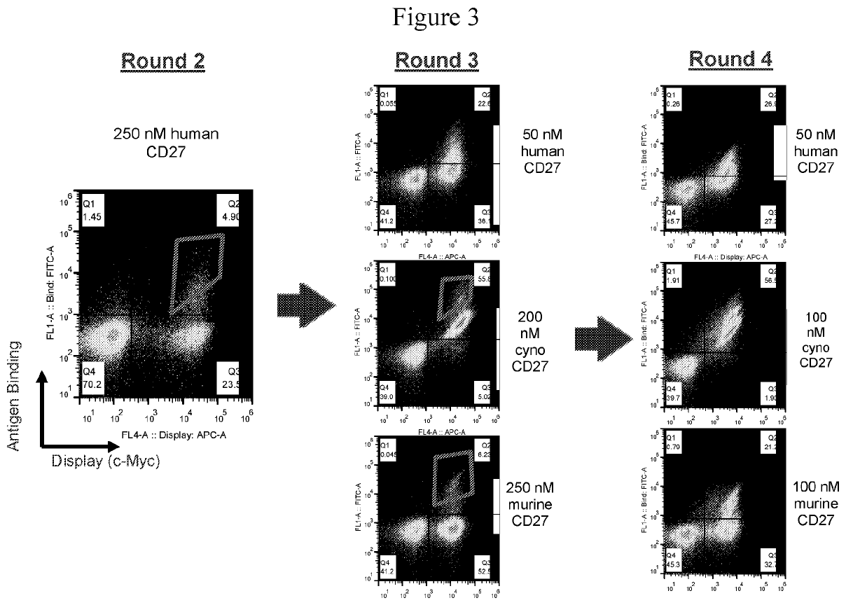

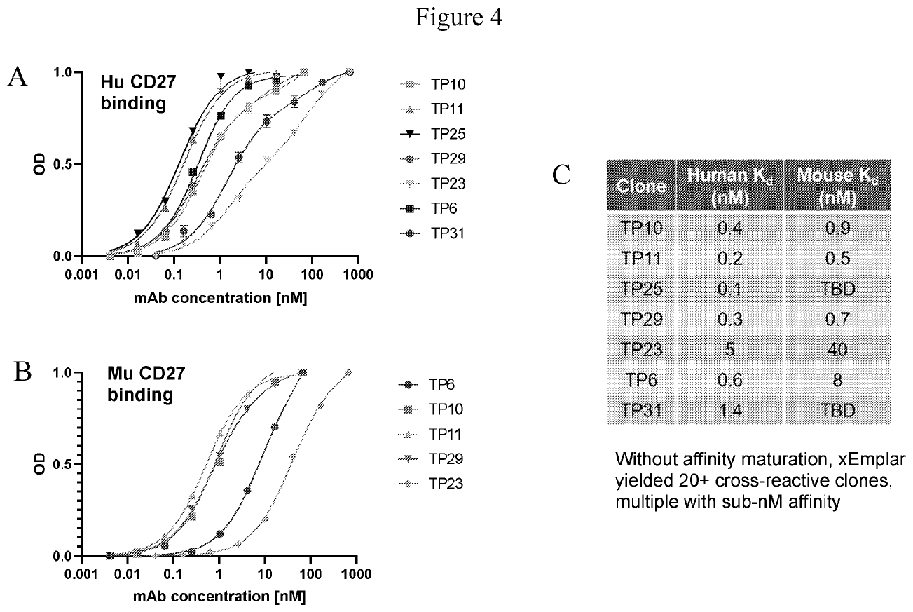

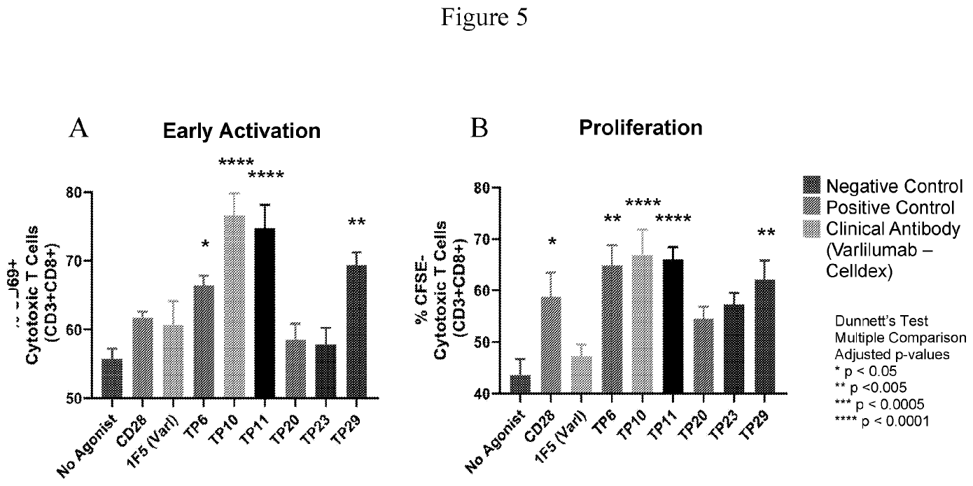

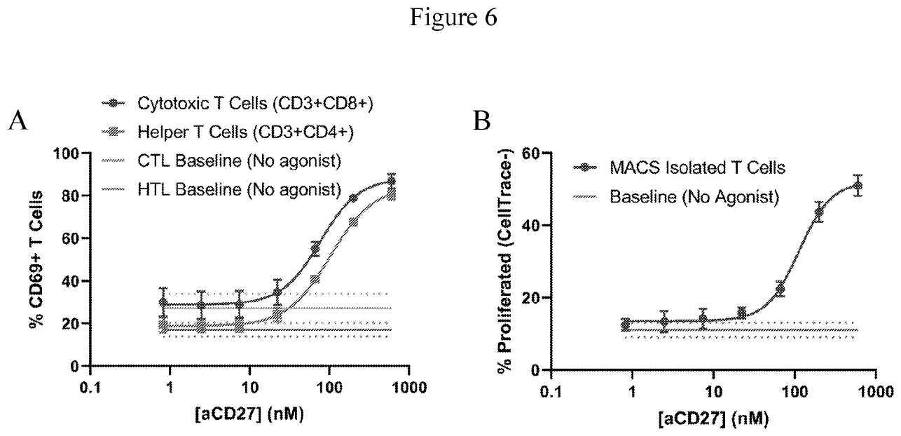

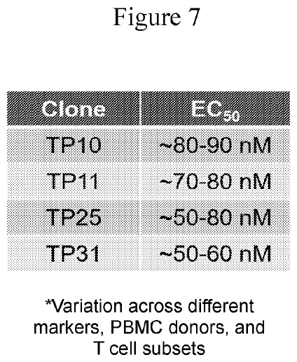

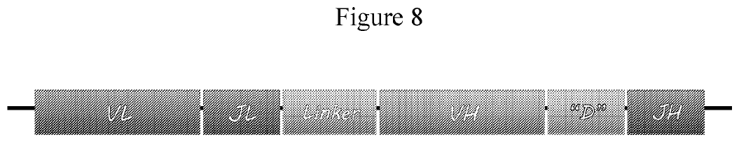

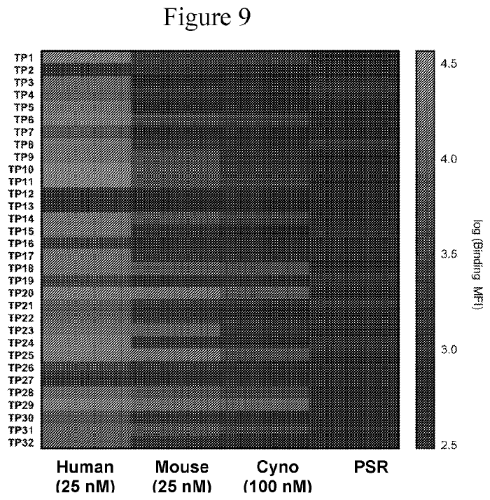

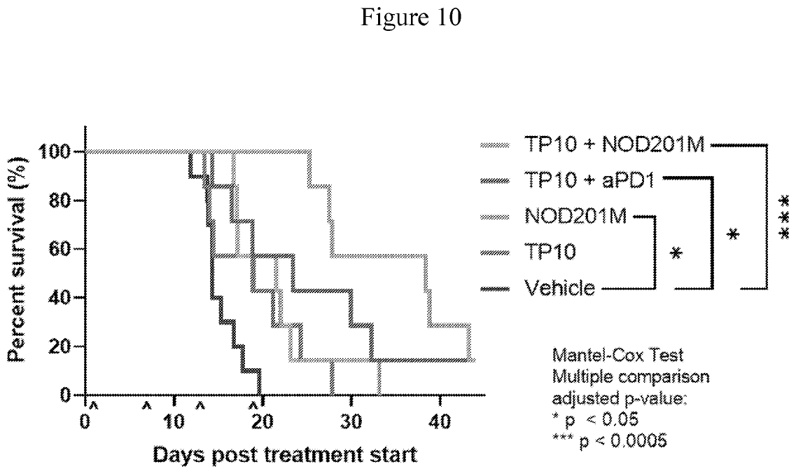

A library of anti-CD27 antibodies was created which mimics the human antibody repertoire. The library incorporates selected scaffolds (VH, VL, JL, JH) which replicate human relative abundance and diversify functionality (FIG. 8). The custom designed diversity is located in the DH segment, the site of the majority of diversity in natural repertoires. The frequencies are based on the natural repertoire, with modifications to enhance developability. There are no Cys, Met, and minimization of motifs which lead to nonspecificity. The library was synthesized in single chain fragment (scFv) format. Rapid isolation of high affinity anti-CD27 populations was performed (FIG. 3). Screening of 100 anti-CD27 antibody clones revealed 32 unique sequences. Individual clones were characterized for binding to human CD27 (25 nM), mouse CD27 (25 nM), and cyno CD27 (100 nM) as well as polyspecificity reagent (PSR). Multiple triple cross-reactive clones were identified that do not bind PSR (FIG. 9). These clones were assessed for binding to human CD27 and mouse CD27 and their K_{d} was determined (FIG. 4). Early activation assays were performed with the selected anti-CD27 antibody clones on 2-3 PBMC donors (FIG. 5A and FIG. 5B). CD25, CD107A and cytokine expression correlated with CD69 and proliferation data. Four of the clones (FIG. 1) were tested for and exhibited dose-dependent activation (EC50˜50-100 nM) as measured from CD69 expression and proliferation (FIG. 6A, FIG. 6B, and FIG. 7). The highest affinity of any of these antibodies from the screen was estimated at 5 nM. The highest affinity (lowest Kd) was 0.1 nM towards human and 0.1 nM towards mouse as well. Of the four with sequences provided, the worst has an affinity of 1.4 nM towards human CD27 and 0.5 nM towards Mouse CD27.

### Example 2: Tumor Testing

### Experimental Setup

The mouse model employed in this example was the BALB/c mouse model. Tumors were implanted 12 days prior to start of treatment with 3×10⁵ CT26 tumor cells.

At study start, the mean tumor size was ˜100 mm³ and ranged in size from 75-144 mm³.

Treatment dosing was performed at days=1, 7, 13, and 19. Anti-PD-1 dosing was performed at days=1, 4, 8, and 11.

The study in this example included 5 Treatment Arms, with 10 animals per arm. The 5 Treatment Arms are listed below:

1. Vehicle—PBS

2. TP10—anti-CD27 antibody candidate

3. XC201—knottin Fc Fusion

4. TP10+anti-PD-1—combination with RMP1-14 clone

5. TP10+NOD201—combination

### Tissue/Organ Sampling

Animals were euthanized and tissues sampled from all 5 experimental groups (Treatment Arms).
3 random animals (#3, 6, and 9)
15 days after first dose (d=16)

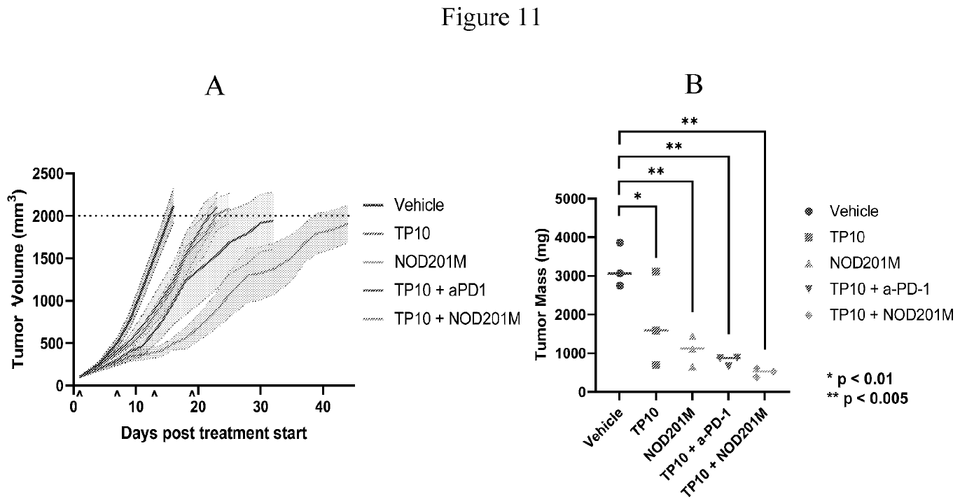

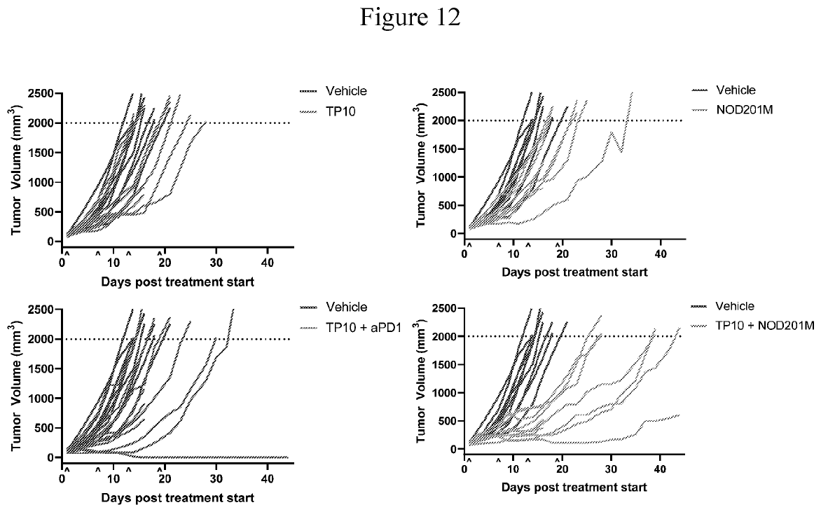

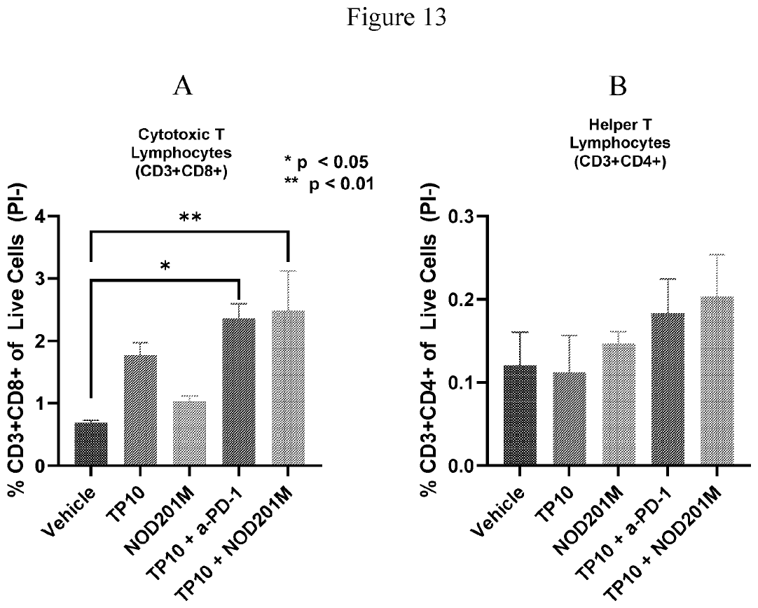

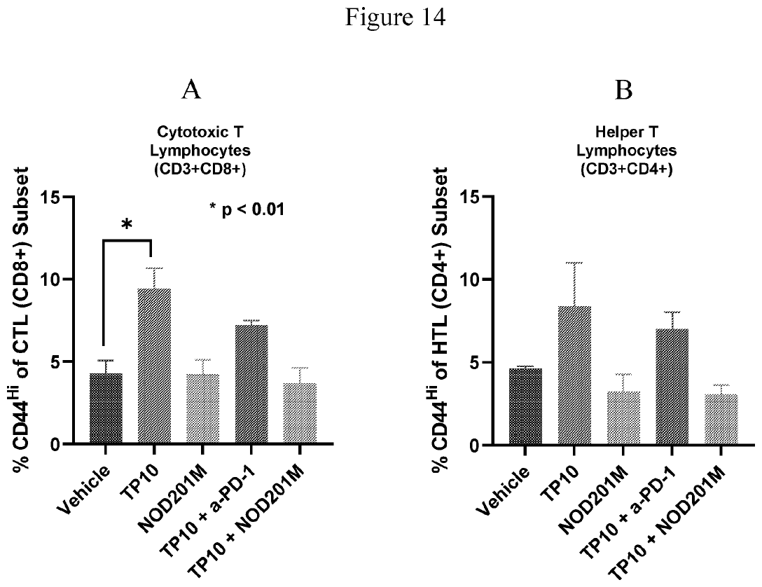

Tumors were examined for weight and flow cytometry performed (6 color) as provided in, for example, FIGS. 11-14.

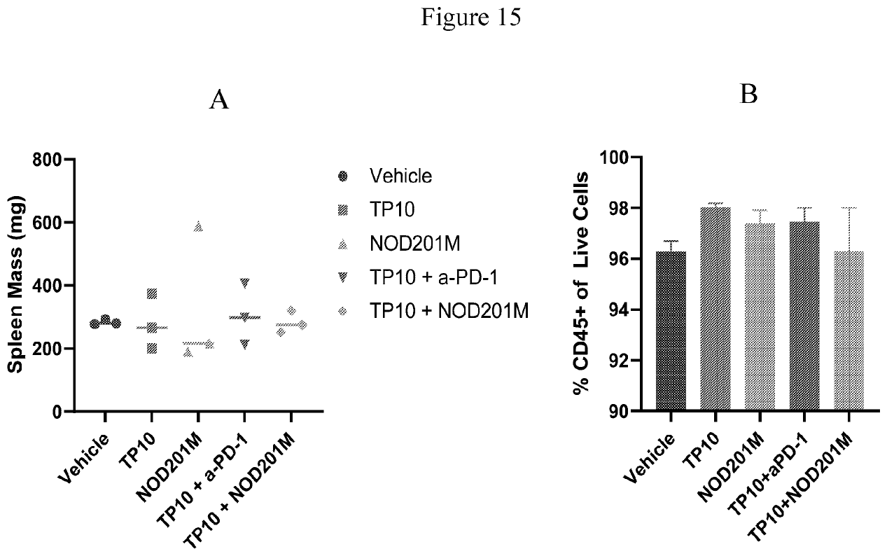

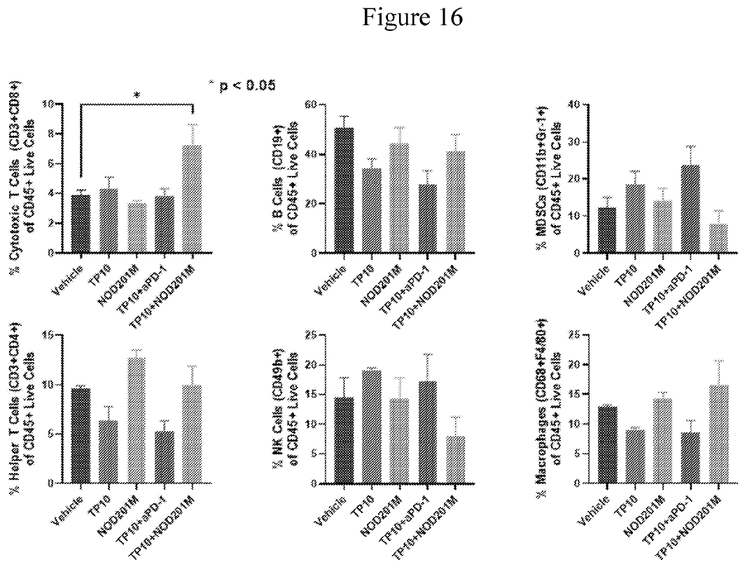

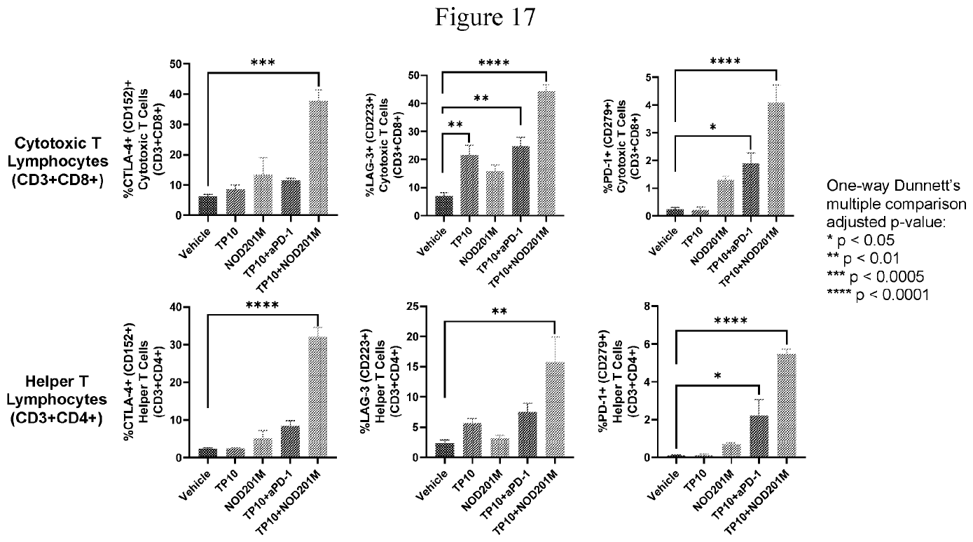

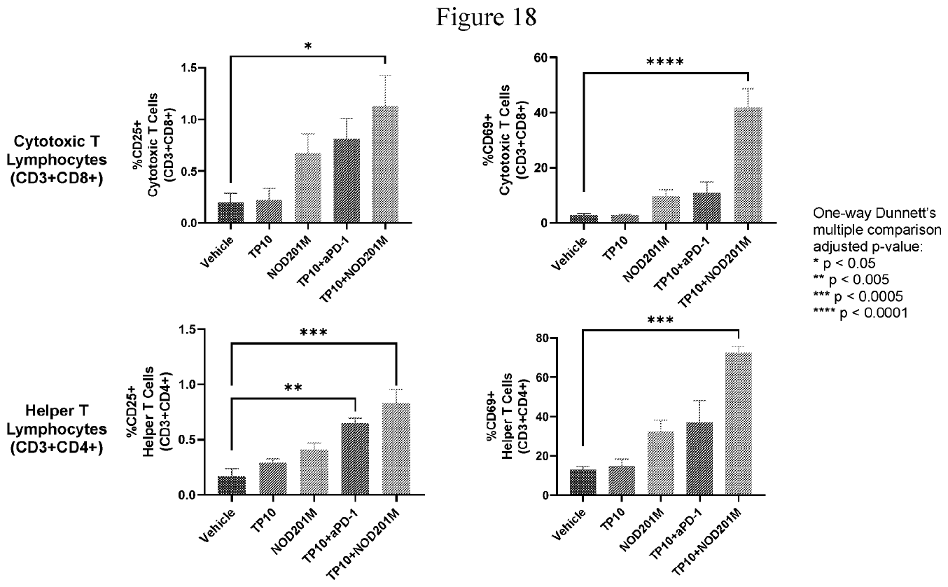

Spleens were examined for weight and flow cytometry performed (18 color) as provided in, for example, FIGS. 15-18.

### Summary

An anti-CD27 antibody (TP10) was tested in a mouse tumor model (CT26), and effective tumor control (e.g., a reduction in tumor growth) was observed when TP10 anti-CD-27 antibody was combined with anti-PD-1 and when TP10 anti-CD-27 antibody was NOD201m, mouse Fc variant of NOD201.

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains. All references cited in this disclosure are incorporated by reference to the same extent as if each reference had been incorporated by reference in its entirety individually.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed is:

1. An anti-CD27 antibody comprising:
a) a heavy chain variable region comprising an amino acid sequence of SEQ ID NO:1 and a light chain variable region comprising an amino acid sequence of SEQ ID NO:5;
b) a heavy chain variable region comprising an amino acid sequence of SEQ ID NO:9 and a light chain variable region comprising an amino acid sequence of SEQ ID NO:13;
c) a heavy chain variable region comprising an amino acid sequence of SEQ ID NO:17 and a light chain variable region comprising an amino acid sequence of SEQ ID NO:21;
d) a heavy chain variable region comprising an amino acid sequence of SEQ ID NO:25 and a light chain variable region comprising an amino acid sequence of SEQ ID NO:29; or
e) a heavy chain variable region comprising an amino acid sequence of SEQ ID NO:105 and a light chain variable region comprising an amino acid sequence of SEQ ID NO:109;
f) a heavy chain variable region comprising an amino acid sequence of SEQ ID NO:113 and a light chain variable region comprising an amino acid sequence of SEQ ID NO:117;
g) a heavy chain variable region comprising an amino acid sequence of SEQ ID NO:225 and a light chain variable region comprising an amino acid sequence of SEQ ID NO:229; or
h) a heavy chain variable region comprising an amino acid sequence of SEQ ID NO:273 and a light chain variable region comprising an amino acid sequence of SEQ ID NO:277.

2. An anti-CD27 antibody comprising:
a) a vhCDR1 comprising SEQ ID NO:2, a vhCDR2 comprising SEQ ID NO:3, a vhCDR3 comprising SEQ ID NO:4, a vlCDR1 comprising SEQ ID NO:6, a vlCDR2 comprising SEQ ID NO:7, and a vlCDR3 comprising SEQ ID NO:8;
b) a vhCDR1 comprising SEQ ID NO:10, a vhCDR2 comprising SEQ ID NO:11, a vhCDR3 comprising SEQ ID NO:12, a vlCDR1 comprising SEQ ID NO:14, a vlCDR2 comprising SEQ ID NO:15, and a vlCDR3 comprising SEQ ID NO:16;
c) a vhCDR1 comprising SEQ ID NO:18, a vhCDR2 comprising SEQ ID NO:19, a vhCDR3 comprising SEQ ID NO:20, a vlCDR1 comprising SEQ ID NO:22, a vlCDR2 comprising SEQ ID NO:23, and a vlCDR3 comprising SEQ ID NO:24;
d) a vhCDR1 comprising SEQ ID NO:26, a vhCDR2 comprising SEQ ID NO:27, a vhCDR3 comprising SEQ ID NO:28, a vlCDR1 comprising SEQ ID NO:30, a vlCDR2 comprising SEQ ID NO:31, and a vlCDR3 comprising SEQ ID NO:32;
e) a vhCDR1 comprising SEQ ID NO:106, a vhCDR2 comprising SEQ ID NO:107, a vhCDR3 comprising SEQ ID NO:108, a vlCDR1 comprising SEQ ID NO:110, a vlCDR2 comprising SEQ ID NO:111, and a vlCDR3 comprising SEQ ID NO:112;
f) a vhCDR1 comprising SEQ ID NO:114, a vhCDR2 comprising SEQ ID NO:115, a vhCDR3 comprising SEQ ID NO:116, a vlCDR1 comprising SEQ ID NO:118, a vlCDR2 comprising SEQ ID NO:119, and a vlCDR3 comprising SEQ ID NO:120;
g) a vhCDR1 comprising SEQ ID NO:226, a vhCDR2 comprising SEQ ID NO:227, a vhCDR3 comprising SEQ ID NO:228, a vlCDR1 comprising SEQ ID NO:230, a vlCDR2 comprising SEQ ID NO:231, and a vlCDR3 comprising SEQ ID NO:232; or
h) a vhCDR1 comprising SEQ ID NO:226, a vhCDR2 comprising SEQ ID NO:227, a vhCDR3 comprising SEQ ID NO:228, a vlCDR1 comprising SEQ ID NO:230, a vlCDR2 comprising SEQ ID NO:231, and a vlCDR3 comprising SEQ ID NO:232.

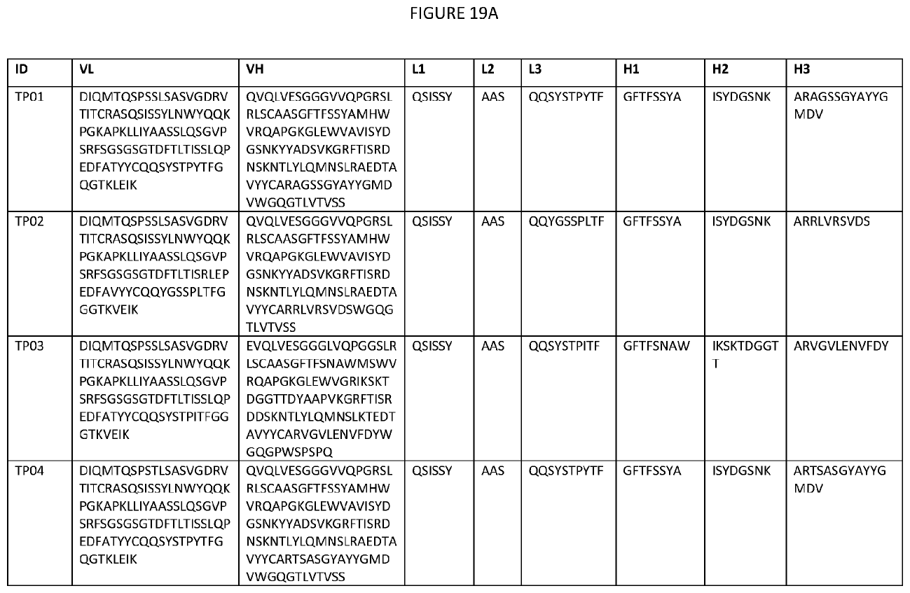

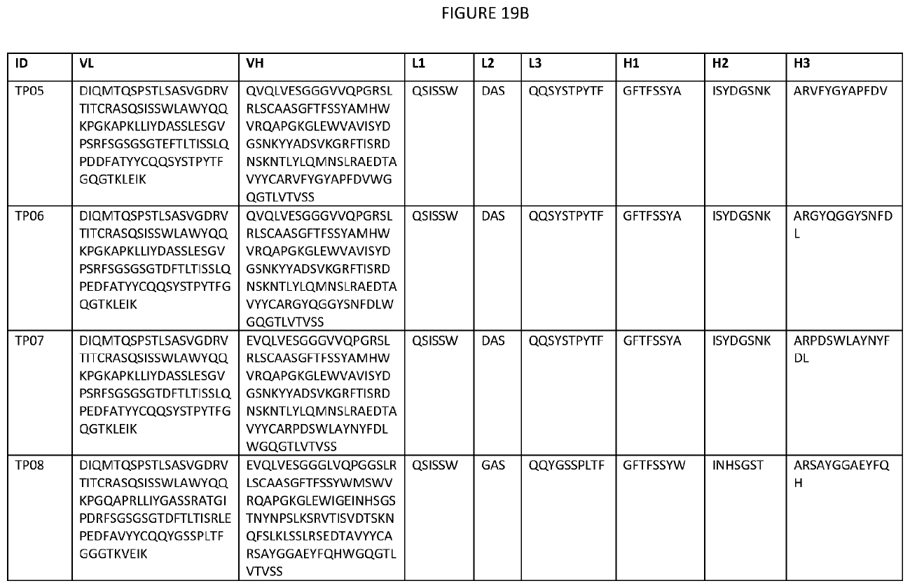

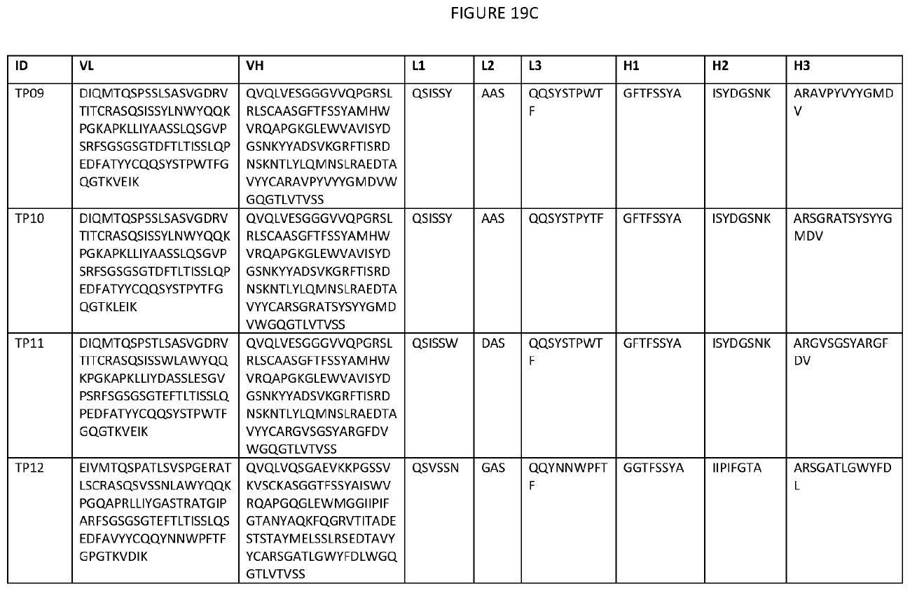

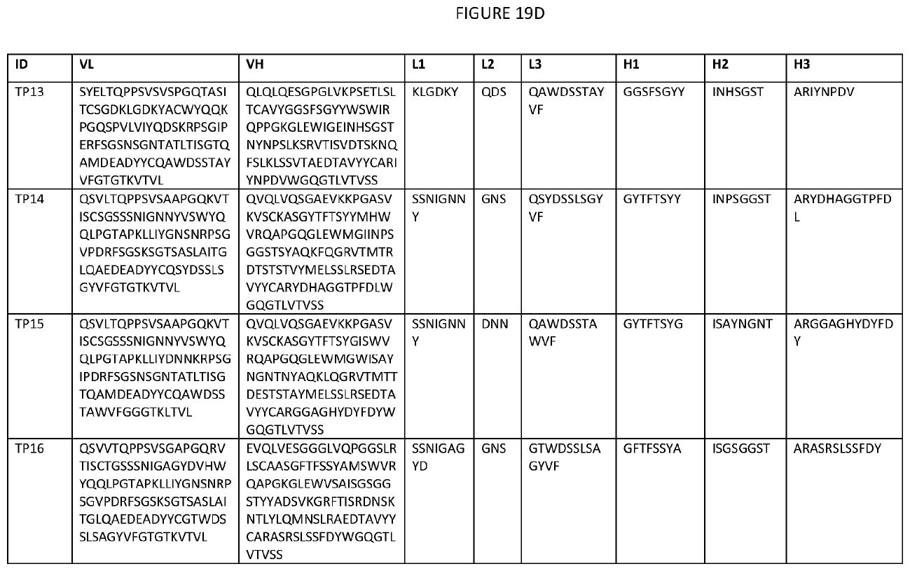

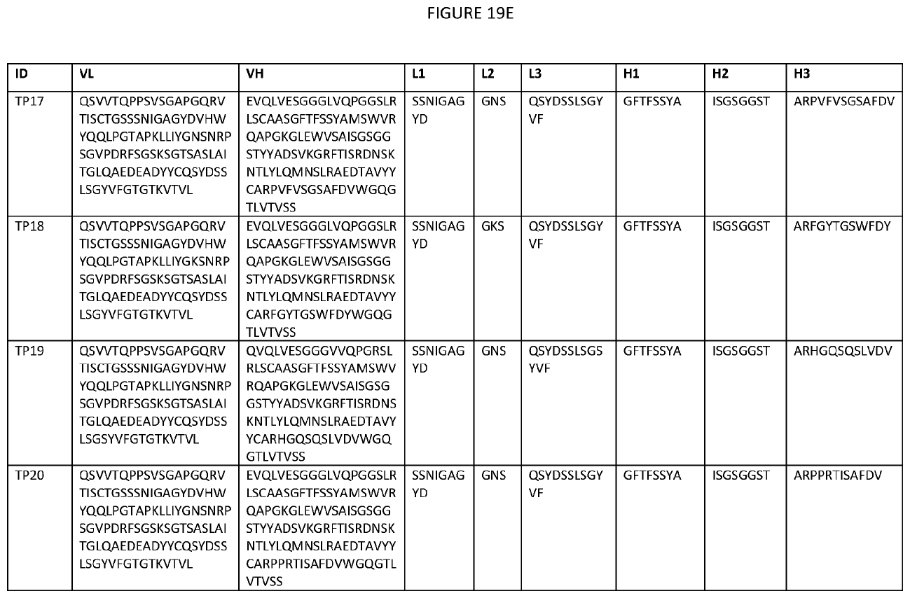

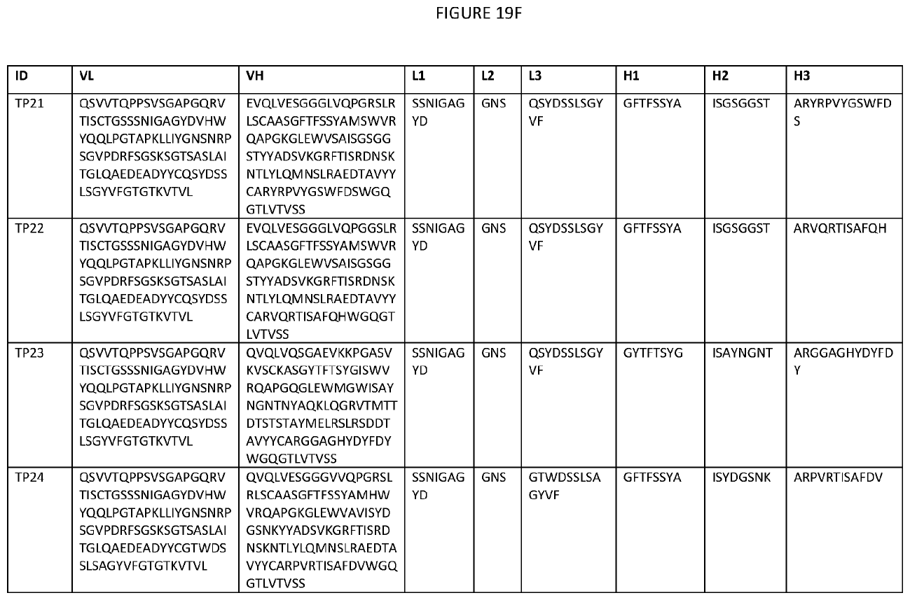

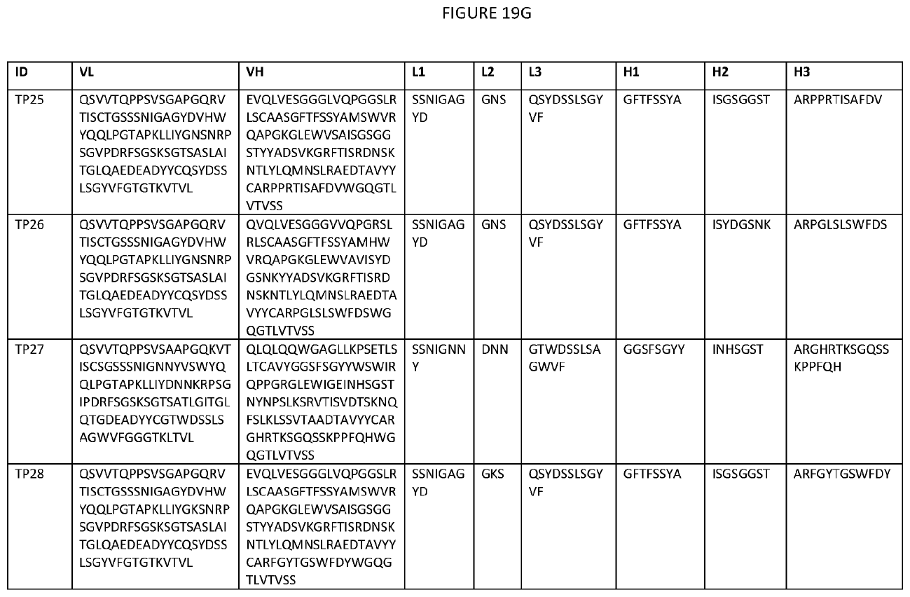

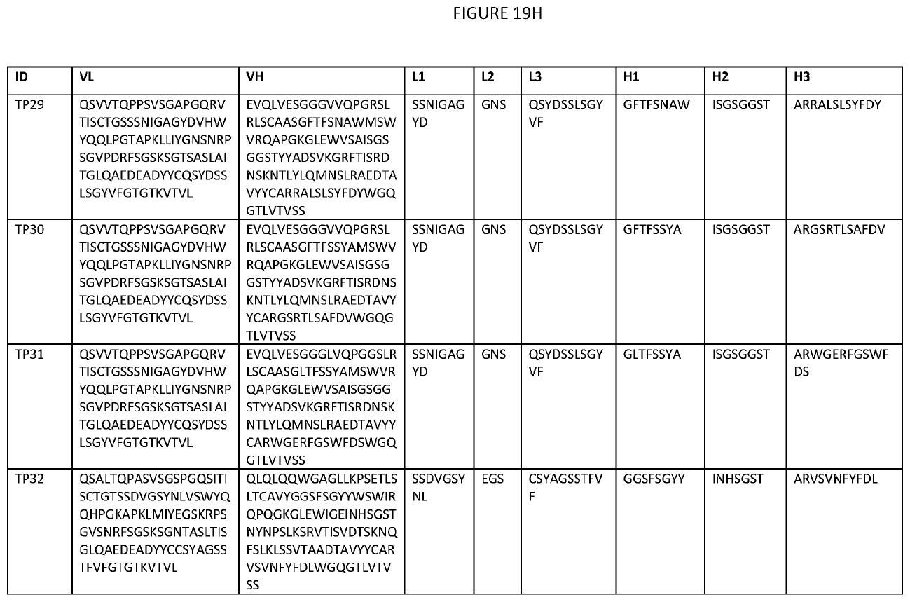

3. An anti-CD27 antibody comprising a vhCDR1, a vhCDR2, a vhCDR3, a vlCDR1, a vlCDR2, and a vlCDR3 as described in FIG. 1 and/or FIG. 19.

4. An anti-CD27 antibody comprising a vhCDR1, a vhCDR2, a vhCDR3, a vlCDR1, a vlCDR2, and a vlCDR3 from and antibody selected from the group consisting of TP01, TP02, TP03, TP04, TP05, TP06, TP07, TP08, TP09, TP10, TP11, TP12, TP13, TP14, TP15, TP16, TP17, TP18, TP19, TP20, TP21, TP22, TP23, TP24, TP25, TP26, TP27, TP28, TP29, TP30, TP31, and TP32.

5. The anti-CD27 antibody according to any one of the previous claims, wherein the antibody comprises a constant region with an amino acid sequence at least 90% identical to a human IgG.

6. The anti-CD27 antibody according to claim 5, wherein the human IgG is selected from the group consisting of IgG1, IgG2, IgG3 and IgG4.

7. The anti-CD27 antibody according to claim 6, wherein the IgG is an IgG1 or IgG4.

8. A nucleic acid composition comprising:
(a) a first nucleic acid encoding a heavy chain variable region according to any one of the preceding claims.
(b) a second nucleic acid encoding a light chain variable region according to any one of the preceding claims.

9. An expression vector composition comprising:
(a) a first expression vector comprising the first nucleic acid of claim 8; and
(b) a second expression vector comprising the second nucleic acid of claim 8.

10. An expression vector composition comprising the nucleic acid composition according to claim 8, wherein the first nucleic acid and the second nucleic acid are contained in a single expression vector.

11. A host cell comprising the expression vector composition of claim 9 or 10.

12. A method of making an antibody comprising culturing the host cell of claim 11 under conditions wherein the antibody is expressed, and recovering the antibody.

13. A composition comprising the antibody according to any one of claims 1-8, and a pharmaceutical acceptable carrier or diluent.

14. A method of modulating an immune response in a subject, the method comprising administering to the subject an effective amount of the antibody according to any one of the claims 1-8 or the composition according to claim 13.

15. The method of claim 14, wherein the antibody comprises a heavy chain variable region comprising an amino acid sequence of SEQ ID NO:1 or SEQ ID NO:105 and a light chain variable region comprising an amino acid sequence of SEQ ID NO:5 or SEQ ID NO:109; and/or a vhCDR1 comprising SEQ ID NO:2, a vhCDR2 comprising SEQ ID NO:3, a vhCDR3 comprising SEQ ID NO:4, a vlCDR1 comprising SEQ ID NO:6, a vlCDR2 comprising SEQ ID NO:7, and a vlCDR3 comprising SEQ ID NO:8; and/or a vhCDR1 comprising SEQ ID NO:106, a vhCDR2 comprising SEQ ID NO:107, a vhCDR3 comprising SEQ ID NO:108, a vlCDR1 comprising SEQ ID NO:110, a vlCDR2 comprising SEQ ID NO:111, and a vlCDR3 comprising SEQ ID NO:112.

16. The method of claim 14, wherein the antibody comprises a heavy chain variable region comprising an amino acid sequence of SEQ ID NO:9 or SEQ ID NO: 113 and a light chain variable region comprising an amino acid sequence of SEQ ID NO:13 or SEQ ID NO:117; and/or a vhCDR1 comprising SEQ ID NO:10, a vhCDR2 comprising SEQ ID NO:11, a vhCDR3 comprising SEQ ID NO:12, a vlCDR1 comprising SEQ ID NO:14, a vlCDR2 comprising SEQ ID NO:15, and a vlCDR3 comprising SEQ ID NO:16; and/or a vhCDR1 comprising SEQ ID NO:114, a vhCDR2 comprising SEQ ID NO:115, a vhCDR3 comprising SEQ ID NO:116, a vlCDR1 comprising SEQ ID NO:118, a vlCDR2 comprising SEQ ID NO:119, and a vlCDR3 comprising SEQ ID NO:120.

17. The method of claim 14, wherein the antibody comprises a heavy chain variable region comprising an amino acid sequence of SEQ ID NO:17 or SEQ ID NO:225 and a light chain variable region comprising an amino acid sequence of SEQ ID NO:21 or SEQ ID NO:229; and/or a vhCDR1 comprising SEQ ID NO:18, a vhCDR2 comprising SEQ ID NO:19, a vhCDR3 comprising SEQ ID NO:20, a vlCDR1 comprising SEQ ID NO:22, a vlCDR2 comprising SEQ ID NO:23, and a vlCDR3 comprising SEQ ID NO:24; and/or a vhCDR1 comprising SEQ ID NO:226, a vhCDR2 comprising SEQ ID NO:227, a vhCDR3 comprising SEQ ID NO:228, a vlCDR1 comprising SEQ ID NO:230, a vlCDR2 comprising SEQ ID NO:231, and a vlCDR3 comprising SEQ ID NO:232.

18. The method of claim 14, wherein the antibody comprises a heavy chain variable region comprising an amino acid sequence of SEQ ID NO:25 and a light chain variable region comprising an amino acid sequence of SEQ ID NO:29; and/or a vhCDR1 comprising SEQ ID NO:26, a vhCDR2 comprising SEQ ID NO:27, a vhCDR3 comprising SEQ ID NO:28, a vlCDR1 comprising SEQ ID NO:30, a vlCDR2 comprising SEQ ID NO:31, and a vlCDR3 comprising SEQ ID NO:32.

19. A method of treating cancer in a subject comprising administering to the subject an effective amount of the antibody according to any one of the claims 1-8 or the composition according to claim 13.

20. The method of claim 19, wherein the cancer expresses CD-27.

21. The method of claim 19 or 20, wherein the cancer is selected from the group consisting of B cell lymphoma, T cell lymphoma, B and T cell lymphoma, colorectal cancer, breast cancer, rectal cancer, lung (including non-small cell lung cancer), non-Hodgkin's lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, multiple myeloma, CD27 Expressing B-cell Malignancies, Hodgkin's Lymphoma, Chronic Lymphocytic Leukemia, Mantle Cell Lymphoma, Marginal Zone B Cell Lymphoma, T-cell Malignancy, Solid Tumors, Metastatic Melanoma, Renal (Clear) Cell Carcinoma, Hormone-refractory Prostate Adenocarcinoma, Ovarian Cancer, Colorectal Adenocarcinoma, Non-small Cell Lung Cancer, Burkett's Lymphoma, Primary Lymphoma of the Central Nervous System, Advanced Breast Carcinoma, Advanced Ovarian Carcinoma, Prostate Cancer, Squamous Cell Carcinoma of the Head and Neck (SCCHN), Ovarian Carcinoma, Colorectal Cancer (CRC), Renal Cell Carcinoma (RCC), Glioblastoma (GBM), Carcinoma, Renal Cell, Kidney Diseases, Kidney Neoplasms, Urogenital Neoplasms, Urologic Diseases, Urologic Neoplasms, Neoplasms, Neoplasms by Histologic Type, Clear-cell Metastatic Renal Cell Carcinoma, Unresectable Stage III or Stage IV Melanoma, Carcinoma, Renal Cell, Kidney Diseases, Kidney Neoplasms, Urogenital Neoplasms, Urologic Diseases, Urologic Neoplasms, Neoplasms by Histologic Type, Neoplasms Clear-cell Metastatic Renal Cell Carcinoma, Melanoma, Triple Negative Breast Cancer, Bladder Cancer, Head and Neck Cancer, Non-small Cell Lung Cancer, Aggressive B-Cell Lymphomas, and B-Cell Lymphomas.

22. The method according to claim 19, wherein the antibody is combined with one or more additional therapeutic agents to treat cancer.

23. The method according to claim 22, wherein the additional therapeutic agents are other immune checkpoint inhibitors.

24. The method of claim 22, wherein the other immune checkpoint inhibitors are selected from the group consisting of PD-1 inhibitor, a PD-L1 inhibitor, a CTLA-4 inhibitor, a TIM-3 inhibitor, and a LAG-3 inhibitor.

25. The method according to claim 22, wherein the additional therapeutic agents are tumor targeting antibodies.

26. The method according to claim 25, wherein the tumor targeting antibodies are selected from the group consisting of rituximab, trastuzumab, and sunitinib.

27. The method according to claim 25, wherein the additional therapeutic agent is an integrin-binding polypeptide-Fc fusion.

28. The method according to claim 25, wherein the integrin-binding polypeptide-Fc fusion is NOD-201.

29. A method for inducing or enhancing an immune response against an antigen in a subject comprising administering to the subject a monoclonal antibody which binds to human CD27, in an amount effective to induce or enhance an immune response against an antigen, wherein the antibody comprises a heavy chain variable region CDR1 comprising SEQ ID NO: 2; a heavy chain variable region CDR2 comprising SEQ ID NO: 3; a heavy chain variable region CDR3 comprising SEQ ID NO: 4; a light chain variable region CDR1 comprising SEQ ID NO: 6; a light chain variable region CDR2 comprising SEQ ID NO: 7; and a light chain variable region CDR3 comprising SEQ ID NO: 8; or wherein the antibody comprises a heavy chain variable region CDR1 comprising SEQ ID NO: 106; a heavy chain variable region CDR2 comprising SEQ ID NO: 107; a heavy chain variable region CDR3 comprising SEQ ID NO: 108; a light chain variable region CDR1 comprising SEQ ID NO: 110; a light chain variable region CDR2 comprising SEQ ID NO: 111; and a light chain variable region CDR3 comprising SEQ ID NO: 112.

30. A method for inducing or enhancing an immune response against an antigen in a subject comprising administering to the subject a monoclonal antibody which binds to human CD27, in an amount effective to induce or enhance an immune response against an antigen, wherein the antibody comprises a heavy chain variable region CDR1 comprising SEQ ID NO: 10; a heavy chain variable region CDR2 comprising SEQ ID NO: 11; a heavy chain variable region CDR3 comprising SEQ ID NO: 12; a light chain variable region CDR1 comprising SEQ ID NO: 14; a light chain variable region CDR2 comprising SEQ ID NO: 15; and a light chain variable region CDR3 comprising SEQ ID NO: 16; or wherein the antibody comprises a heavy chain variable region CDR1 comprising SEQ ID NO: 114; a heavy chain variable region CDR2 comprising SEQ ID NO: 115; a heavy chain variable region CDR3 comprising SEQ ID NO: 116; a light chain variable region CDR1 comprising SEQ ID NO: 118; a light chain variable region CDR2 comprising SEQ ID NO: 119; and a light chain variable region CDR3 comprising SEQ ID NO: 120.

31. A method for inducing or enhancing an immune response against an antigen in a subject comprising administering to the subject a monoclonal antibody which binds to human CD27, in an amount effective to induce or enhance an immune response against an antigen, wherein the antibody comprises a heavy chain variable region CDR1 comprising SEQ ID NO: 18; a heavy chain variable region CDR2 comprising SEQ ID NO: 19; a heavy chain variable region CDR3 comprising SEQ ID NO: 20; a light chain variable region CDR1 comprising SEQ ID NO: 22; a light chain variable region CDR2 comprising SEQ ID NO: 23; and a light chain variable region CDR3 comprising SEQ ID NO: 24; or wherein the antibody comprises a heavy chain variable region CDR1 comprising SEQ ID NO: 226; a heavy chain variable region CDR2 comprising SEQ ID NO: 227; a heavy chain variable region CDR3 comprising SEQ ID NO: 228; a light chain variable region CDR1 comprising SEQ ID NO: 230; a light chain variable region CDR2 comprising SEQ ID NO: 231; and a light chain variable region CDR3 comprising SEQ ID NO: 232.

32. A method for inducing or enhancing an immune response against an antigen in a subject comprising administering to the subject a monoclonal antibody which binds to human CD27, in an amount effective to induce or enhance an immune response against an antigen, wherein the antibody comprises a heavy chain variable region CDR1 comprising SEQ ID NO: 26; a heavy chain variable region CDR2 comprising SEQ ID NO: 27; a heavy chain variable region CDR3 comprising SEQ ID NO: 28; a light chain variable region CDR1 comprising SEQ ID NO: 30; a light chain variable region CDR2 comprising SEQ ID NO: 31; and a light chain variable region CDR3 comprising SEQ ID NO: 32; or wherein the antibody comprises a heavy chain variable region CDR1 comprising SEQ ID NO: 274; a heavy chain variable region CDR2 comprising SEQ ID NO: 275; a heavy chain variable region CDR3 comprising SEQ ID NO: 276; a light chain variable region CDR1 comprising SEQ ID NO: 278; a light chain variable region CDR2 comprising SEQ ID NO: 279; and a light chain variable region CDR3 comprising SEQ ID NO: 280.

33. A method of inhibiting growth of CD27 expressing cells comprising contacting the cells with a monoclonal antibody which binds to human CD27, in an amount effective to inhibit growth of CD27 expressing cells, wherein the antibody comprises a heavy chain variable region CDR1 comprising SEQ ID NO: 2; a heavy chain variable region CDR2 comprising SEQ ID NO: 3; a heavy chain variable region CDR3 comprising SEQ ID NO: 4; a light chain variable region CDR1 comprising SEQ ID NO: 6; a light chain variable region CDR2 comprising SEQ ID NO: 7; and a light chain variable region CDR3 comprising SEQ ID NO: 8; or wherein the antibody comprises a heavy chain variable region CDR1 comprising SEQ ID NO: 106; a heavy chain variable region CDR2 comprising SEQ ID NO: 107; a heavy chain variable region CDR3 comprising SEQ ID NO: 108; a light chain variable region CDR1 comprising SEQ ID NO: 110; a light chain variable region CDR2 comprising SEQ ID NO: 111; and a light chain variable region CDR3 comprising SEQ ID NO: 112.

34. A method of inhibiting growth of CD27 expressing cells comprising contacting the cells with a monoclonal antibody which binds to human CD27, in an amount effective to inhibit growth of CD27 expressing cells, wherein the antibody comprises a heavy chain variable region CDR1 comprising SEQ ID NO: 10; a heavy chain variable region CDR2 comprising SEQ ID NO: 11; a heavy chain variable region CDR3 comprising SEQ ID NO: 12; a light chain variable region CDR1 comprising SEQ ID NO: 14; a light chain variable region CDR2 comprising SEQ ID NO: 15; and a light chain variable region CDR3 comprising SEQ ID NO: 16; or wherein the antibody comprises a heavy chain variable region CDR1 comprising SEQ ID NO: 114; a heavy chain variable region CDR2 comprising SEQ ID NO: 115; a heavy chain variable region CDR3 comprising SEQ ID NO: 116; a light chain variable region CDR1 comprising SEQ ID NO: 118; a light chain variable region CDR2 comprising SEQ ID NO: 119; and a light chain variable region CDR3 comprising SEQ ID NO: 120.

35. A method of inhibiting growth of CD27 expressing cells comprising contacting the cells with a monoclonal antibody which binds to human CD27, in an amount effective to inhibit growth of CD27 expressing cells, wherein the antibody comprises a heavy chain variable region CDR1 comprising SEQ ID NO: 18; a heavy chain variable region CDR2 comprising SEQ ID NO: 19; a heavy chain variable region CDR3 comprising SEQ ID NO: 20; a light chain variable region CDR1 comprising SEQ ID NO: 22; a light chain variable region CDR2 comprising SEQ ID NO: 23; and a light chain variable region CDR3 comprising SEQ ID NO: 24; or wherein the antibody comprises a heavy chain variable region CDR1 comprising SEQ ID NO: 226; a heavy chain variable region CDR2 comprising SEQ ID NO: 227; a heavy chain variable region CDR3 comprising SEQ ID NO: 228; a light chain variable region CDR1 comprising SEQ ID NO: 230; a light chain variable region CDR2 comprising SEQ ID NO: 231; and a light chain variable region CDR3 comprising SEQ ID NO: 232.

36. A method of inhibiting growth of CD27 expressing cells comprising contacting the cells with a monoclonal antibody which binds to human CD27, in an amount effective to inhibit growth of CD27 expressing cells, wherein the antibody comprises a heavy chain variable region CDR1 comprising SEQ ID NO: 26; a heavy chain variable region CDR2 comprising SEQ ID NO: 27; a heavy chain variable region CDR3 comprising SEQ ID NO: 28; a light chain variable region CDR1 comprising SEQ ID NO: 30; a light chain variable region CDR2 comprising SEQ ID NO: 31; and a light chain variable region CDR3 comprising SEQ ID NO: 32; or wherein the antibody comprises a heavy chain variable region CDR1 comprising SEQ ID NO: 274; a heavy chain variable region CDR2 comprising SEQ ID NO: 275; a heavy chain variable region CDR3 comprising SEQ ID NO: 276; a light chain variable region CDR1 comprising SEQ ID NO: 278; a light chain variable region CDR2 comprising SEQ ID NO: 279; and a light chain variable region CDR3 comprising SEQ ID NO: 280.

37. A method of inhibiting the binding of CD70 to CD27 on cells in a subject having a disorder by administering to the subject a monoclonal antibody which binds to human CD27, wherein the antibody comprises a heavy chain variable region CDR1 comprising SEQ ID NO: 2; a heavy chain variable region CDR2 comprising SEQ ID NO: 3; a heavy chain variable region CDR3 comprising SEQ ID NO: 4; a light chain variable region CDR1 comprising SEQ ID NO: 6; a light chain variable region CDR2 comprising SEQ ID NO: 7; and a light chain variable region CDR3 comprising SEQ ID NO: 8; or wherein the antibody comprises a heavy chain variable region CDR1 comprising SEQ ID NO: 106; a heavy chain variable region CDR2 comprising SEQ ID NO: 107; a heavy chain variable region CDR3 comprising SEQ ID NO: 108; a light chain variable region CDR1 comprising SEQ ID NO: 110; a light chain variable region CDR2 comprising SEQ ID NO: 111; and a light chain variable region CDR3 comprising SEQ ID NO: 112.

38. A method of inhibiting the binding of CD70 to CD27 on cells in a subject having a disorder by administering to the subject a monoclonal antibody which binds to human CD27, wherein the antibody comprises a heavy chain variable region CDR1 comprising SEQ ID NO: 10; a heavy chain variable region CDR2 comprising SEQ ID NO: 11; a heavy chain variable region CDR3 comprising SEQ ID NO: 12; a light chain variable region CDR1 comprising SEQ ID NO: 14; a light chain variable region CDR2 comprising SEQ ID NO: 15; and a light chain variable region CDR3 comprising SEQ ID NO: 16; or wherein the antibody comprises a heavy chain variable region CDR1 comprising SEQ ID NO: 114; a heavy chain variable region CDR2 comprising SEQ ID NO: 115; a heavy chain variable region CDR3 comprising SEQ ID NO: 116; a light chain variable region CDR1 comprising SEQ ID NO: 118; a light chain variable region CDR2 comprising SEQ ID NO: 119; and a light chain variable region CDR3 comprising SEQ ID NO: 120.

39. A method of inhibiting the binding of CD70 to CD27 on cells in a subject having a disorder by administering to the subject a monoclonal antibody which binds to human CD27, wherein the antibody comprises a heavy chain variable region CDR1 comprising SEQ ID NO: 18; a heavy chain variable region CDR2 comprising SEQ ID NO: 19; a heavy chain variable region CDR3 comprising SEQ ID NO: 20; a light chain variable region CDR1 comprising SEQ ID NO: 22; a light chain variable region CDR2 comprising SEQ ID NO: 23; and a light chain variable region CDR3 comprising SEQ ID NO: 24; or wherein the antibody comprises a heavy chain variable region CDR1 comprising SEQ ID NO: 226; a heavy chain variable region CDR2 comprising SEQ ID NO: 227; a heavy chain variable region CDR3 comprising SEQ ID NO: 228; a light chain variable region CDR1 comprising SEQ ID NO: 230; a light chain variable region CDR2 comprising SEQ ID NO: 231; and a light chain variable region CDR3 comprising SEQ ID NO: 232.

40. A method of inhibiting the binding of CD70 to CD27 on cells in a subject having a disorder by administering to the subject a monoclonal antibody which binds to human CD27, wherein the antibody comprises a heavy chain variable region CDR1 comprising SEQ ID NO: 26; a heavy chain variable region CDR2 comprising SEQ ID NO: 27; a heavy chain variable region CDR3 comprising SEQ ID NO: 28; a light chain variable region CDR1 comprising SEQ ID NO: 30; a light chain variable region CDR2 comprising SEQ ID NO: 31; and a light chain variable region CDR3 comprising SEQ ID NO: 32; or wherein the antibody comprises a heavy chain variable region CDR1 comprising SEQ ID NO: 274; a heavy chain variable region CDR2 comprising SEQ ID NO: 275; a heavy chain variable region CDR3 comprising SEQ ID NO: 276; a light chain variable region CDR1 comprising SEQ ID NO: 278; a light chain variable region CDR2 comprising SEQ ID NO: 279; and a light chain variable region CDR3 comprising SEQ ID NO: 280.

41. The method of any one of claim 29, 33, or 37, wherein the antibody comprises a heavy chain variable region comprising SEQ ID NO:1 or SEQ ID NO:105.

42. The method of any one of claim 29, 33, or 37, wherein the antibody comprises a light chain variable region comprising SEQ ID NO:5 or SEQ ID NO:109.

43. The method of any one of claim 30, 34, or 38, wherein the antibody comprises a heavy chain variable region comprising SEQ ID NO:9 or SEQ ID NO:113.

44. The method of any one of claim 30, 34, or 38, wherein the antibody comprises a light chain variable region comprising SEQ ID NO:13 or SEQ ID NO:117.

45. The method of any one of claim 31, 35, or 39, wherein the antibody comprises a heavy chain variable region comprising SEQ ID NO:17 or SEQ ID NO:225.

46. The method of any one of claim 31, 35, or 39, wherein the antibody comprises a light chain variable region comprising SEQ ID NO:21 or SEQ ID NO:229.

47. The method of any one of claim 32, 36, or 40, wherein the antibody comprises a heavy chain variable region comprising SEQ ID NO:25 or SEQ ID NO:273.

48. The method of any one of claim 32, 36, or 40, wherein the antibody comprises a light chain variable region comprising SEQ ID NO:29 or SEQ ID NO:277.

49. The method of any one of claim 29, 33, or 37, wherein the antibody comprises a heavy chain variable region comprising SEQ ID NO:1 and a light chain variable region comprising SEQ ID NO:5 or a heavy chain variable region comprising SEQ ID NO:105 and a light chain variable region comprising SEQ ID NO:109.

50. The method of any one of claim 30, 34, or 38, wherein the antibody comprises a heavy chain variable region comprising SEQ ID NO:9 and a light chain variable region comprising SEQ ID NO:13 or a heavy chain variable region comprising SEQ ID NO:113 and a light chain variable region comprising SEQ ID NO:117.

51. The method of any one of claim 31, 35, or 39, wherein the antibody comprises a heavy chain variable region comprising SEQ ID NO:17 and a light chain variable region comprising SEQ ID NO:21 or a heavy chain variable region comprising SEQ ID NO:225 and a light chain variable region comprising SEQ ID NO:229.

52. The method of any one of claim 32, 36, or 40, wherein the antibody comprises a heavy chain variable region comprising SEQ ID NO:25 and a light chain variable region comprising SEQ ID NO:29 or a heavy chain variable region comprising SEQ ID NO:273 and a light chain variable region comprising SEQ ID NO:277.

53. The method of any one of claims 29-40, wherein the immune response is an antigen-specific T cell response.

54. A method for inducing or enhancing an immune response against an antigen in a subject comprising administering to the subject a monoclonal antibody which binds to human CD27, in an amount effective to induce or enhance an immune response against an antigen, wherein the antibody binds to the same epitope as an antibody comprising heavy and light chain variable region sequences as set forth in SEQ ID NO: 1 and SEQ ID NO: 5, or sequences as set forth in SEQ ID NO: 105 and SEQ ID NO: 109, respectively.

55. A method for inducing or enhancing an immune response against an antigen in a subject comprising administering to the subject a monoclonal antibody which binds to human CD27, in an amount effective to induce or enhance an immune response against an antigen, wherein the antibody binds to the same epitope as an antibody comprising heavy and light chain variable region sequences as set forth in SEQ ID NO: 9 and SEQ ID NO: 13, or sequences as set forth in SEQ ID NO: 113 and SEQ ID NO: 117, respectively.

56. A method for inducing or enhancing an immune response against an antigen in a subject comprising administering to the subject a monoclonal antibody which binds to human CD27, in an amount effective to induce or enhance an immune response against an antigen, wherein the antibody binds to the same epitope as an antibody comprising heavy and light chain variable region sequences as set forth in SEQ ID NO: 17 and SEQ ID NO: 21, or sequences as set forth in SEQ ID NO: 225 and SEQ ID NO: 229, respectively.

57. A method for inducing or enhancing an immune response against an antigen in a subject comprising administering to the subject a monoclonal antibody which binds to human CD27, in an amount effective to induce or enhance an immune response against an antigen, wherein the antibody binds to the same epitope as an antibody comprising heavy and light chain variable region sequences as set forth in SEQ ID NO: 25 and SEQ ID NO: 29, or sequences as set forth in SEQ ID NO: 273 and SEQ ID NO: 277, respectively.

58. A method for inducing or enhancing an immune response against an antigen in a subject comprising administering to the subject a monoclonal antibody which binds to human CD27, in an amount effective to induce or enhance an immune response against an antigen, wherein the antibody competes for binding to human CD27 with an antibody comprising heavy and light chain variable region sequences as set forth in SEQ ID NO: 1 and 5, or sequences as set forth in SEQ ID NO: 105 and SEQ ID NO: 109, respectively.

59. A method for inducing or enhancing an immune response against an antigen in a subject comprising administering to the subject a monoclonal antibody which binds to human CD27, in an amount effective to induce or enhance an immune response against an antigen, wherein the antibody competes for binding to human CD27 with an antibody comprising heavy and light chain variable region sequences as set forth in SEQ ID NO: 9 and SEQ ID NO: 13, or sequences as set forth in SEQ ID NO: 113 and SEQ ID NO: 117, respectively.

60. A method for inducing or enhancing an immune response against an antigen in a subject comprising administering to the subject a monoclonal antibody which binds to human CD27, in an amount effective to induce or enhance an immune response against an antigen, wherein the antibody competes for binding to human CD27 with an antibody comprising heavy and light chain variable region sequences as set forth in SEQ ID NO: 17 and 21, or sequences as set forth in SEQ ID NO: 225 and SEQ ID NO: 229, respectively.

61. A method for inducing or enhancing an immune response against an antigen in a subject comprising administering to the subject a monoclonal antibody which binds to human CD27, in an amount effective to induce or enhance an immune response against an antigen, wherein the antibody competes for binding to human CD27 with an antibody comprising heavy and light chain variable region sequences as set forth in SEQ ID NO: 25 and 29, or sequences as set forth in SEQ ID NO: 273 and SEQ ID NO: 277, respectively.

62. A method for inducing or enhancing an immune response against an antigen in a subject comprising administering to the subject a monoclonal antibody which binds to human CD27, in an amount effective to induce or enhance an immune response against an antigen, wherein the antibody comprises heavy and light chain variable region sequences having at least 95% identity to SEQ ID NO: 1 and SEQ ID NO: 5, or sequences as set forth in SEQ ID NO: 105 and SEQ ID NO: 109, respectively.

63. A method for inducing or enhancing an immune response against an antigen in a subject comprising administering to the subject a monoclonal antibody which binds to human CD27, in an amount effective to induce or enhance an immune response against an antigen, wherein the antibody comprises heavy and light chain variable region sequences having at least 95% identity to SEQ ID NO: 9 and SEQ ID NO: 13, or sequences as set forth in SEQ ID NO: 113 and SEQ ID NO: 117, respectively.

64. A method for inducing or enhancing an immune response against an antigen in a subject comprising administering to the subject a monoclonal antibody which binds to human CD27, in an amount effective to induce or enhance an immune response against an antigen, wherein the antibody comprises heavy and light chain variable region sequences having at least 95% identity to SEQ ID NO: 17 and SEQ ID NO: 21, or sequences as set forth in SEQ ID NO: 225 and SEQ ID NO: 229, respectively.

65. A method for inducing or enhancing an immune response against an antigen in a subject comprising administering to the subject a monoclonal antibody which binds to human CD27, in an amount effective to induce or enhance an immune response against an antigen, wherein the antibody comprises heavy and light chain variable region sequences having at least 95% identity to SEQ ID NO: 25 and SEQ ID NO: 29, or sequences as set forth in SEQ ID NO: 273 and SEQ ID NO: 277, respectively.

66. A method for inducing or enhancing an immune response in a subject comprising administering to the subject a monoclonal antibody which binds to human CD27, in an amount effective to induce or enhance an immune response, wherein the antibody comprises a heavy chain variable region CDR1 comprising SEQ ID NO: 2; a heavy chain variable region CDR2 comprising SEQ ID NO: 3; a heavy chain variable region CDR3 comprising SEQ ID NO: 4; a light chain variable region CDR1 comprising SEQ ID NO: 6; a light chain variable region CDR2 comprising SEQ ID NO: 7; and a light chain variable region CDR3 comprising SEQ ID NO: 8; or wherein the antibody comprises a heavy chain variable region CDR1 comprising SEQ ID NO: 106; a heavy chain variable region CDR2 comprising SEQ ID NO: 107; a heavy chain variable region CDR3 comprising SEQ ID NO: 108; a light chain variable region CDR1 comprising SEQ ID NO: 110; a light chain variable region CDR2 comprising SEQ ID NO: 111; and a light chain variable region CDR3 comprising SEQ ID NO: 112.

67. A method for inducing or enhancing an immune response in a subject comprising administering to the subject a monoclonal antibody which binds to human CD27, in an amount effective to induce or enhance an immune response, wherein the antibody comprises a heavy chain variable region CDR1 comprising SEQ ID NO: 10; a heavy chain variable region CDR2 comprising SEQ ID NO: 11; a heavy chain variable region CDR3 comprising SEQ ID NO: 12; a light chain variable region CDR1 comprising SEQ ID NO: 14; a light chain variable region CDR2 comprising SEQ ID NO: 15; and a light chain variable region CDR3 comprising SEQ ID NO: 16; or wherein the antibody comprises a heavy chain variable region CDR1 comprising SEQ ID NO: 114; a heavy chain variable region CDR2 comprising SEQ ID NO: 115; a heavy chain variable region CDR3 comprising SEQ ID NO: 116; a light chain variable region CDR1 comprising SEQ ID NO: 118; a light chain variable region CDR2 comprising SEQ ID NO: 119; and a light chain variable region CDR3 comprising SEQ ID NO: 120.

68. A method for inducing or enhancing an immune response in a subject comprising administering to the subject a monoclonal antibody which binds to human CD27, in an amount effective to induce or enhance an immune response, wherein the antibody comprises a heavy chain variable region CDR1 comprising SEQ ID NO: 18; a heavy chain variable region CDR2 comprising SEQ ID NO: 19; a heavy chain variable region CDR3 comprising SEQ ID NO: 20; a light chain variable region CDR1 comprising SEQ ID NO: 22; a light chain variable region CDR2 comprising SEQ ID NO: 23; and a light chain variable region CDR3 comprising SEQ ID NO: 24; or wherein the antibody comprises a heavy chain variable region CDR1 comprising SEQ ID NO: 226; a heavy chain variable region CDR2 comprising SEQ ID NO: 227; a heavy chain variable region CDR3 comprising SEQ ID NO: 228; a light chain variable region CDR1 comprising SEQ ID NO: 230; a light chain variable region CDR2 comprising SEQ ID NO: 231; and a light chain variable region CDR3 comprising SEQ ID NO: 232.

69. A method for inducing or enhancing an immune response in a subject comprising administering to the subject a monoclonal antibody which binds to human CD27, in an amount effective to induce or enhance an immune response, wherein the antibody comprises a heavy chain variable region CDR1 comprising SEQ ID NO: 26; a heavy chain variable region CDR2 comprising SEQ ID NO: 27; a heavy chain variable region CDR3 comprising SEQ ID NO: 28; a light chain variable region CDR1 comprising SEQ ID NO: 30; a light chain variable region CDR2 comprising SEQ ID NO: 31; and a light chain variable region CDR3 comprising SEQ ID NO: 32; or wherein the antibody comprises a heavy chain variable region CDR1 comprising SEQ ID NO: 274; a heavy chain variable region CDR2 comprising SEQ ID NO: 275; a heavy chain variable region CDR3 comprising SEQ ID NO: 276; a light chain variable region CDR1 comprising SEQ ID NO: 278; a light chain variable region CDR2 comprising SEQ ID NO: 279; and a light chain variable region CDR3 comprising SEQ ID NO: 280.

70. A method of treating a non-cancerous disease in a subject comprising administering to the subject an effective amount of the antibody according to any one of the claims 1-8 or the composition according to claim 13.

71. The method of claim 70, wherein the non-cancer disease is selected from the group consisting of kidney disease or urologic disease.

72. The method of claim 70 or 71, wherein the cells causing the non-cancerous disease are expressing CD-27.

73. A method for inducing or enhancing an immune response against an antigen in a subject comprising administering to the subject a monoclonal antibody which binds to human CD27, in an amount effective to induce or enhance an immune response against an antigen, wherein the antibody comprises a vhCDR1, a vhCDR2, a vhCDR3, a vlCDR1, a vlCDR2, and a vlCDR3 as described in FIG. 1 and/or FIG. 19.

74. A method of inhibiting growth of CD27 expressing cells comprising contacting the cells with a monoclonal antibody which binds to human CD27, in an amount effective to inhibit growth of CD27 expressing cells, wherein the antibody comprises a vhCDR1, a vhCDR2, a vhCDR3, a vlCDR1, a vlCDR2, and a vlCDR3 as described in FIG. 1 and/or FIG. 19.

75. A method of inhibiting the binding of CD70 to CD27 on cells in a subject having a disorder by administering to the subject a monoclonal antibody which binds to human CD27, wherein the antibody comprises a vhCDR1, a vhCDR2, a vhCDR3, a vlCDR1, a vlCDR2, and a vlCDR3 as described in FIG. 1 and/or FIG. 19.

76. A method for inducing or enhancing an immune response against an antigen in a subject comprising administering to the subject a monoclonal antibody which binds to human CD27, in an amount effective to induce or enhance an immune response against an antigen, wherein the antibody binds to the same epitope as an antibody comprising heavy and light chain variable region sequences as set forth in FIG. 1 and/or FIG. 19.

77. A method for inducing or enhancing an immune response against an antigen in a subject comprising administering to the subject a monoclonal antibody which binds to human CD27, in an amount effective to induce or enhance an immune response against an antigen, wherein the antibody competes for binding to human CD27 with an antibody comprising heavy and light chain variable region sequences as set forth in FIG. 1 and/or FIG. 19.

78. A method for inducing or enhancing an immune response against an antigen in a subject comprising administering to the subject a monoclonal antibody which binds to human CD27, in an amount effective to induce or enhance an immune response against an antigen, wherein the antibody comprises a vhCDR1, a vhCDR2, a vhCDR3, a vlCDR1, a vlCDR2, and a vlCDR3 as described in FIG. 1 and/or FIG. 19.

79. A method for inducing or enhancing an immune response in a subject comprising administering to the subject a monoclonal antibody which binds to human CD27, in an amount effective to induce or enhance an immune response, wherein the antibody comprises a vhCDR1, a vhCDR2, a vhCDR3, a vlCDR1, a vlCDR2, and a vlCDR3 as described in FIG. 1 and/or FIG. 19.

80. The method according to any of the preceding claims, wherein the anti-CD27 antibody comprises an anti-CD27 antibody selected from the group consisting of TP01, TP02, TP03, TP04, TP05, TP06, TP07, TP08, TP09, TP10, TP11, TP12, TP13, TP14, TP15, TP16, TP17, TP18, TP19, TP20, TP21, TP22, TP23, TP24, TP25, TP26, TP27, TP28, TP29, TP30, TP31, and TP32.

81. The method according to any of the preceding claims, wherein the anti-CD27 antibody comprises a constant region with an amino acid sequence at least 90% identical to a human IgG.

82. The anti-CD27 antibody according to claim 81, wherein the human IgG is selected from the group consisting of IgG1, IgG2, IgG3, and IgG4.

83. The anti-CD27 antibody according to claim 82, wherein the IgG is an IgG1 or IgG4.


SEQUENCE LISTING (399 sequences)

SEQ ID NO: 1 - protein, 123 aa, Artificial Sequence
  Synthetic TP10 (anti-CD27 antibody) Variable heavy (vh) domain
  misc_feature (89)..(89): Xaa can be any naturally occurring amino acid
QVQLVESGGGVVQPGRSLRLSCAASGFTFSSYAMHWVRQAPGKGLEWVAVISYDGSNKYYADSVKGRFTISRDNSKNTLYLQMNSLRAXDTAVYYCARSGRATSYSYYGMDVWGQGTLVTVSS

SEQ ID NO: 2 - protein, 7 aa, Artificial Sequence
  Synthetic TP10 (anti-CD27 antibody) vhCDR1
GFTFSSY

SEQ ID NO: 3 - protein, 6 aa, Artificial Sequence
  Synthetic TP10 (anti-CD27 antibody) vhCDR2
SYDGSN

SEQ ID NO: 4 - protein, 14 aa, Artificial Sequence
  Synthetic TP10 (anti-CD27 antibody) vhCDR3
SGRATSYSYYGMDV

SEQ ID NO: 5 - protein, 107 aa, Artificial Sequence
  Synthetic TP10 (anti-CD27 antibody) Variable light (vl) domain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIK

SEQ ID NO: 6 - protein, 11 aa, Artificial Sequence
  Synthetic TP10 (anti-CD27 antibody) vlCDR1
RASQSISSYLN

SEQ ID NO: 7 - protein, 7 aa, Artificial Sequence
  Synthetic TP10 (anti-CD27 antibody) vlCDR2
AASSLQS

SEQ ID NO: 8 - protein, 10 aa, Artificial Sequence
  Synthetic TP10 (anti-CD27 antibody) vlCDR3
CQQSYSTPYT

SEQ ID NO: 9 - protein, 121 aa, Artificial Sequence
  Synthetic TP11 (anti-CD27 antibody) Variable heavy (vh) domain
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARGVSGSYARGFDVWGQGTLVTVSS

SEQ ID NO: 10 - protein, 7 aa, Artificial Sequence
  Synthetic TP11 (anti-CD27 antibody) vhCDR1
GFTFSSY

SEQ ID NO: 11 - protein, 7 aa, Artificial Sequence
  Synthetic TP11 (anti-CD27 antibody) vhCDR2
ISYDGSN

SEQ ID NO: 12 - protein, 12 aa, Artificial Sequence
  Synthetic TP11 (anti-CD27 antibody) vhCDR3
GVSGSYARGFDV

SEQ ID NO: 13 - protein, 107 aa, Artificial Sequence
  Synthetic TP11 (anti-CD27 antibody) Variable light (vl) domain
DIQMTQSPSTLSASVGDRVTITCRASQSISSWLAWYQQKPGKAPKLLIYDASSLESGVPSRFSGSGSGTEFTLTISSLQPEDFATYYCQQSYSTPWTFGQGTKVEIK

SEQ ID NO: 14 - protein, 11 aa, Artificial Sequence
  Synthetic TP11 (anti-CD27 antibody) vlCDR1
RASQSISSWLA

SEQ ID NO: 15 - protein, 7 aa, Artificial Sequence
  Synthetic TP11 (anti-CD27 antibody) vlCDR2
DASSLES

SEQ ID NO: 16 - protein, 9 aa, Artificial Sequence
  Synthetic TP11 (anti-CD27 antibody) vlCDR3
QQSYSTPWT

SEQ ID NO: 17 - protein, 119 aa, Artificial Sequence
  Synthetic TP25 (anti-CD27 antibody) Variable heavy (vh) domain
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARPPRTISAFDVWGQGTLVTVSS

SEQ ID NO: 18 - protein, 7 aa, Artificial Sequence
  Synthetic TP25 (anti-CD27 antibody) vhCDR1
GFTFSSY

SEQ ID NO: 19 - protein, 9 aa, Artificial Sequence
  Synthetic TP25 (anti-CD27 antibody) vhCDR2
SAISGSGGS

SEQ ID NO: 20 - protein, 10 aa, Artificial Sequence
  Synthetic TP25 (anti-CD27 antibody) vhCDR3
PPRTISAFDV

SEQ ID NO: 21 - protein, 111 aa, Artificial Sequence
  Synthetic TP25 (anti-CD27 antibody) Variable light (vl) domain
QSVVTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGYVFGTGTKVTVL

SEQ ID NO: 22 - protein, 14 aa, Artificial Sequence
  Synthetic TP25 (anti-CD27 antibody) vlCDR1
TGSSSNIGAGYDVH

SEQ ID NO: 23 - protein, 7 aa, Artificial Sequence
  Synthetic TP25 (anti-CD27 antibody) vlCDR2
GNSNRPS

SEQ ID NO: 24 - protein, 11 aa, Artificial Sequence
  Synthetic TP25 (anti-CD27 antibody) vlCDR3
QSYDSSLSGYV

SEQ ID NO: 25 - protein, 120 aa, Artificial Sequence
  Synthetic TP31 (anti-CD27 antibody) Variable heavy (vh) domain
EVQLVESGGGLVQPGGSLRLSCAASGLTFSSYAMSWVRQAPGKGLEWVSAISGSGGSTYYADSVKGRFTISRDNSKNTLYLQMNSLRAEDTAVYYCARWGERFGSWFDSWGQGTLVTVSS

SEQ ID NO: 26 - protein, 7 aa, Artificial Sequence
  Synthetic TP31 (anti-CD27 antibody) vhCDR1
GLTFSSY

SEQ ID NO: 27 - protein, 9 aa, Artificial Sequence
  Synthetic TP31 (anti-CD27 antibody) vhCDR2
SAISGSGGS

SEQ ID NO: 28 - protein, 11 aa, Artificial Sequence
  Synthetic TP31 (anti-CD27 antibody) vhCDR3
WGERFGSWFDS

SEQ ID NO: 29 - protein, 111 aa, Artificial Sequence; identical to SEQ ID NO: 21
  Synthetic TP31 (anti-CD27 antibody) Variable light (vh) domain

SEQ ID NO: 30 - protein, 14 aa, Artificial Sequence
  Synthetic TP31 (anti-CD27 antibody) vlCDR1
TGSSSNIGAGYDVH

SEQ ID NO: 31 - protein, 7 aa, Artificial Sequence
  Synthetic TP31 (anti-CD27 antibody) vlCDR2
GNSNRPS

SEQ ID NO: 32 - protein, 11 aa, Artificial Sequence
  Synthetic TP31 (anti-CD27 antibody) vlCDR3
QSYDSSLSGYV

SEQ ID NO: 33 - protein, 122 aa, Artificial Sequence
  Synthetic TP01 (anti-CD27 antibody) Variable heavy (vh) domain
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARAGSSGYAYYGMDVWGQGTLVTVSS

SEQ ID NO: 34 - protein, 8 aa, Artificial Sequence
  Synthetic TP01 (anti-CD27 antibody) vhCDR1
GFTFSSYA

SEQ ID NO: 35 - protein, 8 aa, Artificial Sequence
  Synthetic TP01 (anti-CD27 antibody) vhCDR2
ISYDGSNK

SEQ ID NO: 36 - protein, 15 aa, Artificial Sequence
  Synthetic TP01 (anti-CD27 antibody) vhCDR3
ARAGSSGYAYYGMDV

SEQ ID NO: 37 - protein, 107 aa, Artificial Sequence; identical to SEQ ID NO: 5
  Synthetic TP01 (anti-CD27 antibody) Variable light (vl) domain

SEQ ID NO: 38 - protein, 6 aa, Artificial Sequence
  Synthetic TP01 (anti-CD27 antibody) vlCDR1
QSISSY

SEQ ID NO: 39 - protein, 3 aa, Artificial Sequence
  Synthetic TP01 (anti-CD27 antibody) vlCDR2
AAS

SEQ ID NO: 40 - protein, 10 aa, Artificial Sequence
  Synthetic TP01 (anti-CD27 antibody) vlCDR3
QQSYSTPYTF

SEQ ID NO: 41 - protein, 117 aa, Artificial Sequence
  Synthetic TP02 (anti-CD27 antibody) Variable heavy (vh) domain
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARRLVRSVDSWGQGTLVTVSS

SEQ ID NO: 42 - protein, 7 aa, Artificial Sequence
  Synthetic TP02 (anti-CD27 antibody) vhCDR1
GFTFSSY

SEQ ID NO: 43 - protein, 8 aa, Artificial Sequence
  Synthetic TP02 (anti-CD27 antibody) vhCDR2
ISYDGSNK

SEQ ID NO: 44 - protein, 10 aa, Artificial Sequence
  Synthetic TP02 (anti-CD27 antibody) vhCDR3
ARRLVRSVDS

SEQ ID NO: 45 - protein, 107 aa, Artificial Sequence
  Synthetic TP02 (anti-CD27 antibody) Variable light (vl) domain
DIQMTQSPSSLSASVGDRVTITCRASQSISSYLNWYQQKPGKAPKLLIYAASSLQSGVPSRFSGSGSGTDFTLTISRLEPEDFAVYYCQQYGSSPLTFGGGTKVEIK

SEQ ID NO: 46 - protein, 6 aa, Artificial Sequence
  Synthetic TP02 (anti-CD27 antibody) vlCDR1
QSISSY

SEQ ID NO: 47 - protein, 3 aa, Artificial Sequence
  Synthetic TP02 (anti-CD27 antibody) vlCDR2
AAS

SEQ ID NO: 48 - protein, 10 aa, Artificial Sequence
  Synthetic TP02 (anti-CD27 antibody) vlCDR3
QQYGSSPLTF

SEQ ID NO: 49 - protein, 121 aa, Artificial Sequence
  Synthetic TP03 (anti-CD27 antibody) Variable heavy (vh) domain
EVQLVESGGGLVQPGGSLRLSCAASGFTFSNAWMSWVRQAPGKGLEWVGRIKSKTDGGTTDYAAPVKGRFTISRDDSKNTLYLQMNSLKTEDTAVYYCARVGVLENVFDYWGQGPWSPSPQ

SEQ ID NO: 50 - protein, 8 aa, Artificial Sequence
  Synthetic TP03 (anti-CD27 antibody) vhCDR1
GFTFSNAW

SEQ ID NO: 51 - protein, 10 aa, Artificial Sequence
  Synthetic TP03 (anti-CD27 antibody) vhCDR2
IKSKTDGGTT

SEQ ID NO: 52 - protein, 12 aa, Artificial Sequence
  Synthetic TP03 (anti-CD27 antibody) vhCDR3
ARVGVLENVFDY

SEQ ID NO: 53 - protein, 107 aa, Artificial Sequence
  Synthetic TP03 (anti-CD27 antibody) Variable light (vl) domain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPITFGGGTKVEIK

SEQ ID NO: 54 - protein, 6 aa, Artificial Sequence
  Synthetic TP03 (anti-CD27 antibody) vlCDR1
QSISSY

SEQ ID NO: 55 - protein, 3 aa, Artificial Sequence
  Synthetic TP03 (anti-CD27 antibody) vlCDR2
AAS

SEQ ID NO: 56 - protein, 10 aa, Artificial Sequence
  Synthetic TP03 (anti-CD27 antibody) vlCDR3
QQSYSTPITF

SEQ ID NO: 57 - protein, 122 aa, Artificial Sequence
  Synthetic TP04 (anti-CD27 antibody) Variable heavy (vh) domain
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARTSASGYAYYGMDVWGQGTLVTVSS

SEQ ID NO: 58 - protein, 8 aa, Artificial Sequence
  Synthetic TP04 (anti-CD27 antibody) vhCDR1
GFTFSSYA

SEQ ID NO: 59 - protein, 8 aa, Artificial Sequence
  Synthetic TP04 (anti-CD27 antibody) vhCDR2
ISYDGSNK

SEQ ID NO: 60 - protein, 15 aa, Artificial Sequence
  Synthetic TP04 (anti-CD27 antibody) vhCDR3
ARTSASGYAYYGMDV

SEQ ID NO: 61 - protein, 103 aa, Artificial Sequence
  Synthetic TP04 (anti-CD27 antibody) Variable light (vl) domain
DIQMTQSPSTLSASVGDRVTITCRASQSISSYLNWYQQKPGKAPKLLIYAASSLQSGVPSRFSGSGSGTDFTLTISSLQPEDFATYYCQQSYSTPYTFGQGTK

SEQ ID NO: 62 - protein, 6 aa, Artificial Sequence
  Synthetic TP04 (anti-CD27 antibody) vlCDR1
QSISSY

SEQ ID NO: 63 - protein, 3 aa, Artificial Sequence
  Synthetic TP04 (anti-CD27 antibody) vlCDR2
AAS

SEQ ID NO: 64 - protein, 10 aa, Artificial Sequence
  Synthetic TP04 (anti-CD27 antibody) vlCDR3
QQSYSTPYTF

SEQ ID NO: 65 - protein, 119 aa, Artificial Sequence
  Synthetic TP05 (anti-CD27 antibody) Variable heavy (vh) domain
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARVFYGYAPFDVWGQGTLVTVSS

SEQ ID NO: 66 - protein, 8 aa, Artificial Sequence
  Synthetic TP05 (anti-CD27 antibody) vhCDR1
GFTFSSYA

SEQ ID NO: 67 - protein, 8 aa, Artificial Sequence
  Synthetic TP05 (anti-CD27 antibody) vhCDR2
ISYDGSNK

SEQ ID NO: 68 - protein, 12 aa, Artificial Sequence
  Synthetic TP05 (anti-CD27 antibody) vhCDR3
ARVFYGYAPFDV

SEQ ID NO: 69 - protein, 107 aa, Artificial Sequence
  Synthetic TP05 (anti-CD27 antibody) Variable light (vl) domain
DIQMTQSPSTLSASVGDRVTITCRASQSISSWLAWYQQKPGKAPKLLIYDASSLESGVPSRFSGSGSGTEFTLTISSLQPDDFATYYCQQSYSTPYTFGQGTKLEIK

SEQ ID NO: 70 - protein, 6 aa, Artificial Sequence
  Synthetic TP05 (anti-CD27 antibody) vlCDR1
QSISSW

SEQ ID NO: 71 - protein, 3 aa, Artificial Sequence
  Synthetic TP05 (anti-CD27 antibody) vlCDR2
DAS

SEQ ID NO: 72 - protein, 10 aa, Artificial Sequence
  Synthetic TP05 (anti-CD27 antibody) vlCDR3
QQSYSTPYTF

SEQ ID NO: 73 - protein, 120 aa, Artificial Sequence
  Synthetic TP06 (anti-CD27 antibody) Variable heavy (vh) domain
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARGYQGGYSNFDLWGQGTLVTVSS

SEQ ID NO: 74 - protein, 8 aa, Artificial Sequence
  Synthetic TP06 (anti-CD27 antibody) vhCDR1
GFTFSSYA

SEQ ID NO: 75 - protein, 8 aa, Artificial Sequence
  Synthetic TP06 (anti-CD27 antibody) vhCDR2
ISYDGSNK

SEQ ID NO: 76 - protein, 13 aa, Artificial Sequence
  SyntheticTP06 (anti-CD27 antibody) vhCDR3
ARGYQGGYSNFDL

SEQ ID NO: 77 - protein, 107 aa, Artificial Sequence
  Synthetic TP06 (anti-CD27 antibody) Variable light (vl) domain
DIQMTQSPSTLSASVGDRVTITCRASQSISSWLAWYQQKPGKAPKLLIYDASSLESGVPSRFSGSGSGTDFTLTISSLQPEDFATYYCQQSYSTPYTFGQGTKLEIK

SEQ ID NO: 78 - protein, 6 aa, Artificial Sequence
  Synthetic TP06 (anti-CD27 antibody) vlCDR1
QSISSW

SEQ ID NO: 79 - protein, 3 aa, Artificial Sequence
  Synthetic TP06 (anti-CD27 antibody) vlCDR2
DAS

SEQ ID NO: 80 - protein, 10 aa, Artificial Sequence
  Synthetic TP06 (anti-CD27 antibody) vlCDR3
QQSYSTPYTF

SEQ ID NO: 81 - protein, 121 aa, Artificial Sequence
  Synthetic TP07 (anti-CD27 antibody) Variable heavy (vh) domain
EVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARPDSWLAYNYFDLWGQGTLVTVSS

SEQ ID NO: 82 - protein, 8 aa, Artificial Sequence
  Synthetic TP07 (anti-CD27 antibody) vhCDR1
GFTFSSYA

SEQ ID NO: 83 - protein, 8 aa, Artificial Sequence
  Synthetic TP07 (anti-CD27 antibody) vhCDR2
ISYDGSNK

SEQ ID NO: 84 - protein, 14 aa, Artificial Sequence
  Synthetic TP07 (anti-CD27 antibody) vhCDR3
ARPDSWLAYNYFDL

SEQ ID NO: 85 - protein, 107 aa, Artificial Sequence; identical to SEQ ID NO: 77
  Synthetic TP07 (anti-CD27 antibody) Variable light (vl) domain

SEQ ID NO: 86 - protein, 6 aa, Artificial Sequence
  Synthetic TP07 (anti-CD27 antibody) vlCDR1
QSISSW

SEQ ID NO: 87 - protein, 3 aa, Artificial Sequence
  Synthetic TP07 (anti-CD27 antibody) vlCDR2
DAS

SEQ ID NO: 88 - protein, 10 aa, Artificial Sequence
  Synthetic TP07 (anti-CD27 antibody) vlCDR3
QQSYSTPYTF

SEQ ID NO: 89 - protein, 119 aa, Artificial Sequence
  Synthetic TP08 (anti-CD27 antibody) Variable heavy (vh) domain
EVQLVESGGGLVQPGGSLRLSCAASGFTFSSYWMSWVRQAPGKGLEWIGEINHSGSTNYNPSLKSRVTISVDTSKNQFSLKLSSLRSEDTAVYYCARSAYGGAEYFQHWGQGTLVTVSS

SEQ ID NO: 90 - protein, 8 aa, Artificial Sequence
  Synthetic TP08 (anti-CD27 antibody) vhCDR1
GFTFSSYW

SEQ ID NO: 91 - protein, 7 aa, Artificial Sequence
  Synthetic TP08 (anti-CD27 antibody) vhCDR2
INHSGST

SEQ ID NO: 92 - protein, 13 aa, Artificial Sequence
  Synthetic TP08 (anti-CD27 antibody) vhCDR3
ARSAYGGAEYFQH

SEQ ID NO: 93 - protein, 107 aa, Artificial Sequence
  Synthetic TP08 (anti-CD27 antibody) Variable light (vl) domain
DIQMTQSPSTLSASVGDRVTITCRASQSISSWLAWYQQKPGQAPRLLIYGASSRATGIPDRFSGSGSGTDFTLTISRLEPEDFAVYYCQQYGSSPLTFGGGTKVEIK

SEQ ID NO: 94 - protein, 6 aa, Artificial Sequence
  Synthetic TP08 (anti-CD27 antibody) vlCDR1
QSISSW

SEQ ID NO: 95 - protein, 3 aa, Artificial Sequence
  Synthetic TP08 (anti-CD27 antibody) vlCDR2
GAS

SEQ ID NO: 96 - protein, 10 aa, Artificial Sequence
  Synthetic TP08 (anti-CD27 antibody) vlCDR3
QQYGSSPLTF

SEQ ID NO: 97 - protein, 120 aa, Artificial Sequence
  Synthetic TP09 (anti-CD27 antibody) Variable heavy (vh) domain
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARAVPYVYYGMDVWGQGTLVTVSS

SEQ ID NO: 98 - protein, 8 aa, Artificial Sequence
  Synthetic TP09 (anti-CD27 antibody) vhCDR1
GFTFSSYA

SEQ ID NO: 99 - protein, 8 aa, Artificial Sequence
  Synthetic TP09 (anti-CD27 antibody) vhCDR2
ISYDGSNK

SEQ ID NO: 100 - protein, 13 aa, Artificial Sequence
  Synthetic TP09 (anti-CD27 antibody) vhCDR3
ARAVPYVYYGMDV

SEQ ID NO: 101 - protein, 107 aa, Artificial Sequence
  Synthetic TP09 (anti-CD27 antibody) Variable light (vl) domain
DIQMTQSPSSLSASVGDRVTITCRASQSISSYLNWYQQKPGKAPKLLIYAASSLQSGVPSRFSGSGSGTDFTLTISSLQPEDFATYYCQQSYSTPWTFGQGTKVEIK

SEQ ID NO: 102 - protein, 6 aa, Artificial Sequence
  Synthetic TP09 (anti-CD27 antibody) vlCDR1
QSISSY

SEQ ID NO: 103 - protein, 3 aa, Artificial Sequence
  Synthetic TP09 (anti-CD27 antibody) vlCDR2
AAS

SEQ ID NO: 104 - protein, 10 aa, Artificial Sequence
  Synthetic TP09 (anti-CD27 antibody) vlCDR3
QQSYSTPWTF

SEQ ID NO: 105 - protein, 123 aa, Artificial Sequence
  Synthetic TP10 (anti-CD27 antibody) Variable heavy (vh) domain
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARSGRATSYSYYGMDVWGQGTLVTVSS

SEQ ID NO: 106 - protein, 8 aa, Artificial Sequence
  Synthetic TP10 (anti-CD27 antibody) vhCDR1
GFTFSSYA

SEQ ID NO: 107 - protein, 8 aa, Artificial Sequence
  Synthetic TP10 (anti-CD27 antibody) vhCDR2
ISYDGSNK

SEQ ID NO: 108 - protein, 16 aa, Artificial Sequence
  Synthetic TP10 (anti-CD27 antibody) vhCDR3
ARSGRATSYSYYGMDV

SEQ ID NO: 109 - protein, 107 aa, Artificial Sequence; identical to SEQ ID NO: 5
  Synthetic TP10 (anti-CD27 antibody) Variable light (vl) domain

SEQ ID NO: 110 - protein, 6 aa, Artificial Sequence
  Synthetic TP10 (anti-CD27 antibody) vlCDR1
QSISSY

SEQ ID NO: 111 - protein, 3 aa, Artificial Sequence
  Synthetic TP10 (anti-CD27 antibody) vlCDR2
AAS

SEQ ID NO: 112 - protein, 10 aa, Artificial Sequence
  Synthetic TP10 (anti-CD27 antibody) vlCDR3
QQSYSTPYTF

SEQ ID NO: 113 - protein, 121 aa, Artificial Sequence; identical to SEQ ID NO: 9
  Synthetic TP11 (anti-CD27 antibody) Variable heavy (vh) domain

SEQ ID NO: 114 - protein, 8 aa, Artificial Sequence
  Synthetic TP11 (anti-CD27 antibody) vhCDR1
GFTFSSYA

SEQ ID NO: 115 - protein, 8 aa, Artificial Sequence
  Synthetic TP11 (anti-CD27 antibody) vhCDR2
ISYDGSNK

SEQ ID NO: 116 - protein, 14 aa, Artificial Sequence
  Synthetic TP11 (anti-CD27 antibody) vhCDR3
ARGVSGSYARGFDV

SEQ ID NO: 117 - protein, 107 aa, Artificial Sequence; identical to SEQ ID NO: 13
  Synthetic TP11 (anti-CD27 antibody) Variable light (vl) domain

SEQ ID NO: 118 - protein, 6 aa, Artificial Sequence
  Synthetic TP11 (anti-CD27 antibody) vlCDR1
QSISSW

SEQ ID NO: 119 - protein, 3 aa, Artificial Sequence
  Synthetic TP11 (anti-CD27 antibody) vlCDR2
DAS

SEQ ID NO: 120 - protein, 10 aa, Artificial Sequence
  Synthetic TP11 (anti-CD27 antibody) vlCDR3
QQSYSTPWTF

SEQ ID NO: 121 - protein, 120 aa, Artificial Sequence
  Synthetic TP12 (anti-CD27 antibody) Variable heavy (vh) domain
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARSGATLGWYFDLWGQGTLVTVSS

SEQ ID NO: 122 - protein, 8 aa, Artificial Sequence
  Synthetic TP12 (anti-CD27 antibody) vhCDR1
GGTFSSYA

SEQ ID NO: 123 - protein, 8 aa, Artificial Sequence
  Synthetic TP12 (anti-CD27 antibody) vhCDR2
IIPIFGTA

SEQ ID NO: 124 - protein, 13 aa, Artificial Sequence
  Synthetic TP12 (anti-CD27 antibody) vhCDR3
ARSGATLGWYFDL

SEQ ID NO: 125 - protein, 107 aa, Artificial Sequence
  Synthetic TP12 (anti-CD27 antibody) Variable light (vl) domain
EIVMTQSPATLSVSPGERATLSCRASQSVSSNLAWYQQKPGQAPRLLIYGASTRATGIPARFSGSGSGTEFTLTISSLQSEDFAVYYCQQYNNWPFTFGPGTKVDIK

SEQ ID NO: 126 - protein, 6 aa, Artificial Sequence
  Synthetic TP12 (anti-CD27 antibody) vlCDR1
QSVSSN

SEQ ID NO: 127 - protein, 3 aa, Artificial Sequence
  Synthetic TP12 (anti-CD27 antibody) vlCDR2
GAS

SEQ ID NO: 128 - protein, 10 aa, Artificial Sequence
  Synthetic TP12 (anti-CD27 antibody) vlCDR3
QQYNNWPFTF

SEQ ID NO: 129 - protein, 114 aa, Artificial Sequence
  Synthetic TP13 (anti-CD27 antibody) Variable heavy (vh) domain
QLQLQESGPGLVKPSETLSLTCAVYGGSFSGYYWSWIRQPPGKGLEWIGEINHSGSTNYNPSLKSRVTISVDTSKNQFSLKLSSVTAEDTAVYYCARIYNPDVWGQGTLVTVSS

SEQ ID NO: 130 - protein, 8 aa, Artificial Sequence
  Synthetic TP13 (anti-CD27 antibody) vhCDR1
GGSFSGYY

SEQ ID NO: 131 - protein, 7 aa, Artificial Sequence
  Synthetic TP13 (anti-CD27 antibody) vhCDR2
INHSGST

SEQ ID NO: 132 - protein, 8 aa, Artificial Sequence
  Synthetic TP13 (anti-CD27 antibody) vhCDR3
ARIYNPDV

SEQ ID NO: 133 - protein, 107 aa, Artificial Sequence
  Synthetic TP13 (anti-CD27 antibody) Variable light (vl) domain
SYELTQPPSVSVSPGQTASITCSGDKLGDKYACWYQQKPGQSPVLVIYQDSKRPSGIPERFSGSNSGNTATLTISGTQAMDEADYYCQAWDSSTAYVFGTGTKVTVL

SEQ ID NO: 134 - protein, 6 aa, Artificial Sequence
  Synthetic TP13 (anti-CD27 antibody) vlCDR1
KLGDKY

SEQ ID NO: 135 - protein, 3 aa, Artificial Sequence
  Synthetic TP13 (anti-CD27 antibody) vlCDR2
QDS

SEQ ID NO: 136 - protein, 11 aa, Artificial Sequence
  Synthetic TP13 (anti-CD27 antibody) vlCDR3
QAWDSSTAYVF

SEQ ID NO: 137 - protein, 120 aa, Artificial Sequence
  Synthetic TP14 (anti-CD27 antibody) Variable heavy (vh) domain
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARYDHAGGTPFDLWGQGTLVTVSS

SEQ ID NO: 138 - protein, 8 aa, Artificial Sequence
  Synthetic TP14 (anti-CD27 antibody) vhCDR1
GYTFTSYY

SEQ ID NO: 139 - protein, 8 aa, Artificial Sequence
  Synthetic TP14 (anti-CD27 antibody) vhCDR2
INPSGGST

SEQ ID NO: 140 - protein, 13 aa, Artificial Sequence
  Synthetic TP14 (anti-CD27 antibody) vhCDR3
ARYDHAGGTPFDL

SEQ ID NO: 141 - protein, 110 aa, Artificial Sequence
  Synthetic TP14 (anti-CD27 antibody) Variable light (vl) domain
QSVLTQPPSVSAAPGQKVTISCSGSSSNIGNNYVSWYQQLPGTAPKLLIYGNSNRPSGVPDRFSGSKSGTSASLAITGLQAEDEADYYCQSYDSSLSGYVFGTGTKVTVL

SEQ ID NO: 142 - protein, 8 aa, Artificial Sequence
  Synthetic TP14 (anti-CD27 antibody) vlCDR1
SSNIGNNY

SEQ ID NO: 143 - protein, 3 aa, Artificial Sequence
  Synthetic TP14 (anti-CD27 antibody) vlCDR2
GNS

SEQ ID NO: 144 - protein, 12 aa, Artificial Sequence
  Synthetic TP14 (anti-CD27 antibody) vlCDR3
QSYDSSLSGYVF

SEQ ID NO: 145 - protein, 120 aa, Artificial Sequence
  Synthetic TP15 (anti-CD27 antibody) Variable heavy (vh) domain
QVQLVQSGAEVKKPGASVKVSCKASGYTFTSYGISWVRQAPGQGLEWMGWISAYNGNTNYAQKLQGRVTMTTDESTSTAYMELSSLRSEDTAVYYCARGGAGHYDYFDYWGQGTLVTVSS

SEQ ID NO: 146 - protein, 8 aa, Artificial Sequence
  Synthetic TP15 (anti-CD27 antibody) vhCDR1
GYTFTSYG

SEQ ID NO: 147 - protein, 8 aa, Artificial Sequence
  Synthetic TP15 (anti-CD27 antibody) vhCDR2
ISAYNGNT

SEQ ID NO: 148 - protein, 13 aa, Artificial Sequence
  Synthetic TP15 (anti-CD27 antibody) vhCDR3
ARGGAGHYDYFDY

SEQ ID NO: 149 - protein, 109 aa, Artificial Sequence
  Synthetic TP15 (anti-CD27 antibody) Variable light (vl) domain
QSVLTQPPSVSAAPGQKVTISCSGSSSNIGNNYVSWYQQLPGTAPKLLIYDNNKRPSGIPDRFSGSNSGNTATLTISGTQAMDEADYYCQAWDSSTAWVFGGGTKLTVL

SEQ ID NO: 150 - protein, 8 aa, Artificial Sequence
  Synthetic TP15 (anti-CD27 antibody) vlCDR1
SSNIGNNY

SEQ ID NO: 151 - protein, 3 aa, Artificial Sequence
  Synthetic TP15 (anti-CD27 antibody) vlCDR2
DNN

SEQ ID NO: 152 - protein, 11 aa, Artificial Sequence
  Synthetic TP15 (anti-CD27 antibody) vlCDR3
QAWDSSTAWVF

SEQ ID NO: 153 - protein, 119 aa, Artificial Sequence
  Synthetic TP16 (anti-CD27 antibody) Variable heavy (vh) domain
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARASRSLSSFDYWGQGTLVTVSS

SEQ ID NO: 154 - protein, 8 aa, Artificial Sequence
  Synthetic TP16 (anti-CD27 antibody) vhCDR1
GFTFSSYA

SEQ ID NO: 155 - protein, 8 aa, Artificial Sequence
  Synthetic TP16 (anti-CD27 antibody) vhCDR2
ISGSGGST

SEQ ID NO: 156 - protein, 12 aa, Artificial Sequence
  Synthetic TP16 (anti-CD27 antibody) vhCDR3
ARASRSLSSFDY

SEQ ID NO: 157 - protein, 112 aa, Artificial Sequence
  Synthetic TP16 (anti-CD27 antibody) Variable light (vl) domain
QSVVTQPPSVSGAPGQRVTISCTGSSSNIGAGYDVHWYQQLPGTAPKLLIYGNSNRPSGVPDRFSGSKSGTSASLAITGLQAEDEADYYCGTWDSSLSAGYVFGTGTKVTVL

SEQ ID NO: 158 - protein, 9 aa, Artificial Sequence
  Synthetic TP16 (anti-CD27 antibody) vlCDR1
SSNIGAGYD

SEQ ID NO: 159 - protein, 3 aa, Artificial Sequence
  Synthetic TP16 (anti-CD27 antibody) vlCDR2
GNS

SEQ ID NO: 160 - protein, 13 aa, Artificial Sequence
  Synthetic TP16 (anti-CD27 antibody) vlCDR3
GTWDSSLSAGYVF

SEQ ID NO: 161 - protein, 120 aa, Artificial Sequence
  Synthetic TP17 (anti-CD27 antibody) Variable heavy (vh) domain
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARPVFVSGSAFDVWGQGTLVTVSS

SEQ ID NO: 162 - protein, 8 aa, Artificial Sequence
  Synthetic TP17 (anti-CD27 antibody) vhCDR1
GFTFSSYA

SEQ ID NO: 163 - protein, 8 aa, Artificial Sequence
  Synthetic TP17 (anti-CD27 antibody) vhCDR2
ISGSGGST

SEQ ID NO: 164 - protein, 13 aa, Artificial Sequence
  Synthetic TP17 (anti-CD27 antibody) vhCDR3
ARPVFVSGSAFDV

SEQ ID NO: 165 - protein, 111 aa, Artificial Sequence; identical to SEQ ID NO: 21
  Synthetic TP17 (anti-CD27 antibody) Variable light (vl) domain

SEQ ID NO: 166 - protein, 9 aa, Artificial Sequence
  Synthetic TP17 (anti-CD27 antibody) vlCDR1
SSNIGAGYD

SEQ ID NO: 167 - protein, 3 aa, Artificial Sequence
  Synthetic TP17 (anti-CD27 antibody) vlCDR2
GNS

SEQ ID NO: 168 - protein, 12 aa, Artificial Sequence
  Synthetic TP17 (anti-CD27 antibody) vlCDR3
QSYDSSLSGYVF

SEQ ID NO: 169 - protein, 119 aa, Artificial Sequence
  Synthetic TP18 (anti-CD27 antibody) Variable heavy (vh) domain
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARFGYTGSWFDYWGQGTLVTVSS

SEQ ID NO: 170 - protein, 8 aa, Artificial Sequence
  Synthetic TP18 (anti-CD27 antibody) vhCDR1
GFTFSSYA

SEQ ID NO: 171 - protein, 8 aa, Artificial Sequence
  Synthetic TP18 (anti-CD27 antibody) vhCDR2
ISGSGGST

SEQ ID NO: 172 - protein, 12 aa, Artificial Sequence
  Synthetic TP18 (anti-CD27 antibody) vhCDR3
ARFGYTGSWFDY

SEQ ID NO: 173 - protein, 111 aa, Artificial Sequence
  Synthetic TP18 (anti-CD27 antibody) Variable light (vl) domain
QSVVTQPPSVSGAPGQRVTISCTGSSSNIGAGYDVHWYQQLPGTAPKLLIYGKSNRPSGVPDRFSGSKSGTSASLAITGLQAEDEADYYCQSYDSSLSGYVFGTGTKVTVL

SEQ ID NO: 174 - protein, 9 aa, Artificial Sequence
  Synthetic TP18 (anti-CD27 antibody) vlCDR1
SSNIGAGYD

SEQ ID NO: 175 - protein, 3 aa, Artificial Sequence
  Synthetic TP18 (anti-CD27 antibody) vlCDR2
GKS

SEQ ID NO: 176 - protein, 12 aa, Artificial Sequence
  Synthetic TP18 (anti-CD27 antibody) vlCDR3
QSYDSSLSGYVF

SEQ ID NO: 177 - protein, 119 aa, Artificial Sequence
  Synthetic TP19 (anti-CD27 antibody) Variable heavy (vh) domain
QVQLVESGGGVVQPGRSLRLSCAASGFTFSSYAMSWVRQAPGKGLEWVSAISGSGGSTYYADSVKGRFTISRDNSKNTLYLQMNSLRAEDTAVYYCARHGQSQSLVDVWGQGTLVTVSS

SEQ ID NO: 178 - protein, 8 aa, Artificial Sequence
  Synthetic TP19 (anti-CD27 antibody) vhCDR1
GFTFSSYA

SEQ ID NO: 179 - protein, 8 aa, Artificial Sequence
  Synthetic TP19 (anti-CD27 antibody) vhCDR2
ISGSGGST

SEQ ID NO: 180 - protein, 12 aa, Artificial Sequence
  Synthetic TP19 (anti-CD27 antibody) vhCDR3
ARHGQSQSLVDV

SEQ ID NO: 181 - protein, 112 aa, Artificial Sequence
  Synthetic TP19 (anti-CD27 antibody) Variable light (vl) domain
QSVVTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGSYVFGTGTKVTVL

SEQ ID NO: 182 - protein, 9 aa, Artificial Sequence
  Synthetic TP19 (anti-CD27 antibody) vlCDR1
SSNIGAGYD

SEQ ID NO: 183 - protein, 3 aa, Artificial Sequence
  Synthetic TP19 (anti-CD27 antibody) vlCDR2
GNS

SEQ ID NO: 184 - protein, 13 aa, Artificial Sequence
  Synthetic TP19 (anti-CD27 antibody) vlCDR3
QSYDSSLSGSYVF

SEQ ID NO: 185 - protein, 119 aa, Artificial Sequence; identical to SEQ ID NO: 17
  Synthetic TP20 (anti-CD27 antibody) Variable heavy (vh) domain

SEQ ID NO: 186 - protein, 8 aa, Artificial Sequence
  Synthetic TP20 (anti-CD27 antibody) vhCDR1
GFTFSSYA

SEQ ID NO: 187 - protein, 8 aa, Artificial Sequence
  Synthetic TP20 (anti-CD27 antibody) vhCDR2
ISGSGGST

SEQ ID NO: 188 - protein, 12 aa, Artificial Sequence
  Synthetic TP20 (anti-CD27 antibody) vhCDR3
ARPPRTISAFDV

SEQ ID NO: 189 - protein, 111 aa, Artificial Sequence; identical to SEQ ID NO: 21
  Synthetic TP20 (anti-CD27 antibody) Variable light (vl) domain

SEQ ID NO: 190 - protein, 9 aa, Artificial Sequence
  Synthetic TP20 (anti-CD27 antibody) vlCDR1
SSNIGAGYD

SEQ ID NO: 191 - protein, 3 aa, Artificial Sequence
  Synthetic TP20 (anti-CD27 antibody) vlCDR2
GNS

SEQ ID NO: 192 - protein, 12 aa, Artificial Sequence
  Synthetic TP20 (anti-CD27 antibody) vlCDR3
QSYDSSLSGYVF

SEQ ID NO: 193 - protein, 120 aa, Artificial Sequence
  Synthetic TP21 (anti-CD27 antibody) Variable heavy (vh) domain
EVQLVESGGGLVQPGRSLRLSCAASGFTFSSYAMSWVRQAPGKGLEWVSAISGSGGSTYYADSVKGRFTISRDNSKNTLYLQMNSLRAEDTAVYYCARYRPVYGSWFDSWGQGTLVTVSS

SEQ ID NO: 194 - protein, 8 aa, Artificial Sequence
  Synthetic TP21 (anti-CD27 antibody) vhCDR1
GFTFSSYA

SEQ ID NO: 195 - protein, 8 aa, Artificial Sequence
  Synthetic TP21 (anti-CD27 antibody) vhCDR2
ISGSGGST

SEQ ID NO: 196 - protein, 13 aa, Artificial Sequence
  Synthetic TP21 (anti-CD27 antibody) vhCDR3
ARYRPVYGSWFDS

SEQ ID NO: 197 - protein, 111 aa, Artificial Sequence; identical to SEQ ID NO: 21
  Synthetic TP21 (anti-CD27 antibody) Variable light (vl) domain

SEQ ID NO: 198 - protein, 9 aa, Artificial Sequence
  Synthetic TP21 (anti-CD27 antibody) vlCDR1
SSNIGAGYD

SEQ ID NO: 199 - protein, 3 aa, Artificial Sequence
  Synthetic TP21 (anti-CD27 antibody) vlCDR2
GNS

SEQ ID NO: 200 - protein, 12 aa, Artificial Sequence
  Synthetic TP21 (anti-CD27 antibody) vlCDR3
QSYDSSLSGYVF

SEQ ID NO: 201 - protein, 119 aa, Artificial Sequence
  Synthetic TP22 (anti-CD27 antibody) Variable heavy (vh) domain
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARVQRTISAFQHWGQGTLVTVSS

SEQ ID NO: 202 - protein, 8 aa, Artificial Sequence
  Synthetic TP22 (anti-CD27 antibody) vhCDR1
GFTFSSYA

SEQ ID NO: 203 - protein, 8 aa, Artificial Sequence
  Synthetic TP22 (anti-CD27 antibody) vhCDR2
ISGSGGST

SEQ ID NO: 204 - protein, 12 aa, Artificial Sequence
  Synthetic TP22 (anti-CD27 antibody) vhCDR3
ARVQRTISAFQH

SEQ ID NO: 205 - protein, 111 aa, Artificial Sequence; identical to SEQ ID NO: 21
  Synthetic TP22 (anti-CD27 antibody) Variable light (vl) domain

SEQ ID NO: 206 - protein, 9 aa, Artificial Sequence
  Synthetic TP22 (anti-CD27 antibody) vlCDR1
SSNIGAGYD

SEQ ID NO: 207 - protein, 3 aa, Artificial Sequence
  Synthetic TP22 (anti-CD27 antibody) vlCDR2
GNS

SEQ ID NO: 208 - protein, 12 aa, Artificial Sequence
  Synthetic TP22 (anti-CD27 antibody) vlCDR3
QSYDSSLSGYVF

SEQ ID NO: 209 - protein, 120 aa, Artificial Sequence
  Synthetic TP23 (anti-CD27 antibody) Variable heavy (vh) domain
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGGAGHYDYFDYWGQGTLVTVSS

SEQ ID NO: 210 - protein, 8 aa, Artificial Sequence
  Synthetic TP23 (anti-CD27 antibody) vhCDR1
GYTFTSYG

SEQ ID NO: 211 - protein, 8 aa, Artificial Sequence
  Synthetic TP23 (anti-CD27 antibody) vhCDR2
ISAYNGNT

SEQ ID NO: 212 - protein, 13 aa, Artificial Sequence
  Synthetic TP23 (anti-CD27 antibody) vhCDR3
ARGGAGHYDYFDY

SEQ ID NO: 213 - protein, 111 aa, Artificial Sequence; identical to SEQ ID NO: 21
  Synthetic TP23 (anti-CD27 antibody) Variable light (vl) domain

SEQ ID NO: 214 - protein, 9 aa, Artificial Sequence
  Synthetic TP23 (anti-CD27 antibody) vlCDR1
SSNIGAGYD

SEQ ID NO: 215 - protein, 3 aa, Artificial Sequence
  Synthetic TP23 (anti-CD27 antibody) vlCDR2
GNS

SEQ ID NO: 216 - protein, 12 aa, Artificial Sequence
  Synthetic TP23 (anti-CD27 antibody) vlCDR3
QSYDSSLSGYVF

SEQ ID NO: 217 - protein, 119 aa, Artificial Sequence
  Synthetic TP24 (anti-CD27 antibody) Variable heavy (vh) domain
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARPVRTISAFDVWGQGTLVTVSS

SEQ ID NO: 218 - protein, 8 aa, Artificial Sequence
  Synthetic TP24 (anti-CD27 antibody) vhCDR1
GFTFSSYA

SEQ ID NO: 219 - protein, 8 aa, Artificial Sequence
  Synthetic TP24 (anti-CD27 antibody) vhCDR2
ISYDGSNK

SEQ ID NO: 220 - protein, 12 aa, Artificial Sequence
  Synthetic TP24 (anti-CD27 antibody) vhCDR3
ARPVRTISAFDV

SEQ ID NO: 221 - protein, 112 aa, Artificial Sequence; identical to SEQ ID NO: 157
  Synthetic TP24 (anti-CD27 antibody) Variable light (vl) domain

SEQ ID NO: 222 - protein, 9 aa, Artificial Sequence
  Synthetic TP24 (anti-CD27 antibody) vlCDR1
SSNIGAGYD

SEQ ID NO: 223 - protein, 3 aa, Artificial Sequence
  Synthetic TP24 (anti-CD27 antibody) vlCDR2
GNS

SEQ ID NO: 224 - protein, 13 aa, Artificial Sequence
  Synthetic TP24 (anti-CD27 antibody) vlCDR3
GTWDSSLSAGYVF

SEQ ID NO: 225 - protein, 119 aa, Artificial Sequence; identical to SEQ ID NO: 17
  Synthetic TP25 (anti-CD27 antibody) Variable heavy (vh) domain

SEQ ID NO: 226 - protein, 8 aa, Artificial Sequence
  Synthetic TP25 (anti-CD27 antibody) vhCDR1
GFTFSSYA

SEQ ID NO: 227 - protein, 8 aa, Artificial Sequence
  Synthetic TP25 (anti-CD27 antibody) vhCDR2
ISGSGGST

SEQ ID NO: 228 - protein, 12 aa, Artificial Sequence
  Synthetic TP25 (anti-CD27 antibody) vhCDR3
ARPPRTISAFDV

SEQ ID NO: 229 - protein, 111 aa, Artificial Sequence; identical to SEQ ID NO: 21
  Synthetic TP25 (anti-CD27 antibody) Variable light (vl) domain

SEQ ID NO: 230 - protein, 9 aa, Artificial Sequence
  Synthetic TP25 (anti-CD27 antibody) vlCDR1
SSNIGAGYD

SEQ ID NO: 231 - protein, 3 aa, Artificial Sequence
  Synthetic TP25 (anti-CD27 antibody) vlCDR2
GNS

SEQ ID NO: 232 - protein, 12 aa, Artificial Sequence
  Synthetic TP25 (anti-CD27 antibody) vlCDR3
QSYDSSLSGYVF

SEQ ID NO: 233 - protein, 119 aa, Artificial Sequence
  Synthetic TP26 (anti-CD27 antibody) Variable heavy (vh) domain
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARPGLSLSWFDSWGQGTLVTVSS

SEQ ID NO: 234 - protein, 8 aa, Artificial Sequence
  Synthetic TP26 (anti-CD27 antibody) vhCDR1
GFTFSSYA

SEQ ID NO: 235 - protein, 8 aa, Artificial Sequence
  Synthetic TP26 (anti-CD27 antibody) vhCDR2
ISYDGSNK

SEQ ID NO: 236 - protein, 12 aa, Artificial Sequence
  Synthetic TP26 (anti-CD27 antibody) vhCDR3
ARPGLSLSWFDS

SEQ ID NO: 237 - protein, 111 aa, Artificial Sequence; identical to SEQ ID NO: 21
  Synthetic TP26 (anti-CD27 antibody) Variable light (vl) domain

SEQ ID NO: 238 - protein, 9 aa, Artificial Sequence
  Synthetic TP26 (anti-CD27 antibody) vlCDR1
SSNIGAGYD

SEQ ID NO: 239 - protein, 3 aa, Artificial Sequence
  Synthetic TP26 (anti-CD27 antibody) vlCDR2
GNS

SEQ ID NO: 240 - protein, 12 aa, Artificial Sequence
  Synthetic TP26 (anti-CD27 antibody) vlCDR3
QSYDSSLSGYVF

SEQ ID NO: 241 - protein, 124 aa, Artificial Sequence
  Synthetic TP27 (anti-CD27 antibody) Variable heavy (vh) domain
QLQLQQWGAGLLKPSETLSLTCAVYGGSFSGYYWSWIRQPPGRGLEWIGEINHSGSTNYNPSLKSRVTISVDTSKNQFSLKLSSVTAADTAVYYCARGHRTKSGQSSKPPFQHWGQGTLVTVSS

SEQ ID NO: 242 - protein, 8 aa, Artificial Sequence
  Synthetic TP27 (anti-CD27 antibody) vhCDR1
GGSFSGYY

SEQ ID NO: 243 - protein, 7 aa, Artificial Sequence
  Synthetic TP27 (anti-CD27 antibody) vhCDR2
INHSGST

SEQ ID NO: 244 - protein, 18 aa, Artificial Sequence
  Synthetic TP27 (anti-CD27 antibody) vhCDR3
ARGHRTKSGQSSKPPFQH

SEQ ID NO: 245 - protein, 111 aa, Artificial Sequence
  Synthetic TP27 (anti-CD27 antibody) Variable light (vl) domain
QSVVTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAGWVFGGGTKLTVL

SEQ ID NO: 246 - protein, 8 aa, Artificial Sequence
  Synthetic TP27 (anti-CD27 antibody) vlCDR1
SSNIGNNY

SEQ ID NO: 247 - protein, 3 aa, Artificial Sequence
  Synthetic TP27 (anti-CD27 antibody) vlCDR2
DNN

SEQ ID NO: 248 - protein, 13 aa, Artificial Sequence
  Synthetic TP27 (anti-CD27 antibody) vlCDR3
GTWDSSLSAGWVF

SEQ ID NO: 249 - protein, 119 aa, Artificial Sequence; identical to SEQ ID NO: 169
  Synthetic TP28 (anti-CD27 antibody) Variable heavy (vh) domain

SEQ ID NO: 250 - protein, 8 aa, Artificial Sequence
  Synthetic TP28 (anti-CD27 antibody) vhCDR1
GFTFSSYA

SEQ ID NO: 251 - protein, 8 aa, Artificial Sequence
  Synthetic TP28 (anti-CD27 antibody) vhCDR2
ISGSGGST

SEQ ID NO: 252 - protein, 12 aa, Artificial Sequence
  Synthetic TP28 (anti-CD27 antibody) vhCDR3
ARFGYTGSWFDY

SEQ ID NO: 253 - protein, 111 aa, Artificial Sequence; identical to SEQ ID NO: 173
  Synthetic TP28 (anti-CD27 antibody) Variable light (vl) domain

SEQ ID NO: 254 - protein, 9 aa, Artificial Sequence
  Synthetic TP28 (anti-CD27 antibody) vlCDR1
SSNIGAGYD

SEQ ID NO: 255 - protein, 3 aa, Artificial Sequence
  Synthetic TP28 (anti-CD27 antibody) vlCDR2
GKS

SEQ ID NO: 256 - protein, 12 aa, Artificial Sequence
  Synthetic TP28 (anti-CD27 antibody) vlCDR3
QSYDSSLSGYVF

SEQ ID NO: 257 - protein, 119 aa, Artificial Sequence
  Synthetic TP29 (anti-CD27 antibody) Variable heavy (vh) domain
EVQLVESGGGVVQPGRSLRLSCAASGFTFSNAWMSWVRQAPGKGLEWVSAISGSGGSTYYADSVKGRFTISRDNSKNTLYLQMNSLRAEDTAVYYCARRALSLSYFDYWGQGTLVTVSS

SEQ ID NO: 258 - protein, 8 aa, Artificial Sequence
  Synthetic TP29 (anti-CD27 antibody) vhCDR1
GFTFSNAW

SEQ ID NO: 259 - protein, 8 aa, Artificial Sequence
  Synthetic TP29 (anti-CD27 antibody) vhCDR2
ISGSGGST

SEQ ID NO: 260 - protein, 12 aa, Artificial Sequence
  Synthetic TP29 (anti-CD27 antibody) vhCDR3
ARRALSLSYFDY

SEQ ID NO: 261 - protein, 111 aa, Artificial Sequence; identical to SEQ ID NO: 21
  Synthetic TP29 (anti-CD27 antibody) Variable light (vl) domain

SEQ ID NO: 262 - protein, 9 aa, Artificial Sequence
  Synthetic TP29 (anti-CD27 antibody) vlCDR1
SSNIGAGYD

SEQ ID NO: 263 - protein, 3 aa, Artificial Sequence
  Synthetic TP29 (anti-CD27 antibody) vlCDR2
GNS

SEQ ID NO: 264 - protein, 12 aa, Artificial Sequence
  Synthetic TP29 (anti-CD27 antibody) vlCDR3
QSYDSSLSGYVF

SEQ ID NO: 265 - protein, 119 aa, Artificial Sequence
  Synthetic TP30 (anti-CD27 antibody) Variable heavy (vh) domain
EVQLVESGGGVVQPGRSLRLSCAASGFTFSSYAMSWVRQAPGKGLEWVSAISGSGGSTYYADSVKGRFTISRDNSKNTLYLQMNSLRAEDTAVYYCARGSRTLSAFDVWGQGTLVTVSS

SEQ ID NO: 266 - protein, 8 aa, Artificial Sequence
  Synthetic TP30 (anti-CD27 antibody) vhCDR1
GFTFSSYA

SEQ ID NO: 267 - protein, 8 aa, Artificial Sequence
  Synthetic TP30 (anti-CD27 antibody) vhCDR2
ISGSGGST

SEQ ID NO: 268 - protein, 12 aa, Artificial Sequence
  Synthetic TP30 (anti-CD27 antibody) vhCDR3
ARGSRTLSAFDV

SEQ ID NO: 269 - protein, 111 aa, Artificial Sequence; identical to SEQ ID NO: 21
  Synthetic TP30 (anti-CD27 antibody) Variable light (vl) domain

SEQ ID NO: 270 - protein, 9 aa, Artificial Sequence
  Synthetic TP30 (anti-CD27 antibody) vlCDR1
SSNIGAGYD

SEQ ID NO: 271 - protein, 3 aa, Artificial Sequence
  Synthetic TP30 (anti-CD27 antibody) vlCDR2
GNS

SEQ ID NO: 272 - protein, 12 aa, Artificial Sequence
  Synthetic TP30 (anti-CD27 antibody) vlCDR3
QSYDSSLSGYVF

SEQ ID NO: 273 - protein, 120 aa, Artificial Sequence; identical to SEQ ID NO: 25
  Synthetic TP31 (anti-CD27 antibody) Variable heavy (vh) domain

SEQ ID NO: 274 - protein, 8 aa, Artificial Sequence
  Synthetic TP31 (anti-CD27 antibody) vhCDR1
GLTFSSYA

SEQ ID NO: 275 - protein, 8 aa, Artificial Sequence
  Synthetic TP31 (anti-CD27 antibody) vhCDR2
ISGSGGST

SEQ ID NO: 276 - protein, 13 aa, Artificial Sequence
  Synthetic TP31 (anti-CD27 antibody) vhCDR3
ARWGERFGSWFDS

SEQ ID NO: 277 - protein, 111 aa, Artificial Sequence; identical to SEQ ID NO: 21
  Synthetic TP31 (anti-CD27 antibody) Variable light (vl) domain

SEQ ID NO: 278 - protein, 9 aa, Artificial Sequence
  Synthetic TP31 (anti-CD27 antibody) vlCDR1
SSNIGAGYD

SEQ ID NO: 279 - protein, 3 aa, Artificial Sequence
  Synthetic TP31 (anti-CD27 antibody) vlCDR2
GNS

SEQ ID NO: 280 - protein, 12 aa, Artificial Sequence
  Synthetic TP31 (anti-CD27 antibody) vlCDR3
QSYDSSLSGYVF

SEQ ID NO: 281 - protein, 117 aa, Artificial Sequence
  Synthetic TP32 (anti-CD27 antibody) Variable heavy (vh) domain
QLQLQQWGAGLLKPSETLSLTCAVYGGSFSGYYWSWIRQPQGKGLEWIGEINHSGSTNYNPSLKSRVTISVDTSKNQFSLKLSSVTAADTAVYYCARVSVNFYFDLWGQGTLVTVSS

SEQ ID NO: 282 - protein, 8 aa, Artificial Sequence
  Synthetic TP32 (anti-CD27 antibody) vhCDR1
GGSFSGYY

SEQ ID NO: 283 - protein, 7 aa, Artificial Sequence
  Synthetic TP32 (anti-CD27 antibody) vhCDR2
INHSGST

SEQ ID NO: 284 - protein, 10 aa, Artificial Sequence
  Synthetic TP32 (anti-CD27 antibody) vhCDR3
ARVSVNFYFD

SEQ ID NO: 285 - protein, 110 aa, Artificial Sequence
  Synthetic TP32 (anti-CD27 antibody) Variable light (vl) domain
QSALTQPASVSGSPGQSITISCTGTSSDVGSYNLVSWYQQHPGKAPKLMIYEGSKRPSGVSNRFSGSKSGNTASLTISGLQAEDEADYYCCSYAGSSTFVFGTGTKVTVL

SEQ ID NO: 286 - protein, 9 aa, Artificial Sequence
  Synthetic TP32 (anti-CD27 antibody) vlCDR1
SSDVGSYNL

SEQ ID NO: 287 - protein, 3 aa, Artificial Sequence
  Synthetic TP32 (anti-CD27 antibody) vlCDR2
EGS

SEQ ID NO: 288 - protein, 11 aa, Artificial Sequence
  Synthetic TP32 (anti-CD27 antibody) vlCDR3
CSYAGSSTFVF

SEQ ID NO: 289 - protein, 330 aa, Artificial Sequence [sequence omitted]

SEQ ID NO: 290 - protein, 232 aa, Artificial Sequence
  Synthetic Human IgG1 Fc domain
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 291 - protein, 227 aa, Artificial Sequence
  Synthetic Human IgG1 Fc domain Deletion (Alpha-EPKSC) Upper Hinge
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 292 - protein, 749 aa, Artificial Sequence
  Synthetic Human serum albumin
MDMRVPAQLLGLLLLWLPGARCADAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGS

SEQ ID NO: 293 - protein, 726 aa, Artificial Sequence
  Synthetic Mature HSA
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGS

SEQ ID NO: 294 - DNA, 2247 bp, Artificial Sequence
  Synthetic Human serum albumin
atggatatgcgggtgcctgctcagctgctgggactgctgctgctgtggctgcctggggctagatgcgccgatgctcacaaaagcgaagtcgcacacaggttcaaagatctgggggaggaaaactttaaggctctggtgctgattgcattcgcccagtacctgcagcagtgcccctttgaggaccacgtgaaactggtcaacgaagtgactgagttcgccaagacctgcgtggccgacgaatctgctgagaattgtgataaaagtctgcatactctgtttggggataagctgtgtacagtggccactctgcgagaaacctatggagagatggcagactgctgtgccaaacaggaacccgagcggaacgaatgcttcctgcagcataaggacgataaccccaatctgcctcgcctggtgcgacctgaggtggacgtcatgtgtacagccttccacgataatgaggaaacttttctgaagaaatacctgtacgaaatcgctcggagacatccttacttttatgcaccagagctgctgttctttgccaaacgctacaaggccgctttcaccgagtgctgtcaggcagccgataaagctgcatgcctgctgcctaagctggacgaactgagggatgagggcaaggccagctccgctaaacagcgcctgaagtgtgctagcctgcagaaattcggggagcgagccttcaaggcttgggcagtggcacggctgagtcagagattcccaaaggcagaatttgccgaggtctcaaaactggtgaccgacctgacaaaggtgcacaccgaatgctgtcatggcgacctgctggagtgcgccgacgatcgagctgatctggcaaagtatatttgtgagaaccaggactccatctctagtaagctgaaagaatgctgtgagaaaccactgctggaaaagtctcactgcattgccgaagtggagaacgacgagatgccagctgatctgccctcactggccgctgacttcgtcgaaagcaaagatgtgtgtaagaattacgctgaggcaaaggatgtgttcctgggaatgtttctgtacgagtatgccaggcgccacccagactactccgtggtcctgctgctgaggctggctaaaacatatgaaaccacactggagaagtgctgtgcagccgctgatccccatgaatgctatgccaaagtcttcgacgagtttaagcccctggtggaggaacctcagaacctgatcaaacagaattgtgaactgtttgagcagctgggcgagtacaagttccagaacgccctgctggtgcgctataccaagaaagtcccacaggtgtccacacccactctggtggaggtgagccggaatctgggcaaagtggggagtaaatgctgtaagcaccctgaagccaagaggatgccatgcgctgaggattacctgagtgtggtcctgaatcagctgtgtgtcctgcatgaaaaaacacctgtcagcgaccgggtgacaaagtgctgtactgagtcactggtgaaccgacggccctgctttagcgccctggaagtcgatgagacttatgtgcctaaagagttcaacgctgagaccttcacatttcacgcagacatttgtaccctgagcgaaaaggagagacagatcaagaaacagacagccctggtcgaactggtgaagcataaacccaaggccacaaaagagcagctgaaggctgtcatggacgatttcgcagcctttgtggaaaaatgctgtaaggcagacgataaggagacttgctttgccgaggaaggaaagaaactggtggctgcatcccaggcagctctgggactgggaggaggatctgcccctacctcaagctccactaagaaaacccagctgcagctggagcacctgctgctggacctgcagatgattctgaacgggatcaacaattacaaaaatccaaagctgacccggatgctgacattcaagttttatatgcccaagaaagccacagagctgaaacacctgcagtgcctggaggaagagctgaagcctctggaagaggtgctgaacctggcccagagcaagaatttccatctgagaccaagggatctgatctccaacattaatgtgatcgtcctggaactgaagggatctgagactacctttatgtgcgaatacgctgacgagactgcaaccattgtggagttcctgaacagatggatcaccttctgccagtccatcatttctactctgacaggcggggggagc

SEQ ID NO: 295 - protein, 28 aa, Artificial Sequence
  Synthetic EETI-II from Knottin Database
GCPRILMRCKQDSDCLAGCVCGPNGFCG

SEQ ID NO: 296 - protein, 47 aa, Artificial Sequence
  Synthetic AgRP from Knottin Database
GCVRLHESCLGQQVPCCDPCATCYCRFFNAFCYCRKLGTAMNPCSRT

SEQ ID NO: 297 - protein, 48 aa, Artificial Sequence
  Synthetic Omega agatoxin from Knottin Database
EDNCIAEDYGKCTWGGTKCCRGRPCRCSMIGTNCECTPRLIMEGLSFA

SEQ ID NO: 298 - protein, 33 aa, Artificial Sequence
  Synthetic EETI-II Library
  misc_feature (3)..(5): Xaa can be any naturally occurring amino acid
  misc_feature (9)..(13): Xaa can be any naturally occurring amino acid
GCXXXRGDXXXXXCKQDSDCLAGCVCGPNGFCG

SEQ ID NO: 299 - protein, 33 aa, Artificial Sequence
  Synthetic EETI-II K15S Mutation Library
  misc_feature (3)..(5): Xaa can be any naturally occurring amino acid
  misc_feature (9)..(13): Xaa can be any naturally occurring amino acid
GCXXXRGDXXXXXCSQDSDCLAGCVCGPNGFCG

SEQ ID NO: 300 - DNA, 822 bp, Artificial Sequence
  Synthetic 2.5F-(K15S) mIgG2aFc
ggttgtccaagaccaagaggtgataatccaccattgacttgttctcaagattctgattgtttggctggttgtgtttgtggtccaaatggtttttgtggtggtcgactagagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaa

SEQ ID NO: 301 - protein, 271 aa, Artificial Sequence
  Synthetic 2.5F-(K15S) mIgG2aFc
GCPRPRGDNPPLTCSQDSDCLAGCVCGPNGFCGEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 302 - DNA, 822 bp, Artificial Sequence
  Synthetic 2.5D-(K15S) mIgG2aFc
ggttgtccacaaggcagaggtgattgggctccaacttcttgttctcaagattctgattgtttggctggttgtgtttgtggtccaaatggtttttgtggtggtcgactagagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaa

SEQ ID NO: 303 - protein, 271 aa, Artificial Sequence
  Synthetic 2.5D-(K15S) mIgG2aFc
GCPQGRGDWAPTSCSQDSDCLAGCVCGPNGFCGEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 304 - protein, 265 aa, Artificial Sequence
  Synthetic 2.5F-(K15S) hIgG1Fc
GCPRPRGDNPPLTCSQDSDCLAGCVCGPNGFCGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 305 - protein, 260 aa, Artificial Sequence
  Synthetic 2.5F-(K15S) hIgG1Fc Fc Upper Hinge Deletion (Alpha-EPKSC)
GCPRPRGDNPPLTCSQDSDCLAGCVCGPNGFCGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 306 - protein, 265 aa, Artificial Sequence
  Synthetic 2.5D-(K15S) hIgG1Fc
GCPQGRGDWAPTSCSQDSDCLAGCVCGPNGFCGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 307 - protein, 260 aa, Artificial Sequence
  Synthetic 2.5D-(K15S) hIgG1Fc Fc Upper Hinge Deletion (Alpha-EPKSC)
GCPQGRGDWAPTSCSQDSDCLAGCVCGPNGFCGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 308 - protein, 287 aa, Artificial Sequence
  Synthetic hPD-1
MQIPQAPWPVVWAVLQLGWRPGWFLDSPDPWNPPTFFPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 309 - protein, 290 aa, Artificial Sequence
  Synthetic hPD-L1
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 310 - protein, 223 aa, Artificial Sequence
  Synthetic hCTLA-4
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 311 - protein, 525 aa, Artificial Sequence
  Synthetic hLAG-3
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 312 - protein, 300 aa, Artificial Sequence
  Synthetic hTIM-3
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 313 - protein, 532 aa, Artificial Sequence
  Synthetic hB7-H3
MLRRRGSPGMGVHVGAALGALWFCLTGALEVQVPEDPVVALVGTDATLCCSFSPEPGFSLQLNLIWQLTDTKQLVHSFAEGQDQGSAYANRTALFPDLLAQGNASLRLQRVRVADEGSFCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 314 - protein, 166 aa, Artificial Sequence
  Synthetic hB7-H4
MASLGQILFWSIISIIIILAGAIALIIGFGISAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 315 - protein, 33 aa, Artificial Sequence
  Synthetic Integrin Binding Knottin Peptide 1.4A
GCAEPRGDMPWTWCKQDSDCLAGCVCGPNGFCG

SEQ ID NO: 316 - protein, 33 aa, Artificial Sequence
  Synthetic Integrin Binding Knottin Peptide 1.4B
GCVGGRGDWSPKWCKQDSDCPAGCVCGPNGFCG

SEQ ID NO: 317 - protein, 33 aa, Artificial Sequence
  Synthetic Integrin Binding Knottin Peptide 1.4C
GCAELRGDRSYPECKQDSDCLAGCVCGPNGFCG

SEQ ID NO: 318 - protein, 33 aa, Artificial Sequence
  Synthetic Integrin Binding Knottin Peptide 1.4E
GCRLPRGDVPRPHCKQDSDCQAGCVCGPNGFCG

SEQ ID NO: 319 - protein, 33 aa, Artificial Sequence
  Synthetic Integrin Binding Knottin Peptide 1.4H
GCYPLRGDNPYAACKQDSDCRAGCVCGPNGFCG

SEQ ID NO: 320 - protein, 33 aa, Artificial Sequence
  Synthetic Integrin Binding Knottin Peptide 1.5B
GCTIGRGDWAPSECKQDSDCLAGCVCGPNGFCG

SEQ ID NO: 321 - protein, 33 aa, Artificial Sequence
  Synthetic Integrin Binding Knottin Peptide 1.5F
GCHPPRGDNPPVTCKQDSDCLAGCVCGPNGFCG

SEQ ID NO: 322 - protein, 33 aa, Artificial Sequence
  Synthetic Integrin Binding Knottin Peptide 2.3A
GCPEPRGDNPPPSCKQDSDCRAGCVCGPNGFCG

SEQ ID NO: 323 - protein, 33 aa, Artificial Sequence
  Synthetic Integrin Binding Knottin Peptide 2.3B
GCLPPRGDNPPPSCKQDSDCQAGCVCGPNGFCG

SEQ ID NO: 324 - protein, 33 aa, Artificial Sequence
  Synthetic Integrin Binding Knottin Peptide 2.3C
GCHLGRGDWAPVGCKQDSDCPAGCVCGPNGFCG

SEQ ID NO: 325 - protein, 33 aa, Artificial Sequence
  Synthetic Integrin Binding Knottin Peptide 2.3D
GCNVGRGDWAPSECKQDSDCPAGCVCGPNGFCG

SEQ ID NO: 326 - protein, 33 aa, Artificial Sequence
  Synthetic Integrin Binding Knottin Peptide 2.3E
GCFPGRGDWAPSSCKQDSDCRAGCVCGPNGFCG

SEQ ID NO: 327 - protein, 33 aa, Artificial Sequence
  Synthetic Integrin Binding Knottin Peptide 2.3F
GCPLPRGDNPPTECKQDSDCQAGCVCGPNGFCG

SEQ ID NO: 328 - protein, 33 aa, Artificial Sequence
  Synthetic Integrin Binding Knottin Peptide 2.3G
GCSEARGDNPRLSCKQDSDCRAGCVCGPNGFCG

SEQ ID NO: 329 - protein, 32 aa, Artificial Sequence
  Synthetic Integrin Binding Knottin Peptide 2.3H
GCLLGRGDWAPEACKQDSDCRAGCVCPNGFCG

SEQ ID NO: 330 - protein, 33 aa, Artificial Sequence
  Synthetic Integrin Binding Knottin Peptide 2.3I
GCHVGRGDWAPLKCKQDSDCQAGCVCGPNGFCG

SEQ ID NO: 331 - protein, 33 aa, Artificial Sequence
  Synthetic Integrin Binding Knottin Peptide 2.3J
GCVRGRGDWAPPSCKQDSDCPAGCVCGPNGFCG

SEQ ID NO: 332 - protein, 33 aa, Artificial Sequence
  Synthetic Integrin Binding Knottin Peptide 2.4A
GCLGGRGDWAPPACKQDSDCRAGCVCGPNGFCG

SEQ ID NO: 333 - protein, 33 aa, Artificial Sequence
  Synthetic Integrin Binding Knottin Peptide 2.4C
GCFVGRGDWAPLTCKQDSDCQAGCVCGPNGFCG

SEQ ID NO: 334 - protein, 33 aa, Artificial Sequence
  Synthetic Integrin Binding Knottin Peptide 2.4D
GCPVGRGDWSPASCKQDSDCRAGCVCGPNGFCG

SEQ ID NO: 335 - protein, 33 aa, Artificial Sequence
  Synthetic Integrin Binding Knottin Peptide 2.4E
GCPRPRGDNPPLTCKQDSDCLAGCVCGPNGFCG

SEQ ID NO: 336 - protein, 33 aa, Artificial Sequence
  Synthetic Integrin Binding Knottin Peptide 2.4F
GCYQGRGDWSPSSCKQDSDCPAGCVCGPNGFCG

SEQ ID NO: 337 - protein, 33 aa, Artificial Sequence
  Synthetic Integrin Binding Knottin Peptide 2.4G
GCAPGRGDWAPSECKQDSDCQAGCVCGPNGFCG

SEQ ID NO: 338 - protein, 33 aa, Artificial Sequence
  Synthetic Integrin Binding Knottin Peptide 2.4J
GCVQGRGDWSPPSCKQDSDCPAGCVCGPNGFCG

SEQ ID NO: 339 - protein, 33 aa, Artificial Sequence
  Synthetic Integrin Binding Knottin Peptide 2.5A
GCHVGRGDWAPEECKQDSDCQAGCVCGPNGFCG

SEQ ID NO: 340 - protein, 33 aa, Artificial Sequence
  Synthetic Integrin Binding Knottin Peptide 2.5C
GCDGGRGDWAPPACKQDSDCRAGCVCGPNGFCG

SEQ ID NO: 341 - protein, 33 aa, Artificial Sequence
  Synthetic Integrin Binding Knottin Peptide 2.5D
GCPQGRGDWAPTSCKQDSDCRAGCVCGPNGFCG

SEQ ID NO: 342 - protein, 33 aa, Artificial Sequence; identical to SEQ ID NO: 335
  Synthetic Integrin Binding Knottin Peptide 2.5F

SEQ ID NO: 343 - protein, 33 aa, Artificial Sequence
  Synthetic Integrin Binding Knottin Peptide 2.5D K15S Mutant
GCPQGRGDWAPTSCSQDSDCLAGCVCGPNGFCG

SEQ ID NO: 344 - protein, 33 aa, Artificial Sequence
  Synthetic Integrin Binding Knottin Peptide 2.5F K15S Mutant
GCPRPRGDNPPLTCSQDSDCLAGCVCGPNGFCG

SEQ ID NO: 345 - protein, 33 aa, Artificial Sequence
  Synthetic Integrin Binding Knottin Peptide 2.5H
GCPQGRGDWAPEWCKQDSDCPAGCVCGPNGFCG

SEQ ID NO: 346 - protein, 33 aa, Artificial Sequence
  Synthetic Integrin Binding Knottin Peptide 2.5J
GCPRGRGDWSPPACKQDSDCQAGCVCGPNGFCG

SEQ ID NO: 347 - protein, 38 aa, Artificial Sequence
  Synthetic Integrin Binding Knottin Peptide 3A
GCVRLHESCLGQQVPCCDPAATCYCVVRGDWRKRCYCR

SEQ ID NO: 348 - protein, 38 aa, Artificial Sequence
  Synthetic Integrin Binding Knottin Peptide 3B
GCVRLHESCLGQQVPCCDPAATCYCEERGDMLEKCYCR

SEQ ID NO: 349 - protein, 38 aa, Artificial Sequence
  Synthetic Integrin Binding Knottin Peptide 3C
GCVRLHESCLGQQVPCCDPAATCYCETRGDGKEKCYCR

SEQ ID NO: 350 - protein, 38 aa, Artificial Sequence
  Synthetic Integrin Binding Knottin Peptide 3D
GCVRLHESCLGQQVPCCDPAATCYCQWRGDGDVKCYCR

SEQ ID NO: 351 - protein, 38 aa, Artificial Sequence
  Synthetic Integrin Binding Knottin Peptide 3E
GCVRLHESCLGQQVPCCDPAATCYCSRRGDMRERCYCR

SEQ ID NO: 352 - protein, 38 aa, Artificial Sequence
  Synthetic Integrin Binding Knottin Peptide 3F
GCVRLHESCLGQQVPCCDPAATCYCQYRGDGMKHCYCR

SEQ ID NO: 353 - protein, 38 aa, Artificial Sequence
  Synthetic Integrin Binding Knottin Peptide 3G
GCVRLHESCLGQQVPCCDPAATCYCTGRGDTKVLCYCR

SEQ ID NO: 354 - protein, 38 aa, Artificial Sequence
  Synthetic Integrin Binding Knottin Peptide 3H
GCVRLHESCLGQQVPCCDPAATCYCVERGDMKRRCYCR

SEQ ID NO: 355 - protein, 38 aa, Artificial Sequence
  Synthetic Integrin Binding Knottin Peptide 3I
GCVRLHESCLGQQVPCCDPAATCYCTGRGDVRMNCYCR

SEQ ID NO: 356 - protein, 38 aa, Artificial Sequence
  Synthetic Integrin Binding Knottin Peptide 3J
GCVRLHESCLGQQVPCCDPAATCYCVERGDGMSKCYCR

SEQ ID NO: 357 - protein, 38 aa, Artificial Sequence
  Synthetic Integrin Binding Knottin Peptide 4A
GCVRLHESCLGQQVPCCDPAATCYCRGRGDMRRECYCR

SEQ ID NO: 358 - protein, 38 aa, Artificial Sequence
  Synthetic Integrin Binding Knottin Peptide 4B
GCVRLHESCLGQQVPCCDPAATCYCEGRGDVKVNCYCR

SEQ ID NO: 359 - protein, 38 aa, Artificial Sequence
  Synthetic Integrin Binding Knottin Peptide 4C
GCVRLHESCLGQQVPCCDPAATCYCVGRGDEKMSCYCR

SEQ ID NO: 360 - protein, 38 aa, Artificial Sequence
  Synthetic Integrin Binding Knottin Peptide 4D
GCVRLHESCLGQQVPCCDPAATCYCVSRGDMRKRCYCR

SEQ ID NO: 361 - protein, 38 aa, Artificial Sequence
  Synthetic Integrin Binding Knottin Peptide 4E
GCVRLHESCLGQQVPCCDPAATCYCERRGDSVKKCYCR

SEQ ID NO: 362 - protein, 38 aa, Artificial Sequence
  Synthetic Integrin Binding Knottin Peptide 4F
GCVRLHESCLGQQVPCCDPAATCYCEGRGDTRRRCYCR

SEQ ID NO: 363 - protein, 38 aa, Artificial Sequence
  Synthetic Integrin Binding Knottin Peptide 4G
GCVRLHESCLGQQVPCCDPAATCYCEGRGDVVRRCYCR

SEQ ID NO: 364 - protein, 38 aa, Artificial Sequence
  Synthetic Integrin Binding Knottin Peptide 4H
GCVRLHESCLGQQVPCCDPAATCYCKGRGDNKRKCYCR

SEQ ID NO: 365 - protein, 38 aa, Artificial Sequence
  Synthetic Integrin Binding Knottin Peptide 4I
  misc_feature (21)..(21): Xaa can be any naturally occurring amino acid
GCVRLHESCLGQQVPCCDPAXTCYCKGRGDVRRVCYCR

SEQ ID NO: 366 - protein, 38 aa, Artificial Sequence
  Synthetic Integrin Binding Knottin Peptide 4J
GCVRLHESCLGQQVPCCDPAATCYCVGRGDNKVKCYCR

SEQ ID NO: 367 - protein, 38 aa, Artificial Sequence
  Synthetic Integrin Binding Knottin Peptide 5A
GCVRLHESCLGQQVPCCDPAATCYCVGRGDNRLKCYCR

SEQ ID NO: 368 - protein, 38 aa, Artificial Sequence
  Synthetic Integrin Binding Knottin Peptide 5B
GCVRLHESCLGQQVPCCDPAATCYCVERGDGMKKCYCR

SEQ ID NO: 369 - protein, 38 aa, Artificial Sequence
  Synthetic Integrin Binding Knottin Peptide 5C
GCVRLHESCLGQQVPCCDPAATCYCEGRGDMRRRCYCR

SEQ ID NO: 370 - protein, 38 aa, Artificial Sequence
  Synthetic Integrin Binding Knottin Peptide 5D
GCVRLHESCLGQQVPCCDPAATCYCQGRGDGDVKCYCR

SEQ ID NO: 371 - protein, 38 aa, Artificial Sequence
  Synthetic Integrin Binding Knottin Peptide 5E
GCVRLHESCLGQQVPCCDPAATCYCSGRGDNDLVCYCR

SEQ ID NO: 372 - protein, 38 aa, Artificial Sequence
  Synthetic Integrin Binding Knottin Peptide 5F
GCVRLHESCLGQQVPCCDPAATCYCVERGDGMIRCYCR

SEQ ID NO: 373 - protein, 38 aa, Artificial Sequence; identical to SEQ ID NO: 371
  Synthetic Integrin Binding Knottin Peptide 5G

SEQ ID NO: 374 - protein, 38 aa, Artificial Sequence
  Synthetic Integrin Binding Knottin Peptide 5H
GCVRLHESCLGQQVPCCDPAATCYCEGRGDMKMKCYCR

SEQ ID NO: 375 - protein, 38 aa, Artificial Sequence
  Synthetic Integrin Binding Knottin Peptide 5I
GCVRLHESCLGQQVPCCDPAATCYCIGRGDVRRRCYCR

SEQ ID NO: 376 - protein, 38 aa, Artificial Sequence
  Synthetic Integrin Binding Knottin Peptide 5J
GCVRLHESCLGQQVPCCDPAATCYCEERGDGRKKCYCR

SEQ ID NO: 377 - protein, 38 aa, Artificial Sequence
  Synthetic Integrin Binding Knottin Peptide 6B
GCVRLHESCLGQQVPCCDPAATCYCEGRGDRDMKCYCR

SEQ ID NO: 378 - protein, 38 aa, Artificial Sequence
  Synthetic Integrin Binding Knottin Peptide 6C
GCVRLHESCLGQQVPCCDPAATCYCTGRGDEKLRCYCR

SEQ ID NO: 379 - protein, 38 aa, Artificial Sequence
  Synthetic Integrin Binding Knottin Peptide 6E
GCVRLHESCLGQQVPCCDPAATCYCVERGDGNRRCYCR

SEQ ID NO: 380 - protein, 38 aa, Artificial Sequence
  Synthetic Integrin Binding Knottin Peptide 6F
GCVRLHESCLGQQVPCCDPAATCYCESRGDVVRKCYCR

SEQ ID NO: 381 - protein, 38 aa, Artificial Sequence
  Synthetic Integrin Binding Knottin Peptide 7C
GCVRLHESCLGQQVPCCDPAATCYCYGRGDNDLRCYCR

SEQ ID NO: 382 - protein, 33 aa, Artificial Sequence; identical to SEQ ID NO: 344
  Synthetic Integrin Binding Polypeptide NOD201 - 2.5F

SEQ ID NO: 383 - protein, 33 aa, Artificial Sequence; identical to SEQ ID NO: 335
  Synthetic Integrin Binding Polypeptide NOD201modK - 2.5FmodK

SEQ ID NO: 384 - protein, 38 aa, Artificial Sequence
  Synthetic Integrin Binding Polypeptide NOD203 - 2.5F with linker
GCPRPRGDNPPLTCSQDSDCLAGCVCGPNGFCGGGGGS

SEQ ID NO: 385 - protein, 38 aa, Artificial Sequence
  Synthetic Integrin Binding Polypeptide NOD203modK - 2.5FmodK with linker
GCPRPRGDNPPLTCKQDSDCLAGCVCGPNGFCGGGGGS

SEQ ID NO: 386 - protein, 48 aa, Artificial Sequence
  Synthetic Integrin Binding Polypeptide NOD204 - 2.5F with linker
GCPRPRGDNPPLTCSQDSDCLAGCVCGPNGFCGGGGGSGGGGSGGGGS

SEQ ID NO: 387 - protein, 48 aa, Artificial Sequence
  Synthetic Integrin Binding Polypeptide NOD204modK - 2.5FmodK with linker
GCPRPRGDNPPLTCKQDSDCLAGCVCGPNGFCGGGGGSGGGGSGGGGS

SEQ ID NO: 388 - protein, 5 aa, Artificial Sequence
  Synthetic short linker
GGGGS

SEQ ID NO: 389 - protein, 15 aa, Artificial Sequence
  Synthetic long linker
GGGGSGGGGSGGGGS

SEQ ID NO: 390 - protein, 18 aa, Artificial Sequence
  Synthetic signal sequence
MTRLTVLALLAGLLASSR

SEQ ID NO: 391 - protein, 264 aa, Artificial Sequence
  Synthetic Fc fusion NOD201 (human Fc)
GCPRPRGDNPPLTCSQDSDCLAGCVCGPNGFCGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 392 - protein, 264 aa, Artificial Sequence
  Synthetic Fc fusion NOD201X (control sequence - NOD201 with scrambled seq, human Fc)
GCVTGRDGSPASSCSQDSDCLAGCVCGPNGFCGEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 393 - protein, 270 aa, Artificial Sequence
  Synthetic Fc fusion NOD201M (NOD201 with murine Fc domain)
GCPRPRGDNPPLTCSQDSDCLAGCVCGPNGFCGEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLG

SEQ ID NO: 394 - protein, 269 aa, Artificial Sequence
  Synthetic NOD203 complete with short linker
GCPRPRGDNPPLTCSQDSDCLAGCVCGPNGFCGGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 395 - protein, 279 aa, Artificial Sequence
  Synthetic NOD204 complete with long linker
GCPRPRGDNPPLTCSQDSDCLAGCVCGPNGFCGGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 396 - protein, 330 aa, Artificial Sequence [sequence omitted]

SEQ ID NO: 397 - protein, 326 aa, Artificial Sequence
  Synthetic IgG2
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 398 - protein, 377 aa, Artificial Sequence
  Synthetic IgG3
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 399 - protein, 327 aa, Artificial Sequence
  Synthetic IgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK